(19) United States
(12) Patent Application Publication
(10) Pub. No.: US 2006/0099578 A1
(43) Pub. Date: May 11, 2006

(54) Mitochondrial biology expression arrays

(76) Inventors: Douglas C Wallace, Irvine, CA (US); Shawn Levy, Brentwood, TN (US); Keith Kerstann, Atlanta, GA (US); Vincent Procaccio, Irvine, CA (US)

Correspondence Address: GREENLEE WINNER AND SULLIVAN P C, 4875 PEARL EAST CIRCLE, SUITE 200, BOULDER, CO, 80301, US

(21) Appl. No.: 10/488,619

(22) PCT Filed: Aug. 30, 2002
(86) PCT No.: PCT/US02/27886

§ 371 Date: Nov. 9, 2004

(30) Foreign Application Priority Data
Aug. 30, 2001 (US) 60316323
Aug. 31, 2001 (CA) 2356540

(51) Int. Cl. C12Q 1/68; C12M 1/34

(52) U.S. Cl. 435/6; 435/287.2

(57) ABSTRACT

This invention provides a library of genes involved in mitochondrial biology, arrays containing probes for genes involved in mitochondrial biology, methods for making such arrays, and methods of using such arrays. Genes and probe sequences involved in mitochondrial biology in humans and mice are provided. The arrays of this invention are useful for determining mitochondrial biology gene expression profiles. Mitochondrial biology gene expression profiles are useful for determining expression profiles diagnostic of physiological conditions; diagnosing physiological conditions; identifying biochemical pathways, genes, and mutations involved in physiological conditions; identify therapeutic agents useful for preventing and/or treating such physiological conditions; evaluating and/or monitoring the efficacy of such therapies, and creating and identifying animal models of human physiologic conditions. Arrays containing probes for all genes known to be involved in mitochondrial biology are provided, as well as arrays containing subsets of such probes.

### CROSS-REFERENCE TO RELATED APPLICATIONS

This application claims priority to U.S. Patent Application Ser. No. 60/316,323 filed Aug. 30, 2001, and to Canadian Patent Application Serial No. 2,356,540 filed Aug. 31, 2001, both of which is hereby incorporated in their entirety by reference to the extent not inconsistent with the disclosure herein.

### BACKGROUND OF THE INVENTION

Mitochondrial disorders are a complex and polygenic group of conditions with the patient's symptoms varying due to differences in energetic threshold effect of various tissues and the stochastic nature of mtDNA segregation. Consequently, most mitochondrial disorders are best classified by their genetic cause rather than a biochemical or phenotypic profile (Shoffner, J. M., and Wallace, D. C., (1995) “Oxidative phosphorylation diseases,” In The Metabolic and Molecular Basis of Inherited Disease, C. R. Scriver, A. L. Beaudet, W. S. Sly and D. Valle, eds. (New York: McGraw-Hill), pp.1535-1609; Wallace, D. C., (1999) “Mitochondrial diseases in man and mouse” Science 283:1482-1488). Many mitochondrial diseases result from mutations in nuclear genes and a subset of these are known to act by destabilizing the mitochondrial genome. (Graham, B. et al., “A mouse model for mitochondrial myopathy and cardiomyopathy resulting from a deficiency in the heart/skeletal muscle isoform of the adenine nucleotide translocator,” [1997] Nature Genetics 16:226-234; Shoffner, J. M., and Wallace, D. C., “Oxidative phosphorylation diseases. Disorders of two genomes,” [1990] Advances in Human Genetics 19:267-330; Zhu, Z. et al., “SURF1, encoding a factor involved in the biogenesis of cytochrome c oxidase, is mutated in Leigh's syndrome” [1998] Nature Genetics 20:33743).

The analysis of mitochondrial function in cultured cells using somatic cell genetics has been instrumental in the characterization of human mitochondrial disorders. Ethidium bromide and R-6G treatment have been used to create ρ0 and mitochondria-less cell lines to analyze the maternal inheritance and biochemical phenotypes of many human mtDNA mutations (Chomyn, A. et al., “In vitro genetic transfer of protein synthesis and respiration defects to mitochondrial DNA-less cells with myopathy-patient mitochondria,” [1991] Molecular and Cellular Biology 11:2236-2244; Jun, A. S. et al., “Use of transrnitochondrial cybrids to assign a complex I defect to the mitochondrial DNA-encoded NADH dehydrogenase subunit 6 gene mutation at nucleotide pair 14459 that causes Leber hereditary optic neuropathy and dystonia,” [1996] Molecular and Cellular Biology 16:771-777; King, M. P. et al., “Defects in mitochondrial protein synthesis and respiratory chain activity segregate with the tRNA Leu(UUR) mutation associated with mitochondrial myopathy, encephalopathy, lactic acidosis, and stroke-like episodes,” [1992] Molecular and Cellular Biology 12:480490; Trounce, I. et al., “Cytoplasmic transfer of the mtDNA nt 8993 TG [ATP6] point mutation associated with Leigh's syndrome into mtDNA-less cells demonstrates cosegregation with a decrease in state III respiration and ADP/O ratio,” [1994] Proc. Natl. Acad. Sci. U.S.A. 91:8334-8338). The creation of cybrid cell lines with identical nuclear backgrounds but different mtDNA genotypes allows the comparison of one mtDNA mutant to another without the potential interference of nuclear genome polymorphisms. These cybrid lines have generally been analyzed using biochemical techniques such as assaying cellular respiration or respiratory complex specific activities by enzymology. Some gene expression studies have been performed, but they have generally been done on single or small groups of genes (Heddi, A. et al., “Mitochondrial DNA expression in mitochondrial myopathies and coordinated expression of nuclear genes involved in ATP,” [1993] J. Biological Chemistry 268:12156-12163; Heddi, A. et al., “Coordinate induction of energy gene expression in tissues of mitochondrial disease patients” [1999] J Biol Chem 274:22968-76).

Gene expression has been extensively studied. Although the regulation of mRNA abundance by changes in transcription or RNA degradation is by no means the only mechanism that regulates protein levels in a cell, virtually all differences in cell type or state can be correlated to changes in the mRNA abundance of several genes (Alizadeh, A. A. et al., “Distinct types of diffuse large B-cell lymphoma identified by gene expression profiling,” [2000] Nature 403:503-11; DeRisi, J. L. et al., “Exploring the metabolic and genetic control of gene expression on a genomic scale,” [1997] Science 278:680-686; Schena, M. et al., “Quantitative monitoring of gene expression patterns with a complementary DNA microarray” [1995] Science 270:467-70; Schena, M. et al., “Parallel human genome analysis: microarray-based expression monitoring of 1000 genes” [1996] Proc Natl Acad Sci USA 93:10614-9; Wallace D. C., grant abstract #2R01N502328-18; Kerstann, K. W. [2000] American Society for Human Genetics Abstract #1484; Kokoszka, J. E. [2000] American Society of Human Genetics Abstract #1618; Levy, S. E. [2001] American Society of Human Genetics Abstract #1501; Levy, S. E. [2000] “Genetic Alteration of the Mouse Mitochondrial Genome and Effects on Gene Expression,” Ph.D. Thesis, Emory University; Coskun, P. E. [2000] American Society of Human Genetics Abstract #1616; Sligh, J. E. [2000] American Society for Human Genetics Abstract #53; Murdock, D. G. [2000] American Society for Human Genetics Abstract #55; Levy S. E. [2000] Keystone Symposia Abstract 119; Wallace, D. C., Ellison Medical Foundation, Senior Scholar Award in Aging).

DNA microarray analysis has been used to study diffuse large B-cell lymphoma (DLBCL) where microarrays were used to expand the diagnosis of DLBCL (Alizadeh, A. A. et al., “Distinct types of diffuse large B-cell lymphoma identified by gene expression profiling,” [2000] Nature 403:503-11). While standard histological and morphological techniques had defined subsets of DLBCL, array analysis revealed two clinically distinct classes. These two newly discovered classes were indistinguishable by standard pathology, but expression analysis showed a differential expression of hundreds of genes. Correlation of these molecular differences with differences in the progression of the disease and clinical outcome has revealed that these two classes of DLBCL could be considered separate diseases (Alizadeh, A. A. et al., “Distinct types of diffuse large B-cell lymphoma identified by gene expression profiling,” [2000] Nature 403:503-11).

Mitochondrial DNA sequences have been associated with pathologies as described in U.S. Pat. Nos. 5,670,320, 5,296,349, 5,185,244, and 5,494,794. Publications on the subject of mitochondrial biology include: Scheffler I. E. (1999) Mitochondria, Wiley-Liss, New York; Lestienne, P., Ed. (1999) Mitochondrial Diseases: Models and Methods, Springer-Verlag, Berlin; Methods in Enzymology (2000) 322:Section V Mitochondria and Apoptosis, Academic Press, California; Mitochondria and Cell Death (1999) Princeton University Press, New Jersey; Papa S, Ferruciio G, and Tager J Eds. (1999) Frontiers of Cellular Biometics: Molecular Biology, Biochemistry, and Physiopathology, Kluwer Academic/Plenum Publishers, New York; Lemasters J and Nieminen A (2001) Mitochondria in Pathogenesis, Kluwer Academic/Plenum Publishers, New York; MITOMAP, http://www.gen.emory.edu/cgi-gin/MITOMAP; Wallace D. C. (2001) “A mitochondrial paradigm for degenerative diseases and aging,” Novartis Foundation Symposium 235:247-266; Wallace D C “Mitochondrial DNA in Aging and Disease” (August 1997) Scientific American 277:40-47; Wallace D. C. et al., “Mitochondrial biology, degenerative diseases and aging,” (1998) BioFactors 7:187-190; Heddi, A. et al., “Coordinate Induction of Energy Gene Expression in Tissues of Mitochondrial Disease Patients” (1999) JBC 274:22968-22976; Wallace, D. C. “Mitochondrial Diseases in Man and Mouse,” (1999) Science 283:1482-1488; Saraste, M. “Oxidative Phosphorylation at the fin de siecle” (1999) Science 283:1488-1493; Kokoszka et. al., “Increased mitochondrial oxidative stress in the Sod2 (+/−) mouse results in the age-related decline of mitochondrial function culminating in increased apoptosis,” (2001) PNAS 98:2278-2283; Wallace, D. C. (2001) Mental Retardation and Developmental Disabilities 7:158-166; Wallace D. C. (2001) Am. J Med. Gen. 106:71-93; and Wallace, D. C. (2001) EuroMit 5 Abstract.

The analysis of mitochondrial disorders has traditionally consisted of molecular and biochemical descriptions of the defect (Shoffner, J. M., and Wallace, D. C., (1995) “Oxidative phosphorylation diseases,” In The Metabolic and Molecular Basis of Inherited Disease, C. R. Scriver, A. L. Beaudet, W. S. Sly and D. Valle, eds. (New York: McGraw-Hill), pp. 1535-1609). Only a limited number of analyses of changes in oxidative phosphorylation (OXPHOS) genes expression have been performed in humans harboring mtDNA mutations (Heddi, A. et al., “Coordinate Induction of Energy Gene Expression in Tissues of Mitochondrial Disease Patients” (1999) JBC 274:22968-22976). The advent of mouse models for mitochondrial disease created by the inactivation of nuclear-encoded OXPHOS subunits has provided experimental material to study tissue-specific expression changes. (Murdock, D. G. et al., “Up-regulation of nuclear and mitochondrial genes in the skeletal muscle of mice lacking the heart/muscle isoform of the adenine nucleotide translocator,” [1999] J. Biol. Chem. 274:14429-33.)

Nucleic acid arrays have been described, e.g., in U.S. Pat. No. 5,837,832, U.S. Pat. No. 5,807,522, U.S. Pat. No. 6,007,987, U.S. Pat. No. 6,110,426, WO 99/05324, 99/05591, WO 00/58516, WO 95/11995, WO 95/35505A1, WO 99/42813, JP10503841T2, GR3030430T3, ES2134481T3, EP804731B1, DE69509925C0, CA2192095AA, AU2862995A1, AU709276B2, AT180570, EP 1066506, and AU 2780499. Such arrays can be incorporated into computerized methods for analyzing hybridization results when the arrays are contacted with prepared sample nucleotides, e.g., as described in PCT Publication WO 99/05574, and U.S. Pat. Nos. 5,754,524; 6,228,575; 5,593,839; and 5,856,101. Methods for screening for disease markers are also known to the art, e.g., as described in U.S. Pat. Nos. 6,228,586; 6,160,104; 6,083,698; 6,268,398; 6,228,578; and 6,265,174.

All references cited herein are incorporated by reference in their entirety to the extent that they are not inconsistent with the disclosure herein. Citation of the above documents is not an admission that any of them are pertinent prior art.

### SUMMARY OF THE INVENTION

This invention provides a library of genes involved in mitochondrial biology, arrays containing probes for genes involved in mitochondrial biology, methods for making such arrays, and methods of using such arrays. Genes and probe sequences involved in mitochondrial biology in humans and mice are provided. The arrays of this invention are useful for determining mitochondrial biology gene expression profiles. Mitochondrial biology gene expression profiles are useful for determining expression profiles diagnostic of energy metabolism-related physiological conditions; diagnosing such physiological conditions; identifying biochemical pathways, genes, and mutations involved in such physiological conditions; identifying therapeutic agents useful for preventing and/or treating such physiological conditions; evaluating and/or monitoring the efficacy of such therapies; and creating and identifying animal models of human energy metabolism-related physiological conditions. Arrays containing probes for all genes known to be involved in mitochondrial biology are provided, as well as arrays containing subsets of such probes. The mitochondrial biology expression arrays of this invention contain probes of genes not previously recognized to participate in mitochondrial biology.

### BRIEF DESCRIPTION OF THE DRAWINGS

FIG. 1 is a diagram of the mammalian mitochondrion showing mitochonrial energetics, and the relationship between energy production, reactive oxygen species (ROS) generation, and regulation of apoptosis.

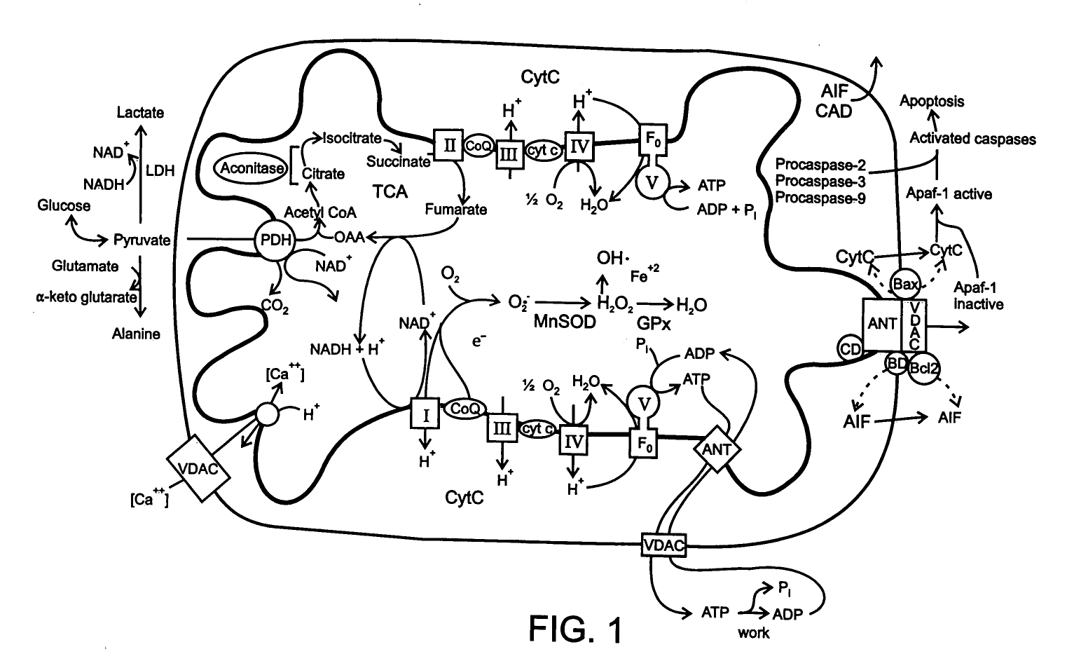

FIG. 2 is a depiction of a hybridized mouse array of this invention. The picture of the hybridized array shows the image generated when the two channels representing the control or reference and experimental targets are overlaid. When viewed in color, the spots appear various shades of red, green and yellow. Red spots indicate a predominance of hybridization to control cDNAs, while green spots indicate the predominance of hybridization to the experimental target sample. Yellow spots indicate an equal hybridization of both samples. Spots that are yellow-green or orange when the array is shown in color are depicted as half yellow and green, or half red and yellow, respectively.

FIG. 3 shows the ρ0 LMEB4 cell line gene expression scatter plot. The scatter plot shows the distribution of gene expression ratio for the ρ0 LMEB4 sample. The diagonal dotted line indicates a ratio of 1 between the two samples. Any spot above the dotted line is up-regulated or more abundant in the ρ0 LMEB4 experimental sample compared to the LM(TK)-control. Any spot below the dotted line is down-regulated or less abundant in the experimental sample compared to the control.

FIG. 4 shows NZB heart gene expression scatter plot. The scatter plot shows the distribution of gene expression ratio for the NZB heart tissue sample. The diagonal dotted line indicates a ratio of 1 between the two samples. Any spot above the dotted line is up-regulated or more abundant in the NZB-mtDNA heart experimental sample compared to the “common” mtDNA control heart. Any spot below the dotted line is down-regulated or less abundant in the experimental sample compared to the control.

### DETAILED DESCRIPTION OF THE INVENTION

An approach to examining the complex interaction between nuclear and cytoplasmic mitochondrial genes is through the use of arrays such as DNA arrays. DNA microarrays provide a means to profile the expression patterns of up to thousands of genes simultaneously, and knowing where and when a gene is expressed often provides insight into its biological function. The pattern of gene expression in a particular tissue or cell type can also provide detailed information about its state or condition.

Currently, DNA microarrays are the most efficient method to monitor correlative changes in gene expression and to investigate complex traits on a molecular level. Expression profiles assembled from multiple interrelated experiments are used to determine hierarchical connections between gene expression patterns underlying complex biological traits. These patterns are used to further define the molecular basis of complex disorders.

The mitochondrion is assembled from approximately 1000 protein-coding nuclear DNA (nDNA) and mitochondrial DNA (mtDNA) genes. Thirteen protein-coding mitochondrial genes are known, as shown in Table 1. The codon usage table of the mtDNA is known. It differs slightly from the universal code. For example, UGA codes for tryptophan instead of termination, AUA codes for methionine instead of isoleucine, and AGA and AGG are terminators instead of coding for arginine.

TABLE 1

| Gene | Map Locus^{a} | Abbreviation | Location^{b} |
|---|---|---|---|
| NADH dehydrogenase 1 | MTND1 | ND1 | 3307-4262 |
| NADH dehydrogenase 2 | MTND2 | ND2 | 4470-5511 |
| NADH dehydrogenase 3 | MTND3 | ND3 | 10059-10404 |
| NADH dehydrogenase 4L | MTND4L | ND4L | 10470-10766 |
| NADH dehydrogenase 4 | MTND4 | ND4 | 10760-12137 |
| NADH dehydrogenase 5 | MTND5 | ND5 | 12337-14148 |
| NADH dehydrogenase 6 | MTND6 | ND6 | 14149-14673 |
| Cytochrome b | MTCYB | Cytb | 14747-15887 |
| Cytochrome c oxidase I | MTCO1 | COI | 5904-7445 |
| Cytochrome c oxidase II | MTCO2 | COII | 7586-8269 |
| Cytochrome c oxidase III | MTCO3 | COIII | 9207-9990 |
| ATP synthase 6 | MTATP6 | ATP6 | 8527-9207 |
| ATP synthase 8 | MTATP8 | ATP8 | 8366-8572 |

^{a,b}As defined on MitoMap, http://www.gen.emory.edu/cgi-bin/MITOMAP, which is numbered relative to the Cambridge Sequence (Genbank accession no. J01415 and Andrews et al. (1999), A Reanalysis and Revision of the Cambridge Reference Sequence for Human Mitochondrial DNA, Nature Genetics 23: 147.

As used herein “gene” refers to a unigene cluster, an expressed sequence, or a sequence that is transcribed and translated into a protein. Another word used in the art for “gene” is “locus.” The National Institutes of Health (NIH) have instituted the term “gene cluster” to refer to non-redundant sets of gene clusters. A stretch of DNA may be transcribed into several splice variants that share sequences, and these would be designated as belonging to one unigene cluster. As used herein “splice variant” refers to one version of several transcripts that are transcribed from one gene. As used herein “housekeeping gene” refers to a gene that is expressed at a similar level in almost all cell types.

As used herein “genes involved in mitochondrial biology” refers to mitochondrial genes and nuclear genes involved in cellular structures and functions such as intermediary metabolism, OXPHOS, mitochondrial transport, cellular bioenergetics, cellular biogenesis, cell cycle control, DNA replication, energy, metabolism, heat shock, stress, cellular matrix, cellular structural proteins, protein synthesis and translational control, signal transduction, transcription and transcriptional regulation, chromatin structure, reactive bxygen species (ROS) biology, and apoptosis.

“mtDNA” means mitochondrial DNA. “nDNA” means nuclear DNA.

As used herein “mitochondrial biology expression profile” refers to the expression patterns of genes involved in mitochondrial biology, such as is detected by probes derived from those genes, in a sample. The profile can be said to be of the sample or of the source from which the sample is derived. A profile may be measured independently, but a profile may also measured relative to a standard or control or other sample. A complete mitochondrial biology expression profile includes data on all genes known to be involved in mitochondrial biology for the species from which the sample is derived. The mitochondrial biology expression profile for a selected physiological condition is at least the expression pattern of genes determined to have altered expression diagnostic of that physiological condition, but the expression pattern of additional genes involved in mitochondrial biology may also be included.

As used herein “array” refers to an ordered set of isolated nucleic acid molecules or spots consisting of pluralities of substantially identical isolated nucleic acid molecules. Preferably the molecules are attached to a substrate. The spots or molecules are ordered so that the location of each (on the substrate) is known and the identity of each is known. Arrays on a micro scale can be called microarrays. Microarrays on solid substrates, such as glass or other ceramic slides, can be called gene chips or chips.

As used herein, an “isolated nucleic acid” is a nucleic acid outside of the context in which it is found in nature. An isolated nucleic acid is a nucleic acid the structure of which is not identical to that of any naturally occurring nucleic acid molecule. The term covers, for example: (a) a DNA which has the sequence of part of a naturally-occurring genomic DNA molecule but is not flanked by both of the coding or noncoding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally-occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein, or a modified gene having a sequence not found in nature.

As used herein “probe” refers to an isolated nucleic acid that is suitable for hybridizing to other nucleic acids when placed on a solid substrate. Probes for arrays can be as short as 20-30 nucleotides and up to as long as several thousand nucleotides. Probes can be single-stranded or double stranded. A probe usually comprises at least a partially known sequence that is used to investigate or interrogate the presence, absence, and/or amount of a complementing sequence. On the arrays of this invention, a probe is of such a sequence and the hybridization conditions of such stringency that each probe hybridizes substantially to only one type of nucleic acid per target sample.

As used herein, “target” or “target sample” refers to the collection of nucleic acids, e.g., reverse transcribed and labeled cDNA used as a prepared sample for array analysis. The target is interrogated by the probes of the array. A “target” or “target sample” may be a mixture of several prepared samples that are combined. For example, an experimental target sample may be combined with a differently labeled control sample and hybridized to an array, the combined samples being referred to as the “target” interrogated by the probes of the array. As used herein, “interrogated” means tested. Probes, targets, and hybridization conditions are chosen such that the probes are capable of interrogating the target, i.e., of hybridizing to complementary sequences in the target sample.

As used herein “physiological condition” refers to a healthy or unhealthy physiological state. As used herein “optimize an array for diagnosis” refers to selecting probes for an array such that only probes from genes necessary for diagnosis of one or more physiological conditions are included.

As used herein “printing” refers to the process of applying probes to a solid substrate, e.g., or applying arrays of probes to a solid substrate to make a gene chip. As used herein “glass slide” refers to a small piece of glass of the same dimensions as a standard microscope slide. As used herein, “prepared substrate” refers to a substrate that is prepared with a substance capable of serving as an attachment medium for attaching the probes to the substrate, such as poly Lysine.

As used herein “selective hybridization” refers to hybridization at moderate to high stringency such that only sequences of an appropriate homology can remain bound. Selective hybridization is hybridization performed at stringency conditions such that probes only hybridize to target sample nucleic acids that they are intended to hybridize with. Depending on the sequences of the probes and the target, the hybridization conditions are chosen to be appropriately selective. For example, if human sequences are used as probes for interrogating a human sample, selective hybridization could be at high stringency because, allowing for neutral polymorphism in humans, the sequences would be about 99-100% identical. When applying a chimpanzee target prepared sample to an array containing human sequence probes, selective hybridization would be at a lower stringency. Since hybridizing a target to an array is performed at one chosen hybridization stringency, probes are chosen so that they can undergo selective hybridization with the appropriate target molecules at the same hybridization stringency. As used herein “homology” refers to nucleotide sequence identity to a sequence, a molecule, or its complement.

As used herein “mouse sample” refers to a sample derived from a mouse or a cell line derived from a mouse. Similarly, as used herein, “human sample” refers to a sample derived from a human or a cell line derived from a human. Samples preferably contain total RNA or messenger RNA (mRNA). As used herein “total RNA” refers to a combination of several types of RNA, including MRNA, from a cell or a group of cell. As used herein, “MRNA” refers to messenger RNA or RNA that has a 3′ poly A tail. As used herein, a “prepared sample” or a “target” refers to a sample that has been labeled in preparation for array hybridization. A “prepared sample” or “target” is reverse transcribed and fluorescently labeled. As used herein “standard” refers to a sample or a dataset that is commonly used for comparison to unknown samples so that the unknown samples or datasets can be standardized for comparison to each other. As used herein, “control sample” and “reference sample” refer to samples that are used for comparison against an experimental sample.

As used herein, “clone” refers to an isolated nucleic acid molecule that may be stored in an organism such as E. coli. A clone is usually made of a vector and an insert. The insert usually contains a sequence of interest.

For mitochondrial diseases, the accuracy of current biochemical and phenotypic techniques has proven quite limited in distinguishing and diagnosing the various disorders. Recent technical and analytical advancements make it practical to analyze and quantitate the expression patterns of thousands of genes at once using arrays such as DNA microarrays. This invention applies these array techniques to the study of mitochondrial gene expression, in the design of specialized microarrays containing genes involved in mitochondrial biology. The arrays of this invention contain probes for genes not previously recognized to participate in mitochondrial biology.

Genes, or expressed sequences, involved in mitochondrial biology are involved in cellular structures and functions such as intermediary metabolism, OXPHOS, transport, cellular bioenergetics, cellular biogenesis, cell cycle control, DNA replication, energy, metabolism, heat shock, stress, cellular matrix, cellular structural proteins, protein synthesis and translational control, signal transduction, transcription and transcriptional regulation, chromatin structure, reactive oxygen species (ROS) biology and apoptosis. Alterations in mitochondrial functions are associated with a variety of physiological conditions including degenerative diseases. These functions are involved in many degenerative diseases. This invention provides a compilation of sequences involved in human and mouse mitochondrial biology.

The genes in the arrays of this invention were identified by a variety of techniques including searching databanks for sequences related to genes involved in processes similar to mitochondrial biology such as homologues of prokaryotic genes, and screening mitochondrial mutant cell lines and animal lines for genes having altered expression patterns. When a relevant gene was identified for one species, such as the mouse, the homologue for a second species, such as human, if known, was then included on the list of genes involved in mitochondrial biology for the second species. Mitochondrial mutant cell lines are cell lines that have at least one mutation in a gene involved in mitochondrial biology.

The microarrays or gene chips of this invention comprise probes placed in known positions on a solid substrate. A useful solid substrate is a specialized glass microscope slide. The arrays of this invention include arrays containing probes that detect some or all expressed sequences involved in mitochondrial biology in a selected species.

Arrays of this invention may contain control probes as well as probes for genes involved in mitochondrial biology. Controls that can be included on the arrays of this invention include hybridization controls and scanning controls. The controls can be positive or negative controls. One type of hybridization control is spotting the same probe for a gene involved in mitochondrial biology several times on one chip, each spot having different amounts of probe. This allows for the amount of probe of a given sequence to be optimized. Spotting too little probe may lead to a maximum hybridization signal resulting in a loss of data. Dimethyl sulfoxide (DMSO) can be used as a negative hybridization and scanning control. A spot of DMSO should give no signal. If there is any signal at a DMSO spot, the problem could be at hybridization or scanning steps. Plant sequences having sufficiently low homology with human and mouse sequences can be utilized as negative hybridization and scanning controls. Plant sequences should not give any signal. A signal at a plant spot could indicate a problem with hybridization, i.e. too low a hybridization stringency was used, or with scanning, i.e., the chip was inserted into the scanner at the incorrect orientation. Poly A can be used as a positive hybridization specificity/non specificity control. A poly A spot should always give intense hybridization. No signal at a poly A spot could be the result of use of too high a hybridization stringency. Cy3 or Cy5 incorporated into a PCR product can be a positive scanning control. A spot on an array of a PCR product, or any other nucleic acid, that includes fluorescent label, should always give a signal, and if this sequence has no homology with any other sequence in the target, there should only be a signal of the label included in the nucleic acid. Control probes and probes for genes involved in mitochondrial biology can be duplicated, triplicated, etc. on the chip as printing controls. Controls for arrays can be purchased from Stratagene (SpotReport™, La Jolla, Calif., USA).

Standard targets and reference targets are also useful with the arrays of this invention, as is known in the art. When a prepared sample target to be interrogated is applied to an array of this invention, the results of the test are measured, i.e. by scanning, and recorded. These results can be compared directly to other test results using a similar array. However, it is much more accurate to include a differently labeled standard target in the hybridization mix with the prepared sample target. The results of the experimental sample target are then standardized, so that they can be compared accurately to the results of hybridizations of other sample targets. If ten different prepared sample targets are hybridized to arrays of this invention, simultaneously with the same prepared standard target, then the results of the ten sample targets can be accurately compared to each other. A prepared reference or control target for comparison can also be particularly pertinent to the experiment being performed. A prepared reference target could be a target sample derived from the same cell type from an animal of the same sex, age, and nuclear background as the experimental target sample, except for one difference, such as a different phenotype or treatment. Comparing the results of the experimental target with the results of an appropriate reference target yields a profile associated with the one difference being tested. When the hybridization results of a first sample are compared to the hybridization results of a second sample, the comparison can occur while the hybridization results of the first sample are being measured and recorded, or afterwards, by comparing the measured and recorded hybridization results of the two samples.

Probes on an array may be as short as about 20-30 nucleotides long or as long as the entire gene or clone from which they are derived, which may be up to several kilobases. A probe sequence may be identical (have 100% homology) to the portion of the gene it hybridizes to or it may be a mutated sequence. Mutated probes have less than 100% homology, such as about 98% homology, about 95% homology, about 90% homology, about 80% homology, or about 75% homology, or less, with the portions of the genes to which they hybridize. Arrays are designed such that all probes on an array can hybridize to their corresponding genes at about the same hybridization stringency. Probes for arrays used for interrogating samples usually do not contain sequences such as repetitive sequences that would hybridize substantially with nucleic acids derived from more than one gene, i.e., transcripts or cDNAs. Probes for arrays should be unique at the hybridization stringencies used. Statistically, to be unique in the total human genome, probes should be at least about fifteen nucleotides long. A unique probe is only able to hybridize with one type of nucleic acid per target. A probe is not unique if at the hybridization stringency used, it hybridizes with nucleic acids derived from two different genes, i.e. related genes. The homology of the sequence of the probe to the gene and the hybridization stringency used help determine whether a probe is unique when testing a selected sample. Probes also may not hybridize with different nucleic acids derived from the same gene, i.e., splice variants. The location in the gene of the sequence used for the probe also helps determines whether a probe is unique when testing a selected sample. If the splice variants of a gene are known, ideally several different probes sequences are chosen from that gene for an array, such that each probe can only hybridize to nucleic acid derived from one of the splice variants. References for sequences of probes useful for arrays of this invention are compiled in Tables 3-5 and in the sequence listings. Other equivalent probes derived from the gene sequences from which the Tables 3-5 probes are derived, are also useful for the arrays of this invention. Arrays of this invention are used at hybridization conditions allowing for selective hybridization. At conditions of selective hybridization, probes hybridize with nucleic acid from only one gene. When an array is simultaneously hybridized with two targets or two prepared samples, each probe may hybridize with a nucleic acid in each prepared sample or target. When these two nucleic acids are from the same unigene cluster, the probe is said to hybridize with one gene, despite the fact that these nucleic acids may contain different labels.

Sequences of genes involved in mitochondrial biology from other species can be used to make probes that are useful in the arrays of this invention as long as they hybridize at about the same hybridization stringency as other probes on an array. Sequences that are only able to hybridize at a substantially lower stringency, such as plant sequences, are useful as negative controls.

The arrays of this invention can be utilized to determine profiles for related species by modifying the hybridization stringency appropriately. Sequence homology between organisms is known in the art. For example, human and chimpanzee sequences are about 98% identical. Consequently, human arrays are useful for profiling chimpanzees, with an appropriate lowering of the hybridization stringency. Hybridization stringency can be lowered by modifying hybridization components such as salt concentrations and hybridization and/or wash temperatures, as is known in the art.

The sequences useful for the arrays of this invention are useful for designing arrays for other species as well. To create an array for a new organism, the known sequences from the new organism, including expressed sequence tags (ESTs), are compared, by methods known to the art, with the sequences known to already be useful for other mitochondrial biology arrays. Sequence comparisons may be performed at the nucleic acid or polypeptide level. Homologous and analogous sequences from the new organism are thereby identified and selected for the new organism's mitochondrial array. The probes on the arrays of this invention are also useful as probes for identifying candidates for the new organism's array using molecular biology techniques that are standard in the art such as screening libraries.

All sequences given herein are meant to encompass the complementary strand, as well as double-stranded polynucleotides comprising the given sequence.

Microarrays of this invention can contain as few as two probes to as many as all the probes diagnostic of the selected physiological condition to be tested. Microarrays of this invention may also contain probes for all genes involved in mitochondrial biology. The arrays of this invention may contain probes for at least about five genes, at least about ten genes, at least about twenty-five genes, at least about fifty genes, at least about 100 genes, at least about 500 genes, or at least about 1000 genes. The mouse array may contain probes for at least about 950 genes and the human array may contain probes for at least about 600 genes. Arrays of this invention may comprise more than about five spots, more than about ten spots, more than about twenty-five spots, more than about one hundred spots, more than about 500 spots, or more than about 1000 spots.

Using microarrays may require amplification of target sequences (generation of multiple copies of the same sequence) of sequences of interest, such as by PCR or reverse transcription. As the nucleic acid is copied, it is tagged with a fluorescent label that emits light like a light bulb. The labeled nucleic acid is introduced to the microarray and allowed to react for a period of time. This nucleic acid sticks to, or hybridizes, with the probes on the array when the probe is sufficiently complementary to the labeled, amplified, sample nucleic acid. The extra nucleic acid is washed off of the array, leaving behind only the nucleic acid that has bound to the probes. By obtaining an image of the array with a fluorescent scanner and using software to analyze the hybridized array image, it can be determined if, and to what extent, genes are switched on and off, or whether or not sequences are present, by comparing fluorescent intensities at specific locations on the array. The intensity of the signal indicates to what extent a sequence is present. In expression arrays, high fluorescent signals indicate that many copies of a gene are present in a sample, and lower fluorescent signal shows a gene is less active. By selecting appropriate hybridization conditions and probes, this technique is useful for detecting single nucleotide polymorphisms (SNPs) and for sequencing. Methods of designing and using microarrays are continuously being improved (Relogio, A. et al. (2002) Nuc. Acids. Res. 30(1 l):e51; Iwasaki, H et al. (2002) DNA Res. 9(2):59-62; and Lindroos, K. et al. (2002) Nuc. Acids. Res. 30(14):E70).

Arrays of this invention may be made by any array synthesis methods known in the art such as spotting technology or solid phase synthesis. Preferably the arrays of this invention are synthesized by solid phase synthesis using a combination of photolithography and combinatorial chemistry. Some of the key elements of probe selection and array design are common to the production of all arrays. Strategies to optimize probe hybridization, for example, are invariably included in the process of probe selection. Hybridization under particular pH, salt, and temperature conditions can be optimized by taking into account melting temperatures and by using empirical rules that correlate with desired hybridization behaviors. Computer models may be used for predicting the intensity and concentration-dependence of probe hybridization.

Arrays, also called DNA microarrays or DNA chips, are fabricated by high-speed robotics, generally on glass but sometimes on nylon substrates, for which probes (Phimister, B. (1999) Nature Genetics 21s: 1-60) with known identity are used to determine complementary binding. An experiment with a single DNA chip can provide researchers information on thousands of genes simultaneously. There are several steps in the design and implementation of a DNA array experiment. Many strategies have been investigated at each of these steps: 1) DNA types; 2) Chip fabrication; 3) Sample preparation; 4) Assay; 5) Readout; and 6) Software (informatics).

There are two major application forms for the array technology: 1) Determination of expression level (abundance) of genes; and 2) Identification of sequence (gene/gene mutation). There appear to be two variants of the array technology, in terms of intellectual property, of arrayed DNA sequence with known identity: Format I consists of probe cDNA (500˜5,000 bases long) immobilized to a solid surface such as glass using robot spotting and exposed to a set of targets either separately or in a mixture. This method, “traditionally” called DNA microarray, is widely considered as having been developed at Stanford University. (R. Ekins and F. W. Chu “Microarrays: their origins and applications,” [1999] Trends in Biotechnology, 17:217-218). Format II consists of an array of oligonucleotide (20˜80-mer oligos) or peptide nucleic acid (PNA) probes synthesized either in situ (on-chip) or by conventional synthesis followed by on-chip immobilization. The array is exposed to labeled sample DNA, hybridized, and the identity/abundance of complementary sequences is determined. This method, “historically” called DNA chips, was developed at Affymetrix, Inc., which sells its photolithographically fabricated products under the GeneChip® trademark. Many companies are manufacturing oligonucleotide-based chips using alternative in-situ synthesis or depositioning technologies.

Probes on arrays can be hybridized with fluorescently-labeled target polynucleotides and the hybridized array can be scanned by means of scanning fluorescence microscopy. The fluorescence patterns are then analyzed by an algorithm that determines the extent of mismatch content, identifies polymorphisms, and provides some general sequencing information (M. Chee et al., [1996] Science 274:610). Selectivity is afforded in this system by low stringency washes to rinse away non-selectively adsorbed materials. Subsequent analysis of relative binding signals from array elements determines where base-pair mismatches may exist. This method then relies on conventional chemical methods to maximize stringency, and automated pattern recognition processing is used to discriminate between fully complementary and partially complementary binding.

Devices such as standard nucleic acid microarrays or gene chips, require data processing algorithms and the use of sample redundancy (i.e., many of the same types of array elements for statistically significant data interpretation and avoidance of anomalies) to provide semi-quantitative analysis of polymorphisms or levels of mismatch between the target sequence and sequences immobilized on the device surface. Such algorithms and software useful for statistical analysis are known to the art.

Using microarrays first requires amplification (generation of multiple copies of the same gene) of genes of interest, such as by reverse transcription. As the nucleic acid is copied, it is tagged with a fluorescent label that emits light like a light bulb. The labeled nucleic acid is introduced to the microarray and allowed to react for a period of time. This nucleic acid sticks to, or hybridizes, with the probes on the array when the probe is sufficiently complementary to the nucleic acid in the prepared sample. The extra nucleic acid is washed off of the array, leaving behind only the nucleic acid that has bound to the probes. By obtaining an image of the array with a fluorescent scanner and using software to analyze the hybridized array image, it can be determined if and to what extent genes are switched on and off, or whether or not sequences are present, by comparing fluorescent intensities at specific locations on the array. High fluorescent signals indicate that many copies of a gene are present in a prepared sample, and lower fluorescent signal shows a gene is less active. Expression levels for various genes under different conditions can be directly compared, such as for a cancer cell and a normal cell. Similarly, it can be determined what genes are turned on and off in response to certain stimuli such as a drug. Such information is valuable because it identifies genes in disease pathways and also is predictive of either efficacy or toxicity of drugs.

Detecting a particular polymorphism can be accomplished using two probes. One probe is designed to be perfectly complementary to a target sequence, and a partner probe is generated that is identical except for a single base mismatch in its center. In the Affymetrix system, these probe pairs are called the Perfect Match probe (PM) and the Mismatch probe (MM. They allow for the quantitation and subtraction of signals caused by non-specific cross-hybridization. The difference in hybridization signals between the partners, as well as their intensity ratios, serve as indicators of specific target abundance, and consequently of the sequence.

Arrays can rely on multiple probes to interrogate individual nucleotides in a sequence. The identity of a target base can be deduced using four identical probes that vary only in the target position, each containing one of the four possible bases. Alternatively, the presence of a consensus sequence can be tested using one or two probes representing specific alleles. To genotype heterozygous or genetically mixed samples, arrays with many probes can be created to provide redundant information, resulting in unequivocal genotyping.

Probes fixed on solid substrates and targets (nucleotide sequences in the sample) are combined in a hybridization buffer solution and held at an appropriate temperature until annealing occurs. Thereafter, the substrate is washed free of extraneous materials, leaving the nucleic acids on the target bound to the fixed probe molecules allowing for detection and quantitation by methods known in the art such as by autoradiograph, liquid scintillation counting, and/or fluorescence. As improvements are made in hybridization and detection techniques, they can be readily applied by one of ordinary skill in the art. As is well known in the art, if the probe molecules and target molecules hybridize by forming a strong non-covalent bond between the two molecules, it can be reasonably assumed that the probe and target nucleic acid are essentially identical, or almost completely complementary if the annealing and washing steps are carried out under conditions of high stringency. The detectable label provides a means for determining whether hybridization has occurred.

When using oligonucleotides or polynucleotides as hybridization probes, the probes may be labeled. In arrays of this invention, the target may instead be labeled by means known to the art. Target may be labeled with radioactive or non-radioactive labels. Targets preferably contain fluorescent labels.

Various degrees of stringency of hybridization can be employed. The more stringent the conditions are, the greater the complementarity that is required for duplex formation. Stringency can be controlled by temperature, probe concentration, probe length, ionic strength, time, and the like. Hybridization experiments are often conducted under moderate to high stringency conditions by techniques well know in the art, as described, for example in Keller, G. H., and M. M. Manak (1987) DNA Probes, Stockton Press, New York, N.Y., pp. 169-170, hereby incorporated by reference. However, sequencing arrays typically use lower hybridization stringencies, as is known in the art.

Moderate to high stringency conditions for hybridization are known to the art. An example of high stringency conditions for a blot are hybridizing at 68° C. in 5×SSC/5× Denhardt's solution/0.1% SDS, and washing in 0.2×SSC/0.1% SDS at room temperature. An example of conditions of moderate stringency are hybridizing at 680 C in 5×SSC/5× Denhardt's solution/0.1% SDS and washing at 42° C. in 3×SSC. The parameters of temperature and salt concentration can be varied to achieve the desired level of sequence identity between probe and target nucleic acid. See, e.g., Sambrook et al. (1989) vide infra or Ausubel et al. (1995) Current Protocols in Molecular Biology, John Wiley & Sons, New York, N.Y., for further guidance on hybridization conditions.

The melting temperature is described by the following formula (Beltz, G. A. et al., [1983] Methods of Enzymology, R. Wu, L. Grossman and K. Moldave [Eds.] Academic Press, New York 100:266-285).
Tm=81.5o C+16.6 Log[Na+]+0.41(+G+C)−0.61(% formamide)−600/length of duplex in base pairs.

Washes can typically be carried out as follows: twice at room temperature for 15 minutes in I×SSPE, 0.1% SDS (low stringency wash), and once at TM-20o C for 15 minutes in 0.2×SSPE, 0.1% SDS (moderate stringency wash).

Nucleic acid useful in this invention can be created by Polymerase Chain Reaction (PCR) amplification. PCR products can be confirmed by agarose gel electrophoresis. PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. [1985] Science 230:1350-1354). PCR is used to enzymatically amplify a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Since the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes that can be used are known to those skilled in the art.

Polynucleotide sequences of the present invention can be truncated and/or mutated such that certain of the resulting fragments and/or mutants of the original full-length sequence can retain the desired characteristics of the full-length sequence. A wide variety of restriction enzymes that are suitable for generating fragments from larger nucleic acid molecules are well known. In addition, it is well known that Bal31 exonuclease can be conveniently used for time-controlled limited digestion of DNA. See, for example, Maniatis (1982) Molecular Cloning: A Laboratory Manual, Cold Spring Harbor Laboratory, New York, pages 135-139, incorporated herein by reference. See also Wei et al. (1983) J. Biol. Chem. 258:13006-13512. By use of Bal3l exonuclease (commonly referred to as “erase-a-base” procedures), the ordinarily skilled artisan can remove nucleotides from either or both ends of the subject nucleic acids to generate a wide spectrum of fragments that are functionally equivalent to the subject nucleotide sequences. One of ordinary skill in the art can, in this manner, generate hundreds of fragments of controlled, varying lengths from locations all along the original molecule. The ordinarily skilled artisan can routinely test or screen the generated fragments for their characteristics and determine the utility of the fragments as taught herein. It is also well known that the mutant sequences can be easily produced with site-directed mutagenesis. See, for example, Larionov, O. A. and Nikiforov, V. G. (1982) Genetika 18(3):349-59; and Shortle, D. et al., (1981) Annu. Rev. Genet. 15:265-94, both incorporated herein by reference. The skilled artisan can routinely produce deletion-, insertion-, or substitution-type mutations and identify those resulting mutants that contain the desired characteristics of wild-type sequences, or fragments thereof.

Thus, mutational, insertional, and deletional variants of the disclosed nucleotide sequences can be readily prepared by methods which are well known to those skilled in the art. These variants can be used in the same manner as the exemplified primer sequences so long as the variants have substantial sequence homology with the original sequence. As used herein, substantial sequence homology refers to homology that is sufficient to enable the variant polynucleotide to function in the same capacity as the polynucleotide from which the probe was derived. Homology is greater than 80%, greater than 85%, greater than 90%, or greater than 95%. The degree of homology or identity needed for the variant to fimction in its intended capacity depends upon the intended use of the sequence. It is well within the skill of a person trained in this art to make mutational, insertional, and deletional mutations that are equivalent in function or are designed to improve the function of the sequence or otherwise provide a methodological advantage.

Percent sequence identity of two nucleic acids may be determined using the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (NBLAST and XBLAST) are used. See http://www.ncbi.nih.gov.

Standard techniques for cloning, DNA isolation, amplification and purification, for enzymatic reactions involving DNA ligase, DNA polymerase, restriction endonucleases and the like, and various separation techniques useful herein are those known and commonly employed by those skilled in the art. A number of standard techniques are described in Sambrook et al. (1989) Molecular Cloning, Second Edition, Cold Spring Harbor Laboratory, Plainview, New York; Maniatis et al. (1982) Molecular Cloning, Cold Spring Harbor Laboratory, Plainview, New York; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, New York; Old and Primrose (1981) Principles of Gene Manipulation, University of California Press, Berkeley; Schleif and Wensink (1982) Practical Methods in Molecular Biology; Glover (Ed.) (1985) DNA Cloning Vol. I and II, IRL Press, Oxford, UK; Hames and Higgins (Eds.) (1985) Nucleic Acid Hybridization, IRL Press, Oxford, UK; Setlow and Hollaender (1979) Genetic Engineering: Principles and Methods, Vols. 14, Plenum Press, New York; and Ausubel et al. (1992) Current Protocols in Molecular Biology, Greene/Wiley, New York, N.Y. Abbreviations and nomenclature, where employed, are deemed standard in the field and commonly used in professional journals such as those cited herein.

The arrays of this invention are useful for defining expression signatures or profiles for mitochondrial diseases, as well as distinguishing clinical disorders that result from OXPHOS dysfunction, oxidative stress, apoptosis, and aging. The microarrays of this invention are useful for providing profiles for whole classes of mitochondrial diseases that have common underlying pathophysiological mechanisms. The data obtained from using these arrays are useful in the identification of pathways involved in these diseases and in the design of efficient therapies for treating these diseases.

The arrays of this invention are useful for determining mitochondrial biology expression profiles and for sample evaluation using those profiles. The arrays of this invention are useful for diagnosis, for identifying pathways, genes, and mutations involved in physiological conditions, for creating animal models of human physiological conditions, and for designing curative and preventative therapies and evaluating their effectiveness.

The arrays of this invention are useful for determining mitochondrial biology expression profiles of organisms, such as humans, mice, and closely related species; tissues and organs of such organisms; cell types of such organisms; and cell lines derived from such organisms. An individual can be tested at any age, including as a fetus, neonate, infant, child, adolescent, mature adult, senior, and deceased. Using standard targets, the arrays of this invention are useful for comparing mitochondrial biology profiles of different individuals or cells.

The arrays of this invention are useful for determining the profile associated with a physiological condition such as an energy-metabolism-related physiological condition. Physiological conditions can be healthy conditions or pathological conditions. Examples of healthy conditions in humans are centenaria and physical fitness. An example of a pathological condition in humans is Leigh's syndrome (LS). By determining profiles from individuals, with and without such physiological conditions, and comparing them, the mitochondrial biology profile representative and descriptive of the physiological condition can be determined, such as for humans in Examples 4-5. Profiles can similarly be determined for cells lines with phenotypes or genotypes associated with physiological conditions, such as in Examples 13-15. Profiles can also be determined for non-human animals, including mouse strains, with physiological conditions as in Examples 8-12, 16, and 19. The arrays of this invention are useful for determining the range of normal variation of expression of genes involved in mitochondrial biology, as in Example 20. When the arrays of this invention are used to determine a profile associated with a physiological condition, prepared target samples or pooled prepared target samples, of individuals with and without the physiological condition, but otherwise similar, are hybridized to an array of this invention. The hybridization of the prepared samples are measured and compared to, if possible, determine a profile associated with the physiological condition. The profile may be optimized by statistical analysis, as is known in the art, to only contain profile data on probes necessary for diagnosing the physiological condition.

The profile associated with a physiological condition can then be used for diagnosis or evaluation using the arrays of this invention, such as in Example 7. The profile of the physiological condition can be analyzed and the analysis used to optimize an array for diagnosis of the physiological condition. An optimized array for diagnosis of a physiological condition minimally contains at least one probe for the one or more genes that have altered expression levels in the context of the physiological condition, and probes for enough genes to eliminate other likely diagnoses. Diagnosis involves collecting a sample from an individual who might have the physiological condition, and determining the profile of the prepared sample using an array of this invention, using an array containing probes for all genes involved in mitochondrial biology or fewer probes with at least as many probes as necessary for an array optimized for diagnosis of the physiological condition. The profile of the individual is then compared to the profile of the physiological condition, and the comparison is analyzed to determine the likelihood that the individual has the physiological condition. Arrays of this invention can also be used for screening individuals who are not suspected of having the particular physiological condition. A sample is collected from such an individual, prepared, and the mitochondrial biology profile of the individual is determined using an array of this invention, e.g., an array containing probes for all genes involved in mitochondrial biology. The profile of this individual is then compared to known mitochondrial biology profiles of one or more physiological conditions that the individual may have, to determine if the profile of the individual is indicative of a diagnosable physiological condition. As demonstrated in Example 16, the arrays of this invention are also useful for detecting profiles indicative of physiological conditions before the appearance of other symptoms.

The profile of, or associated with, a physiological condition is also useful for identifying biochemical pathways affected by the physiological condition and genes involved in causation of the physiological condition. If a profile of a physiological condition demonstrates alteration in the expression of a gene, that gene is a candidate for sequencing to identify a mutation causing the physiological condition. If a profile demonstrates alteration of expression of several genes, then genes known to regulate those are candidates for sequencing to identify a mutation causing the physiological condition. Example 3 describes using the arrays of this invention for the identification of mutations associated with physiological conditions.

The profile of a physiological condition is useful for creating and/or identifying animal models of human physiological conditions. The profile of a physiological condition may suggest types of mutations, such as knockouts, to create in order to mimic the physiological condition in an animal. The arrays of this invention are also useful for screening genetically engineered or other mutated populations to identify an individual animal having a similar profile, and thus associated with the physiological condition.

The same individual can be profiled, using arrays of this invention, repeatedly over time or after exposure to various environmental conditions, thereby determining the effects of time or exposure. Equivalent individuals can also be profiled, using the arrays of this invention, at different ages or after exposure to different environmental conditions, thereby determining the effects of time or exposure. For example, a control group of mice of a particular genotype and of a particular age can be compared, using the arrays of this invention, to a group of experimental mice of the same genotype and age, that has been exposed to a certain environmental hazard, to determine the effects of the environmental hazard. Cell lines, as well as organisms, can be profiled after exposure to different environmental conditions, as in Example 15. Arrays of this invention are also useful for determining the effects of aging. Examples 8 and 19 demonstrate differences in profiles at different ages.

Therapy is an environmental condition, the effects of which can be tested using the arrays of this invention. Identification of the pathways affected in a physiological condition allows identification of therapies useful to treat individuals having the physiological condition. For example, if profiles are determined for the effects of classes of therapeutic agents, as new physiological conditions are profiled, relevant therapeutic agents can be easily identified. The profile of a physiological condition is useful for testing candidate therapies for treating individuals with the physiological condition. Any individual, with or without the physiological condition, an animal model of the physiological condition in humans, or a cell line representative of an individual with the physiological condition, can be treated with a candidate therapy. A sample for profiling is collected after treatment, prepared, the profile is determined using an array of this invention, and compared to the profile of the same individual before treatment or to equivalent individuals or cells without treatment to determine the effect of the treatment. Therapies reversing the effects of the physiological condition can thereby be identified. Preventative therapies and therapies causing desired physiological conditions can similarly be identified.

The arrays of this invention are useful for monitoring the effectiveness of a therapy for a particular individual as well as for a population. The profile of a diagnosed individual can be determined, the individual given a therapy, and then the profile of the individual determined again, using the arrays of this invention. The therapy can be modified and the profile retested, until a satisfactory treated profile is obtained.

Arrays containing probes hybridizing at moderate to high stringency with human genes involved in mitochondrial biology are used for assaying prepared samples from humans, human cell lines, and prepared samples from closely related species. Arrays containing probes hybridizing at moderate to high stringency with mouse genes involved in mitochondrial biology are used for assaying prepared samples from mice, mouse cell lines, and prepared samples from closely related species.

The arrays of this invention are made using probes for genes involved in mitochondrial biology. Probes can be selected and generated from the lists of clones and sequences in Tables 3-5, or from sequences and clones representing genes involved in mitochondrial biology not listed in these tables. Probes can be generated in vitro by nucleic acid synthesis, PCR, cloning techniques or other techniques known in the art. Flanking or vector sequence may be minimized in the probe. Probes generated from Research Genetics clones (ResGen/Invitrogen, Carlsbad, Calif.) can be amplified by PCR as described in Example 22. Optionally, control probes are also selected for the arrays of this invention. Examples of clones and sequences for making control probes are listed in Table 6, SEQ ID NOS:3041-3044. If housekeeping genes are chosen as positive controls, usually they are derived from the same species as the non-control probes. Housekeeping gene probes are available from Stratagene (Spot Report™, La Jolla, Calif., USA).

Examples of housekeeping genes are shown in Table 2. Housekeeping genes generally have a consistent amount of expression in all cells. Using the arrays of this invention, the expression of the 25 housekeeping genes listed in Table 2 were compared in 4 cell lines, LMEB4, NZB, 501-1, and the LM(TK)-cell line grown in media supplemented with glucose, pyruvate, and uridine (GUP). Some variability was present between cell lines. Housekeeping genes were also tested in 6 different mouse tissue samples (brain, heart, liver, kidney, spleen and muscle) in two strains of mice, CAP^{R} and NZB. Variation was again present, but slight.

TABLE 2

| Description | Functional Class |
|---|---|
| Actin-gamma | Structural gene |
| A272 Capping protein | Structrual gene |
| Glyceraldehyde phosphate dehydrogenase | Metabolism-glycolysis |
| DNA ligase I | DNA repair/synthesis |
| β-actin | Structural |
| Alkaline Phosphatase | Unclassified |
| 40s Ribosomal protein S15 | Protein synthesis |
| Hypozanthine phosphoribosyl transferase | Metabolism-nucleotide |
| (HPRT) | |
| Ribosomal protein L15 | Protein synthesis |
| Ribosomal Protein S29 | Protein translation |
| Acient ubiquinating protein | Metabolism-protein |
| Glyceraldehyde 3-phosphate dehydrogenase | Metabolism-glycolysis |
| Actin-α (skeletal muscle) | Structural |
| Murine ornithine decarboxylase | Metabolism-amino acid |
| calcium binding protein Cab45 | Calcium homeostasis |
| Ribosomal protein L1A | Protein synthesis |
| RNA splicing protein | RNA processing |
| Actin-gamma (smooth muscle) | Structural gene |
| E2F transcription factor | Transcriptional regulation |
| Ubiquitin | Unclassified |
| Myosin 1 | Structural |
| HPRT | Metabolism-nucleotide |
| Phospholipase A2 (14-3-3 zeta/delta) | Signal transduction |
| HPRT | Metabolism-nucleotide |
| Ribosomal protein L3 | Protein synthesis |

Arrays can be printed on solid substrates, e.g., glass microscope slides. Before printing, slides are prepared to provide a substrate for binding as in Example 23. Arrays can be printed using any printing techniques and machines known in the art. Printing involves placing the probes on the substrate, attaching the probes to the substrate, and blocking the substrate to prevent non-specific hybridization, as described in Example 24.

Samples useful for analyses using the arrays of this invention include total RNA samples and mRNA samples. RNA samples can be prepared as described in Example 25. An RNA sample is reverse transcribed into cDNA and simultaneously labeled, i.e. with one member of a two-color fluorescent system, such as Cy3-dCTP/Cy5-dCTP as described in Example 26. The arrays are hybridized with the prepared sample and washed at appropriate stringencies accounting for the choices of sample and probes of the array. The hybridization stringency can be higher when the probe sequence has higher homology with the gene it interrogates and when the probe is larger. A reference target, standard target, or other sample target for direct comparison may be prepared and hybridized simultaneously to the same array. A prepared sample will not degrade during hybridization and is labeled. Prepared samples are reverse transcribed and fluorescently labeled.

Hybridization results can be measured and analyzed using equipment and software available in the art as described in Example 27. Before finalizing data, preliminary results are preferably normalized by methods known in the art. An example of normalization appears in Example 29. Analysis includes determination of statistical significance. Measurement may include normalization and analysis, including statistical analysis. Resulting data are typically stored in computer files.

Mitochondrial biology expression microarrays are useful for detecting alterations in gene expression caused by alterations in mitochondrial biology. Although commercially available total genome expression arrays from companies such as Incyte Pharmaceuticals or Affymetrix contain probes for ten to twenty times as many genes as the arrays of this invention, the commercially available arrays have limitations. Several genes and probes that have been included on the arrays of this invention are not available on the commercial arrays. The commercial arrays are also very expensive and the large data sets resulting from them can be rather cumbersome to analyze and manipulate. The smaller, more focused arrays of this invention allow the expression patterns of hundreds of mitochondrial genes to be monitored quickly and efficiently. This study shows that a custom-designed microarray for mitochondrial biology expression studies, including probes for nuclear as well as mitochondrial genes, is an effective tool for the analysis of gene expression changes caused by alterations in functions resulting from a mutation in a gene involved in mitochondrial biology or other changes in metabolic state. The cell line experiments in Examples 13-17 and 20 have been particularly informative in demonstrating the specificity and sensitivity of the arrays of this invention while the mouse tissue experiments in Examples 8-12 and 16-17 have shown the consistency of the arrays of this invention.

Clones used to generate probes are listed in Tables 3-5. Clones range from about 1 kb to about 4 kb. The inserts of most clones have been sequenced on the 5′ and 3′ ends. Sequences of the 5 and 3′ ends of the clones are usually about 200 nt to about 800 nt and are provided herein. Probes may be generated via several methods. For example, the clones listed in Tables 3-5 may be obtained commercially, the inserts purified and used as probes. Alternatively, a 5′ or 3 sequence given in the sequence listings hereof may be used to design an oligonucleotide which may be synthesized and used to probe a library to identify a cDNA or genomic clone that is equivalent to the clone used to generate the original sequence. This newly identified cDNA or genomic equivalent clone may be used to generate a probe. Alternatively, a pair of sequences from the sequence listings, representing the 5 and 3′ ends of one clone, may be used to design PCR primers, which may be used to PCR amplify an isolated nucleic acid that is quivalent to the insert of the corresponding clone from which the 5′ and 3′ were derived. This isolated nucleic acid may be used as a probe. Probes should not contain a vector sequence that hybridizes with any sequence in a sample. Methods for designing PCR primers and designing oligonucleotides for screening libraries are known in the art.

### EXAMPLES

### Example 1

Human Mitochondrial Biology Array

A human mitochondrial biology array is made from clones representing 650 expressed sequences involved in mitochondrial biology. The clones used to make probes that are placed on the array are shown in Table 3 which references SEQ ID NOS: 1-994 provided herein setting forth the 5′ and 3′ sequences from these clones. The clones identified in Table 3 are used to make a set of probes called Human Probe Set #1. Control sequences are also placed this array. Controls include, but are not limited to blanks, DMSO, probes derived from plant sequences, sequence(s) not involved in mitochondrial biology, and poly adenine (40-60 nucleotides long).

TABLE 3

| SEQ | | | | | |
|---|---|---|---|---|---|
| ID | ResGen | | | | UniGene |
| NO | Clone ID | Gene | Complete Gene Name | GenBank | Cluster ID |
| 1 | | mtDNA - 12S ribosome | Mitochondrial DNA 12S | | |
| 2 | | mtDNA - 16S ribosome | Mitochondrial DNA 16s | | |
| 3 | | mtDNA - ATP6 | Mitochondrial DNA ATP6 | | |
| 4 | | mtDNA - ATP8 | Mitochondrial DNA ATP8 | | |
| 5 | | mtDNA - COX1 | Mitochondrial DNA COX1 | | |
| 6 | | mtDNA - COX2 | Mitochondrial DNA COX2 | | |
| 7 | | mtDNA - COX3 | Mitochondrial DNA COX3 | | |
| 8 | | mtDNA - CYT B | Mitochondrial DNA CYTb | | |
| 9 | | mtDNA - ND1 | Mitochondrial DNA ND1 | | |
| 10 | | mtDNA - ND2 | Mitochondrial DNA ND2 | | |
| 11 | | mtDNA - ND3 | Mitochondrial DNA ND3 | | |
| 12 | | mtDNA - ND4 | Mitochondrial DNA ND4 | | |
| 13 | | mtDNA - ND4L | Mitochondrial DNA ND4L | | |
| 14 | | mtDNA - ND5 | Mitochondrial DNA ND5 | | |
| 15 | | mtDNA - ND6 | Mitochondrial DNA ND6 | | |
| 16 | 213890 | DECR1 | 2,4-dienoyl CoA reductase | H72937 | Hs.81548 |
| 17 | 213890 | DECR1 | 2,4-dienoyl CoA reductase | H72938 | Hs.81548 |
| 18 | 588911 | OAS1 | 2′,5′-oligoadenylate synthetase 1 | AA146773 | Hs.82396 |
| 19 | 588911 | OAS1 | 2′,5′-oligoadenylate synthetase 1 | AA146772 | Hs.82396 |
| 20 | 1576254 | 2′,5′-oligoadenylate | 2′,5′-oligoadenylate synthetase 1 | AA954880 | Hs.82396 |
| | | synthetase 1 | | | |
| 21 | 1057786 | OAS2 (splice-variant) | 2′-5′oligoadenylate synthetase 2 | AA568217 | |
| 22 | 2190112 | A38234 | 2-OXOGLUTARATE | AI610813 | |
| | | | DEHYDROGENASE E1 COMPONENT | | |
| 23 | 814444 | CRSP9 | 33 kDa transcriptional co-activator | AA459244 | Hs.7558 |
| 24 | 814444 | CRSP9 | 33 kDa transcriptional co-activator | AA459465 | Hs.7558 |
| 25 | 896949 | HMGCR | 3-hydroxy-3-methylglutaryl-Coenzyme A | AA779417 | |
| | | | reductase | | |
| 26 | 290111 | HMGCS1 | 3-hydroxy-3-methylglutaryl-Coenzyme A | N62195 | Hs.77910 |
| | | | synthase 1 (soluble) | | |
| 27 | 290111 | HMGCS1 | 3-hydroxy-3-methylglutaryl-Coenzyme A | N76492 | Hs.77910 |
| | | | synthase 1 (soluble) | | |
| 28 | 757222 | HMGCS2 | 3-hydroxy-3-methylglutaryl-Coenzyme A | AA496148 | |
| | | | synthase 2 (mitochondrial) | | |
| 29 | 757222 | HMGCS2 | 3-hydroxy-3-methylglutaryl-Coenzyme A | AA496149 | |
| | | | synthase 2 (mitochondrial) | | |
| 30 | 109310 | HAAO | 3-hydroxyanthranilate 3,4-dioxygenase | T80846 | |
| 31 | 109310 | HAAO | 3-hydroxyanthranilate 3,4-dioxygenase | T80921 | |
| 32 | 1635163 | HAAO | 3-hydroxyanthranilate 3,4-dioxygenase | AI005031 | |
| 33 | 66564 | BDH | 3-hydroxybutyrate dehydrogenase (heart, | T67057 | Hs.76893 |
| | | | mitochondrial) | | |
| 34 | 66564 | BDH | 3-hydroxybutyrate dehydrogenase (heart, | T67058 | Hs.76893 |
| | | | mitochondrial) | | |
| 35 | 838366 | HMGCL | 3-hydroxymethyl-3-methylglutaryl- | AA458779 | Hs.831 |
| | | | Coenzyme A lyase | | |
| | | | (hydroxymethylglutaricaciduria) | | |
| 36 | 838366 | HMGCL | 3-hydroxymethyl-3-methylglutaryl- | AA458172 | Hs.831 |
| | | | Coenzyme A lyase | | |
| | | | (hydroxymethylglutaricaciduria) | | |
| 37 | 28469 | OXCT | 3-oxoacid CoA transferase | R13381 | |
| 38 | 28469 | OXCT | 3-oxoacid CoA transferase | R40897 | |
| 39 | 591540 | PHGDH | 3-phosphoglycerate dehydrogenase | AA158735 | |
| 40 | 591540 | PHGDH | 3-phosphoglycerate dehydrogenase | AA159852 | |
| 41 | 266720 | PDPK1 | 3-phosphoinositide dependent protein | N22904 | |
| | | | kinase-1 | | |
| 42 | 266720 | PDPK1 | 3-phosphoinositide dependent protein | N31292 | |
| | | | kinase-1 | | |
| 43 | 666169 | MTR | 5-methyltetrahydrofolate-homocysteine | AA233640 | |
| | | | methyltransferase | | |
| 44 | 666169 | MTR | 5-methyltetrahydrofolate-homocysteine | AA233650 | |
| | | | methyltransferase | | |
| 45 | 814765 | AKAP1 | A kinase anchor protein, 149 kD | AA454947 | Hs.78921 |
| 46 | 814765 | AKAP1 | A kinase anchor protein, 149 kD | AA455326 | Hs.78921 |
| 47 | 2364633 | A32422 | A32422 | AI744652 | |
| 48 | 2308263 | A40487 | A40487 | AI671604 | |
| 49 | 2240514 | AAP1′ | AAP1′ | AI637909 | |
| 50 | 2266774 | ABF2 | ABF2 | AI590841 | |
| 51 | 36393 | ACAT2 | acetyl-Coenzyme A acetyltransferase 2 | R25823 | |
| | | | (acetoacetyl Coenzyme A thiolase) | | |
| 52 | 36393 | ACAT2 | acetyl-Coenzyme A acetyltransferase 2 | R46821 | |
| | | | (acetoacetyl Coenzyme A thiolase) | | |
| 53 | 45376 | ACAA2 | acetyl-Coenzyme A acyltransferase 2 | H07926 | Hs.32500 |
| | | | (mitochondrial 3-oxoacyl-Coenzyme A | | |
| | | | thiolase) | | |
| 54 | 45376 | ACAA2 | acetyl-Coenzyme A acyltransferase 2 | H08029 | Hs.32500 |
| | | | (mitochondrial 3-oxoacyl-Coenzyme A | | |
| | | | thiolase) | | |
| 55 | 262932 | ACO2 | Aconitase 2, mitochondrial | H99699 | Hs.75900 |
| 56 | 366511 | No Data | Actin, alpha 1, skeletal muscle | AA026609 | |
| 57 | 366511 | No Data | Actin, alpha 1, skeletal muscle | AA026720 | |
| 58 | 867606 | ACTB | actin, beta | AA780815 | |
| 59 | 428215 | ACTL6 | actin-like 6 | AA001745 | Hs.103180 |
| 60 | 428215 | ACTL6 | actin-like 6 | AA001815 | Hs.103180 |
| 61 | 896962 | ACADS | acyl-Coenzyme A dehydrogenase, C-2 to | AA676663 | Hs.127610 |
| | | | C-3 short chain | | |
| 62 | 298155 | ACADM | Acyl-Coenzyme A dehydrogenase, C-4 to | N70794 | Hs.79158 |
| | | | C-12 straight chain | | |
| 63 | 140131 | ACADL | acyl-Coenzyme A dehydrogenase, long | R66005 | Hs.1209 |
| | | | chain | | |
| 64 | 140131 | ACADL | acyl-Coenzyme A dehydrogenase, long | R66006 | Hs.1209 |
| | | | chain | | |
| 65 | 243100 | ACADSB | acyl-Coenzyme A dehydrogenase, | H95792 | Hs.81934 |
| | | | short/branched chain | | |
| 66 | 243100 | ACADSB | acyl-Coenzyme A dehydrogenase, | H96140 | Hs.81934 |
| | | | short/branched chain | | |
| 67 | 810358 | ACADVL | Acyl-Coenzyme A dehydrogenase, very | AA464163 | Hs.82208 |
| | | | long chain | | |
| 68 | 810358 | ACADVL | Acyl-Coenzyme A dehydrogenase, very | AA464228 | Hs.82208 |
| | | | long chain | | |
| 69 | 85450 | ACOX2 | acyl-Coenzyme A oxidase 2, branched | T71713 | Hs.9795 |
| | | | chain | | |
| 70 | 85450 | ACOX2 | acyl-Coenzyme A oxidase 2, branched | T71782 | Hs.9795 |
| | | | chain | | |
| 71 | 772304 | ANT2 | Adenine nucleotide translocator 2 | AA404486 | Hs.79172 |
| | | | (fibroblast) | | |
| 72 | 772304 | ANT2 | Adenine nucleotide translocator 2 | AA405477 | Hs.79172 |
| | | | (fibroblast) | | |
| 73 | 755855 | ANT3 | adenine nucleotide translocator 3 (liver) | AA496376 | Hs.164280 |
| 74 | 755855 | ANT3 | adenine nucleotide translocator 3 (liver) | AA496654 | Hs.164280 |
| 75 | 853570 | ANT3 | Adenine nucleotide translocator 3 (liver) | AA663439 | Hs.164280 |
| 76 | 868757 | AK1 | Adenylate kinase 1 | AA775325 | Hs.76240 |
| 77 | 2010933 | KAD2_HUMAN | ADENYLATE KINASE ISOENZYME 2 | AI361029 | |
| 78 | 40026 | ANT1 | ADP, ATP CARRIER PROTEIN, | R53337 | Hs.2043 |
| | | | HEART/SKELETAL MUSCLE | | |
| | | | ISOFORM T1 | | |
| 79 | 40026 | ANT1 | ADP, ATP CARRIER PROTEIN, | R53942 | Hs.2043 |
| | | | HEART/SKELETAL MUSCLE | | |
| | | | ISOFORM T1 | | |
| 80 | 46248 | ADPRT | ADP-ribosyltransferase (NAD+; poly | H09923 | Hs.177766 |
| | | | (ADP-ribose) polymerase) | | |
| 81 | 46248 | ADPRT | ADP-ribosyltransferase (NAD+; poly | H09924 | Hs.177766 |
| | | | (ADP-ribose) polymerase) | | |
| 82 | 589276 | AFG3L2 | AFG3 (ATPase family gene 3, yeast)-like 2 | AA147320 | Hs.29385 |
| 83 | 589276 | AFG3L2 | AFG3 (ATPase family gene 3, yeast)-like 2 | AA147413 | Hs.29385 |
| 84 | 1018253 | AIF | AIF | AA570483 | |
| 85 | 855624 | ALDH1 | aldehyde dehydrogenase 1, soluble | AA664101 | Hs.76392 |
| 86 | 47853 | ALDH4 | aldehyde dehydrogenase 4 (glutamate | H11346 | Hs.77448 |
| | | | gamma-semialdehyde dehydrogenase; | | |
| | | | pyrroline-5-carboxylate dehydrogenase) | | |
| 87 | 47853 | ALDH4 | aldehyde dehydrogenase 4 (glutamate | H11369 | Hs.77448 |
| | | | gamma-semialdehyde dehydrogenase; | | |
| | | | pyrroline-5-carboxylate dehydrogenase) | | |
| 88 | 197657 | ALDH5 | ALDEHYDE DEHYDROGENASE, | R93550 | Hs.169517 |
| | | | MITOCHONDRIAL X PRECURSOR | | |
| 89 | 197657 | ALDH5 | ALDEHYDE DEHYDROGENASE, | R93551 | Hs.169517 |
| | | | MITOCHONDRIAL X PRECURSOR | | |
| 90 | 1917741 | AOX1 | aldehyde oxidase 1 | AI343711 | Hs.81047 |
| 91 | 2154324 | AGPS | alkylglycerone phosphate synthase | AI445035 | |
| 92 | 1520618 | GABT_HUMAN | AMINOBUTYRATE | AA910669 | |
| | | | AMINOTRANSFERASE | | |
| 93 | 813651 | ALAS1 | aminolevulinate, delta-, synthase 1 | AA447761 | Hs.78712 |
| 94 | 813651 | ALAS1 | aminolevulinate, delta-, synthase 1 | AA453691 | Hs.78712 |
| 95 | 753346 | ALAS2 | aminolevulinate, delta-, synthase 2 | AA406485 | Hs.79103 |
| | | | (sideroblastic/hypochromic anemia) | | |
| 96 | 753346 | ALAS2 | aminolevulinate, delta-, synthase 2 | AA410346 | Hs.79103 |
| | | | (sideroblastic/hypochromic anemia) | | |
| 97 | 248631 | AMT | aminomethyltransferase (glycine cleavage | N59532 | Hs.102 |
| | | | system protein T) | | |
| 98 | 248631 | AMT | aminomethyltransferase (glycine cleavage | N78273 | Hs.102 |
| | | | system protein T) | | |
| 99 | 1556306 | ANT3 | ANT3 | AA916851 | |
| 100 | 471597 | API5L1 | API5-like 1 | AA035435 | Hs.227913 |
| 101 | 471597 | API5L1 | API5-like 1 | AA035436 | Hs.227913 |
| 102 | 127032 | API2 | apoptosis inhibitor 2 | R07870 | Hs.127799 |
| 103 | 127032 | API2 | apoptosis inhibitor 2 | R07927 | Hs.127799 |
| 104 | 2285739 | API3 | apoptosis inhibitor 3 | AI628066 | |
| 105 | 927606 | ARAL-1 | ARAL-1 | AA535370 | |
| 106 | 1704180 | XNHUDM | ASPARTATE AMINOTRANSFERASE | AI096615 | |
| 107 | 360778 | ATM | Ataxia telangiectasia mutated (includes | AA016254 | Hs.194382 |
| | | | complementation groups A, C and D) | | |
| 108 | 360778 | ATM | Ataxia telangiectasia mutated (includes | AA016988 | Hs.194382 |
| | | | complementation groups A, C and D) | | |
| 109 | 845519 | ATP5C1 | ATP SYNTHASE GAMMA CHAIN, | AA644234 | Hs.155433 |
| | | | MITOCHONDRIAL PRECURSOR | | |
| 110 | 813712 | ATP5F1 | ATP synthase, H+ transporting, | AA453765 | Hs.181101 |
| | | | mitochondrial F0 complex, subunit b, | | |
| | | | isoform 1 | | |
| 111 | 813712 | ATP5F1 | ATP synthase, H+ transporting, | AA453849 | Hs.181101 |
| | | | mitochondrial F0 complex, subunit b, | | |
| | | | isoform 1 | | |
| 112 | 193106 | ATP5G3 | ATP synthase, H+ transporting, | H47080 | Hs.429 |
| | | | mitochondrial F0 complex, subunit c | | |
| | | | (subunit 9) isoform 3 | | |
| 113 | 193106 | ATP5G3 | ATP synthase, H+ transporting, | H47164 | Hs.429 |
| | | | mitochondrial F0 complex, subunit c | | |
| | | | (subunit 9) isoform 3 | | |
| 114 | 487373 | ATP5G1 | ATP synthase, H+ transporting, | AA046701 | Hs.80986 |
| | | | mitochondrial F0 complex, subunit c | | |
| | | | (subunit 9), isoform 1 | | |
| 115 | 487373 | ATP5G1 | ATP synthase, H+ transporting, | AA046489 | Hs.80986 |
| | | | mitochondrial F0 complex, subunit c | | |
| | | | (subunit 9), isoform 1 | | |
| 116 | 809876 | ATP5G2 | ATP synthase, H+ transporting, | AA455126 | Hs.89399 |
| | | | mitochondrial F0 complex, subunit c | | |
| | | | (subunit 9), isoform 2 | | |
| 117 | 809876 | ATP5G2 | ATP synthase, H+ transporting, | AA464312 | Hs.89399 |
| | | | mitochondrial F0 complex, subunit c | | |
| | | | (subunit 9), isoform 2 | | |
| 118 | 825312 | ATP5J | ATP synthase, H+ transporting, | AA504540 | Hs.73851 |
| | | | mitochondrial F0 complex, subunit F6 | | |
| 119 | 825312 | ATP5J | ATP synthase, H+ transporting, | AA504465 | Hs.73851 |
| | | | mitochondrial F0 complex, subunit F6 | | |
| 120 | 392622 | ATP5B | ATP synthase, H+ transporting, | AA708298 | Hs.25 |
| | | | mitochondrial F1 complex, beta | | |
| | | | polypeptide | | |
| 121 | 856650 | ATP5D | ATP synthase, H+ transporting, | AA669314 | Hs.89761 |
| | | | mitochondrial F1 complex, delta subunit | | |
| 122 | 1472150 | ATP5O | ATP synthase, H+ transporting, | AA873577 | Hs.76572 |
| | | | mitochondrial F1 complex, O subunit | | |
| | | | (oligomycin sensitivity conferring | | |
| | | | protein) | | |
| 123 | 2098508 | ATP2B2 | ATP2B2 | AI421603 | Hs.89512 |
| 124 | 1753047 | ATP50 | ATP50 | AI184610 | |
| 125 | 1173869 | ATP5A1 | ATP5A1 | AA640573 | |
| 126 | 964121 | ATP5C1 | ATP5C1 | AA507388 | |
| 127 | 1736058 | ATP5F1 | ATP5F1 | AI126623 | |
| 128 | 307873 | ATP2B4 | ATPase, Ca++ transporting, plasma | N93024 | Hs.995 |
| | | | membrane 4 | | |
| 129 | 307873 | ATP2B4 | ATPase, Ca++ transporting, plasma | W21376 | Hs.995 |
| | | | membrane 4 | | |
| 130 | 1435103 | ATP2A3 | ATPase, Ca++ transporting, ubiquitous | AA857542 | Hs.5541 |
| 131 | 266312 | ATP7B | ATPase, Cu++ transporting, beta | N26536 | Hs.84999 |
| | | | polypeptide (Wilson disease) | | |
| 132 | 266312 | ATP7B | ATPase, Cu++ transporting, beta | N35647 | Hs.84999 |
| | | | polypeptide (Wilson disease) | | |
| 133 | 384078 | ATP6DV | ATPase, H+ transporting, lysosomal | AA702541 | Hs.106876 |
| | | | (vacuolar proton pump) 31 kD | | |
| 134 | 825170 | ATP6A1 | ATPase, H+ transporting, lysosomal | AA504160 | Hs.255352 |
| | | | (vacuolar proton pump), alpha | | |
| | | | polypeptide, 70 kD, isoform 1 | | |
| 135 | 825170 | ATP6A1 | ATPase, H+ transporting, lysosomal | AA504159 | Hs.255352 |
| | | | (vacuolar proton pump), alpha | | |
| | | | polypeptide, 70 kD, isoform 1 | | |
| 136 | 1323203 | ATP6B2 | ATPase, H+ transporting, lysosomal | AA877194 | Hs.1697 |
| | | | (vacuolar proton pump), beta polypeptide, | | |
| | | | 56/58 kD, isoform 2 | | |
| 137 | 461522 | ABCB7 | ATP-binding cassette, sub-family B | AA705237 | Hs.125856 |
| | | | (MDR/TAP), member 7 | | |
| 138 | 1709773 | MCX1 | ATP-DEPENDENT CLP PROTEASE | AI131257 | |
| 139 | 1467799 | KIAA0705 | atrophin-1 interacting protein 1 | AA883236 | Hs.22599 |
| 140 | 852273 | AZF1 | AZF1 | AA772863 | |
| 141 | 2367249 | B42665 | B42665 | AI741963 | |
| 142 | 194384 | BTF3 | Basic transcription factor 3 | R83000 | Hs.101025 |
| 143 | 194384 | BTF3 | Basic transcription factor 3 | R82957 | Hs.101025 |
| 144 | 342181 | BCL2 | B-cell CLL/lymphoma 2 | W63749 | Hs.79241 |
| 145 | 342181 | BCL2 | B-cell CLL/lymphoma 2 | W61100 | Hs.79241 |
| 146 | 826182 | BCL6 | B-cell CLL/lymphoma 6 (zinc finger | AA521434 | |
| | | | protein 51) | | |
| 147 | 814899 | BNIP3L | BCL2/adenovirus E1B 19 kD-interacting | AA465697 | Hs.132955 |
| | | | protein 3-like | | |
| 148 | 235938 | BAK1 | BCL2-antagonist/killer 1 | H52672 | |
| 149 | 235938 | BAK1 | BCL2-antagonist/killer 1 | H52673 | |
| 150 | 2125819 | BAX | BCL2-associated X protein | AI565203 | |
| 151 | 1916575 | BIK | BCL2-interacting killer (apoptosis- | AI347538 | |
| | | | inducing) | | |
| 152 | 1568561 | BCL2L1 | BCL2-like 1 | AA931820 | Hs.180372 |
| 153 | 2297154 | BCS1 | BCS1 | AI670836 | |
| 154 | 813444 | BZRP | benzodiazapine receptor (peripheral) | AA455945 | Hs.202 |
| 155 | 813444 | BZRP | benzodiazapine receptor (peripheral) | AA455554 | Hs.202 |
| 156 | 627125 | BID | BH3 interacting domain death agonist | AA190401 | |
| 157 | 627125 | BID | BH3 interacting domain death agonist | AA190546 | |
| 158 | 1573108 | BCKDK | branched chain alpha-ketoacid | AA970731 | Hs.20644 |
| | | | dehydrogenase kinase | | |
| 159 | 756490 | BCAT2 | branched chain aminotransferase 2, | AA436410 | Hs.101408 |
| | | | mitochondrial | | |
| 160 | 756490 | BCAT2 | branched chain aminotransferase 2, | AA481353 | Hs.101408 |
| | | | mitochondrial | | |
| 161 | 740801 | BCKDHA | branched chain keto acid dehydrogenase | AA477298 | Hs.78950 |
| | | | E1, alpha polypeptide (maple syrup urine | | |
| | | | disease) | | |
| 162 | 740801 | BCKDHA | branched chain keto acid dehydrogenase | AA477297 | Hs.78950 |
| | | | E1, alpha polypeptide (maple syrup urine | | |
| | | | disease) | | |
| 163 | 770835 | BCKDHB | Branched chain keto acid dehydrogenase | AA427739 | Hs.1265 |
| | | | E1, beta polypeptide (maple syrup urine | | |
| | | | disease) | | |
| 164 | 770835 | BCKDHB | Branched chain keto acid dehydrogenase | AA434304 | Hs.1265 |
| | | | E1, beta polypeptide (maple syrup urine | | |
| | | | disease) | | |
| 165 | 129431 | EST | BRCA2(?) | R11316 | Hs.188591 |
| 166 | 129431 | EST | BRCA2(?) | R11315 | Hs.188591 |
| 167 | 83605 | CPS1 | carbamoyl-phosphate synthetase 1, | T61078 | Hs.50966 |
| | | | mitochondrial | | |
| 168 | 83605 | CPS1 | carbamoyl-phosphate synthetase 1, | T61180 | Hs.50966 |
| | | | mitochondrial | | |
| 169 | 1675950 | CRHU5 | CARBONIC ANHYDRASE V | AI052226 | |
| | | | PRECURSOR | | |
| 170 | 744417 | CRAT | Carnitine acetyltransferase | AA621218 | Hs.12068 |
| 171 | 133565 | CPT1A | carnitine palmitoyltransferase I, liver | R28631 | Hs.29331 |
| 172 | 133565 | CPT1A | carnitine palmitoyltransferase I, liver | R32561 | Hs.29331 |
| 173 | 415978 | CPT1A | carnitine palmitoyltransferase I, liver | W85710 | Hs.29331 |
| 174 | 415978 | CPT1A | carnitine palmitoyltransferase I, liver | W86378 | Hs.29331 |
| 175 | 120106 | CASP1 | caspase 1, apoptosis-related cysteine | T95052 | |
| | | | protease (interleukin 1, beta, convertase) | | |
| 176 | 120106 | CASP1 | caspase 1, apoptosis-related cysteine | T95149 | |
| | | | protease (interleukin 1, beta, convertase) | | |
| 177 | 30170 | CASP3 | caspase 3, apoptosis-related cysteine | R14760 | |
| | | | protease | | |
| 178 | 30170 | CASP3 | caspase 3, apoptosis-related cysteine | R42530 | |
| | | | protease | | |
| 179 | 429574 | CASP3 | caspase 3, apoptosis-related cysteine | AA011445 | |
| | | | protease | | |
| 180 | 429574 | CASP3 | caspase 3, apoptosis-related cysteine | AA011446 | |
| | | | protease | | |
| 181 | 745143 | CASP6 | caspase 6, apoptosis-related cysteine | AA626710 | |
| | | | protease | | |
| 182 | 279691 | CD2AP | CD2-associated protein | N48329 | Hs.30490 |
| 183 | 279691 | CD2AP | CD2-associated protein | N49054 | Hs.30490 |
| 184 | 1558965 | CDC2L | cholinesterase-related cell division | AA917769 | Hs.155266 |
| | | | controller | | |
| 185 | 324885 | C11ORF4 | chromosome 11 open reading frame 4 | W48701 | Hs.75859 |
| 186 | 324885 | C11ORF4 | chromosome 11 open reading frame 4 | W48700 | Hs.75859 |
| 187 | 1573778 | C11ORF13 | chromosome 11 open reading frame 13 | AA970526 | Hs.72925 |
| 188 | 110772 | C14ORF2 | chromosome 14 open reading frame 2 | T90621 | Hs.109052 |
| 189 | 110772 | C14ORF2 | chromosome 14 open reading frame 2 | T83147 | Hs.109052 |
| 190 | 897448 | C2ORF1 | chromosome 2 open reading frame 1 | AA489478 | Hs.14454 |
| 191 | 1060841 | CIT1 | CITRATE SYNTHASE, | AA568724 | |
| | | | MITOCHONDRIAL PRECURSOR | | |
| 192 | 310519 | F10 | COAGULATION FACTOR X | N98524 | Hs.47913 |
| | | | PRECURSOR | | |
| 193 | 310519 | F10 | COAGULATION FACTOR X | W31088 | Hs.47913 |
| | | | PRECURSOR | | |
| 194 | 436062 | CPO | coproporphyrinogen oxidase | AA700808 | Hs.89866 |
| | | | (coproporphyria, harderoporphyria) | | |
| 195 | 161476 | COQ5 | COQ5 | H25602 | |
| 196 | 161476 | COQ5 | COQ5 | H25556 | |
| 197 | 2338136 | COX11.1-11.2 | COX11.1-11.2 | AI703310 | |
| 198 | 1902314 | COX15.1 | COX15.1 | AI301929 | |
| 199 | 1318021 | COX5A | COX5A | AA769095 | |
| 200 | 2326019 | COX5B | COX5B | AI688757 | Hs.1342 |
| 201 | 2301230 | COX5B | COX5B | AI699318 | |
| 202 | 2097122 | COX6A1 | COX6A1 | AI421088 | |
| 203 | 986164 | COX6A2 | COX6A2 | AA548887 | |
| 204 | 2277616 | COX6B | COX6B | AI690478 | |
| 205 | 971851 | COX7A1 | COX7A1 | AA515958 | |
| 206 | 937944 | COX7A2 | COX7A2 | AA563616 | |
| 207 | 2019469 | CKMT1 | creatine kinase, mitochondrial 1 | AI369378 | Hs.153998 |
| | | | (ubiquitous) | | |
| 208 | 795965 | CKMT2 | Creatine kinase, mitochondrial 2 | AA460480 | Hs.80691 |
| | | | (sarcomeric) | | |
| 209 | 795965 | CKMT2 | Creatine kinase, mitochondrial 2 | AA461048 | Hs.80691 |
| | | | (sarcomeric) | | |
| 210 | 771327 | NIFS | cysteine desulfurase | AA476245 | Hs.194692 |
| 211 | 771327 | NIFS | cysteine desulfurase | AA476244 | Hs.194692 |
| 212 | 196189 | CYB5 | Cytochrome b-5 | R92281 | Hs.83834 |
| 213 | 196189 | CYB5 | Cytochrome b-5 | R91950 | Hs.83834 |
| 214 | 840894 | COX6A1 | CYTOCHROME C OXIDASE | AA482243 | Hs.180714 |
| | | | POLYPEPTIDE VIA-LIVER | | |
| | | | PRECURSOR | | |
| 215 | 840894 | COX6A1 | CYTOCHROME C OXIDASE | AA482340 | Hs.180714 |
| | | | POLYPEPTIDE VIA-LIVER | | |
| | | | PRECURSOR | | |
| 216 | 824068 | COX5A | cytochrome c oxidase subunit Va | AA490735 | |
| 217 | 824068 | COX5A | cytochrome c oxidase subunit Va | AA491224 | |
| 218 | 298965 | COX6B | Cytochrome c oxidase subunit VIb | N71160 | Hs.174031 |
| 219 | 298965 | COX6B | Cytochrome c oxidase subunit VIb | W05541 | Hs.174031 |
| 220 | 1472754 | COX6B | cytochrome c oxidase subunit VIb | AA872391 | Hs.174031 |
| 221 | 838568 | COX6C | cytochrome c oxidase subunit VIc | AA456931 | Hs.74649 |
| 222 | 838568 | COX6C | cytochrome c oxidase subunit VIc | AA457006 | Hs.74649 |
| 223 | 1475803 | COX7A1 | cytochrome c oxidase subunit VIIa | AA872125 | Hs.114346 |
| | | | polypeptide 1 (muscle) | | |
| 224 | 1601947 | COX7A2 | cytochrome c oxidase subunit VIIa | AI002403 | Hs.182684 |
| | | | polypeptide 2 (liver) | | |
| 225 | 884511 | COX7B | cytochrome c oxidase subunit VIIb | AA629999 | Hs.75752 |
| 226 | 884480 | COX7C | cytochrome c oxidase subunit VIIc | AA629719 | Hs.3462 |
| 227 | 1469230 | COX8 | cytochrome c oxidase subunit VIII | AA862813 | Hs.81097 |
| 228 | 160126 | COX10 | Cytochrome c oxidase subunit X (heme | H21868 | Hs.77513 |
| | | | A: farnesyltransferase | | |
| 229 | 160126 | COX10 | Cytochrome c oxidase subunit X (heme | H21869 | Hs.77513 |
| | | | A: farnesyltransferase | | |
| 230 | 1455394 | CYC1 | cytochrome c-1 | AA865265 | Hs.697 |
| 231 | 194949 | CYP3A7 | Cytochrome P450 IIIA7 (P450-HFLa) | R91078 | Hs.172323 |
| 232 | 194949 | CYP3A7 | Cytochrome P450 IIIA7 (P450-HFLa) | R91077 | Hs.172323 |
| 233 | 1724630 | S14367 | CYTOCHROME P450 XIA1 | AI183397 | |
| 234 | 85561 | CYP2A7 | cytochrome P450, subfamily IIA | T73031 | Hs.252937 |
| | | | (phenobarbital-inducible), polypeptide 7 | | |
| 235 | 85561 | CYP2A7 | cytochrome P450, subfamily IIA | T72259 | Hs.252937 |
| | | | (phenobarbital-inducible), polypeptide 7 | | |
| 236 | 246619 | CYP2C8 | Cytochrome P450, subfamily IIC | N53136 | |
| | | | (mephenytoin 4-hydroxylase) | | |
| 237 | 246619 | CYP2C8 | Cytochrome P450, subfamily IIC | N58566 | |
| | | | (mephenytoin 4-hydroxylase) | | |
| 238 | 195712 | CYP2C9 | Cytochrome P450, subfamily IIC | R89492 | Hs.167529 |
| | | | (mephenytoin 4-hydroxylase), | | |
| | | | polypeptide 9 | | |
| 239 | 195712 | CYP2C9 | Cytochrome P450, subfamily IIC | R89491 | Hs.167529 |
| | | | (mephenytoin 4-hydroxylase), | | |
| | | | polypeptide 9 | | |
| 240 | 1467195 | CYP11B1 | cytochrome P450, subfamily XIB (steroid | AA884709 | Hs.2610 |
| | | | 11-beta-hydroxylase), polypeptide 1 | | |
| 241 | 149737 | EST | Cytochrome P450, subfamily XIX | H00592 | Hs.141142 |
| | | | (aromatization of androgens) | | |
| 242 | 149737 | EST | Cytochrome P450, subfamily XIX | R82738 | Hs.141142 |
| | | | (aromatization of androgens) | | |
| 243 | 284620 | EST | Cytochrome P450, subfamily XXI | N64794 | Hs.124918 |
| | | | (steroid 21-hydroxylase, congenital | | |
| | | | adrenal hyperplasia) | | |
| 244 | 284620 | EST | Cytochrome P450, subfamily XXI | N77388 | Hs.124918 |
| | | | (steroid 21-hydroxylase, congenital | | |
| | | | adrenal hyperplasia) | | |
| 245 | 266146 | CYP24 | cytochrome P450, subfamily XXIV | N21576 | Hs.89663 |
| | | | (vitamin D 24-hydroxylase) | | |
| 246 | 266146 | CYP24 | cytochrome P450, subfamily XXIV | N30976 | Hs.89663 |
| | | | (vitamin D 24-hydroxylase) | | |
| 247 | 295843 | CYP27A1 | cytochrome P450, subfamily XXVIIA | N66957 | Hs.82568 |
| | | | (steroid 27-hydroxylase, | | |
| | | | cerebrotendinous xanthomatosis), | | |
| | | | polypeptide 1 | | |
| 248 | 2043415 | DAPK1 | death-associated protein kinase 1 | AI371096 | Hs.153924 |
| 249 | 2364396 | DEHUH2 | DEHUH2 | AI740677 | |
| 250 | 125722 | DGUOK | deoxyguanosine kinase | R07560 | Hs.77494 |
| 251 | 125722 | DGUOK | deoxyguanosine kinase | R07506 | Hs.77494 |
| 252 | 2096376 | S25665 | DIHYDROLIPOAMIDE | AI419467 | |
| | | | ACETYLTRANSFERASE | | |
| | | | COMPONENT | | |
| 253 | 813648 | DLD | dihydrolipoamide dehydrogenase (E3 | AA453679 | Hs.74635 |
| | | | component of pyruvate dehydrogenase | | |
| | | | complex, 2-oxo-glutarate complex, | | |
| | | | branched chain keto acid dehydrogenase | | |
| | | | complex) | | |
| 254 | 813648 | DLD | dihydrolipoamide dehydrogenase (E3 | AA447748 | Hs.74635 |
| | | | component of pyruvate dehydrogenase | | |
| | | | complex, 2-oxo-glutarate complex, | | |
| | | | branched chain keto acid dehydrogenase | | |
| | | | complex) | | |
| 255 | 271006 | DLAT | dihydrolipoamide S-acetyltransferase (E2 | N29901 | Hs.115285 |
| | | | component of pyruvate dehydrogenase | | |
| | | | complex) | | |
| 256 | 271006 | DLAT | dihydrolipoamide S-acetyltransferase (E2 | N42953 | Hs.115285 |
| | | | component of pyruvate dehydrogenase | | |
| | | | complex) | | |
| 257 | 815564 | DLST | Dihydrolipoamide S-succinyltransferase | AA456824 | Hs.196416 |
| | | | (E2 component of 2-oxo-glutarate | | |
| | | | complex) | | |
| 258 | 815564 | DLST | Dihydrolipoamide S-succinyltransferase | AA456899 | Hs.196416 |
| | | | (E2 component of 2-oxo-glutarate | | |
| | | | complex) | | |
| 259 | 1308945 | DIHYDROLIPOAMIDE | DIHYDROLIPOAMIDE | AA748401 | |
| | | SUCCINYLTRANSFERASE | SUCCINYLTRANSFERASE | | |
| 260 | 1308945 | DIHYDROLIPOAMIDE | DIHYDROLIPOAMIDE | AA746285 | |
| | | SUCCINYLTRANSFERASE | SUCCINYLTRANSFERASE | | |
| 261 | 417385 | Dihydroorotate | Dihydroorotate dehydrogenase | W88472 | Hs.125846 |
| | | dehydrogenase | | | |
| 262 | 417385 | Dihydroorotate | Dihydroorotate dehydrogenase | W89035 | Hs.125846 |
| | | dehydrogenase | | | |
| 263 | 611027 | dihydroorotate | dihydroorotate dehydrogenase | AA173122 | Hs.94925 |
| | | dehydrogenase | | | |
| 264 | 611027 | dihydroorotate | dihydroorotate dehydrogenase | AA173225 | Hs.94925 |
| | | dehydrogenase | | | |
| 265 | 884539 | DKFZP566D143 | DKFZP566D143 protein | AA629804 | |
| 266 | 630013 | MSH2 | DNA repair protein MSH2 | AA219060 | Hs.78934 |
| 267 | 630013 | MSH2 | DNA repair protein MSH2 | AA219061 | Hs.78934 |
| 268 | 666425 | PLCG1 | DNA topoisomerase I | AA232856 | Hs.317 |
| 269 | 666425 | PLCG1 | DNA topoisomerase I | AA233029 | Hs.317 |
| 270 | 810787 | HSPF1 | DNAJ PROTEIN HOMOLOG 1 | AA481758 | Hs.82646 |
| 271 | 810787 | HSPF1 | DNAJ PROTEIN HOMOLOG 1 | AA481022 | Hs.82646 |
| 272 | 1914863 | DYSF | dysferlin, limb girdle muscular dystrophy | AI310142 | Hs.143897 |
| | | | 2B (autosomal recessive) | | |
| 273 | 773399 | DMD | dystrophin (muscular dystrophy, | AA425649 | Hs.169470 |
| | | | Duchenne and Becker types), includes | | |
| | | | DXS142, DXS164, DXS206, DXS230, | | |
| | | | DXS239, DXS268, DXS269, DXS270, | | |
| | | | DXS272 | | |
| 274 | 773399 | DMD | dystrophin (muscular dystrophy, | AA427831 | Hs.169470 |
| | | | Duchenne and Becker types), includes | | |
| | | | DXS142, DXS164, DXS206, DXS230, | | |
| | | | DXS239, DXS268, DXS269, DXS270, | | |
| | | | DXS272 | | |
| 275 | 796197 | DMD | Dystrophin (muscular dystrophy, | AA461118 | Hs.169470 |
| | | | Duchenne and Becker types), includes | | |
| | | | DXS142, DXS164, DXS206, DXS230, | | |
| | | | DXS239, DXS268, DXS269, DXS270, | | |
| | | | DXS272 | | |
| 276 | 796197 | DMD | Dystrophin (muscular dystrophy, | AA461435 | Hs.169470 |
| | | | Duchenne and Becker types), includes | | |
| | | | DXS142, DXS164, DXS206, DXS230, | | |
| | | | DXS239, DXS268, DXS269, DXS270, | | |
| | | | DXS272 | | |
| 277 | 781017 | EGR2 | early growth response 2 (Krox-20 | AA446027 | Hs.1395 |
| | | | (Drosophila) homolog) | | |
| 278 | 781017 | EGR2 | early growth response 2 (Krox-20 | AA446300 | Hs.1395 |
| | | | (Drosophila) homolog) | | |
| 279 | 180512 | ENC1 | ectodermal-neural cortex (with BTB-like | R85090 | Hs.104925 |
| | | | domain) | | |
| 280 | 1744035 | A31998 | ELECTRON TRANSFER | AI192719 | |
| | | | FLAVOPROTEIN ALPHA-SUBUNIT | | |
| 281 | 2274670 | S32482 | ELECTRON TRANSFER | AI683530 | |
| | | | FLAVOPROTEIN BETA-SUBUNIT | | |
| 282 | 2267229 | S62767 | ELONGATION FACTOR TU | AI609398 | |
| 283 | 745542 | ECHS1 | enoyl Coenzyme A hydratase, short | AA626255 | Hs.76394 |
| | | | chain, 1, mitochondrial | | |
| 284 | 344272 | EMP3 | epithelial membrane protein 3 | W73810 | Hs.9999 |
| 285 | 344272 | EMP3 | epithelial membrane protein 3 | W73748 | Hs.9999 |
| 286 | 253725 | EST | EST | N21972 | Hs.43052 |
| 287 | 287569 | EST | EST | N62122 | Hs.83313 |
| 288 | 287569 | EST | EST | N78351 | Hs.83313 |
| 289 | 489755 | EST | EST | AA099554 | Hs.246174 |
| 290 | 489755 | EST | EST | AA101991 | Hs.246174 |
| 291 | 609989 | No Data | EST | AA169176 | |
| 292 | 609989 | No Data | EST | AA169296 | |
| 293 | 511012 | AGPS | EST - putative alkylglycerone phosphate | AA099787 | |
| | | | synthase | | |
| 294 | 511012 | AGPS | EST - putative alkylglycerone phosphate | AA102257 | |
| | | | synthase | | |
| 295 | 449504 | EST | EST, Weakly similar to predicted using | AA777928 | Hs.121993 |
| | | | Genefinder [C. elegans] | | |
| 296 | 47005 | EST | ESTs | H09825 | Hs.6818 |
| 297 | 47005 | EST | ESTs | H09920 | Hs.6818 |
| 298 | 79655 | EST | ESTs | T62655 | Hs.11039 |
| 299 | 79655 | EST | ESTs | T62509 | Hs.11039 |
| 300 | 126229 | EST | ESTs | R06313 | Hs.77677 |
| 301 | 126229 | EST | ESTs | R06258 | Hs.77677 |
| 302 | 129606 | EST | ESTs | R16545 | Hs.70333 |
| 303 | 129606 | EST | ESTs | R16603 | Hs.70333 |
| 304 | 165837 | EST | ESTs | R86713 | Hs.87595 |
| 305 | 165837 | EST | ESTs | R86712 | Hs.87595 |
| 306 | 248669 | EST | ESTs | N59553 | Hs.8941 |
| 307 | 248669 | EST | ESTs | N78295 | Hs.8941 |
| 308 | 254004 | EST | ESTs | N22302 | Hs.177861 |
| 309 | 254004 | EST | ESTs | N75187 | Hs.177861 |
| 310 | 259462 | LOC54675 | ESTs | N29545 | Hs.3569 |
| 311 | 290505 | EST | ESTs | N67991 | Hs.30487 |
| 312 | 290505 | EST | ESTs | N80413 | Hs.30487 |
| 313 | 341901 | EST | ESTs | W61374 | Hs.11317 |
| 314 | 429942 | EST | ESTs | AA034062 | Hs.38750 |
| 315 | 784214 | EST | ESTs | AA446865 | Hs.14018 |
| 316 | 784214 | EST | ESTs | AA446980 | Hs.14018 |
| 317 | 898222 | EST | ESTs | AA598602 | Hs.13434 |
| 318 | 1636741 | ESTs | ESTs | AI017846 | Hs.169539 |
| 319 | 113206 | EST | ESTs, Highly similar to ARGINYL- | T83996 | Hs.15395 |
| | | | TRNA SYNTHETASE, | | |
| | | | MITOCHONDRIAL PRECURSOR | | |
| | | | [Saccharomyces cerevisiae] | | |
| 320 | 113206 | EST | ESTs, Highly similar to ARGINYL- | T83997 | Hs.15395 |
| | | | TRNA SYNTHETASE, | | |
| | | | MITOCHONDRIAL PRECURSOR | | |
| | | | [Saccharomyces cerevisiae] | | |
| 321 | 825386 | ATP5JD | ESTs, Highly similar to ATP | AA504246 | Hs.64593 |
| | | | SYNTHASE D CHAIN, | | |
| | | | MITOCHONDRIAL [Bos taurus] | | |
| 322 | 782439 | ATP5I | ESTs, Highly similar to ATP | AA431433 | Hs.85539 |
| | | | SYNTHASE E CHAIN, | | |
| | | | MITOCHONDRIAL [Cricetulus | | |
| | | | longicaudatus] | | |
| 323 | 782439 | ATP5I | ESTs, Highly similar to ATP | AA431836 | Hs.85539 |
| | | | SYNTHASE E CHAIN, | | |
| | | | MITOCHONDRIAL [Cricetulus | | |
| | | | longicaudatus] | | |
| 324 | 434968 | ATP5E | ESTs, Highly similar to ATP | AA700688 | Hs.177530 |
| | | | SYNTHASE EPSILON CHAIN, | | |
| | | | MITOCHONDRIAL PRECURSOR [Bos | | |
| | | | taurus] | | |
| 325 | 82874 | EST | ESTs, Highly similar to ATPASE | T69273 | Hs.241336 |
| | | | INHIBITOR, MITOCHONDRIAL | | |
| | | | PRECURSOR [Rattus norvegicus] | | |
| 326 | 82874 | EST | ESTs, Highly similar to ATPASE | T69348 | Hs.241336 |
| | | | INHIBITOR, MITOCHONDRIAL | | |
| | | | PRECURSOR [Rattus norvegicus] | | |
| 327 | 290753 | CS | ESTs, Highly similar to CITRATE | N67639 | Hs.239760 |
| | | | SYNTHASE, MITOCHONDRIAL | | |
| | | | PRECURSOR [Sus scrofa] | | |
| 328 | 290753 | CS | ESTs, Highly similar to CITRATE | W01297 | Hs.239760 |
| | | | SYNTHASE, MITOCHONDRIAL | | |
| | | | PRECURSOR [Sus scrofa] | | |
| 329 | 731308 | CS | ESTs, Highly similar to CITRATE | AA416759 | Hs.239760 |
| | | | SYNTHASE, MITOCHONDRIAL | | |
| | | | PRECURSOR [Sus scrofa] | | |
| 330 | 731308 | CS | ESTs, Highly similar to CITRATE | AA416746 | Hs.239760 |
| | | | SYNTHASE, MITOCHONDRIAL | | |
| | | | PRECURSOR [Sus scrofa] | | |
| 331 | 283943 | EST | ESTs, Highly similar to ELONGATION | N50802 | Hs.41066 |
| | | | FACTOR G, MITOCHONDRIAL | | |
| | | | PRECURSOR [Rattus norvegicus] | | |
| 332 | 283943 | EST | ESTs, Highly similar to ELONGATION | N55159 | Hs.41066 |
| | | | FACTOR G, MITOCHONDRIAL | | |
| | | | PRECURSOR [Rattus norvegicus] | | |
| 333 | 359723 | EST | ESTs, Highly similar to ELONGATION | AA011122 | Hs.41066 |
| | | | FACTOR G, MITOCHONDRIAL | | |
| | | | PRECURSOR [Rattus norvegicus] | | |
| 334 | 359723 | EST | ESTs, Highly similar to ELONGATION | AA010761 | Hs.41066 |
| | | | FACTOR G, MITOCHONDRIAL | | |
| | | | PRECURSOR [Rattus norvegicus] | | |
| 335 | 430733 | EST | ESTs, Highly similar to | AA677960 | Hs.3585 |
| | | | HYPOTHETICAL 16.5 KD PROTEIN | | |
| | | | IN PAS8-EGT2 INTERGENIC REGION | | |
| | | | [Saccharomyces cerevisiae] | | |
| 336 | 1114960 | ETFDH | ESTs, Highly similar to | AA602015 | Hs.30661 |
| | | | HYPOTHETICAL 29.2 KD PROTEIN | | |
| | | | IN PHD1-PTM1 INTERGENIC | | |
| | | | REGION [Saccharomyces cerevisiae] | | |
| 337 | 1030791 | EST | ESTs, Highly similar to | AA609009 | Hs.63304 |
| | | | HYPOTHETICAL 44.9 KD PROTEIN | | |
| | | | IN ERG7-NMD2 INTERGENIC | | |
| | | | REGION [Saccharomyces cerevisiae] | | |
| 338 | 950700 | PP | ESTs, Highly similar to INORGANIC | AA608572 | Hs.184011 |
| | | | PYROPHOSPHATASE [Bos taurus] | | |
| 339 | 322218 | EST | ESTs, Highly similar to ISOLEUCYL- | W37993 | Hs.55609 |
| | | | TRNA SYNTHETASE, | | |
| | | | MITOCHONDRIAL [Saccharomyces | | |
| | | | cerevisiae] | | |
| 340 | 322218 | EST | ESTs, Highly similar to ISOLEUCYL- | W37992 | Hs.55609 |
| | | | TRNA SYNTHETASE, | | |
| | | | MITOCHONDRIAL [Saccharomyces | | |
| | | | cerevisiae] | | |
| 341 | 417801 | LOC51264 | ESTs, Highly similar to | W88752 | Hs.7736 |
| | | | MITOCHONDRIAL 60S RIBOSOMAL | | |
| | | | PROTEIN L2 PRECURSOR | | |
| | | | [Saccharomyces cerevisiae] | | |
| 342 | 417801 | LOC51264 | ESTs, Highly similar to | W88848 | Hs.7736 |
| | | | MITOCHONDRIAL 60S RIBOSOMAL | | |
| | | | PROTEIN L2 PRECURSOR | | |
| | | | [Saccharomyces cerevisiae] | | |
| 343 | 753602 | EST | ESTs, Highly similar to | AA479944 | Hs.42484 |
| | | | MITOCHONDRIAL CARRIER | | |
| | | | PROTEIN RIM2 [Saccharomyces | | |
| | | | cerevisiae] | | |
| 344 | 753602 | EST | ESTs, Highly similar to | AA478847 | Hs.42484 |
| | | | MITOCHONDRIAL CARRIER | | |
| | | | PROTEIN RIM2 [Saccharomyces | | |
| | | | cerevisiae] | | |
| 345 | 629916 | TIM17B | ESTs, Highly similar to | AA219179 | Hs.19105 |
| | | | MITOCHONDRIAL IMPORT INNER | | |
| | | | MEMBRANE TRANSLOCASE | | |
| | | | SUBUNIT TIM17 [Saccharomyces | | |
| | | | cerevisiae] | | |
| 346 | 629916 | TIM17B | ESTs, Highly similar to | AA219178 | Hs.19105 |
| | | | MITOCHONDRIAL IMPORT INNER | | |
| | | | MEMBRANE TRANSLOCASE | | |
| | | | SUBUNIT TIM17 [Saccharomyces | | |
| | | | cerevisiae] | | |
| 347 | 191826 | EST | ESTs, Highly similar to | H40449 | Hs.34401 |
| | | | MITOCHONDRIAL RNA SPLICING | | |
| | | | PROTEIN MRS3 [Saccharomyces | | |
| | | | cerevisiae] | | |
| 348 | 191826 | EST | ESTs, Highly similar to | H40448 | Hs.34401 |
| | | | MITOCHONDRIAL RNA SPLICING | | |
| | | | PROTEIN MRS3 [Saccharomyces | | |
| | | | cerevisiae] | | |
| 349 | 70201 | EST | ESTs, Highly similar to | T50082 | Hs.34401 |
| | | | MITOCHONDRIAL RNA SPLICING | | |
| | | | PROTEIN MSR4 [Saccharomyces | | |
| | | | cerevisiae] | | |
| 350 | 70201 | EST | ESTs, Highly similar to | T50019 | Hs.34401 |
| | | | MITOCHONDRIAL RNA SPLICING | | |
| | | | PROTEIN MSR4 [Saccharomyces | | |
| | | | cerevisiae] | | |
| 351 | 488386 | EST | ESTs, Highly similar to | AA046639 | Hs.34401 |
| | | | MITOCHONDRIAL RNA SPLICING | | |
| | | | PROTEIN MSR4 [Saccharomyces | | |
| | | | cerevisiae] | | |
| 352 | 488386 | EST | ESTs, Highly similar to | AA046778 | Hs.34401 |
| | | | MITOCHONDRIAL RNA SPLICING | | |
| | | | PROTEIN MSR4 [Saccharomyces | | |
| | | | cerevisiae] | | |
| 353 | 611467 | NDUFS6 | ESTs, Highly similar to | AA176453 | Hs.49767 |
| | | | MITOCHONDRIAL TRIFUNCTONAL | | |
| | | | ENZYME ALPHA SUBUNIT | | |
| | | | PRECURSOR [Homo sapiens] | | |
| 354 | 611467 | NDUFS6 | ESTs, Highly similar to | AA176931 | Hs.49767 |
| | | | MITOCHONDRIAL TRIFUNCTONAL | | |
| | | | ENZYME ALPHA SUBUNIT | | |
| | | | PRECURSOR [Homo sapiens] | | |
| 355 | 179336 | SDHA | ESTs, Highly similar to SUCCINATE | H50345 | Hs.469 |
| | | | DEHYDROGENASE [Homo sapiens] | | |
| 356 | 179336 | SDHA | ESTs, Highly similar to SUCCINATE | H50378 | Hs.469 |
| | | | DEHYDROGENASE [Homo sapiens] | | |
| 357 | 843335 | SUCLG1 | ESTs, Highly similar to SUCCINYL- | AA485965 | Hs.7043 |
| | | | COA LIGASE [Rattus norvegicus] | | |
| 358 | 843335 | SUCLG1 | ESTs, Highly similar to SUCCINYL- | AA489575 | Hs.7043 |
| | | | COA LIGASE [Rattus norvegicus] | | |
| 359 | 825229 | LOC51017 | ESTs, Highly similar to CGI-113 protein | AA504139 | Hs.19077 |
| | | | [H. sapiens] | | |
| 360 | 825229 | LOC51017 | ESTs, Highly similar to CGI-113 protein | AA504401 | Hs.19077 |
| | | | [H. sapiens] | | |
| 361 | 814271 | Est | ESTs, Highly similar to CGI-116 protein | AA459002 | Hs.18885 |
| | | | [H. sapiens] | | |
| 362 | 814271 | Est | ESTs, Highly similar to CGI-116 protein | AA459222 | Hs.18885 |
| | | | [H. sapiens] | | |
| 363 | 811062 | LOC51629 | ESTs, Highly similar to CGI-69 protein | AA485441 | Hs.237924 |
| | | | [H. sapiens] | | |
| 364 | 811062 | LOC51629 | ESTs, Highly similar to CGI-69 protein | AA485607 | Hs.237924 |
| | | | [H. sapiens] | | |
| 365 | 417803 | EST | ESTs, Highly similar to hypothetical | W88753 | Hs.166406 |
| | | | protein [H. sapiens] | | |
| 366 | 417803 | EST | ESTs, Highly similar to hypothetical | W88859 | Hs.166406 |
| | | | protein [H. sapiens] | | |
| 367 | 878316 | ESTs | ESTs, Highly similar to small zinc finger- | AA670296 | Hs.109571 |
| | | | like protein [H. sapiens] | | |
| 368 | 504689 | EST | ESTs, Moderately similar to 3- | AA149172 | Hs.55781 |
| | | | OXOACYL-[ACYL-CARRIER- | | |
| | | | PROTEIN] SYNTHASE II [Escherichia | | |
| | | | coli] | | |
| 369 | 504689 | EST | ESTs, Moderately similar to 3- | AA149171 | Hs.55781 |
| | | | OXOACYL-[ACYL-CARRIER- | | |
| | | | PROTEIN] SYNTHASE II [Escherichia | | |
| | | | coli] | | |
| 370 | 238907 | D6S52E | ESTs, Moderately similar to ABC1 | H67876 | Hs.243960 |
| | | | PROTEIN PRECURSOR | | |
| | | | [Saccharomyces cerevisiae] | | |
| 371 | 238907 | D6S52E | ESTs, Moderately similar to ABC1 | H67202 | Hs.243960 |
| | | | PROTEIN PRECURSOR | | |
| | | | [Saccharomyces cerevisiae] | | |
| 372 | 435314 | EST | ESTs, Moderately similar to 5- | AA699919 | Hs.114018 |
| | | | AMINOLEVULINIC ACID SYNTHASE | | |
| | | | MITOCHONDRIAL PRECURSOR, | | |
| | | | ERYTHROID-SPECIFIC [H. sapiens] | | |
| 373 | 839027 | EST | ESTs, Moderately similar to CGI-24 | AA487499 | |
| | | | protein [H. sapiens] | | |
| 374 | 839027 | EST | ESTs, Moderately similar to CGI-24 | AA487715 | |
| | | | protein [H. sapiens] | | |
| 375 | 321354 | EST | ESTs, Moderately similar to MSG1- | W32403 | Hs.18349 |
| | | | related protein [H. sapiens] | | |
| 376 | 321354 | EST | ESTs, Moderately similar to MSG1- | W32980 | Hs.18349 |
| | | | related protein [H. sapiens] | | |
| 377 | 824911 | EST | ESTs, Weakly similar to/prediction | AA489022 | Hs.5080 |
| 378 | 824911 | EST | ESTs, Weakly similar to/prediction | AA489118 | Hs.5080 |
| 379 | 85384 | EST | ESTs, Weakly similar to anon2A5 | T71965 | Hs.10964 |
| | | | [D. melanogaster] | | |
| 380 | 85384 | EST | ESTs, Weakly similar to anon2A5 | T72105 | Hs.10964 |
| | | | [D. melanogaster] | | |
| 381 | 757265 | EST | ESTs, Weakly similar to aralar1 | AA426113 | Hs.183047 |
| | | | [H. sapiens] | | |
| 382 | 490753 | FLJ20420 | ESTs, Weakly similar to head-elevated | AA133166 | Hs.6693 |
| | | | expression in 0.9 kb [D. melanogaster] | | |
| 383 | 343555 | EST | ESTs, Weakly similar to mitochondrial | W69379 | Hs.62669 |
| | | | inner membrane protease 1 [S. cerevisiae] | | |
| 384 | 343555 | EST | ESTs, Weakly similar to mitochondrial | W69378 | Hs.62669 |
| | | | inner membrane protease 1 [S. cerevisiae] | | |
| 385 | 198312 | KIAA0719 | ESTs, Weakly similar to | R94191 | Hs.21198 |
| | | | MITOCHONDRIAL PRECURSOR | | |
| | | | PROTEINS IMPORT RECEPTOR | | |
| | | | [Neurospora crassa] | | |
| 386 | 198312 | KIAA0719 | ESTs, Weakly similar to | R94290 | Hs.21198 |
| | | | MITOCHONDRIAL PRECURSOR | | |
| | | | PROTEINS IMPORT RECEPTOR | | |
| | | | [Neurospora crassa] | | |
| 387 | 511257 | KIAA0719 | ESTs, Weakly similar to | AA088722 | Hs.21198 |
| | | | MITOCHONDRIAL PRECURSOR | | |
| | | | PROTEINS IMPORT RECEPTOR | | |
| | | | [Neurospora crassa] | | |
| 388 | 511257 | KIAA0719 | ESTs, Weakly similar to | AA088799 | Hs.21198 |
| | | | MITOCHONDRIAL PRECURSOR | | |
| | | | PROTEINS IMPORT RECEPTOR | | |
| | | | [Neurospora crassa] | | |
| 389 | 324651 | EST | ESTs, Weakly similar to | W47099 | Hs.19513 |
| | | | MITOCHONDRIAL RESPIRATORY | | |
| | | | FUNCTION PROTEIN 1 | | |
| | | | [Saccharomyces cerevisiae] | | |
| 390 | 324651 | EST | ESTs, Weakly similar to | W47223 | Hs.19513 |
| | | | MITOCHONDRIAL RESPIRATORY | | |
| | | | FUNCTION PROTEIN 1 | | |
| | | | [Saccharomyces cerevisiae] | | |
| 391 | 429222 | EST | ESTs, Weakly similar to MSF1 | AA004210 | Hs.3945 |
| | | | PROTEIN [S. cerevisiae] | | |
| 392 | 429222 | EST | ESTs, Weakly similar to MSF1 | AA007411 | Hs.3945 |
| | | | PROTEIN [S. cerevisiae] | | |
| 393 | 754537 | EST | ESTs, Weakly similar to myosin beta | AA406291 | |
| | | | heavy chain, cardiac and skeletal muscle | | |
| | | | [H. sapiens] | | |
| 394 | 754537 | EST | ESTs, Weakly similar to myosin beta | AA421979 | |
| | | | heavy chain, cardiac and skeletal muscle | | |
| | | | [H. sapiens] | | |
| 395 | 897557 | EST | ESTs, Weakly similar to myosin beta | AA497024 | Hs.179817 |
| | | | heavy chain, cardiac and skeletal muscle | | |
| | | | [H. sapiens] | | |
| 396 | 897557 | EST | ESTs, Weakly similar to myosin beta | AA489606 | Hs.179817 |
| | | | heavy chain, cardiac and skeletal muscle | | |
| | | | [H. sapiens] | | |
| 397 | 812169 | EST | ESTs, Weakly similar to PROBABLE | AA456042 | Hs.7807 |
| | | | MITOCHONDRIAL 40S RIBOSOMAL | | |
| | | | PROTEIN S5 [S. cerevisiae] | | |
| 398 | 208531 | EST | ESTs, Weakly similar to PUTATIVE | H61979 | Hs.7994 |
| | | | MITOCHONDRIAL CARRIER | | |
| | | | YBR291C [Saccharomyces cerevisiae] | | |
| 399 | 208531 | EST | ESTs, Weakly similar to PUTATIVE | H61978 | Hs.7994 |
| | | | MITOCHONDRIAL CARRIER | | |
| | | | YBR291C [Saccharomyces cerevisiae] | | |
| 400 | 435509 | tRNAleu | ESTs, Weakly similar to similar to | AA701379 | |
| | | | leucyl-tRNA synthetase [C. elegans] | | |
| 401 | 43662 | EST | ESTs, Weakly similar to similar to | H05645 | Hs.21262 |
| | | | mitochrondrial carrier protein [C. elegans] | | |
| 402 | 43662 | EST | ESTs, Weakly similar to similar to | H05644 | Hs.21262 |
| | | | mitochrondrial carrier protein [C. elegans] | | |
| 403 | 53385 | EST | ESTs, Weakly similar to Similar to | R16231 | Hs.106620 |
| | | | NAD(P) transhydrogenase, mitochondrial | | |
| | | | [C. elegans] | | |
| 404 | 53385 | EST | ESTs, Weakly similar to Similar to | R16232 | Hs.106620 |
| | | | NAD(P) transhydrogenase, mitochondrial | | |
| | | | [C. elegans] | | |
| 405 | 84880 | EST | ESTs, Weakly similar to VISC PROTEIN | T74882 | Hs.12239 |
| | | | [Escherichia coli] | | |
| 406 | 84880 | EST | ESTs, Weakly similar to VISC PROTEIN | T74939 | Hs.12239 |
| | | | [Escherichia coli] | | |
| 407 | 845419 | FANCA | Fanconi anemia, complementation group A | AA644129 | Hs.86297 |
| 408 | 624634 | FDX1 | ferredoxin 1 | AA187349 | Hs.744 |
| 409 | 624634 | FDX1 | ferredoxin 1 | AA188427 | Hs.744 |
| 410 | 365149 | FECH | ferrochelatase (protoporphyria) | AA025142 | Hs.26 |
| 411 | 365149 | FECH | ferrochelatase (protoporphyria) | AA025157 | Hs.26 |
| 412 | 1469138 | FGA | Fibrinogen, A alpha polypeptide | AA865707 | Hs.90765 |
| 413 | 131839 | FOLR1 | folate receptor 1 (adult) | R24635 | Hs.73769 |
| 414 | 131839 | FOLR1 | folate receptor 1 (adult) | R24530 | Hs.73769 |
| 415 | 146605 | FPRL1 | formyl peptide receptor-like 1 | R80041 | Hs.99855 |
| 416 | 146605 | FPRL1 | formyl peptide receptor-like 1 | R79948 | Hs.99855 |
| 417 | 490600 | FOSL2 | FOS-like antigen 2 | AA101617 | Hs.155210 |
| 418 | 490600 | FOSL2 | FOS-like antigen 2 | AA101616 | Hs.155210 |
| 419 | 279790 | FMR1 | Fragile X mental retardation 1 | N48355 | Hs.89764 |
| 420 | 279790 | FMR1 | Fragile X mental retardation 1 | N49132 | Hs.89764 |
| 421 | 669419 | FRDA | Friedreich ataxia | AA253413 | Hs.95998 |
| 422 | 669419 | FRDA | Friedreich ataxia | AA253388 | Hs.95998 |
| 423 | 469412 | FH | Fumarate hydratase | AA026917 | Hs.75653 |
| 424 | 469412 | FH | Fumarate hydratase | AA026918 | Hs.75653 |
| 425 | 842839 | FUS | fusion, derived from t(12; 16) malignant | AA486284 | |
| | | | liposarcoma | | |
| 426 | 842839 | FUS | fusion, derived from t(12; 16) malignant | AA489305 | |
| | | | liposarcoma | | |
| 427 | 81289 | ACTG2 | Gamma-actin, enteric smooth muscle | T60048 | Hs.77443 |
| | | | form | | |
| 428 | 288663 | GJB1 | gap junction protein, beta 1, 32 kD | N62394 | |
| | | | (connexin 32, Charcot-Marie-Tooth | | |
| | | | neuropathy, X-linked) | | |
| 429 | 288663 | GJB1 | gap junction protein, beta 1, 32 kD | N79360 | |
| | | | (connexin 32, Charcot-Marie-Tooth | | |
| | | | neuropathy, X-linked) | | |
| 430 | 2338827 | GCDH_HUMAN | GCDH_HUMAN | AI693352 | |
| 431 | 66534 | GCKR | glucokinase (hexokinase 4) regulatory | T67007 | Hs.89771 |
| | | | protein | | |
| 432 | 66534 | GCKR | glucokinase (hexokinase 4) regulatory | T67006 | Hs.89771 |
| | | | protein | | |
| 433 | 471498 | GNS | glucosamine (N-acetyl)-6-sulfatase | AA035347 | Hs.2703 |
| | | | (Sanfilippo disease IIID) | | |
| 434 | 122636 | G6PC | Glucose-6-phosphatase | T98886 | Hs.242 |
| 435 | 122636 | G6PC | Glucose-6-phosphatase | T98887 | Hs.242 |
| 436 | 166236 | G6PD | glucose-6-phosphate dehydrogenase | R87497 | |
| 437 | 166236 | G6PD | glucose-6-phosphate dehydrogenase | R88192 | |
| 438 | 753775 | GMPR | Glucose-6-phosphate dehydrogenase | AA406242 | Hs.1435 |
| 439 | 753775 | GMPR | Glucose-6-phosphate dehydrogenase | AA410375 | Hs.1435 |
| 440 | 361565 | GLUD1 | glutamate dehydrogenase 1 | AA018372 | Hs.77508 |
| 441 | 361565 | GLUD1 | glutamate dehydrogenase 1 | AA017175 | Hs.77508 |
| 442 | 51702 | GOT1 | glutamic-oxaloacetic transaminase 1, | H22856 | Hs.597 |
| | | | soluble (aspartate aminotransferase 1) | | |
| 443 | 51702 | GOT1 | glutamic-oxaloacetic transaminase 1, | H22855 | Hs.597 |
| | | | soluble (aspartate aminotransferase 1) | | |
| 444 | 841370 | GOT2 | Glutamic-oxaloacetic transaminase 2, | AA487739 | Hs.170197 |
| | | | mitochondrial (aspartate aminotransferase | | |
| | | | 2) | | |
| 445 | 841370 | GOT2 | Glutamic-oxaloacetic transaminase 2, | AA487521 | Hs.170197 |
| | | | mitochondrial (aspartate aminotransferase | | |
| | | | 2) | | |
| 446 | 810999 | GPX1 | glutathione peroxidase 1 | AA485362 | Hs.76686 |
| 447 | 810999 | GPX1 | glutathione peroxidase 1 | AA485517 | Hs.76686 |
| 448 | 587847 | GPX2 | Glutathione peroxidase 2, gastrointestinal | AA135289 | Hs.2704 |
| 449 | 587847 | GPX2 | Glutathione peroxidase 2, gastrointestinal | AA135152 | Hs.2704 |
| 450 | 855523 | GPX3 | Glutathione peroxidase 3 (plasma) | AA664180 | Hs.172153 |
| 451 | 1555659 | GPX3 | glutathione peroxidase 3 (plasma) | AI147534 | Hs.172153 |
| 452 | 448619 | GSR | Glutathione reductase | AA777289 | Hs.121524 |
| 453 | 811792 | GSS | Glutathione synthetase | AA463458 | Hs.82327 |
| 454 | 628418 | GPD1 | glycerol-3-phosphate dehydrogenase 1 | AA192547 | Hs.255718 |
| | | | (soluble) | | |
| 455 | 628418 | GPD1 | glycerol-3-phosphate dehydrogenase 1 | AA193116 | Hs.255718 |
| | | | (soluble) | | |
| 456 | 428756 | GPD2 | glycerol-3-phosphate dehydrogenase 2 | AA005219 | Hs.93201 |
| | | | (mitochondrial) | | |
| 457 | 428756 | GPD2 | glycerol-3-phosphate dehydrogenase 2 | AA005218 | Hs.93201 |
| | | | (mitochondrial) | | |
| 458 | 42558 | GATM | glycine amidinotransferase (L- | R61229 | Hs.75335 |
| | | | arginine: glycine amidinotransferase) | | |
| 459 | 42558 | GATM | glycine amidinotransferase (L- | R61228 | Hs.75335 |
| | | | arginine: glycine amidinotransferase) | | |
| 460 | 134748 | GCSH | glycine cleavage system protein H | R28294 | Hs.77631 |
| | | | (aminomethyl carrier) | | |
| 461 | 134748 | GCSH | glycine cleavage system protein H | R28081 | Hs.77631 |
| | | | (aminomethyl carrier) | | |
| 462 | 248261 | GLDC | glycine dehydrogenase (decarboxylating; | N78083 | Hs.27 |
| | | | glycine decarboxylase, glycine cleavage | | |
| | | | system protein P) | | |
| 463 | 248261 | GLDC | glycine dehydrogenase (decarboxylating; | N58494 | Hs.27 |
| | | | glycine decarboxylase, glycine cleavage | | |
| | | | system protein P) | | |
| 464 | 45632 | GYS1 | glycogen synthase 1 (muscle) | H08446 | Hs.772 |
| 465 | 45632 | GYS1 | glycogen synthase 1 (muscle) | H08732 | Hs.772 |
| 466 | 245920 | GYS2 | glycogen synthase 2 (liver) | N72934 | Hs.82614 |
| 467 | 245920 | GYS2 | glycogen synthase 2 (liver) | N52282 | Hs.82614 |
| 468 | 1502027 | MGE1 | GrpE-like protein cochaperone mRNA | AA887226 | |
| 469 | 2250688 | GT | GT | AI659294 | |
| 470 | 841008 | GBP1 | Guanylate binding protein 1, interferon- | AA486849 | Hs.62661 |
| | | | inducible, 67 kD | | |
| 471 | 841008 | GBP1 | Guanylate binding protein 1, interferon- | AA486850 | Hs.62661 |
| | | | inducible, 67 kD | | |
| 472 | 796341 | CLCN3 | H. sapiens RNA for CLCN3 | AA461332 | Hs.174139 |
| 473 | 796341 | CLCN3 | H. sapiens RNA for CLCN3 | AA459750 | Hs.174139 |
| 474 | 300237 | No Data | H. sapiens DAP-3 mRNA | N78611 | |
| 475 | 300237 | No Data | H. sapiens DAP-3 mRNA | W07332 | |
| 476 | 341246 | CLPP | H. sapiens mRNA for CLPP | W58658 | Hs.74362 |
| 477 | 341246 | CLPP | H. sapiens mRNA for CLPP | W58337 | Hs.74362 |
| 478 | 1031185 | MCSP | H. sapiens mRNA for mitochondrial | AA609976 | Hs.111850 |
| | | | capsule selenoprotein | | |
| 479 | 259842 | PMPCB | H. sapiens mRNA for M-phase | N29844 | Hs.184211 |
| | | | phosphoprotein, mpp11 | | |
| 480 | 259842 | PMPCB | H. sapiens mRNA for M-phase | N57262 | Hs.184211 |
| | | | phosphoprotein, mpp11 | | |
| 481 | 810942 | IDH3G | H. sapiens mRNA for NAD (H)-specific | AA459380 | Hs.75253 |
| | | | isocitrate dehydrogenase gamma subunit | | |
| | | | precursor | | |
| 482 | 810942 | IDH3G | H. sapiens mRNA for NAD (H)-specific | AA459606 | Hs.75253 |
| | | | isocitrate dehydrogenase gamma subunit | | |
| | | | precursor | | |
| 483 | 530282 | NDUFA1 | H. sapiens mRNA for NADH | AA111999 | Hs.74823 |
| | | | dehydrogenase | | |
| 484 | 530282 | NDUFA1 | H. sapiens mRNA for NADH | AA083784 | Hs.74823 |
| | | | dehydrogenase | | |
| 485 | 813815 | NR1I3 | H. sapiens mRNA for orphan nuclear | AA447727 | Hs.83623 |
| | | | hormone receptor | | |
| 486 | 813815 | NR1I3 | H. sapiens mRNA for orphan nuclear | AA447889 | Hs.83623 |
| | | | hormone receptor | | |
| 487 | 810959 | ARHGDIA | H. sapiens mRNA for rho GDP- | AA459400 | Hs.159161 |
| | | | dissociation Inhibitor 1 | | |
| 488 | 810959 | ARHGDIA | H. sapiens mRNA for rho GDP- | AA459625 | Hs.159161 |
| | | | dissociation Inhibitor 1 | | |
| 489 | 506032 | TIM17 | H. sapiens mRNA for TIM17 preprotein | AA708446 | Hs.20716 |
| | | | translocase | | |
| 490 | 814526 | EST | H. sapiens seb4D mRNA | AA459363 | |
| 491 | 814526 | EST | H. sapiens seb4D mRNA | AA459588 | |
| 492 | 814460 | SURF5 | H. sapiens SURF-5 mRNA | AA459247 | Hs.78354 |
| 493 | 814460 | SURF5 | H. sapiens SURF-5 mRNA | AA459472 | Hs.78354 |
| 494 | 1671299 | HRK | harakiri, BCL2-interacting protein | AI083676 | Hs.87247 |
| | | | (contains only BH3 domain) | | |
| 495 | 1417985 | EST | heat shock 10 kD protein 1 (chaperonin | AA878786 | Hs.203620 |
| | | | 10) | | |
| 496 | 1417985 | EST | heat shock 10 kD protein 1 (chaperonin | AI791247 | Hs.203620 |
| | | | 10) | | |
| 497 | 1870305 | HSPB2 | heat shock 27 kD protein 2 | AI245337 | Hs.78846 |
| 498 | 1354618 | HSPF2 | heat shock 40 kD protein 2 | AA830392 | Hs.172847 |
| 499 | 755904 | HSPA1A | HEAT SHOCK 70 KD PROTEIN 1 | AA496544 | Hs.8997 |
| 500 | 1604342 | HSPA9B | heat shock 70 kD protein 9B (mortalin-2) | AA987644 | Hs.3069 |
| 501 | 884719 | HSPA10 | HEAT SHOCK COGNATE 71 KD | AA629567 | Hs.180414 |
| | | | PROTEIN | | |
| 502 | 253009 | HSPCA | HEAT SHOCK PROTEIN HSP 90- | H88540 | Hs.180532 |
| | | | ALPHA | | |
| 503 | 253009 | HSPCA | HEAT SHOCK PROTEIN HSP 90- | H88588 | Hs.180532 |
| | | | ALPHA | | |
| 504 | 824031 | HSJ2 | heat shock protein, DNAJ-like 2 | AA490946 | |
| 505 | 824031 | HSJ2 | heat shock protein, DNAJ-like 2 | AA491132 | |
| 506 | 1606894 | HSF4 | heat shock transcription factor 4 | AA999776 | Hs.75486 |
| 507 | 562101 | HERC1 | hect (homologous to the E6-AP (UBE3A) | AA211496 | Hs.255561 |
| | | | carboxyl terminus) domain and RCC1 | | |
| | | | (CHC1)-like domain (RLD) 1 | | |
| 508 | 562101 | HERC1 | hect (homologous to the E6-AP (UBE3A) | AA211544 | Hs.255561 |
| | | | carboxyl terminus) domain and RCC1 | | |
| | | | (CHC1)-like domain (RLD) 1 | | |
| 509 | 85259 | HMOX1 | heme oxygenase (decycling) 1 | T71757 | Hs.202833 |
| 510 | 85259 | HMOX1 | heme oxygenase (decycling) 1 | T71606 | Hs.202833 |
| 511 | 745116 | HMOX2 | Heme oxygenase (decycling) 2 | AA626370 | Hs.83853 |
| 512 | 433307 | HNF6A | hepatocyte nuclear factor 6, alpha | AA699732 | Hs.73168 |
| 513 | 840158 | HK1 | Hexokinase 1 | AA485271 | Hs.118625 |
| 514 | 840158 | HK1 | Hexokinase 1 | AA485272 | Hs.118625 |
| 515 | 1338040 | C46157 | HEXOKINASE D, LIVER ISOZYMES | AA811712 | |
| 516 | 211780 | No Data | Hexosaminidase B (beta polypeptide) | H71868 | |
| 517 | 211780 | No Data | Hexosaminidase B (beta polypeptide) | H71081 | |
| 518 | 767441 | HEXB | hexosaminidase B (beta polypeptide) | AA417946 | Hs.51043 |
| 519 | 767441 | HEXB | hexosaminidase B (beta polypeptide) | AA418121 | Hs.51043 |
| 520 | 970591 | HMG1 | High-mobility group (nonhistone | AA683085 | Hs.189509 |
| | | | chromosomal) protein 1 | | |
| 521 | 363103 | HMG2 | High-mobility group (nonhistone | AA019511 | Hs.80684 |
| | | | chromosomal) protein 2 | | |
| 522 | 363103 | HMG2 | High-mobility group (nonhistone | AA019203 | Hs.80684 |
| | | | chromosomal) protein 2 | | |
| 523 | 80924 | HO3 | histidyl-tRNA synthetase | T70104 | Hs.77798 |
| 524 | 80924 | HO3 | histidyl-tRNA synthetase | T70037 | Hs.77798 |
| 525 | 812246 | HLCS | holocarboxylase synthetase (biotin- | AA455043 | Hs.79375 |
| | | | [proprionyl-Coenzyme A-carboxylase | | |
| | | | (ATP-hydrolysing)] ligase) | | |
| 526 | 712577 | HCCS | HOLOCYTOCHROME C-TYPE | AA281548 | Hs.88859 |
| | | | SYNTHETASE | | |
| 527 | 712577 | HCCS | HOLOCYTOCHROME C-TYPE | AA281549 | Hs.88859 |
| | | | SYNTHETASE | | |
| 528 | 611075 | HOXA1 | homeo box A1 | AA173231 | |
| 529 | 611075 | HOXA1 | homeo box A1 | AA173290 | |
| 530 | 244146 | APAF1 | Homo sapiens apoptotic protease | N51014 | Hs.77579 |
| | | | activating factor 1 (Apaf-1) mRNA, | | |
| | | | complete cds | | |
| 531 | 244146 | APAF1 | Homo sapiens apoptotic protease | N72045 | Hs.77579 |
| | | | activating factor 1 (Apaf-1) mRNA, | | |
| | | | complete cds | | |
| 532 | 288796 | BNIP2 | Homo sapiens BCL2/adenovirus E1B | N62514 | Hs.155596 |
| | | | 19 kD-interacting protein 2 (BNIP2) | | |
| | | | mRNA, complete cds | | |
| 533 | 432620 | NOD1 | Homo sapiens caspase recruitment | AA699441 | |
| | | | domain 4 (NOD1) mRNA | | |
| 534 | 853506 | 23956 Mrna | Homo sapiens clone 23956 mRNA, | AA663549 | Hs.8128 |
| | | | partial cds | | |
| 535 | 288888 | EST | Homo sapiens clone 640 unknown | N62617 | Hs.44563 |
| | | | mRNA, complete sequence | | |
| 536 | 288888 | EST | Homo sapiens clone 640 unknown | N78388 | Hs.44563 |
| | | | mRNA, complete sequence | | |
| 537 | 271102 | CCS | Homo sapiens copper chaperone for | N30404 | Hs.5002 |
| | | | superoxide dismutase (CCS) mRNA, | | |
| | | | complete cds | | |
| 538 | 489823 | COX17 | Homo sapiens COX17 mRNA, complete | AA099855 | Hs.16297 |
| | | | cds | | |
| 539 | 489823 | COX17 | Homo sapiens COX17 mRNA, complete | AA099320 | Hs.16297 |
| | | | cds | | |
| 540 | 487348 | DYMPLE | Homo sapiens dynamin-like protein | AA045529 | Hs.180628 |
| | | | mRNA, complete cds | | |
| 541 | 487348 | DYMPLE | Homo sapiens dynamin-like protein | AA040651 | Hs.180628 |
| | | | mRNA, complete cds | | |
| 542 | 359982 | BNIP3 | Homo sapiens E1B 19K/Bcl-2-binding | AA063521 | Hs.79428 |
| | | | protein Nip3 mRNA, nuclear gene | | |
| | | | encoding mitochondrial protein, complete | | |
| | | | cds | | |
| 543 | 359982 | BNIP3 | Homo sapiens E1B 19K/Bcl-2-binding | AA035669 | Hs.79428 |
| | | | protein Nip3 mRNA, nuclear gene | | |
| | | | encoding mitochondrial protein, complete | | |
| | | | cds | | |
| 544 | 395436 | HUEL | Homo sapiens embryonic lung protein | AA757427 | Hs.44053 |
| | | | (HUEL) mRNA, complete cds | | |
| 545 | 1472184 | HUEL | Homo sapiens embryonic lung protein | AA873264 | Hs.44053 |
| | | | (HUEL) mRNA, complete cds | | |
| 546 | 129644 | SSH3BP1 | Homo sapiens eps8 binding protein e3B1 | R16667 | Hs.24752 |
| | | | mRNA, complete cds | | |
| 547 | 129644 | SSH3BP1 | Homo sapiens eps8 binding protein e3B1 | R16666 | Hs.24752 |
| | | | mRNA, complete cds | | |
| 548 | 563574 | FRG1 | Homo sapiens FRG1 mRNA, complete | AA113339 | Hs.203772 |
| | | | cds | | |
| 549 | 563574 | FRG1 | Homo sapiens FRG1 mRNA, complete | AA112636 | Hs.203772 |
| | | | cds | | |
| 550 | 83279 | TIM23 | Homo sapiens inner mitochondrial | T68317 | Hs.11866 |
| | | | membrane translocase Tim23 (TIM23) | | |
| | | | mRNA, nuclear gene encoding | | |
| | | | mitochondrial protein, complete cds | | |
| 551 | 83279 | TIM23 | Homo sapiens inner mitochondrial | T68386 | Hs.11866 |
| | | | membrane translocase Tim23 (TIM23) | | |
| | | | mRNA, nuclear gene encoding | | |
| | | | mitochondrial protein, complete cds | | |
| 552 | 455123 | KIAA0429 | Homo sapiens KIAA0430 mRNA, | AA676805 | Hs.77694 |
| | | | complete cds | | |
| 553 | 300590 | APACD | Homo sapiens mRNA for ATP binding | N80741 | Hs.153884 |
| | | | protein, complete cds | | |
| 554 | 300590 | APACD | Homo sapiens mRNA for ATP binding | W07537 | Hs.153884 |
| | | | protein, complete cds | | |
| 555 | 129146 | COX7RP | Homo sapiens mRNA for COX7RP, | R10896 | Hs.30888 |
| | | | complete cds | | |
| 556 | 129146 | COX7RP | Homo sapiens mRNA for COX7RP, | R10947 | Hs.30888 |
| | | | complete cds | | |
| 557 | 745314 | HIRIPS | Homo sapiens mRNA for HIRA- | AA625581 | Hs.17368 |
| | | | interacting protein HIRIP5 | | |
| 558 | 810316 | KIAA0631 | Homo sapiens mRNA for KIAA0631 | AA464206 | Hs.75154 |
| | | | protein, partial cds | | |
| 559 | 810316 | KIAA0631 | Homo sapiens mRNA for KIAA0631 | AA464139 | Hs.75154 |
| | | | protein, partial cds | | |
| 560 | 490778 | QP-C | Homo sapiens mRNA for low molecular | AA133191 | Hs.3709 |
| | | | mass ubiquinone-binding protein, | | |
| | | | complete cds | | |
| 561 | 131653 | RPMS12 | Homo sapiens mRNA for mitochondrial | R23752 | Hs.9964 |
| | | | ribosomal protein S12 | | |
| 562 | 131653 | RPMS12 | Homo sapiens mRNA for mitochondrial | R23806 | Hs.9964 |
| | | | ribosomal protein S12 | | |
| 563 | 344825 | LAS | Homo sapiens mRNA for putative lipoic | W72965 | Hs.53531 |
| | | | acid synthetase, partial | | |
| 564 | 344825 | LAS | Homo sapiens mRNA for putative lipoic | W76202 | Hs.53531 |
| | | | acid synthetase, partial | | |
| 565 | 345621 | CXX1 | Homo sapiens mRNA for putatively | W72596 | Hs.255532 |
| | | | prenylated protein | | |
| 566 | 345621 | CXX1 | Homo sapiens mRNA for putatively | W76590 | Hs.255532 |
| | | | prenylated protein | | |
| 567 | 450574 | SCA7 | Homo sapiens mRNA for spinocerebellar | AA704255 | Hs.108447 |
| | | | ataxia 7 | | |
| 568 | 123474 | SCD | Homo sapiens mRNA for stearoyl-CoA | R00707 | Hs.119597 |
| | | | desaturase | | |
| 569 | 123474 | SCD | Homo sapiens mRNA for stearoyl-CoA | R00706 | Hs.119597 |
| | | | desaturase | | |
| 570 | 855890 | cDNA | Homo sapiens mRNA; cDNA | AA630320 | Hs.255914 |
| | | | DKFZp586C0722 (from clone | | |
| | | | DKFZp586C0722) | | |
| 571 | 770043 | NDUFV1 | Homo sapiens NADH: ubiquinone | AA427570 | Hs.7744 |
| | | | dehydrogenase 51 kDa subunit | | |
| | | | (NDUFV1) mRNA, nuclear gene | | |
| | | | encoding mitochondrial protein, complete | | |
| | | | cds | | |
| 572 | 770043 | NDUFV1 | Homo sapiens NADH: ubiquinone | AA427652 | Hs.7744 |
| | | | dehydrogenase 51 kDa subunit | | |
| | | | (NDUFV1) mRNA, nuclear gene | | |
| | | | encoding mitochondrial protein, complete | | |
| | | | cds | | |
| 573 | 859228 | IDH1 | Homo sapiens NADP-dependent | AA666366 | Hs.11223 |
| | | | isocitrate dehydrogenase (IDH) mRNA, | | |
| | | | complete cds | | |
| 574 | 124753 | ABCD4 | Homo sapiens peroxisomal membrane | R02189 | Hs.94395 |
| | | | protein 69 (PMP69) mRNA, complete cds | | |
| 575 | 293104 | PHYH | Homo sapiens peroxisomal phytanoyl- | N91990 | Hs.172887 |
| | | | CoA alpha-hydroxylase (PAHX) mRNA, | | |
| | | | complete cds | | |
| 576 | 293104 | PHYH | Homo sapiens peroxisomal phytanoyl- | N63845 | Hs.172887 |
| | | | CoA alpha-hydroxylase (PAHX) mRNA, | | |
| | | | complete cds | | |
| 577 | 1629264 | hTIM44 | Homo sapiens putative mitochondrial | AI003393 | Hs.123178 |
| | | | inner membrane protein import receptor | | |
| | | | (hTIM44) mRNA, nuclear gene encoding | | |
| | | | mitochondrial protein, complete cds | | |
| 578 | 399532 | RPA40 | Homo sapiens RNA polymerase I subunit | AA733038 | Hs.5409 |
| | | | hRPA39 mRNA, complete cds | | |
| 579 | 787938 | SLC4A4 | Homo sapiens sodium bicarbonate | AA452278 | Hs.5462 |
| | | | cotransporter (HNBC1) mRNA, complete | | |
| | | | cds | | |
| 580 | 138189 | WFS1 | Homo sapiens transmembrane protein | R53910 | Hs.26077 |
| | | | (WFS1) mRNA, complete cds | | |
| 581 | 138189 | WFS1 | Homo sapiens transmembrane protein | R53911 | Hs.26077 |
| | | | (WFS1) mRNA, complete cds | | |
| 582 | 266106 | YWHAE | Human 14-3-3 epsilon mRNA, complete | N21624 | Hs.79474 |
| | | | cds | | |
| 583 | 266106 | YWHAE | Human 14-3-3 epsilon mRNA, complete | N28863 | Hs.79474 |
| | | | cds | | |
| 584 | 45464 | AK2 | Human adenylate kinase 2 (adk2) mRNA, | H09730 | Hs.171811 |
| | | | complete cds | | |
| 585 | 45464 | AK2 | Human adenylate kinase 2 (adk2) mRNA, | H10488 | Hs.171811 |
| | | | complete cds | | |
| 586 | 795543 | AOE372 | Human antioxidant enzyme AOE37-2 | AA459663 | Hs.83383 |
| | | | mRNA, complete cds | | |
| 587 | 241481 | CASP10 | Human apoptotic cysteine protease Mch4 | H80712 | Hs.5353 |
| | | | (Mch4) mRNA, complete cds | | |
| 588 | 241481 | CASP10 | Human apoptotic cysteine protease Mch4 | H80711 | Hs.5353 |
| | | | (Mch4) mRNA, complete cds | | |
| 589 | 897774 | APRT | Human APRT gene for adenine | AA598510 | Hs.28914 |
| | | | phosphoribosyltransferase | | |
| 590 | 810552 | REA | Human B-cell receptor associated protein | AA464669 | Hs.7771 |
| | | | (hBAP) mRNA, partial cds | | |
| 591 | 810552 | REA | Human B-cell receptor associated protein | AA464567 | Hs.7771 |
| | | | (hBAP) mRNA, partial cds | | |
| 592 | 2326129 | BBC3 | Human Bcl-2 binding component 3 | AI688112 | Hs.87246 |
| | | | (bbc3) mRNA, partial cds | | |
| 593 | 795729 | BAD | Human Bcl-2 binding component 6 | AA460291 | Hs.76366 |
| | | | (bbc6) mRNA, complete cds | | |
| 594 | 795729 | BAD | Human Bcl-2 binding component 6 | AA461579 | Hs.76366 |
| | | | (bbc6) mRNA, complete cds | | |
| 595 | 814478 | BCL2A1 | Human Bcl-2 related (Bfl-1) mRNA, | AA459263 | Hs.227817 |
| | | | complete cds | | |
| 596 | 814478 | BCL2A1 | Human Bcl-2 related (Bfl-1) mRNA, | AA459491 | Hs.227817 |
| | | | complete cds | | |
| 597 | 212198 | TP53BP2 | Human Bcl2, p53 binding protein | H69153 | Hs.44585 |
| | | | Bbp/53BP2 (BBP/53BP2) mRNA, | | |
| | | | complete cds | | |
| 598 | 212198 | TP53BP2 | Human Bcl2, p53 binding protein | H69077 | Hs.44585 |
| | | | Bbp/53BP2 (BBP/53BP2) mRNA, | | |
| | | | complete cds | | |
| 599 | 432581 | Novel Gene | Human BRCA2 region, mRNA sequence | AA699390 | Hs.184938 |
| | | | CG012 | | |
| 600 | 433111 | BAC clone CIT987SK-A- | Human Chromosome 16 BAC clone | AA680407 | Hs.47278 |
| | | 735G6 | CIT987SK-A-735G6 | | |
| 601 | 782635 | NDUFAB1 | Human Chromosome 16 BAC clone | AA447569 | Hs.5556 |
| | | | CIT987SK-A-735G6 | | |
| 602 | 782635 | NDUFAB1 | Human Chromosome 16 BAC clone | AA448553 | Hs.5556 |
| | | | CIT987SK-A-735G6 | | |
| 603 | 669471 | 23589 mRNA | Human clone 23589 mRNA sequence | AA234889 | Hs.11506 |
| 604 | 669471 | 23589 mRNA | Human clone 23589 mRNA sequence | AA253479 | Hs.11506 |
| 605 | 771173 | 23732 mRNA | Human clone 23732 mRNA, partial cds | AA443497 | Hs.81281 |
| 606 | 771173 | 23732 mRNA | Human clone 23732 mRNA, partial cds | AA429483 | Hs.81281 |
| 607 | 745606 | 23759 mRNA | Human clone 23759 mRNA, partial cds | AA626336 | Hs.118666 |
| 608 | 323500 | CASP6 | Human cysteine protease Mch2 isoform | W45688 | Hs.3280 |
| | | | alpha (Mch2) mRNA, complete cds | | |
| 609 | 323500 | CASP6 | Human cysteine protease Mch2 isoform | W44316 | Hs.3280 |
| | | | alpha (Mch2) mRNA, complete cds | | |
| 610 | 71420 | CYP2C9 | Human cytochrome P4502C9 (CYP2C9) | T47787 | |
| | | | mRNA, clone 65 | | |
| 611 | 71420 | CYP2C9 | Human cytochrome P4502C9 (CYP2C9) | T47788 | |
| | | | mRNA, clone 65 | | |
| 612 | 754490 | SLC25A16 | Human GT mitochondrial solute carrier | AA411554 | Hs.180408 |
| | | | protein homologue mRNA, complete cds | | |
| 613 | 754490 | SLC25A16 | Human GT mitochondrial solute carrier | AA410572 | Hs.180408 |
| | | | protein homologue mRNA, complete cds | | |
| 614 | 1734348 | Go-alpha | Human guanine nucleotide-binding | AI188097 | Hs.169647 |
| | | | regulatory protein (Go-alpha) gene | | |
| 615 | 826204 | FLII | Human homolog of D. melanogaster | AA521453 | Hs.83849 |
| | | | flightless-I gene product mRNA, partial | | |
| | | | cds | | |
| 616 | 144777 | HAX1 | Human HS1 binding protein HAX-1 | R76263 | Hs.15318 |
| | | | mRNA, nuclear gene encoding | | |
| | | | mitochondrial protein, complete cds | | |
| 617 | 144777 | HAX1 | Human HS1 binding protein HAX-1 | R76544 | Hs.15318 |
| | | | mRNA, nuclear gene encoding | | |
| | | | mitochondrial protein, complete cds | | |
| 618 | 261481 | CUL3 | Human Hs-cul-3 mRNA, partial cds | H98621 | Hs.78946 |
| 619 | 261481 | CUL3 | Human Hs-cul-3 mRNA, partial cds | N25142 | Hs.78946 |
| 620 | 233581 | HIP2 | Human huntingtin interacting protein | H78483 | Hs.155485 |
| | | | (HIP2) mRNA, complete cds | | |
| 621 | 233581 | HIP2 | Human huntingtin interacting protein | H78385 | Hs.155485 |
| | | | (HIP2) mRNA, complete cds | | |
| 622 | 363086 | CKMT1 | Human mitochondrial creatine kinase | AA019482 | Hs.153998 |
| | | | (CKMT) gene, complete cds | | |
| 623 | 363086 | CKMT1 | Human mitochondrial creatine kinase | AA019332 | Hs.153998 |
| | | | (CKMT) gene, complete cds | | |
| 624 | 1390584 | MIPEP | Human mitochondrial intermediate | AA843592 | Hs.68583 |
| | | | peptidase precursor (MIPEP) mRNA, | | |
| | | | mitochondrial gene encoding | | |
| | | | mitochondrial protein, complete cds | | |
| 625 | 134269 | POLRMT | Human mitochondrial RNA polymerase | R31115 | Hs.153880 |
| | | | mRNA, nuclear gene encoding | | |
| | | | mitochondrial protein, complete cds | | |
| 626 | 134269 | POLRMT | Human mitochondrial RNA polymerase | R31174 | Hs.153880 |
| | | | mRNA, nuclear gene encoding | | |
| | | | mitochondrial protein, complete cds | | |
| 627 | 781050 | PFDN5 | Human mRNA for c-myc binding protein, | AA446453 | Hs.80686 |
| | | | complete cds | | |
| 628 | 781050 | PFDN5 | Human mRNA for c-myc binding protein, | AA430010 | Hs.80686 |
| | | | complete cds | | |
| 629 | 451706 | POLA | Human mRNA for DNA polymerase | AA707650 | Hs.74090 |
| | | | alpha-subunit | | |
| 630 | 41541 | EST | Human mRNA for KIAA0017 gene, | R52789 | Hs.253023 |
| | | | complete cds | | |
| 631 | 41541 | EST | Human mRNA for KIAA0017 gene, | R52788 | Hs.253023 |
| | | | complete cds | | |
| 632 | 842818 | KARS | Human mRNA for KIAA0070 gene, | AA486374 | Hs.3100 |
| | | | partial cds | | |
| 633 | 842818 | KARS | Human mRNA for KIAA0070 gene, | AA486220 | Hs.3100 |
| | | | partial cds | | |
| 634 | 280496 | KIAA0123 | Human mRNA for KIAA0123 gene, | N51632 | Hs.75353 |
| | | | partial cds | | |
| 635 | 859627 | KIAA0185 | Human mRNA for KIAA0185 gene, | AA666405 | Hs.255573 |
| | | | partial cds | | |
| 636 | 783698 | KIAA0188 | Human mRNA for KIAA0188 gene, | AA446822 | Hs.81412 |
| | | | partial cds | | |
| 637 | 783698 | KIAA0188 | Human mRNA for KIAA0188 gene, | AA446821 | Hs.81412 |
| | | | partial cds | | |
| 638 | 347031 | KIAA0195 | Human mRNA for KIAA0195 gene, | W79511 | Hs.80540 |
| | | | complete cds | | |
| 639 | 347031 | KIAA0195 | Human mRNA for KIAA0195 gene, | W79398 | Hs.80540 |
| | | | complete cds | | |
| 640 | 753248 | KIAA0381 | Human mRNA for KIAA0381 gene, | AA406231 | Hs.100113 |
| | | | partial cds | | |
| 641 | 753248 | KIAA0381 | Human mRNA for KIAA0381 gene, | AA406503 | Hs.100113 |
| | | | partial cds | | |
| 642 | 108208 | HADHB | Human mRNA for mitochondrial 3- | T69767 | Hs.146812 |
| | | | ketoacyl-CoA thiolase beta-subunit of | | |
| | | | trifunctional protein, complete cds | | |
| 643 | 108208 | HADHB | Human mRNA for mitochondrial 3- | T70752 | Hs.146812 |
| | | | ketoacyl-CoA thiolase beta-subunit of | | |
| | | | trifunctional protein, complete cds | | |
| 644 | 869538 | NDUFA4 | Human NADH: ubiquinone | AA680322 | Hs.108661 |
| | | | oxidoreductase MLRQ subunit mRNA, | | |
| | | | complete cds | | |
| 645 | 810452 | TOM34 | Human putative outer mitochondrial | AA457118 | Hs.76927 |
| | | | membrane 34 kDa translocase hTOM34 | | |
| | | | mRNA, complete cds | | |
| 646 | 127197 | CXORF6 | Human Xq28 mRNA, complete cds | R08270 | Hs.20136 |
| 647 | 127197 | CXORF6 | Human Xq28 mRNA, complete cds | R08271 | Hs.20136 |
| 648 | 811108 | TRIP6 | Human zyxin related protein ZRP-1 | AA485677 | Hs.119498 |
| | | | mRNA, complete cds | | |
| 649 | 811108 | TRIP6 | Human zyxin related protein ZRP-1 | AA485807 | Hs.119498 |
| | | | mRNA, complete cds | | |
| 650 | 79828 | HD | Huntingtin (Huntington disease) | T64094 | Hs.79391 |
| 651 | 79828 | HD | Huntingtin (Huntington disease) | T64015 | Hs.79391 |
| 652 | 1752540 | HIP1 | huntingtin interacting protein 1 | AI150389 | |
| 653 | 1566230 | HYAL3 | hyaluronoglucosaminidase 3 | AI140794 | Hs.129910 |
| 654 | 2095653 | GLO4 | HYDROXYACYLGLUTATHIONE | AI420642 | |
| | | | HYDROLASE | | |
| 655 | 141966 | HSD3B1 | hydroxy-delta-5-steroid dehydrogenase, 3 | R68803 | Hs.38586 |
| | | | beta- and steroid delta-isomerase 1 | | |
| 656 | 141966 | HSD3B1 | hydroxy-delta-5-steroid dehydrogenase, 3 | R68906 | Hs.38586 |
| | | | beta- and steroid delta-isomerase 1 | | |
| 657 | 126243 | HMBS | Hydroxymethylbilane synthase | R06321 | Hs.82609 |
| 658 | 126243 | HMBS | Hydroxymethylbilane synthase | R06263 | Hs.82609 |
| 659 | 321163 | HSU79253 | hypothetical protein | W53000 | Hs.56155 |
| 660 | 321163 | HSU79253 | hypothetical protein | W52999 | Hs.56155 |
| 661 | 280507 | HPRT1 | Hypoxanthine phosphoribosyltransferase | N47312 | Hs.82314 |
| | | | 1 (Lesch-Nyhan syndrome) | | |
| 662 | 280507 | HPRT1 | Hypoxanthine phosphoribosyltransferase | N47311 | Hs.82314 |
| | | | 1 (Lesch-Nyhan syndrome) | | |
| 663 | 2216141 | IFM1 | IFM1 | AI654459 | |
| 664 | 767034 | ILVBL | ilvB (bacterial acetolactate synthase)-like | AA424288 | Hs.78880 |
| 665 | 767034 | ILVBL | ilvB (bacterial acetolactate synthase)-like | AA451741 | Hs.78880 |
| 666 | 1606837 | IMPDH2 | IMP (inosine monophosphate) | AA996028 | Hs.75432 |
| | | | dehydrogenase 2 | | |
| 667 | 50754 | MTIF2 | INITIATION FACTOR IF-2, | H18070 | Hs.149894 |
| | | | MITOCHONDRIAL PRECURSOR | | |
| 668 | 111981 | G1P3 | interferon, alpha-inducible protein (clone | T84633 | Hs.179972 |
| | | | IFI-6-16) | | |
| 669 | 111981 | G1P3 | interferon, alpha-inducible protein (clone | T91807 | Hs.179972 |
| | | | IFI-6-16) | | |
| 670 | 491763 | IL1B | Interleukin 1, beta | AA150507 | Hs.126256 |
| 671 | 491763 | IL1B | Interleukin 1, beta | AA156711 | Hs.126256 |
| 672 | 2119594 | IDH1 | ISOCITRATE DEHYDROGENASE | AI399657 | |
| 673 | 869375 | IDH2 | Isocitrate dehydrogenase 2 (NADP+), | AA679907 | Hs.252546 |
| | | | mitochondrial | | |
| 674 | 2168168 | ETFB | isocitrate dehydrogenase 3 (NAD+) alpha | AI565774 | |
| | | | (IDH3A), | | |
| 675 | 755474 | IARS | isoleucine-tRNA synthetase | AA410636 | |
| 676 | 755474 | IARS | isoleucine-tRNA synthetase | AA419192 | |
| 677 | 810325 | IVD | isovaleryl Coenzyme A dehydrogenase | AA464149 | Hs.77510 |
| 678 | 810325 | IVD | isovaleryl Coenzyme A dehydrogenase | AA464216 | Hs.77510 |
| 679 | 2148505 | JC4913 | JC4913 | AI469831 | |
| 680 | 809707 | JUNB | jun B proto-oncogene | AA454711 | Hs.198951 |
| 681 | 809707 | JUNB | jun B proto-oncogene | AA456366 | Hs.198951 |
| 682 | 1474284 | KAI1 | kangai 1 (suppression of tumorigenicity | AA922309 | Hs.25409 |
| | | | 6, prostate; CD82 antigen (R2 leukocyte | | |
| | | | antigen, antigen detected by monoclonal | | |
| | | | and antibody IA4)) | | |
| 683 | 845441 | KIAA0016 | KIAA0016 gene product | AA644550 | Hs.75187 |
| 684 | 2241563 | KIHUA3 | KIHUA3 | AI632351 | |
| 685 | 21738 | HADHSC | L-3-hydroxyacyl-Coenzyme A | T65482 | Hs.7153 |
| | | | dehydrogenase, short chain | | |
| 686 | 21738 | HADHSC | L-3-hydroxyacyl-Coenzyme A | T65407 | Hs.7153 |
| | | | dehydrogenase, short chain | | |
| 687 | 884644 | LAMR1 | Laminin receptor (2H5 epitope) | AA629897 | Hs.181357 |
| 688 | 704360 | LCFA_HUMAN | LCFA_HUMAN | AA279565 | |
| 689 | 1256792 | MAD4 | Mad4 homolog | AA875977 | Hs.102402 |
| 690 | 1908746 | MDH1 | MALATE DEHYDROGENASE | AI302237 | |
| 691 | 1636908 | MDH2 | malate dehydrogenase 2, NAD | AI000271 | Hs.111076 |
| | | | (mitochondrial) | | |
| 692 | 2010949 | ME2 | malic enzyme 2, mitochondrial | AI361039 | Hs.75342 |
| 693 | 896921 | ME3 | malic enzyme, NADP+-dependent, | AA779401 | Hs.2838 |
| | | | mitochondrial | | |
| 694 | 814211 | M6PR | Mannose-6-phosphate receptor (cation | AA465223 | Hs.75709 |
| | | | dependent) | | |
| 695 | 814211 | M6PR | Mannose-6-phosphate receptor (cation | AA465578 | Hs.75709 |
| | | | dependent) | | |
| 696 | 589115 | MMP1 | matrix metalloproteinase 1 (interstitial | AA143201 | |
| | | | collagenase) | | |
| 697 | 589115 | MMP1 | matrix metalloproteinase 1 (interstitial | AA143331 | |
| | | | collagenase) | | |
| 698 | 1574438 | MMP11 | matrix metalloproteinase 11 (stromelysin | AA954935 | Hs.155324 |
| | | | 3) | | |
| 699 | 196612 | MMP12 | matrix metalloproteinase 12 (macrophage | R92994 | Hs.1695 |
| | | | elastase) | | |
| 700 | 196612 | MMP12 | matrix metalloproteinase 12 (macrophage | R93037 | Hs.1695 |
| | | | elastase) | | |
| 701 | 251047 | MMP17 | matrix metalloproteinase 17 (membrane- | H97792 | Hs.159581 |
| | | | inserted) | | |
| 702 | 784589 | MMP15 | Matrix metalloproteinase 2 | AA443300 | Hs.80343 |
| 703 | 784589 | MMP15 | Matrix metalloproteinase 2 | AA443433 | Hs.80343 |
| 704 | 470393 | MMP7 | matrix metalloproteinase 7 (matrilysin, | AA031513 | Hs.2256 |
| | | | uterine) | | |
| 705 | 470393 | MMP7 | matrix metalloproteinase 7 (matrilysin, | AA031514 | Hs.2256 |
| | | | uterine) | | |
| 706 | 2213770 | CCHU | Matrix metalloproteinase 7 (matrilysin, | AI582304 | |
| | | | uterine) | | |
| 707 | 22040 | MMP9 | matrix metalloproteinase 9 (gelatinase B, | T72581 | |
| | | | 92 kD gelatinase, 92 kD type IV | | |
| | | | collagenase) | | |
| 708 | 22040 | MMP9 | matrix metalloproteinase 9 (gelatinase B, | T64837 | |
| | | | 92 kD gelatinase, 92 kD type IV | | |
| | | | collagenase) | | |
| 709 | 773266 | MMP9 | matrix metalloproteinase 9 (gelatinase B, | AA425227 | Hs.151738 |
| | | | 92 kD gelatinase, 92 kD type IV | | |
| | | | collagenase) | | |
| 710 | 773266 | MMP9 | matrix metalloproteinase 9 (gelatinase B, | AA425503 | Hs.151738 |
| | | | 92 kD gelatinase, 92 kD type IV | | |
| | | | collagenase) | | |
| 711 | 2091860 | MMPL1 | matrix metalloproteinase-like 1 | AI382081 | Hs.198265 |
| 712 | 240766 | Data unavailable | METALLOPROTEINASE INHIBITOR | H80214 | |
| | | | 1 PRECURSOR | | |
| 713 | 240766 | Data unavailable | METALLOPROTEINASE INHIBITOR | H80215 | |
| | | | 1 PRECURSOR | | |
| 714 | 2165301 | DEHUMT | methylene tetrahydrofolate | AI497794 | |
| | | | dehydrogenase (NAD+ dependent), | | |
| | | | methenyltetrahydrofolate cyclohydrolase | | |
| 715 | 1473506 | MMSA_HUMAN | METHYLMALONATE- | AA915881 | |
| | | | SEMIALDEHYDE | | |
| | | | DEHYDROGENASE | | |
| 716 | 2091562 | S40622 | METHYLMALONYL-COA MUTASE | AI377286 | |
| | | | PRECURSOR | | |
| 717 | 278570 | MITF | Microphthalmia-associated transcription | N66177 | Hs.166017 |
| | | | factor | | |
| 718 | 278570 | MITF | Microphthalmia-associated transcription | N99168 | Hs.166017 |
| | | | factor | | |
| 719 | 768443 | MGST1 | microsomal glutathione S-transferase 1 | AA495936 | Hs.790 |
| 720 | 768443 | MGST1 | microsomal glutathione S-transferase 1 | AA495935 | Hs.790 |
| 721 | 629896 | MAP1B | MICROTUBULE-ASSOCIATED | AA219045 | Hs.103042 |
| | | | PROTEIN 1B | | |
| 722 | 1084029 | A56650 | MITOCHONDRIAL 2- | AA594052 | |
| | | | OXOGLUTARATE/MALATE | | |
| | | | CARRIER PROTEIN | | |
| 723 | 44255 | RPML3 | MITOCHONDRIAL 60S RIBOSOMAL | H06113 | Hs.79086 |
| | | | PROTEIN L3 | | |
| 724 | 44255 | RPML3 | MITOCHONDRIAL 60S RIBOSOMAL | H05820 | Hs.79086 |
| | | | PROTEIN L3 | | |
| 725 | 433553 | CA5 | Mitochondrial carbonic anhydrase | AA699469 | Hs.177446 |
| 726 | 1175538 | A39018 | MITOCHONDRIAL CARNITINE | AA641442 | |
| | | | PALMITOYLTRANSFERASE II | | |
| | | | PRECURSOR | | |
| 727 | 324618 | TSFM | MITOCHONDRIAL ELONGATION | W47015 | Hs.3273 |
| | | | FACTOR TS PRECURSOR | | |
| 728 | 324618 | TSFM | MITOCHONDRIAL ELONGATION | W47014 | Hs.3273 |
| | | | FACTOR TS PRECURSOR | | |
| 729 | 2066585 | LON-PEN | MITOCHONDRIAL LON PROTEASE | AI377406 | |
| | | | HOMOLOG | | |
| 730 | 133099 | MMP1 Precursor | MITOCHONDRIAL MATRIX | R26234 | |
| | | | PROTEIN P1 PRECURSOR | | |
| 731 | 133099 | MMP1 Precursor | MITOCHONDRIAL MATRIX | R26235 | |
| | | | PROTEIN P1 PRECURSOR | | |
| 732 | 448491 | mrp2 | MITOCHONDRIAL RIBOSOMAL | AA777627 | |
| | | | PROTEIN S14 | | |
| 733 | 1587031 | HSPA9 | MITOCHONDRIAL STRESS-70 | AA948223 | |
| | | | PROTEIN | | |
| 734 | 814346 | MTIF2 | mitochondrial translational initiation | AA458825 | Hs.149894 |
| | | | factor 2 | | |
| 735 | 814346 | MTIF2 | mitochondrial translational initiation | AA459016 | Hs.149894 |
| | | | factor 2 | | |
| 736 | 1916771 | MTRF1 | mitochondrial translational release factor 1 | AI347695 | Hs.80683 |
| 737 | 1880757 | MAP3K5 | mitogen-activated protein kinase kinase | AI268273 | Hs.151988 |
| | | | kinase 5 | | |
| 738 | 2244621 | MRF1 | MRF1 | AI656905 | |
| 739 | 645006 | MRP4 | MRP4 | AA197284 | |
| 740 | 645006 | MRP4 | MRP4 | AA205815 | |
| 741 | 1933324 | MPZ | myelin protein zero (Charcot-Marie- | AI351956 | Hs.93883 |
| | | | Tooth neuropathy 1B) | | |
| 742 | 302190 | MLL | myeloid/lymphoid or mixed-lineage | W16724 | Hs.199160 |
| | | | leukemia (trithorax (Drosophila) | | |
| | | | homolog) | | |
| 743 | 302190 | MLL | myeloid/lymphoid or mixed-lineage | N77807 | Hs.199160 |
| | | | leukemia (trithorax (Drosophila) | | |
| | | | homolog) | | |
| 744 | 842989 | MYL6 | MYOSIN LIGHT CHAIN ALKALI, | AA488346 | Hs.77385 |
| | | | NON-MUSCLE ISOFORM | | |
| 745 | 842989 | MYL6 | MYOSIN LIGHT CHAIN ALKALI, | AA488477 | Hs.77385 |
| | | | NON-MUSCLE ISOFORM | | |
| 746 | 814615 | MTHFD2 | NAD-dependent methylene | AA480995 | Hs.154672 |
| | | | tetrahydrofolate dehydrogenase | | |
| | | | cyclohydrolase | | |
| 747 | 814615 | MTHFD2 | NAD-dependent methylene | AA480994 | Hs.154672 |
| | | | tetrahydrofolate dehydrogenase | | |
| | | | cyclohydrolase | | |
| 748 | 773287 | NDUFA2 | NADH dehydrogenase (ubiquinone) 1 | AA425211 | Hs.163867 |
| | | | alpha subcomplex, 2 (8 kD, B8) | | |
| 749 | 773287 | NDUFA2 | NADH dehydrogenase (ubiquinone) 1 | AA425534 | Hs.163867 |
| | | | alpha subcomplex, 2 (8 kD, B8) | | |
| 750 | 1635681 | NDUFA2 | NADH dehydrogenase (ubiquinone) 1 | AI017426 | Hs.163867 |
| | | | alpha subcomplex, 2 (8 kD, B8) | | |
| 751 | 950578 | NDUFA5 | NADH dehydrogenase (ubiquinone) 1 | AA608515 | Hs.83916 |
| | | | alpha subcomplex, 5 (13 kD, B13) | | |
| 752 | 897987 | NDUFA9 | NADH dehydrogenase (ubiquinone) 1 | AA598884 | Hs.75227 |
| | | | alpha subcomplex, 9 (39 kD) | | |
| 753 | 450896 | NDUFB4 | NADH dehydrognase (ubiquinone) 1 | AA704675 | |
| | | | beta subcomplex, 4 (15 kD, B15) | | |
| 754 | 562409 | NDUFS5 | NADH dehydrogenase (ubiquinone) 1 | AA214053 | Hs.80595 |
| | | | beta subcomplex, 4 (15 kD, B15) | | |
| 755 | 562409 | NDUFS5 | NADH dehydrogenase (ubiquinone) 1 | AA214154 | Hs.80595 |
| | | | beta subcomplex, 4 (15 kD, B15) | | |
| 756 | 307933 | NDUFB5 | NADH dehydrogenase (ubiquinone) 1 | N93053 | Hs.19236 |
| | | | beta subcomplex, 5 (16 kD, SGDH) | | |
| 757 | 307933 | NDUFB5 | NADH dehydrogenase (ubiquinone) 1 | W21390 | Hs.19236 |
| | | | beta subcomplex, 5 (16 kD, SGDH) | | |
| 758 | 470861 | NDUFB6 | NADH dehydrogenase (ubiquinone) 1 | AA034268 | Hs.109646 |
| | | | beta subcomplex, 6 (17 kD, B17) | | |
| 759 | 470861 | NDUFB6 | NADH dehydrogenase (ubiquinone) 1 | AA032077 | Hs.109646 |
| | | | beta subcomplex, 6 (17 kD, B17) | | |
| 760 | 771089 | NDUFB7 | NADH dehydrogenase (ubiquinone) 1 | AA428058 | Hs.661 |
| | | | beta subcomplex, 7 (18 kD, B18) | | |
| 761 | 771089 | NDUFB7 | NADH dehydrogenase (ubiquinone) 1 | AA429046 | Hs.661 |
| | | | beta subcomplex, 7 (18 kD, B18) | | |
| 762 | 796513 | NDUFC1 | NADH dehydrogenase (ubiquinone) 1, | AA460251 | Hs.84549 |
| | | | subcomplex unknown, 1 (6 kD, KFYI) | | |
| 763 | 796513 | NDUFC1 | NADH dehydrogenase (ubiquinone) 1, | AA463815 | Hs.84549 |
| | | | subcomplex unknown, 1 (6 kD, KFYI) | | |
| 764 | 753457 | NDUFS1 | NADH dehydrogenase (ubiquinone) Fe—S | AA406535 | Hs.8248 |
| | | | protein 1 (75 kD) (NADH-coenzyme Q | | |
| | | | reductase) | | |
| 765 | 753457 | NDUFS1 | NADH dehydrogenase (ubiquinone) Fe—S | AA406536 | Hs.8248 |
| | | | protein 1 (75 kD) (NADH-coenzyme Q | | |
| | | | reductase) | | |
| 766 | 743811 | NDUFS3 | NADH dehydrogenase (ubiquinone) Fe—S | AA634381 | Hs.5273 |
| | | | protein 3 (30 kD) (NADH-coenzyme Q | | |
| | | | reductase) | | |
| 767 | 377152 | NDUFS4 | NADH dehydrogenase (ubiquinone) Fe—S | AA055101 | Hs.10758 |
| | | | protein 4 (18 kD) (NADH-coenzyme Q | | |
| | | | reductase) | | |
| 768 | 377152 | NDUFS4 | NADH dehydrogenase (ubiquinone) Fe—S | AA055102 | Hs.10758 |
| | | | protein 4 (18 kD) (NADH-coenzyme Q | | |
| | | | reductase) | | |
| 769 | 502141 | NDUFS8 | NADH dehydrogenase (ubiquinone) Fe—S | AA127014 | Hs.90443 |
| | | | protein 8 (23 kD) (NADH-coenzyme Q | | |
| | | | reductase) | | |
| 770 | 502141 | NDUFS8 | NADH dehydrogenase (ubiquinone) Fe—S | AA128218 | Hs.90443 |
| | | | protein 8 (23 kD) (NADH-coenzyme Q | | |
| | | | reductase) | | |
| 771 | 1486260 | NDUFV2 | NADH dehydrogenase (ubiquinone) | AA922326 | Hs.51299 |
| | | | flavoprotein 2 (24 kD) | | |
| 772 | 1676983 | RDHUB5 | NADH-CYTOCHROME B5 | AI076798 | |
| | | | REDUCTASE | | |
| 773 | 1632011 | NPR2 | natriuretic peptide receptor B/guanylate | AA994689 | Hs.78518 |
| | | | cyclase B (atrionatriuretic peptide | | |
| | | | receptor B) | | |
| 774 | 994950 | NDUFA10 | NDUFA10 | AA555087 | |
| 775 | 1588691 | NDUFA2 | NDUFA2 | AA975360 | |
| 776 | 929358 | NDUFA3 | NDUFA3 | AA492039 | |
| 777 | 2118546 | NDUFA6 | NDUFA6 | AI524304 | |
| 778 | 1270489 | NDUFA7 | NDUFA7 | AA748476 | |
| 779 | 171756 | NDUFA8 | NDUFA8 | H18333 | |
| 780 | 171756 | NDUFA8 | NDUFA8 | H18364 | |
| 781 | 1473135 | NDUFAB1 | NDUFAB1 | AA873566 | |
| 782 | 998616 | NDUFB1 | NDUFB1 | AA535762 | |
| 783 | 2170527 | NDUFB10 | NDUFB10 | AI571333 | |
| 784 | 1881693 | NDUFB2 | NDUFB2 | AI290799 | |
| 785 | 2103675 | NDUFB3 | NDUFB3 | AI401200 | |
| 786 | 2266937 | NDUFB8 | NDUFB8 | AI608733 | |
| 787 | 984496 | NDUFB9 | NDUFB9 | AA526075 | |
| 788 | 2277817 | NDUFC2 | NDUFC2 | AI653801 | |
| 789 | 1019401 | NDUFS2 | NDUFS2 | AA551149 | |
| 790 | 1585499 | NDUFS5 | NDUFS5 | AA974058 | |
| 791 | 51826 | NNT | nicotinamide nucleotide transhydrogenase | H22944 | Hs.18136 |
| 792 | 51826 | NNT | nicotinamide nucleotide transhydrogenase | H24126 | Hs.18136 |
| 793 | 1160732 | NOS2A | Nitric oxide synthase 2A (inducible, | AA877840 | Hs.193788 |
| | | | hepatocytes) | | |
| 794 | 150466 | EST | Nitric oxide synthase 3 (endothelial cell) | H01039 | Hs.237163 |
| 795 | 150466 | EST | Nitric oxide synthase 3 (endothelial cell) | H01788 | Hs.237163 |
| 796 | 1468820 | NOS3 | nitric oxide synthase 3 (endothelial cell) | AA884967 | Hs.166373 |
| 797 | 755750 | NME2 | non-metastatic cells 2, protein (NM23B) | AA496628 | Hs.255790 |
| | | | expressed in | | |
| 798 | 755750 | NME2 | non-metastatic cells 2, protein (NM23B) | AA496512 | Hs.255790 |
| | | | expressed in | | |
| 799 | 207808 | NFATC3 | nuclear factor of activated T-cells, | H59048 | Hs.172674 |
| | | | cytoplasmic 3 | | |
| 800 | 207808 | NFATC3 | nuclear factor of activated T-cells, | H59047 | Hs.172674 |
| | | | cytoplasmic 3 | | |
| 801 | 1870662 | A54868 | nuclear respiratory factor 1 (NRF1) | AI245773 | |
| 802 | 567414 | NFYB | nuclear transcription factor Y, beta | AA130846 | |
| 803 | 783696 | OAT | ornithine aminotransferase (gyrate | AA446819 | Hs.75485 |
| | | | atrophy) | | |
| 804 | 783696 | OAT | ornithine aminotransferase (gyrate | AA446820 | Hs.75485 |
| | | | atrophy) | | |
| 805 | 796646 | ODC1 | Ornithine decarboxylase 1 | AA460115 | Hs.75212 |
| 806 | 796646 | ODC1 | Ornithine decarboxylase 1 | AA461467 | Hs.75212 |
| 807 | 1637751 | OXA1L | oxidase (cytochrome c) assembly 1-like | AI001180 | Hs.151134 |
| 808 | 1704170 | OXA1HS | oxoglutarate dehydrogenase (lipoamide) | AI096611 | |
| 809 | 233479 | No Data | Oxoglutarate dehydrogenase (lipoamide) | H78910 | |
| 810 | 233479 | No Data | Oxoglutarate dehydrogenase (lipoamide) | H80138 | |
| 811 | 1371793 | OGDH | oxoglutarate dehydrogenase (lipoamide) | AA856769 | Hs.168669 |
| 812 | 1632231 | UQCRC1 | P31930 UBIQUINOL-CYTOCHROME- | AI005342 | |
| | | | C REDUCTASE COMPLEX CORE | | |
| | | | PROTEIN I PRECURSOR | | |
| 813 | 162775 | DDX5 | P68 PROTEIN | H27564 | Hs.76053 |
| 814 | 162775 | DDX5 | P68 PROTEIN | H27646 | Hs.76053 |
| 815 | 43884 | PPIF | PEPTIDYL-PROLYL CIS-TRANS | H05580 | Hs.173125 |
| | | | ISOMERASE, MITOCHONDRIAL | | |
| | | | PRECURSOR | | |
| 816 | 43884 | PPIF | PEPTIDYL-PROLYL CIS-TRANS | H05115 | Hs.173125 |
| | | | ISOMERASE, MITOCHONDRIAL | | |
| | | | PRECURSOR | | |
| 817 | 774726 | PPIF | peptidylprolyl isomerase F (cyclophilin | AA442081 | Hs.173125 |
| | | | F) | | |
| 818 | 774726 | PPIF | peptidylprolyl isomerase F (cyclophilin | AA442184 | Hs.173125 |
| | | | F) | | |
| 819 | 133273 | PMP22 | peripheral myelin protein 22 | R26960 | Hs.103724 |
| 820 | 133273 | PMP22 | peripheral myelin protein 22 | R26732 | Hs.103724 |
| 821 | 788518 | PXMP3 | Peroxisomal membrane protein 3 (35 kD, | AA452566 | |
| | | | Zellweger syndrome) | | |
| 822 | 788518 | PXMP3 | Peroxisomal membrane protein 3 (35 kD, | AA452747 | |
| | | | Zellweger syndrome) | | |
| 823 | 1476157 | PXR1 | peroxisome receptor 1 | AA873073 | Hs.158084 |
| 824 | 455263 | PET112L | PET112 (yeast homolog)-like | AA677572 | Hs.11127 |
| 825 | 951683 | PHC | PHC | AA629980 | |
| 826 | 843109 | FARS1 | phenylalanine-tRNA synthetase | AA488691 | Hs.57969 |
| 827 | 843109 | FARS1 | phenylalanine-tRNA synthetase | AA486503 | Hs.57969 |
| 828 | 842784 | PHC | phosphate carrier, mitochondrial | AA486305 | Hs.78713 |
| 829 | 842784 | PHC | phosphate carrier, mitochondrial | AA486200 | Hs.78713 |
| 830 | 346009 | PFKL | Phosphofructokinase (liver type) | W72140 | Hs.155455 |
| 831 | 346009 | PFKL | Phosphofructokinase (liver type) | W77881 | Hs.155455 |
| 832 | 489626 | PFKM | Phosphofructokinase, muscle | AA099169 | Hs.75160 |
| 833 | 489626 | PFKM | Phosphofructokinase, muscle | AA101919 | Hs.75160 |
| 834 | 183194 | | phospholipase A2, group IIA (platelets, | H44953 | |
| | | | synovial fluid) | | |
| 835 | 183194 | | phospholipase A2, group IIA (platelets, | H45000 | |
| | | | synovial fluid) | | |
| 836 | 77915 | PLA2G2A | phospholipase A2, group IIA (platelets, | T61323 | Hs.76422 |
| | | | synovial fluid) | | |
| 837 | 77915 | PLA2G2A | phospholipase A2, group IIA (platelets, | T61271 | Hs.76422 |
| | | | synovial fluid) | | |
| 838 | 2367279 | PN0673 | PN0673 | AI741984 | |
| 839 | 780947 | POLD1 | Polymerase (DNA directed), delta 1, | AA429661 | Hs.99890 |
| | | | catalytic subunit (125 kD) | | |
| 840 | 780947 | POLD1 | Polymerase (DNA directed), delta 1, | AA446151 | Hs.99890 |
| | | | catalytic subunit (125 kD) | | |
| 841 | 626206 | POLG | polymerase (DNA directed), gamma | AA188761 | Hs.80961 |
| 842 | 626206 | POLG | polymerase (DNA directed), gamma | AA188629 | Hs.80961 |
| 843 | 827129 | POLRMT | polymerase (RNA) mitochondrial (DNA | AA521239 | Hs.153880 |
| | | | directed) | | |
| 844 | 2144562 | PRAX-1 | PRAX-1 | AI452994 | |
| 845 | 826211 | PDCD2 | Programmed cell death 2 | AA521466 | Hs.41639 |
| 846 | 814337 | PCCB | propionyl Coenzyme A carboxylase, beta | AA459122 | Hs.63788 |
| | | | polypeptide | | |
| 847 | 814337 | PCCB | propionyl Coenzyme A carboxylase, beta | AA459341 | Hs.63788 |
| | | | polypeptide | | |
| 848 | 950710 | PCCA | Propionyl-coA carboxylase alpha chain | AA608575 | Hs.80741 |
| 849 | 824568 | KLK3 | Prostate specific antigen | AA490981 | Hs.171995 |
| 850 | 843134 | PBP | Prostatic binding protein | AA486514 | Hs.80423 |
| 851 | 843134 | PBP | Prostatic binding protein | AA485909 | Hs.80423 |
| 852 | 261971 | PRSM1 | protease, metallo, 1, 33 kD | N27227 | Hs.183138 |
| 853 | 261971 | PRSM1 | protease, metallo, 1, 33 kD | H98666 | Hs.183138 |
| 854 | 375728 | PRSM1 | protease, metallo, 1, 33 kD | AA033807 | Hs.183138 |
| 855 | 51041 | Est | protoporphyrinogen oxidase | H18633 | Hs.123641 |
| 856 | 51041 | Est | protoporphyrinogen oxidase | H18716 | Hs.123641 |
| 857 | 247160 | PPOX | protoporphyrinogen oxidase | N57891 | Hs.100016 |
| 858 | 504452 | PPOX | protoporphyrinogen oxidase | AA151249 | Hs.100016 |
| 859 | 504452 | PPOX | protoporphyrinogen oxidase | AA151248 | Hs.100016 |
| 860 | 501939 | HHCMA56 | putative oxidoreductase | AA128041 | Hs.519 |
| 861 | 501939 | HHCMA56 | putative oxidoreductase | AA128086 | Hs.519 |
| 862 | 108378 | PC | pyruvate carboxylase | T77729 | Hs.89890 |
| 863 | 108378 | PC | pyruvate carboxylase | T77728 | Hs.89890 |
| 864 | 80374 | PDHA1 | Pyruvate dehydrogenase (lipoamide) | T65833 | Hs.1023 |
| | | | alpha 1 | | |
| 865 | 80374 | PDHA1 | Pyruvate dehydrogenase (lipoamide) | T65758 | Hs.1023 |
| | | | alpha 1 | | |
| 866 | 826077 | PDHB | pyruvate dehydrogenase (lipoamide) beta | AA521401 | Hs.979 |
| 867 | 279665 | PDX1 | Pyruvate dehydrogenase complex, lipoyl- | N48320 | Hs.74642 |
| | | | containing component X; E3-binding | | |
| | | | protein | | |
| 868 | 279665 | PDX1 | Pyruvate dehydrogenase complex, lipoyl- | N49046 | Hs.74642 |
| | | | containing component X; E3-binding | | |
| | | | protein | | |
| 869 | 1542260 | PDHA2 | PYRUVATE DEHYDROGENASE E1 | AA927400 | |
| | | | COMPONENT, ALPHA | | |
| 870 | 1645668 | PDK1 | pyruvate dehydrogenase kinase, | AI026814 | Hs.159477 |
| | | | isoenzyme 1 | | |
| 871 | 66686 | No Data | QM PROTEIN | T67270 | |
| 872 | 66686 | No Data | QM PROTEIN | T67271 | |
| 873 | 590727 | RENT1 | regulator of nonsense transcripts 1 | AA156342 | Hs.12719 |
| 874 | 590727 | RENT1 | regulator of nonsense transcripts 1 | AA156376 | Hs.12719 |
| 875 | 624627 | RRM2 | Ribonucleotide reductase M2 polypeptide | AA187351 | Hs.75319 |
| 876 | 624627 | RRM2 | Ribonucleotide reductase M2 polypeptide | AA188430 | Hs.75319 |
| 877 | 788334 | RPL23L | ribosomal protein L23-like | AA453015 | Hs.3254 |
| 878 | 730124 | RPL7 | Ribosomal protein L7 | AA412470 | Hs.153 |
| 879 | 730124 | RPL7 | Ribosomal protein L7 | AA412344 | Hs.153 |
| 880 | 809578 | RPS5 | Ribosomal protein S5 | AA456616 | Hs.76194 |
| 881 | 809578 | RPS5 | Ribosomal protein S5 | AA455795 | Hs.76194 |
| 882 | 214133 | FSRG1 | RING3 PROTEIN | H72520 | Hs.75243 |
| 883 | 214133 | FSRG1 | RING3 PROTEIN | H72918 | Hs.75243 |
| 884 | 110744 | SCO2 | SCO (cytochrome oxidase deficient, | T90560 | Hs.180903 |
| | | | yeast) homolog 2 | | |
| 885 | 110744 | SCO2 | SCO (cytochrome oxidase deficient, | T83097 | Hs.180903 |
| | | | yeast) homolog 2 | | |
| 886 | 646657 | SCO1 | SCO1 (yeast homolog) cytochrome | AA205413 | Hs.14511 |
| | | | oxidase deficient 1 | | |
| 887 | 646657 | SCO1 | SCO1 (yeast homolog) cytochrome | AA205579 | Hs.14511 |
| | | | oxidase deficient 1 | | |
| 888 | 1074487 | SDHD | SDHD | AA579646 | |
| 889 | 951117 | SHMT2 | serine hydroxymethyltransferase 2 | AA620477 | |
| | | | (mitochondrial) | | |
| 890 | 2138479 | XNHUSP | SERINE--PYRUVATE | AI521720 | |
| | | | AMINOTRANSFERASE | | |
| 891 | 125183 | SSBP | single-stranded DNA-binding protein | R05693 | Hs.923 |
| 892 | 125183 | SSBP | single-stranded DNA-binding protein | R05694 | Hs.923 |
| 893 | 813678 | SLC1A3 | solute carrier family 1 (glial high affinity | AA453823 | Hs.75379 |
| | | | glutamate transporter), member 3 | | |
| 894 | 813678 | SLC1A3 | solute carrier family 1 (glial high affinity | AA453742 | Hs.75379 |
| | | | glutamate transporter), member 3 | | |
| 895 | 586990 | SLC11A2 | solute carrier family 11 (proton-coupled | AA133656 | Hs.57435 |
| | | | divalent metal ion transporters), member 2 | | |
| 896 | 586990 | SLC11A2 | solute carrier family 11 (proton-coupled | AA133655 | Hs.57435 |
| | | | divalent metal ion transporters), member 2 | | |
| 897 | 2029995 | SLC12A7 | solute carrier family 12 | AI492956 | Hs.172613 |
| | | | (potassium/chloride transporters), | | |
| | | | member 7 | | |
| 898 | 190732 | SLC2A5 | Solute carrier family 2 (facilitated glucose | H38650 | Hs.33084 |
| | | | transporter), member 5 | | |
| 899 | 190732 | SLC2A5 | Solute carrier family 2 (facilitated glucose | H38733 | Hs.33084 |
| | | | transporter), member 5 | | |
| 900 | 758304 | SLC25A14 | solute carrier family 25 (mitochondrial | AA404241 | Hs.194686 |
| | | | carrier, brain), member 14 | | |
| 901 | 758304 | SLC25A14 | solute carrier family 25 (mitochondrial | AA401224 | Hs.194686 |
| | | | carrier, brain), member 14 | | |
| 902 | 897107 | SLC25A1 | solute carrier family 25 (mitochondrial | AA676877 | Hs.111024 |
| | | | carrier; citrate transporter), member 1 | | |
| 903 | 878413 | SLC25A11 | solute carrier family 25 (mitochondrial | AA670357 | Hs.184877 |
| | | | carrier; oxoglutarate carrier), member 11 | | |
| 904 | 433350 | SORD | Sorbitol dehydrogenase | AA700604 | Hs.878 |
| 905 | 1256737 | SPG7 | spastic paraplegia 7, paraplegin (pure and | AA876165 | Hs.78497 |
| | | | complicated autosomal recessive) | | |
| 906 | 682528 | SCA1 | spinocerebellar ataxia 1 | AA256507 | Hs.74520 |
| | | | (olivopontocerebellar ataxia 1, autosomal | | |
| | | | dominant, ataxin 1) | | |
| 907 | 682528 | SCA1 | spinocerebellar ataxia 1 | AA256508 | Hs.74520 |
| | | | (olivopontocerebellar ataxia 1, autosomal | | |
| | | | dominant, ataxin 1) | | |
| 908 | 128875 | SCA2 | Spinocerebellar ataxia 2 | R10604 | Hs.76253 |
| | | | (olivopontocerebellar ataxia 2, autosomal | | |
| | | | dominant) | | |
| 909 | 128875 | SCA2 | Spinocerebellar ataxia 2 | R10603 | Hs.76253 |
| | | | (olivopontocerebellar ataxia 2, autosomal | | |
| | | | dominant) | | |
| 910 | 447167 | SFRS8 | splicing factor, arginine/serine-rich 8 | AA702973 | Hs.84229 |
| | | | (suppressor-of-white-apricot, Drosophila | | |
| | | | homolog) | | |
| 911 | 1934125 | B34181 | steroid 11-beta-hydroxylase | AI334352 | |
| 912 | 859858 | STAR | steroidogenic acute regulatory protein | AA679454 | Hs.3132 |
| 913 | 855395 | SCP2 | sterol carrier protein 2 | AA664009 | Hs.75760 |
| 914 | 824699 | SUCA_HUMAN | SUCA_HUMAN | AA482206 | |
| 915 | 80915 | SDHA | Succinate dehydrogenase 2, flavoprotein | T70043 | |
| | | | (Fp) subunit | | |
| 916 | 80915 | SDHA | Succinate dehydrogenase 2, flavoprotein | T70109 | |
| | | | (Fp) subunit | | |
| 917 | 797016 | SDHB | succinate dehydrogenase complex, | AA463565 | Hs.64 |
| | | | subunit B, iron sulfur (Ip) | | |
| 918 | 797016 | SDHB | succinate dehydrogenase complex, | AA463510 | Hs.64 |
| | | | subunit B, iron sulfur (Ip) | | |
| 919 | 366132 | SDHC | succinate dehydrogenase complex, | AA062805 | Hs.3577 |
| | | | subunit C, integral membrane protein, | | |
| | | | 15 kD | | |
| 920 | 504145 | LSC2 | SUCCINATE-COA LIGASE | AA132000 | |
| 921 | 504145 | LSC2 | SUCCINATE-COA LIGASE | AA131817 | |
| 922 | 845630 | SUCLA2 | succinate-CoA ligase, ADP-forming, beta | AA644577 | Hs.182217 |
| | | | subunit | | |
| 923 | 1571111 | SCOT_HUMAN | Succinyl CoA: 3-oxoacid CoA transferase | AA936982 | |
| 924 | 950489 | SOD1 | Superoxide dismutase 1 (Cu/Zn) | AA599127 | Hs.75428 |
| 925 | 1572467 | SOD2 | Superoxide dismutase 2 | AA934677 | |
| 926 | 346860 | SOD2 | superoxide dismutase 2, mitochondrial | W78148 | Hs.177781 |
| 927 | 346860 | SOD2 | superoxide dismutase 2, mitochondrial | W79913 | Hs.177781 |
| 928 | 840708 | SOD2 | Superoxide dismutase 2, mitochondrial | AA488084 | Hs.177781 |
| 929 | 840708 | SOD2 | Superoxide dismutase 2, mitochondrial | AA487750 | Hs.177781 |
| 930 | 1343732 | SOD3 | Superoxide dismutase 3, extracellular | AA725564 | Hs.2420 |
| 931 | 433474 | SURF1 | Surfeit 1 | AA699560 | Hs.3196 |
| 932 | 2114302 | surfeit 1 | surfeit 1 | AI417847 | Hs.3196 |
| 933 | 782797 | SMN1 | survival of motor neuron 1, telomeric | AA448194 | Hs.77306 |
| 934 | 704299 | TAZ | tafazzin (cardiomyopathy, dilated 3A (X- | AA279440 | Hs.79021 |
| | | | linked); endocardial fibroelastosis 2; | | |
| | | | Barth syndrome) | | |
| 935 | 789376 | TXNRD1 | thioredoxin reductase 1 | AA453335 | Hs.13046 |
| 936 | 789376 | TXNRD1 | thioredoxin reductase 1 | AA464849 | Hs.13046 |
| 937 | 796000 | MPST | thiosulfate sulfurtransferase (rhodanese) | AA461065 | Hs.74097 |
| 938 | 796000 | MPST | thiosulfate sulfurtransferase (rhodanese) | AA460495 | Hs.74097 |
| 939 | 856167 | TARS | threonyl-tRNA synthetase | AA630628 | Hs.84131 |
| 940 | 205185 | THBD | Thrombomodulin | H59861 | Hs.2030 |
| 941 | 205185 | THBD | Thrombomodulin | H60674 | Hs.2030 |
| 942 | 246300 | TIAL1 | TIA1 cytotoxic granule-associated RNA- | N59426 | Hs.182741 |
| | | | binding protein-like 1 | | |
| 943 | 842846 | TIMP2 | tissue inhibitor of metalloproteinase 2 | AA486280 | Hs.246948 |
| 944 | 489519 | TIMP3 | tissue inhibitor of metalloproteinase 3 | AA099153 | Hs.245188 |
| | | | (Sorsby fundus dystrophy, | | |
| | | | pseudoinflammatory) | | |
| 945 | 489519 | TIMP3 | tissue inhibitor of metalloproteinase 3 | AA099251 | Hs.245188 |
| | | | (Sorsby fundus dystrophy, | | |
| | | | pseudoinflammatory) | | |
| 946 | 754106 | TIMP3 | tissue inhibitor of metalloproteinase 3 | AA479202 | Hs.245188 |
| | | | (Sorsby fundus dystrophy, | | |
| | | | pseudoinflammatory) | | |
| 947 | 754106 | TIMP3 | tissue inhibitor of metalloproteinase 3 | AA478662 | Hs.245188 |
| | | | (Sorsby fundus dystrophy, | | |
| | | | pseudoinflammatory) | | |
| 948 | 433481 | TCF2 | transcription factor 2, hepatic; LF-B3; | AA699573 | Hs.169853 |
| | | | variant hepatic nuclear factor | | |
| 949 | 785845 | TCF6L1 | Transcription factor 6-like 1 | AA449118 | Hs.75133 |
| | | | (mitochondrial transcription factor 1-like) | | |
| 950 | 785845 | TCF6L1 | Transcription factor 6-like 1 | AA449551 | Hs.75133 |
| | | | (mitochondrial transcription factor 1-like) | | |
| 951 | 841334 | STIP1 | TRANSFORMATION-SENSITIVE | AA487635 | Hs.75612 |
| | | | PROTEIN IEF SSP 3521 | | |
| 952 | 841334 | STIP1 | TRANSFORMATION-SENSITIVE | AA487427 | Hs.75612 |
| | | | PROTEIN IEF SSP 3521 | | |
| 953 | 2114841 | U66035 | translocase of inner mitochondrial | AI432207 | |
| | | | membrane 8 | | |
| 954 | 970880 | TPM4 | tropomyosin 4 | AA774983 | Hs.102824 |
| 955 | 341328 | TPM1 | Tropomyosin alpha chain (skeletal | W58092 | Hs.77899 |
| | | | muscle) | | |
| 956 | 341328 | TPM1 | Tropomyosin alpha chain (skeletal | W58009 | Hs.77899 |
| | | | muscle) | | |
| 957 | 32621 | WARS2 | tryptophanyl tRNA synthetase 2 | R43272 | Hs.227274 |
| | | | (mitochondrial) | | |
| 958 | 32621 | WARS2 | tryptophanyl tRNA synthetase 2 | R18903 | Hs.227274 |
| | | | (mitochondrial) | | |
| 959 | 684582 | WARS2 | tryptophanyl tRNA synthetase 2 | AA251354 | Hs.227274 |
| | | | (mitochondrial) | | |
| 960 | 684582 | WARS2 | tryptophanyl tRNA synthetase 2 | AA251468 | Hs.227274 |
| | | | (mitochondrial) | | |
| 961 | 594079 | TID1 | tumorous imaginal discs (Drosophila) | AA169872 | Hs.6216 |
| | | | homolog | | |
| 962 | 594079 | TID1 | tumorous imaginal discs (Drosophila) | AA169567 | Hs.6216 |
| | | | homolog | | |
| 963 | 1569989 | YWHAB | tyrosine 3-monooxygenase/tryptophan 5- | AA962407 | Hs.182238 |
| | | | monooxygenase activation protein, beta | | |
| | | | polypeptide | | |
| 964 | 1591788 | YWHAZ | tyrosine 3-monooxygenase/tryptophan 5- | AA976477 | Hs.75103 |
| | | | monooxygenase activation protein, zeta | | |
| | | | polypeptide | | |
| 965 | 25499 | UQCRH | UBIQUINOL-CYTOCHROME C | R11698 | Hs.73818 |
| | | | REDUCTASE COMPLEX 11 KD | | |
| | | | PROTEIN PRECURSOR | | |
| 966 | 25499 | UQCRH | UBIQUINOL-CYTOCHROME C | R17676 | Hs.73818 |
| | | | REDUCTASE COMPLEX 11 KD | | |
| | | | PROTEIN PRECURSOR | | |
| 967 | 855843 | UQCRB | UBIQUINOL-CYTOCHROME C | AA664284 | Hs.131255 |
| | | | REDUCTASE COMPLEX 14 KD | | |
| | | | PROTEIN | | |
| 968 | 1160934 | UQCRC2 | UBIQUINOL-CYTOCHROME C | AA877491 | |
| | | | REDUCTASE CORE PROTEIN 2 | | |
| | | | PRECURSOR | | |
| 969 | 782800 | UQCRFS1 | UBIQUINOL-CYTOCHROME C | AA448184 | Hs.3712 |
| | | | REDUCTASE IRON-SULFUR | | |
| | | | SUBUNIT PRECURSOR | | |
| 970 | 1955460 | MAS2 | UCR1_HUMAN P31930 UBIQUINOL- | AI365986 | |
| | | | CYTOCHROME-C REDUCTASE | | |
| | | | COMPLEX CORE PROTEIN I | | |
| 971 | 236034 | UCP2 | uncoupling protein 2 (mitochondrial, | H61243 | Hs.80658 |
| | | | proton carrier) | | |
| 972 | 236034 | UCP2 | uncoupling protein 2 (mitochondrial, | H61242 | Hs.80658 |
| | | | proton carrier) | | |
| 973 | 628529 | EST | uncoupling protein 3 (mitochondrial, | AA192553 | Hs.76640 |
| | | | proton carrier) | | |
| 974 | 628529 | EST | uncoupling protein 3 (mitochondrial, | AA192136 | Hs.76640 |
| | | | proton carrier) | | |
| 975 | 197932 | UQCRB | UQCRB | R96352 | Hs.77385 |
| 976 | 197932 | UQCRB | UQCRB | R96395 | Hs.77385 |
| 977 | 49464 | UNG | uracil-DNA glycosylase | H15111 | Hs.78853 |
| 978 | 49464 | UNG | uracil-DNA glycosylase | H15112 | Hs.78853 |
| 979 | 760148 | UROD | Uroporphyrinogen decarboxylase | AA424441 | Hs.78601 |
| 980 | 760148 | UROD | Uroporphyrinogen decarboxylase | AA424344 | Hs.78601 |
| 981 | 34778 | VEGF | vascular endothelial growth factor | R45059 | |
| 982 | 34778 | VEGF | vascular endothelial growth factor | R19956 | |
| 983 | 1762200 | VDAC1 | VDAC1 | AI205945 | |
| 984 | 1911533 | VDAC3 | VDAC3 | AI268057 | |
| 985 | 486221 | VDAC1 | Voltage-dependent anion channel 1 | AA044059 | Hs.149155 |
| 986 | 486221 | VDAC1 | Voltage-dependent anion channel 1 | AA044113 | Hs.149155 |
| 987 | 1434908 | VDAC2 | Voltage-dependent anion channel 2 | AA857093 | Hs.78902 |
| 988 | 796759 | VDAC3 | voltage-dependent anion channel 3 | AA460728 | Hs.7381 |
| 989 | 796759 | VDAC3 | voltage-dependent anion channel 3 | AA460900 | Hs.7381 |
| 990 | 815683 | WBSCR9 | Williams-Beuren syndrome chromosome | AA485132 | Hs.194688 |
| | | | region 9 | | |
| 991 | 815683 | WBSCR9 | Williams-Beuren syndrome chromosome | AA485131 | Hs.194688 |
| | | | region 9 | | |
| 992 | 878676 | XRCC5 | X-ray repair complementing defective | AA775355 | Hs.84981 |
| | | | repair in Chinese hamster cells 5 (double- | | |
| | | | strand-break rejoining; Ku autoantigen, | | |
| | | | 80 kD) | | |
| 993 | 269381 | ZNF148 | zinc finger protein 148 (pHZ-52) | N26148 | |
| 994 | 745503 | ZNF9 | zinc finger protein 9 (a cellular retroviral | AA625995 | Hs.2110 |
| | | | nucleic acid binding protein) | | |

### Example 2

Mouse Mitochondrial Biology Array

A mouse mitochondrial biology array is made from clones representing expressed sequences. The clones placed on the array are shown in Table 4 which references sequence ID NOS:995-3040 provided herein setting forth the 5′ and 3′ sequences from these clones. See Tanaka, T. S. et al., (2000) “Genome-wide expression profiling of mid-gestation placenta and embryo using 15 k mouse developmental cDNA microarray” Proc. Natl. Acad. Sci. USA 97:9127-9132. Equivalent clones useful as probes are listed in Table 5. The clones listed in Table 4 are preferable to the clones listed in Table 5. The clones identified in Table 4 are used to make a set of probes called Mouse Probe Set #2. The clones identified in Table 5 are used to make a set of probes called Mouse Probe Set #3. Control sequences are also placed this array. Controls include, but are not limited to blanks, DMSO, probes derived from plant sequences, sequence(s) not involved in mitochondrial biology, and poly adenine (40-60 nucleotides long). Sequences used to make probes for the mouse mitochondrial genes can also be found in GenBank Accession No. J01420, which provides the complete mouse mitochondrial genome. Preferably, the probes used for ATP8 and ATP6 do not cross-hybridize with each other.

TABLE 4

| SEQ ID NO: | NIA Clone No. | Gene | Clone_description | GenBank Accession No. | Unigene Cluster ID | Function |
|---|---|---|---|---|---|---|
| 995 | H3001A07 | Mm.42805 | | | Mm.42805 | Energy/Metabolism |
| 996 | H3001C04 | major histocompatibility locus class III | Mus musculus major histocompatibility | | Mm.7447 | Transcription/Chromatin |
| 997 | | region: butyrophilin-like protein gene, | locus class III region: butyrophilin-like | | | |
| | | partial cds; Notch4, PBX2, RAGE, | protein gene, partial cds; Notch4, | | | |
| | | lysophatidic acid acyl transferase-alpha, | PBX2, RAGE, lysophatidic acid acyl | | | |
| | | palmitoyl-protein thioestera | transferase-alpha, palmitoyl-protein thioestera | | | |
| 998 | H3001F12 | heparan sulfate (glucosamine) 3-O- | Mus musculus heparan sulfate | BG063066 | Mm.100135 | Energy/Metabolism |
| 999 | | sulfotransferase 1 (Hs3st1), | (glucosamine) 3-O-sulfotransferase 1 | | | |
| | | | (Hs3st1), mRNA | | | |
| 1000 | H3001H04 | Rat farnesyltransferase beta subunit | Rat farnesyltransferase beta subunit | BG076556 | Mm.28696 | Energy/Metabolism |
| 1001 | | | mRNA, complete cds | | | |
| 1002 | H3001H05 | CPN10-like protein (Cpn10-rs1) gene | Mus musculus CPN10-like protein | BG063078 | Mm.12970 | Heat Shock/Stress |
| 1003 | | | (Cpn10-rs1) gene, complete cds | | | |
| 1004 | H3002A01 | NADH dehydrogenase (ubiquinone) 1 | Homo sapiens NADH dehydrogenase | BG076562 | Mm.29683 | Energy/Metabolism |
| 1005 | | beta subcomplex, 7 (18 kD, B18) | (ubiquinone) 1 beta subcomplex, 7 | | | |
| | | (NDUFB7), | (18 kD, B18) (NDUFB7), mRNA | | | |
| 1006 | H3002B09 | cytochrome c gene (MC1) | Mouse cytochrome c gene (MC1) | BG063100 | Mm.35389 | Energy/Metabolism |
| 1007 | | | | | | |
| 1008 | H3002D03 | gene prolyl oligopeptidase, exon 11, 12, 13, 14, | Mus musculus gene for prolyl | BG062950 | Mm.90005 | Energy/Metabolism |
| 1009 | | 15 and cds | oligopeptidase, exon 11, 12, 13, 14, 15 | | | |
| | | | and complete cds | | | |
| 1010 | H3002E05 | “Mus musculus RAB1, member RAS | | BG076435 | Mm.14530 | Signal Transduction |
| 1011 | | oncogene family (Rab1), mRNA” | | | | |
| 1012 | H3002E06 | dihydrolipoamide branched chain | Mus musculus dihydrolipoamide | BG076436 | Mm.3636 | Energy/Metabolism |
| 1013 | | transacylase E2 (Dbt), | branched chain transacylase E2 (Dbt), mRNA | | | |
| 1014 | H3002E12 | “Mus musculus aurora-related kinase 2 | | BG076440 | Mm.3488 | Signal Transduction |
| 1015 | | (ARK2) mRNA, complete cds” | | | | |
| 1016 | H3002G11 | glutamate-cysteine ligase catalytic | Mus musculus glutamate-cysteine | BG076460 | Mm.4368 | Energy/Metabolism |
| 1017 | | subunit (GLCLC) | ligase catalytic subunit (GLCLC) | | | |
| | | | mRNA, complete cds | | | |
| 1018 | H3003D05 | DHPS | Homo sapiens deoxyhypusine synthase | BG063127 | Mm.28091 | Energy/Metabolism |
| 1019 | | | (DHPS), mRNA | | | |
| 1020 | H3003D08 | BAG2 | Homo sapiens BCL2-associated | BG076597 | Mm.22449 | Apoptosis |
| 1021 | | | athanogene 2 (BAG2), mRNA | | | |
| 1022 | H3003F02 | ribonucleotide reductase | Mouse DNA for M2 subunit of mouse | BG076613 | Mm.99 | Energy/Metabolism |
| 1023 | | | ribonucleotide reductase (EC 1.17.4.1) | | | |
| 1024 | H3003F06 | GluDH | Mus musculus glutamate dehydrogenase | BG076616 | Mm.10600 | Energy/Metabolism |
| 1025 | | | (Glud), mRNA | | | |
| 1026 | H3003F07 | “Mus musculus calmodulin (Calm), | | BG076617 | Mm.2648 | Signal Transduction |
| 1027 | | mRNA” | | | | |
| 1028 | H3003H01 | “Mus musculus adenylate cyclase 7 | | BG076632 | Mm.141400 | Signal Transduction |
| 1029 | | (Adcy7), mRNA” | | | | |
| 1030 | H3004A02 | ISCU1 (ISCU), alternatively spliced | Homo sapiens ISCU1 (ISCU) mRNA, | BG076641 | Mm.29497 | Energy/Metabolism |
| 1031 | | | complete cds, alternatively spliced | | | |
| 1032 | H3004A04 | mannosyl (alpha-1,3-)-glycoprotein | Homo sapiens mannosyl (alpha-1,3-)- | BG076643 | Mm.86759 | Energy/Metabolism |
| 1033 | | beta-1,4-N- | glycoprotein beta-1,4-N- | | | |
| | | acetylglucosaminyltransferase, | acetylglucosaminyltransferase, | | | |
| | | isoenzyme B (MGAT4B), | isoenzyme B (MGAT4B), mRNA | | | |
| 1034 | H3004A09 | sarco(endo)plasmic reticulum calcium | Mus musculus sarco(endo)plasmic | BG076647 | Mm.100531 | Energy/Metabolism |
| 1035 | | ATPase (SERCA2) gene, promoter | reticulum calcium ATPase (SERCA2) | | | |
| | | region, exons 1-3, and partial cds | gene, promoter region, exons 1-3, and | | | |
| | | | partial cds | | | |
| 1036 | H3004B06 | “Mouse mRNA for peripheral-type | | BG076653 | Mm.1508 | Signal Transduction |
| 1037 | | benzodiazepine receptor, complete cds” | | | | |
| 1038 | H3004C06 | | | | Mm.34776 | Heat Shock/Stress |
| 1039 | | | | | | |
| 1040 | H3004E03 | “Mus musculus group V phospholipase | | BG063219 | Mm.153446 | Signal Transduction |
| 1041 | | A2 mRNA, complete cds” | | | | |
| 1042 | H3004F07 | “Mus musculus protein tyrosine | | BG076689 | Mm.6355 | Signal Transduction |
| 1043 | | phosphatase 4a2 (Ptp4a2), mRNA” | | | | |
| 1044 | H3004G09 | orotidine-5-monophosphate | Mouse orotidine-5-monophosphate | BG076701 | Mm.13145 | Energy/Metabolism |
| 1045 | | decarboxylase, 3 end | decarboxylase mRNA, 3 end | | | |
| 1046 | H3004H01 | TF-1 apoptosis related protein 19 | Mus musculus TF-1 apoptosis related | BG076705 | Mm.21650 | Apoptosis |
| 1047 | | (Tfar19), | protein 19 (Tfar19), mRNA | | | |
| 1048 | H3004H06 | protein arginine N-methyltransferase 1 | Mus musculus protein arginine N- | BG076710 | Mm.27545 | Energy/Metabolism |
| 1049 | | (Mrmt1), alternatively spliced | methyltransferase 1 (Mrmt1) mRNA, | | | |
| | | | complete cds, alternatively spliced | | | |
| 1050 | H3005A04 | H19 and muscle-specific Nctc1 genes, | Mus musculus H19 and muscle-specific | BG076718 | Mm.12970 | Protein |
| 1051 | | sequence | Nctc1 genes, complete sequence | | | Synthesis/Translational |
| | | | | | | Control |
| 1052 | H3005A06 | “Mus musculus mammalian relative of | | BG076720 | Mm.2701 | Heat Shock/Stress |
| 1053 | | DnaJ (Mrj-pending), mRNA” | | | | |
| 1054 | H3005B03 | thioredoxin reductase 1 (Txnrd1), | Mus musculus thioredoxin reductase 1 | BG076728 | Mm.44552 | Energy/Metabolism |
| 1055 | | | (Txnrd1), mRNA | | | |
| 1056 | H3005B06 | amidophosphoribosyltransferase | Rattus norvegicus mRNA for | BG076730 | Mm.27743 | Energy/Metabolism |
| 1057 | | | amidophosphoribosyltransferase | | | |
| 1058 | H3005D01 | dipeptidylpeptidase 4 (Dpp4), | Mus musculus dipeptidylpeptidase 4 | BG076746 | Mm.1151 | Energy/Metabolism |
| 1059 | | | (Dpp4), mRNA | | | |
| 1060 | H3005D03 | orotidine-5-monophosphate | Mouse orotidine-5-monophosphate | BG076748 | Mm.802 | Energy/Metabolism |
| 1061 | | decarboxylase, 3 end | decarboxylase mRNA, 3 end | | | |
| 1062 | H3005E09 | “Homo sapiens antiquitin 1 (ATQ1), | | BG076761 | Mm.30250 | Signal Transduction |
| 1063 | | mRNA” | | | | |
| 1064 | H3005E10 | M. musculus sodium/potassium ATPase | M. musculus mRNA for | BG076762 | Mm.4550 | Energy/Metabolism |
| 1065 | | beta subunit | sodium/potassium ATPase beta subunit | | | |
| 1066 | H3005F06 | “Mus musculus pelle-like protein | | BG076768 | Mm.38241 | Signal Transduction |
| 1067 | | kinase mRNA, complete cds” | | | | |
| 1068 | H3005G01 | glycerol-3-phosphate dehydrogenase | Mouse mRNA for glycerol-3-phosphate | BG076772 | Mm.3711 | Energy/Metabolism |
| 1069 | | | dehydrogenase, complete cds | | | |
| 1070 | H3005G06 | peptidylprolyl isomerase B (Ppib), | Mus musculus peptidylprolyl isomerase | BG076776 | Mm.2412 | Energy/Metabolism |
| 1071 | | | B (Ppib), mRNA | | | |
| 1072 | H3005G12 | methionyl tRNA synthetase | Homo sapiens mRNA for methionyl | BG063326 | Mm.28173 | Energy/Metabolism |
| 1073 | | | tRNA synthetase, complete cds | | | |
| 1074 | H3006E03 | cytochrome c-1 gene | Human cytochrome c-1 gene, complete | BG076814 | Mm.29196 | Apoptosis |
| 1075 | | | cds | | | |
| 1076 | H3006E12 | UDP-glucose: glycoprotein | Homo sapiens UDP- | BG076821 | Mm.45651 | Energy/Metabolism |
| 1077 | | glucosyltransferase 1 (HUGT1), | glucose: glycoprotein | | | |
| | | | glucosyltransferase 1 (HUGT1), mRNA | | | |
| 1078 | H3006G03 | puromycin-sensitive aminopeptidase | Mus musculus puromycin-sensitive | BG076835 | Mm.29824 | Energy/Metabolism |
| 1079 | | (Psa), | aminopeptidase (Psa), mRNA | | | |
| 1080 | H3006H05 | “Mus musculus calmodulin synthesis | | BG076847 | Mm.18041 | Signal Transduction |
| 1081 | | (CaM) cDNA, complete cds” | | | | |
| 1082 | H3006H10 | “Mus musculus circadian locomoter | | BG063409 | Mm.3552 | Signal Transduction |
| 1083 | | output cycles kaput (Clock), mRNA” | | | | |
| 1084 | H3007E02 | glutathione S-transferase pi class | Mus musculus glutathione S-transferase | BG076872 | Mm.426 | Energy/Metabolism |
| 1085 | | (mGSTpiA) and (mGSTpiB) genes | pi class (mGSTpiA) and (mGSTpiB) | | | |
| | | | genes, complete cds | | | |
| 1086 | H3007G07 | Mm.29713 | | | Mm.29713 | Energy/Metabolism |
| 1087 | | | | | | |
| 1088 | H3007G08 | histidyl tRNA synthetase (Hars), | Mus musculus histidyl tRNA synthetase | BG076882 | Mm.10528 | Energy/Metabolism |
| 1089 | | | (Hars), mRNA | | | |
| 1090 | H3008A02 | H. sapiens gene 2-oxoglutarate carrier | H. sapiens gene for 2-oxoglutarate | BG076887 | Mm.28466 | Energy/Metabolism |
| 1091 | | protein | carrier protein | | | |
| 1092 | H3008A05 | Mus musculus GTP-binding protein | | BG076890 | Mm.6698 | Signal Transduction |
| 1093 | | (mSara) homologue mRNA, complete | | | | |
| | | cds | | | | |
| 1094 | H3008B05 | Mus musculus dystroglycan 1 (DAG1) | | BG063514 | Mm.7524 | Matrix/Structural |
| 1095 | | gene, exons 1 and 2 and complete cds | | | | Proteins |
| 1096 | H3008B06 | ferritin heavy chain (Fth), | Mus musculus ferritin heavy chain | BG063515 | Mm.1776 | Energy/Metabolism |
| 1097 | | | (Fth), mRNA | | | |
| 1098 | H3008B07 | H3008B07 | | | | Energy/Metabolism |
| 1099 | | | | | | |
| 1100 | H3008B10 | ATPase, H+ transporting, lysosomal I | Mus musculus ATPase, H+ | BG063518 | Mm.19185 | Energy/Metabolism |
| 1101 | | (Atp6i), | transporting, lysosomal I (Atp6i), mRNA | | | |
| 1102 | H3008C02 | “Mus musculus PKCq-interacting | | BG076907 | Mm.29675 | Signal Transduction |
| 1103 | | protein PICOT (PICOT) mRNA, | | | | |
| | | complete cds” | | | | |
| 1104 | H3008C07 | tryptophan-2,3-dioxygenase (TDO), | Mus musculus tryptophan-2,3- | BG076911 | Mm.21545 | Energy/Metabolism |
| 1105 | | | dioxygenase (TDO), mRNA | | | |
| 1106 | H3008C09 | “HSP90 = heat shock protein [mice, | | AW538216 | Mm.2180 | Heat Shock/Stress |
| 1107 | | heart, mRNA Partial, 806 nt]” | | | | |
| 1108 | H3008C12 | adenylosuccinate lyase (Adsl), | Mus musculus adenylosuccinate lyase | BG076914 | Mm.38151 | Energy/Metabolism |
| 1109 | | | | (Adsl), mRNA | | |
| 1110 | H3008D12 | H3008D12 | | | | Energy/Metabolism |
| 1111 | | | | | | |
| 1112 | H3008E02 | “Mus musculus caseinolytic protease, | | BG076923 | Mm.15243 | Signal Transduction |
| 1113 | | ATP-dependent, (E. coli) proteolytic | | | | |
| | | subunit homolog (Clpp), mRNA” | | | | |
| 1114 | H3008F09 | Mm.3991 | | | Mm.3991 | Energy/Metabolism |
| 1115 | | | | | | |
| 1116 | H3008G05 | Btk locus, alpha-D-galactosidase A | Mus musculus Btk locus, alpha-D- | | Mm.30034 | Matrix/Structural |
| 1117 | | (Ags), ribosomal protein (L44L), and | galactosidase A (Ags), ribosomal | | | Proteins |
| | | Brutons tyrosine kinase (Btk) genes | protein (L44L), and Brutons tyrosine | | | |
| | | | kinase (Btk) genes, complete cds | | | |
| 1118 | H3008H06 | M. musculus gene cathepsin D, exons 6-9 | M. musculus gene for cathepsin D, exons | AW538365 | | Apoptosis |
| 1119 | | | 6-9 | | | |
| 1120 | H3008H07 | serine palmitoyl transferase, subunit II | Homo sapiens serine palmitoyl | | Mm.29034 | |
| 1121 | | gene; and unknown genes | transferase, subunit II gene, complete | | | |
| | | | cds; and unknown genes | | | |
| 1122 | H3009A09 | Slc20a1 | Mus musculus gibbon ape leukemia | BG063583 | Mm.16757 | Energy/Metabolism |
| 1123 | | | virus receptor (Slc20a1) gene, exon 10 | | | |
| | | | and complete cds | | | |
| 1124 | H3009B04 | Fen1 | Mus musculus flap structure specific | BG063590 | Mm.2952 | Energy/Metabolism |
| 1125 | | | endonuclease 1 (Fen1), mRNA | | | |
| 1126 | H3009C03 | Tgm1 | Mus musculus transglutaminase 1, K | BG076966 | Mm.41964 | Energy/Metabolism |
| 1127 | | | polypeptide (Tgm1), mRNA | | | |
| 1128 | H3009C07 | “Mus musculus heat shock protein, 86 kDa | | BG063605 | Mm.1843 | Heat Shock/Stress |
| 1129 | | 1 (Hsp86-1), mRNA” | | | | |
| 1130 | H3009D02 | ATPase delta | Rattus norvegicus delta subunit of F1F0 | BG076975 | Mm.22514 | Energy/Metabolism |
| 1131 | | | ATPase gene, complete cds | | | |
| 1132 | H3009E08 | Cox8a | Mus musculus cytochrome c oxidase, | BG076988 | Mm.14022 | Energy/Metabolism |
| 1133 | | | subunit VIIIa (Cox8a), mRNA | | | |
| 1134 | H3009G03 | “Mus musculus lithium-sensitive myoinositol | | BG063641 | Mm.8089 | Signal Transduction |
| 1135 | | monophosphatase A1 (IMPA1) | | | | |
| | | mRNA, complete cds” | | | | |
| 1136 | H3010C12 | mannosyl (alpha-1,6-)-glycoprotein | Homo sapiens mannosyl (alpha-1,6-)- | BG077070 | Mm.24293 | Energy/Metabolism |
| 1137 | | beta-1,2-N- | glycoprotein beta-1,2-N- | | | |
| | | acetylglucosaminyltransferase | acetylglucosaminyltransferase | | | |
| | | (MGAT2), | (MGAT2), mRNA | | | |
| 1138 | H3010D10 | tumor necrosis factor alpha converting | Mus musculus tumor necrosis factor | BG077079 | Mm.105014 | Apoptosis |
| 1139 | | enzyme (TACE) gene, exon 3, sequence | alpha converting enzyme (TACE) gene, | | | |
| | | | exon 3, complete sequence | | | |
| 1140 | H3010F06 | H3010F06 | | | | Energy/Metabolism |
| 1141 | | | | | | |
| 1142 | H3010F07 | programmed cell death 6 interacting | Mus musculus programmed cell death 6 | BG077095 | Mm.29816 | Apoptosis |
| 1143 | | protein (Pdcd6ip), | interacting protein (Pdcd6ip), mRNA | | | |
| 1144 | H3011A03 | protein-L-isoaspartate (D-aspartate) O- | Mus musculus protein-L-isoaspartate | BG077121 | Mm.25293 | Energy/Metabolism |
| 1145 | | methyltransferase 1 (Pcmt1), | (D-aspartate) O-methyltransferase 1 | | | |
| | | | (Pcmt1), mRNA | | | |
| 1146 | H3011B01 | H. sapiens mitogen inducible gene mig-2 | H. sapiens mitogen inducible gene mig- | | Mm.29842 | Signal Transduction |
| 1147 | | | 2, complete CDS | | | |
| 1148 | H3011C08 | H3011C08 | | | | Energy/Metabolism |
| 1149 | | | | | | |
| 1150 | H3011E06 | mTim17a | | | Mm.2368 | |
| 1151 | | | | | | |
| 1152 | H3011E10 | Sid329 (Sid329), | Mus musculus Sid329 (Sid329), mRNA | | Mm.21743 | |
| 1153 | | | | | | |
| 1154 | H3011H09 | “House mouse mRNA for MAP kinase | | BG077180 | Mm.57203 | Signal Transduction |
| 1155 | | kinase 3b, complete cds” | | | | |
| 1156 | H3012A05 | serine palmitoyl transferase | Homo sapiens serine palmitoyl | | | |
| 1157 | | | transferase, subunit II gene, complete | | | |
| | | | cds; and unknown genes | | | |
| 1158 | H3012A07 | “Mus musculus heat shock 70 protein | | BG077186 | Mm.34184 | Heat Shock/Stress |
| 1159 | | (Hsc70) gene, complete cds” | | | | |
| 1160 | H3012C06 | “Mus musculus scavenger receptor class | | BG077202 | Mm.4603 | Heat Shock/Stress |
| 1161 | | B type I (mSR-BI) mRNA, complete | | | | |
| | | cds” | | | | |
| 1162 | H3012F03 | GLO1 | Homo sapiens glyoxalase I (GLO1), | BG077218 | Mm.17362 | Energy/Metabolism |
| 1163 | | | mRNA | | | |
| 1164 | H3012F10 | “Mus musculus c-src tyrosine kinase | | BG077222 | Mm.21974 | Signal Transduction |
| 1165 | | (Csk), mRNA” | | | | |
| 1166 | H3012H04 | “Mus musculus protein kinase Lkb1 | | BG063794 | Mm.29947 | Signal Transduction |
| 1167 | | (Lkb1) and R29144/1 genes, partial cds” | | | | |
| 1168 | H3013B02 | M. musculus vacuolar adenosine | M. musculus mRNA for vacuolar | BG077252 | Mm.10727 | Energy/Metabolism |
| 1169 | | triphosphatase subunit B | adenosine triphosphatase subunit B | | | |
| 1170 | H3013B08 | pyridoxal (pyridoxine, vitamin B6) | Homo sapiens pyridoxal (pyridoxine, | BG063905 | Mm.26103 | Energy/Metabolism |
| | | kinase (PDXK), | vitamin B6) kinase (PDXK), mRNA | | | |
| 1171 | H3013C04 | “Mus musculus mRNA, complete cds” | | AW539669 | Mm.9846 | Heat Shock/Stress |
| 1172 | | | | | | |
| 1173 | H3013D03 | “Mus musculus RAB10, member RAS | | BG077272 | Mm.21985 | Signal Transduction |
| 1174 | | oncogene family (Rab10), mRNA” | | | | |
| 1175 | H3013D11 | Mouse metallothionein II (MT-II) gene | | BG063925 | Mm.89170 | Heat Shock/Stress |
| 1176 | | | | | | |
| 1177 | H3013E04 | adenylyl cyclase-associated protein | Homo sapiens adenylyl cyclase- | | Mm.43660 | |
| 1178 | | (CAP), | associated protein (CAP), mRNA | | | |
| 1179 | H3013E07 | dolichyl-phosphate alpha-N- | Mus musculus dolichyl-phosphate | BG063933 | Mm.18353 | Energy/Metabolism |
| 1180 | | acetylglucosaminephosphotransferase 2 | alpha-N- | | | |
| | | (Dpagt2), | acetylglucosaminephosphotransferase 2 | | | |
| | | | (Dpagt2), mRNA | | | |
| 1181 | H3013F01 | delta-aminolevulinate dehydratase (Lv), | Mus musculus delta-aminolevulinate | BG063937 | Mm.6988 | Energy/Metabolism |
| 1182 | | | dehydratase (Lv), mRNA | | | |
| 1183 | H3013F02 | “Mus musculus aurora-related kinase 1 | | BG077290 | Mm.11738 | Signal Transduction |
| 1184 | | (ARK1) mRNA, complete cds” | | | | |
| 1185 | H3013G10 | survivin40, survivin121, and | Mus musculus survivin40, survivin121, | BG077309 | Mm.8552 | Apoptosis |
| 1186 | | survivin140 genes, alternative splice | and survivin140 genes, alternative splice | | | |
| | | products | products, complete cds | | | |
| 1187 | H3014A05 | partial aminopeptidase B (APB gene) | Homo sapiens partial mRNA for | BG077324 | Mm.29706 | Energy/Metabolism |
| 1188 | | | aminopeptidase B (APB gene) | | | |
| 1189 | H3014A06 | “Rattus norvegicus protein associating | | | Mm.45272 | Heat Shock/Stress |
| | | with small stress protein PASS1 (Pass1) | | | | |
| | | mRNA, complete cds” | | | | |
| 1190 | H3014A11 | queuine tRNA-ribosyltransferase | Mus musculus queuine tRNA- | BG077329 | Mm.24178 | Energy/Metabolism |
| 1191 | | (LOC60507), | ribosyltransferase (LOC60507), mRNA | | | |
| 1192 | H3014B06 | Mm.29849 | | | Mm.29849 | Energy/Metabolism |
| 1193 | | | | | | |
| 1194 | H3014C08 | Mus musculus surfeit gene 4 (Surf4), | | BG063996 | Mm.2795 | |
| 1195 | | mRNA | | | | |
| 1196 | H3014C10 | | | | | Heat Shock/Stress |
| 1197 | | | | | | |
| 1198 | H3014C12 | solute carrier family 28 (sodium- | Mus musculus solute carrier family 28 | BG077361 | Mm.29510 | Energy/Metabolism |
| 1199 | | coupled nucleoside transporter), | (sodium-coupled nucleoside | | | |
| | | member 2 (Slc28a2), | transporter), member 2 (Slc28a2), | | | |
| | | | mRNA | | | |
| 1200 | H3014E06 | UDP-Gal: betaGlcNAc beta 1,3- | Mus musculus UDP-Gal: betaGlcNAc | BG077355 | Mm.11132 | Energy/Metabolism |
| 1201 | | galactosyltransferase, polypeptide 4 | beta 1,3-galactosyltransferase, | | | |
| | | (B3galt4), | polypeptide 4 (B3galt4), mRNA | | | |
| 1202 | H3014E07 | signal peptidase 21 kDa subunit | Rattus norvegicus mRNA for signal | BG077356 | Mm.27800 | Energy/Metabolism |
| 1203 | | | peptidase 21 kDa subunit, complete cds | | | |
| 1204 | H3014F05 | “Mus musculus S100 calcium-binding | | | Mm.6523 | Signal Transduction |
| | | protein A13 (S100a13), mRNA” | | | | |
| 1205 | H3014F06 | UDP-Gal: betaGlcNAc beta 1,4- | Mus musculus UDP-Gal: betaGlcNAc | BG077374 | Mm.15622 | Energy/Metabolism |
| 1206 | | galactosyltransferase, polypeptide 1 | beta 1,4-galactosyltransferase, | | | |
| | | (B4galt1), | polypeptide 1 (B4galt1), mRNA | | | |
| 1207 | H3014G07 | phosphoprotein enriched in astrocytes | Mus musculus phosphoprotein enriched | BG064035 | Mm.544 | Apoptosis |
| 1208 | | 15 (Pea15), | in astrocytes 15 (Pea15), mRNA | | | |
| 1209 | H3014G08 | protease (prosome, macropain) 26S | Mus musculus protease (prosome, | BG077385 | Mm.665 | Energy/Metabolism |
| 1210 | | subunit, ATPase 5 (Psmc5), | macropain) 26S subunit, ATPase 5 | | | |
| | | | (Psmc5), mRNA | | | |
| 1211 | H3014G09 | protease (prosome, macropain) 26S | Mus musculus protease (prosome, | | Mm.665 | Energy/Metabolism |
| 1212 | | subunit, ATPase 5 (Psmc5), | macropain) 26S subunit, ATPase 5 | | | |
| | | | (Psmc5), mRNA | | | |
| 1213 | H3014H07 | tyrosyl-tRNA synthetase (YARS), | Homo sapiens tyrosyl-tRNA synthetase (YARS), | BG077395 | Mm.20353 | Energy/Metabolism |
| 1214 | | | mRNA | | | |
| 1215 | H3015A01 | major histocompatibility locus class III | Mus musculus major histocompatibility | | Mm.27416 | |
| 1216 | | region: butyrophilin-like protein gene, | locus class III region: butyrophilin-like | | | |
| | | partial cds; Notch4, PBX2, RAGE, | protein gene, partial cds; Notch4, | | | |
| | | lysophatidic acid acyl transferase-alpha, | PBX2, RAGE, lysophatidic acid acyl | | | |
| | | palmitoyl-protein thioestera | transferase-alpha, palmitoyl-protein | | | |
| | | | thioestera | | | |
| 1217 | H3015B03 | hypothetical protein FLJ10535 | Homo sapiens hypothetical protein | BG077413 | Mm.24187 | Energy/Metabolism |
| 1218 | | (FLJ10535), | FLJ10535 (FLJ10535), mRNA | | | |
| 1219 | H3015C02 | methionine adenosyltransferase alpha | Homo sapiens methionine | BG064075 | Mm.29815 | Energy/Metabolism |
| 1220 | | subunit gene fragment | adenosyltransferase alpha subunit gene | | | |
| | | | fragment | | | |
| 1221 | H3015D05 | MSTP029 | Homo sapiens MSTP029 mRNA, | BG077438 | Mm.30147 | Energy/Metabolism |
| 1222 | | | complete cds | | | |
| 1223 | H3015E06 | Tnf receptor-associated factor 1 (Traf1), | Mus musculus Tnf receptor-associated | BG064103 | Mm.12898 | Apoptosis |
| 1224 | | | factor 1 (Traf1), mRNA | | | |
| 1225 | H3016C10 | | | | | Heat Shock/Stress |
| 1226 | | | | | | |
| 1227 | H3016D01 | Mm.20201 | | | Mm.20201 | Energy/Metabolism |
| 1228 | | | | | | |
| 1229 | H3016D08 | BCL2/adenovirus E1B 19 kDa- | Mus musculus BCL2/adenovirus E1B | BG077518 | Mm.29820 | Apoptosis |
| 1230 | | interacting protein 3-like (Bnip31), | 19 kDa-interacting protein 3-like | | | |
| | | | (Bnip31), mRNA | | | |
| 1231 | H3016E10 | Mus musculus mG28K mRNA for | | BG077528 | Mm.28954 | |
| 1232 | | GTP-binding protein like 1, complete | | | | |
| | | cds | | | | |
| 1233 | H3016F03 | Tim44 | Rattus norvegicus mRNA for Tim44, | | Mm.34791 | Matrix/Structural |
| 1234 | | | complete cds | | | Proteins |
| 1235 | H3016F07 | M. musculus glutamyl-tRNA synthetase | M. musculus mRNA for glutamyl-tRNA | BG064194 | Mm.27190 | Energy/Metabolism |
| 1236 | | | synthetase | | | |
| 1237 | H3016F11 | | | | | Heat Shock/Stress |
| 1238 | H3017A02 | ornithine decarboxylase antizyme 2 | Mus musculus ornithine decarboxylase | BG077570 | Mm.675 | Energy/Metabolism |
| 1239 | | (Oaz2), | antizyme 2 (Oaz2), mRNA | | | |
| 1240 | H3017C11 | “Mus musculus RAN guanine | | BG077598 | Mm.143774 | Signal Transduction |
| 1241 | | nucleotide release factor (Rangnrf- | | | | |
| | | pending), mRNA” | | | | |
| 1242 | H3017D04 | “Homo sapiens hect (homologous to the | | BG064250 | Mm.102717 | Heat Shock/Stress |
| 1243 | | E6-AP (UBE3A) carboxyl terminus) | | | | |
| | | domain and RCC1 (CHC1)-like domain | | | | |
| | | (RLD) 1 (HERC1), mRNA” | | | | |
| 1244 | H3017E04 | “Mus musculus cathepsin Z precursor | | BG077611 | Mm.115 | Signal Transduction |
| 1245 | | (Ctsz) mRNA, complete cds” | | | | |
| 1246 | H3017E11 | “Mus musculus retinoblastoma binding | | AW544081 | Mm.1603 | Heat Shock/Stress |
| 1247 | | protein 7 (Rbbp7), mRNA” | | | | |
| 1248 | H3017G06 | phenylalanyl tRNA synthetase beta | Mus musculus phenylalanyl tRNA | BG077633 | Mm.28922 | Energy/Metabolism |
| 1249 | | subunit (Frsb) | synthetase beta subunit (Frsb) mRNA, | | | |
| | | | complete cds | | | |
| 1250 | H3018A02 | B4galt3 | Mus musculus beta-1,4- | BG077551 | Mm.150720 | Energy/Metabolism |
| 1251 | | | galactosyltransferase III (B4galt3), | | | |
| | | | mRNA | | | |
| 1252 | H3018A04 | APB | Homo sapiens partial mRNA for | BG077553 | | Energy/Metabolism |
| 1253 | | | aminopeptidase B (APB gene) | | | |
| 1254 | H3018A08 | “Mus musculus Cctq gene for | | BG064304 | Mm.17989 | Heat Shock/Stress |
| 1255 | | chaperonin containing TCP-1 theta | | | | |
| | | subunit, complete cds” | | | | |
| 1256 | H3018A11 | Cyp17 | Mus musculus cytochrome P450, 17 | BG064307 | Mm.1262 | Energy/Metabolism |
| 1257 | | | (Cyp17), mRNA | | | |
| 1258 | H3018B08 | thimet oligopeptidase | Mus musculus thimet oligopeptidase | BG077656 | Mm.26995 | Energy/Metabolism |
| 1259 | | | mRNA, complete cds | | | |
| 1260 | H3018F06 | | | | | Heat Shock/Stress |
| 1261 | | | | | | |
| 1262 | H3018G08 | UDP-galactose 4 epimerase (GALE) | Homo sapiens UDP-galactose 4 | BG077710 | Mm.20363 | Energy/Metabolism |
| 1263 | | | epimerase (GALE) gene, complete cds | | | |
| 1264 | H3019A07 | ATP binding protein associated with | Homo sapiens ATP binding protein | | Mm.28438 | Energy/Metabolism |
| | | cell differentiation (APACD), | associated with cell differentiation | | | |
| | | | (APACD), mRNA | | | |
| 1265 | H3019B01 | ATPase, Na+/K+ transporting, beta 1 | Mus musculus ATPase, Na+/K+ | BG077733 | Mm.4550 | Energy/Metabolism |
| 1266 | | polypeptide (Atp1b1), | transporting, beta 1 polypeptide | | | |
| | | | (Atp1b1), mRNA | | | |
| 1267 | H3019B03 | acyl-coenzyme A: cholesterol | acyl-coenzyme A: cholesterol | BG077735 | Mm.28099 | Matrix/Structural |
| 1268 | | acyltransferase [mice, peritoneal | acyltransferase [mice, peritoneal | | | Proteins |
| | | macrophages,, 3041 nt] | macrophages, mRNA, 3041 nt] | | | |
| 1269 | H3019B10 | Mus musculus microtubule-associated | | BG077742 | Mm.12625 | Matrix/Structural |
| 1270 | | protein 4 (Mtap4), mRNA | | | | Proteins |
| 1271 | H3019C06 | CGI-107 protein (LOC51012), | Homo sapiens CGI-107 protein | | Mm.30065 | |
| 1272 | | | (LOC51012), mRNA | | | |
| 1273 | H3019C11 | core1 UDP-galactose: N- | Mus musculus core1 UDP-galactose: N- | BG077752 | Mm.102752 | Energy/Metabolism |
| 1274 | | acetylgalactosamine-alpha-R beta 1,3- | acetylgalactosamine-alpha-R beta 1,3- | | | |
| | | galactosyltransferase (C1galt1) | galactosyltransferase (C1galt1) mRNA, | | | |
| | | | complete cds | | | |
| 1275 | H3019C12 | “Mus musculus GTP-binding protein | | BG077753 | Mm.6698 | Signal Transduction |
| 1276 | | (mSara) homologue mRNA, complete | | | | |
| | | cds” | | | | |
| 1277 | H3019D09 | “Mus musculus protein kinase C | | BG077758 | Mm.425 | Signal Transduction |
| 1278 | | inhibitor (mPKCI) mRNA, complete | | | | |
| | | cds” | | | | |
| 1279 | H3019E05 | Mm.38994 | | | Mm.38994 | Energy/Metabolism |
| 1280 | | | | | | |
| 1281 | H3019E12 | F1F0-type ATP synthase subunit d | Homo sapiens F1F0-type ATP synthase | BG077769 | Mm.102755 | Energy/Metabolism |
| 1282 | | | subunit d mRNA, complete cds | | | |
| 1283 | H3019F05 | Mouse tyrosine kinase (c-abl) mRNA | | BG077774 | Mm.1318 | Signal Transduction |
| 1284 | | | | | | |
| 1285 | H3019F06 | domesticus tumor necrosis factor | Mus musculus domesticus mRNA for | BG077775 | Mm.103353 | Apoptosis |
| 1286 | | receptor p60 homologue 1 (Tnfrh1 | tumor necrosis factor receptor p60 | | | |
| | | gene) | homologue 1 (Tnfrh1 gene) | | | |
| 1287 | H3019F07 | “Mus musculus serine/threonine kinase | | BG077776 | Mm.8235 | Signal Transduction |
| 1288 | | 10 (Stk10), mRNA” | | | | |
| 1289 | H3019F10 | phosphomannomutase 2 (Pmm2), | | | | Energy/Metabolism |
| 1290 | | | | | | |
| 1291 | H3019G05 | alkaline phosphatase 2, liver (Akp2), | Mus musculus phosphomannomutase 2 | BG077784 | Mm.9699 | Energy/Metabolism |
| 1292 | | | (Pmm2), mRNA | | | |
| 1293 | H3019H07 | ATPase-like vacuolar proton channel | Mus musculus alkaline phosphatase 2, | BG077796 | Mm.1265 | Energy/Metabolism |
| 1294 | | (Atpl), | liver (Akp2), mRNA | | | |
| 1295 | H3019H11 | H3019F10 | Mus musculus ATPase-like vacuolar | BG077799 | Mm.30155 | Energy/Metabolism |
| 1296 | | | proton channel (Atpl), mRNA | | | |
| 1297 | H3020A02 | nucleoside diphosphatase (ER-UDPase | Mus musculus mRNA for nucleoside | BG077800 | Mm.10211 | Energy/Metabolism |
| 1298 | | gene) | diphosphatase (ER-UDPase gene) | | | |
| 1299 | H3020A07 | serine palmitoyl transferase, subunit II | Homo sapiens serine palmitoyl | | Mm.22626 | |
| 1300 | | gene; and unknown genes | transferase, subunit II gene, complete | | | |
| | | | cds; and unknown genes | | | |
| 1301 | H3020B08 | ferritin light chain 1 (Ftl1), | Mus musculus ferritin light chain 1 | BG077812 | Mm.7500 | Energy/Metabolism |
| 1302 | | | (Ftl1), mRNA | | | |
| 1303 | H3020C02 | “Mouse metallothionein-I (MT-I) gene, | | BG077818 | Mm.2041 | Heat Shock/Stress |
| 1304 | | 5′ end” | | | | |
| 1305 | H3020D10 | isocitrate dehydrogenase 3 (NAD+) | Homo sapiens isocitrate dehydrogenase | BG077913 | Mm.29051 | Energy/Metabolism |
| 1306 | | alpha (IDH3A), | 3 (NAD+) alpha (IDH3A), mRNA | | | |
| 1307 | H3020E01 | “Mus musculus wagneri gene for 105- | | BG064500 | Mm.34828 | Heat Shock/Stress |
| 1308 | | kDa heat shock protein, exon 18 and | | | | |
| | | complete cds” | | | | |
| 1309 | H3020H04 | squalene epoxidase (Sqle), | Mus musculus squalene epoxidase | BG077950 | Mm.22663 | Energy/Metabolism |
| 1310 | | | (Sqle), mRNA | | | |
| 1311 | H3020H07 | BALB/c GDP-dissociation inhibitor | Mus musculus BALB/c GDP- | BG077953 | Mm.28084 | Energy/Metabolism |
| 1312 | | (GDI-1), partial cds | dissociation inhibitor (GDI-1) mRNA, | | | |
| | | | partial cds | | | |
| 1313 | H3020H10 | man 6-P receptor (46MPR) | Mouse man 6-P receptor (46MPR) | BG064540 | Mm.1358 | Energy/Metabolism |
| 1314 | | | mRNA, complete cds | | | |
| 1315 | H3021A02 | programmed cell death 6 (Pdcd6), | Mus musculus programmed cell death 6 | BG077957 | Mm.24254 | Apoptosis |
| 1316 | | | (Pdcd6), mRNA | | | |
| 1317 | H3021A11 | small zinc finger-like protein (Tim13) | Mus musculus small zinc finger-like | BG077964 | Mm.22201 | Energy/Metabolism |
| 1318 | | | protein (Tim13) mRNA, complete cds | | | |
| 1319 | H3021A12 | M. musculus (clone S5) WRS | M. musculus (clone S5) WRS mRNA for | BG077965 | Mm.38433 | Energy/Metabolism |
| 1320 | | tryptophan--tRNA ligase | tryptophan--tRNA ligase | | | |
| 1321 | H3021B05 | M. musculus CI-23 kD gene | M. musculus CI-23 kD gene | BG077969 | Mm.44227 | Energy/Metabolism |
| 1322 | | | | | | |
| 1323 | H3021B08 | mitochondrial carrier peroxisomal | | | Mm.306 | |
| 1324 | | membrane protein, 34 kDa member | | | | |
| | | 17 PMP34 | | | | |
| 1325 | H3021C09 | H3021C09 | | | | Apoptosis |
| 1326 | | | | | | |
| 1327 | H3021D07 | “Mus musculus proliferin related protein | | BG077825 | Mm.3258 | Signal Transduction |
| 1328 | | (Plfr), mRNA” | | | | |
| 1329 | H3021E11 | vacuolar adenosine triphosphatase | Mus musculus vacuolar adenosine | BG064589 | Mm.29771 | Energy/Metabolism |
| 1330 | | subunit A gene | triphosphatase subunit A gene, complete | | | |
| | | | cds | | | |
| 1331 | H3021F01 | aspartyl aminopeptidase (Dnpep), | Mus musculus aspartyl aminopeptidase | BG077839 | Mm.24680 | Energy/Metabolism |
| 1332 | | | (Dnpep), mRNA | | | |
| 1333 | H3021F08 | “Mus musculus chaperonin subunit 7 | | BG077843 | Mm.914 | Heat Shock/Stress |
| 1334 | | (eta) (Cct7), mRNA” | | | | |
| 1335 | H3021G03 | “Mus musculus serine/threonine protein | | BG077848 | Mm.1970 | Signal Transduction |
| 1336 | | phosphatase type 1 alpha mRNA, | | | | |
| | | complete cds” | | | | |
| 1337 | H3021H03 | “Mus musculus Ras suppressor protein 1 | | BG077860 | Mm.905 | Signal Transduction |
| 1338 | | (Rsu1), mRNA” | | | | |
| 1339 | H3022A04 | Cricetulus griseus | Cricetulus griseus mRNA for | BG064623 | Mm.28864 | Energy/Metabolism |
| 1340 | | Phosphatidylglycerophosphate synthase | Phosphatidylglycerophosphate synthase, | | | |
| | | | complete cds | | | |
| 1341 | H3022A05 | “Mus musculus RAB23, member RAS | | BG077872 | Mm.40644 | Signal Transduction |
| 1342 | | oncogene family (Rab23), mRNA” | | | | |
| 1343 | H3022B12 | ribonuclease P (14 kD) (RPP14), | Homo sapiens ribonuclease P (14 kD) | BG064641 | Mm.41801 | Energy/Metabolism |
| 1344 | | | (RPP14), mRNA | | | |
| 1345 | H3022D02 | dolichyl-phosphate beta- | Homo sapiens dolichyl-phosphate beta- | BG064654 | Mm.27890 | Energy/Metabolism |
| 1346 | | glucosyltransferase | glucosyltransferase mRNA, complete | | | |
| | | | cds | | | |
| 1347 | H3022D03 | galactokinase gene | Mus musculus galactokinase gene, | BG064655 | Mm.2820 | Energy/Metabolism |
| 1348 | | | complete cds | | | |
| 1349 | H3022D09 | beta-glucuronidase gene | Mouse beta-glucuronidase gene, | BG077995 | Mm.3317 | Energy/Metabolism |
| 1350 | | | complete cds | | | |
| 1351 | H3022E01 | GLUT4 vesicle protein, partial cds | Mus musculus GLUT4 vesicle protein | BG064664 | Mm.29010 | Energy/Metabolism |
| 1352 | | | mRNA, partial cds | | | |
| 1353 | H3022E09 | a disintegrin-like and metalloprotease | Homo sapiens a disintegrin-like and | BG078006 | Mm.102791 | Energy/Metabolism |
| 1354 | | (reprolysin type) with thrombospondin | metalloprotease (reprolysin type) with | | | |
| | | type 1 motif, 4 (ADAMTS4), | thrombospondin type 1 motif, 4 | | | |
| | | | (ADAMTS4), mRNA | | | |
| 1355 | H3022F06 | malic enzyme, supernatant (Mod1), | Mus musculus malic enzyme, | BG064680 | Mm.29998 | Energy/Metabolism |
| 1356 | | | supematant (Mod1), mRNA | | | |
| 1357 | H3022G02 | 26S protease ATPase (mss1), partial | Mus musculus 26S protease ATPase | BG078023 | Mm.2462 | Energy/Metabolism |
| 1358 | | cds | (mss1) mRNA, partial cds | | | |
| 1359 | H3022G03 | 26S protease ATPase (mss1), partial | Mus musculus 26S protease ATPase | BG078024 | Mm.2462 | Energy/Metabolism |
| 1360 | | cds | (mss1) mRNA, partial cds | | | |
| 1361 | H3022H03 | serine/threonine kinase 17b (apoptosis- | Homo sapiens serine/threonine kinase | BG078034 | Mm.25559 | Apoptosis |
| 1362 | | inducing) (STK17B), | 17b (apoptosis-inducing) (STK17B), | | | |
| | | | mRNA | | | |
| 1363 | H3022H06 | “Mouse serine/threonine phosphatase- | | BG064691 | Mm.849 | Signal Transduction |
| 1364 | | 2C (PP2C) mRNA, complete cds” | | | | |
| 1365 | H3023A10 | folylpolyglutamate synthetase precursor | Mus musculus folylpolyglutamate | BG064717 | Mm.3830 | Energy/Metabolism |
| 1366 | | (Fpgs) | synthetase precursor (Fpgs) mRNA, | | | |
| | | | complete cds | | | |
| 1367 | H3023B01 | | | | | Heat Shock/Stress |
| 1368 | | | | | | |
| 1369 | H3023B11 | “Mouse mRNA for HSP60 protein | | BG064728 | Mm.1777 | Heat Shock/Stress |
| 1370 | | (clones 3T3-7, -9, and -M1)” | | | | |
| 1371 | H3023B12 | Mouse cDNA for heat shock protein 65 | | BG064729 | Mm.1777 | Heat Shock/Stress |
| 1372 | | | | | | |
| 1373 | H3023C05 | Homo sapiens nuclear receptor binding | | BG064733 | Mm.22029 | |
| 1374 | | protein (NRBP), mRNA | | | | |
| 1375 | H3023C07 | peroxiredoxin 5 (Prdx5), | Mus musculus peroxiredoxin 5 (Prdx5), | BG064735 | Mm.6587 | Energy/Metabolism |
| 1376 | | | mRNA | | | |
| 1377 | H3023C08 | ribonucleotide reductase M1 (Rrm1), | Mus musculus ribonucleotide reductase | BG064736 | Mm.656 | Energy/Metabolism |
| 1378 | | | M1 (Rrm1), mRNA | | | |
| 1379 | H3023C09 | Mm.29735 | | | Mm.29735 | Energy/Metabolism |
| 1380 | | | | | | |
| 1381 | H3023D06 | Mus musculus phosphoglycerate kinase | | BG064745 | Mm.188 | Signal Transduction |
| 1382 | | (Pgk1-ps1) processed pseudogene | | | | |
| 1383 | H3023D08 | glutamate pyruvate transaminase (GPT) | Human glutamate pyruvate | BG064747 | Mm.30130 | Energy/Metabolism |
| 1384 | | gene | transaminase (GPT) gene, complete cds | | | |
| 1385 | H3023D09 | partial xylosyltransferase II (XT-II | Mus musculus partial mRNA for | BG078081 | Mm.100638 | Energy/Metabolism |
| 1386 | | gene) | xylosyltransferase II (XT-II gene) | | | |
| 1387 | H3023E11 | SDHD gene small subunit of | Homo sapiens SDHD gene for small | BG078095 | Mm.10406 | Energy/Metabolism |
| 1388 | | cytochrome b of succinate | subunit of cytochrome b of succinate | | | |
| | | dehydrogenase | dehydrogenase, complete cds | | | |
| 1389 | H3023F02 | “Mus musculus phosphatidylinositol-4- | | BG064765 | Mm.3191 | Signal Transduction |
| 1390 | | phosphate 5-kinase, type 1 beta | | | | |
| | | (Pip5k1b), mRNA” | | | | |
| 1391 | H3023F07 | “Mus musculus Ccte gene for | | BG064769 | Mm.1813 | Heat Shock/Stress |
| 1392 | | chaperonin containing TCP-1 epsilon | | | | |
| | | subunit, complete cds” | | | | |
| 1393 | H3023F08 | “Mus musculus Ccte gene for | | BG064770 | Mm.1813 | Heat Shock/Stress |
| 1394 | | chaperonin containing TCP-1 epsilon | | | | |
| | | subunit, complete cds” | | | | |
| 1395 | H3023F09 | glucosamine-6-phosphate deaminase | Mus musculus glucosamine-6- | BG064771 | Mm.22374 | Energy/Metabolism |
| 1396 | | (Gnpda) | phosphate deaminase (Gnpda) mRNA, | | | |
| | | | complete cds | | | |
| 1397 | H3023F10 | “Mus musculus heat shock protein, 86 | | BG064772 | Mm.1843 | Heat Shock/Stress |
| 1398 | | kDa 1 (Hsp86-1), mRNA” | | | | |
| 1399 | H3023F12 | “Mus musculus heat shock protein, 86 | | BG064774 | Mm.1843 | Heat Shock/Stress |
| 1400 | | kDa 1 (Hsp86-1), mRNA” | | | | |
| 1401 | H3023G01 | “Mus musculus heat shock protein, 86 | | BG064775 | Mm.1843 | Heat Shock/Stress |
| 1402 | | kDa 1 (Hsp86-1), mRNA” | | | | |
| 1403 | H3023G05 | phosphoribosyl pyrophosphate | Mus musculus phosphoribosyl | BG064779 | Mm.25198 | Energy/Metabolism |
| | | synthetase 1 (Prps1), | pyrophosphate synthetase 1 (Prps1), | | | |
| | | | mRNA | | | |
| 1404 | H3023H09 | ferritin L-subunit gene exons 1-4 | Mus musculus ferritin L-subunit gene | BG064794 | Mm.7500 | Energy/Metabolism |
| 1405 | | | exons 1-4, complete cds | | | |
| 1406 | H3023H10 | ferritin L-subunit gene exons 1-4 | Mus musculus ferritin L-subunit gene | BG064795 | Mm.7500 | Energy/Metabolism |
| 1407 | | | exons 1-4, complete cds | | | |
| 1408 | H3023H11 | ferritin L-subunit gene exons 1-4 | Mus musculus ferritin L-subunit gene | BG064796 | Mm.7500 | Energy/Metabolism |
| 1409 | | | exons 1-4, complete cds | | | |
| 1410 | H3023H12 | lactate dehydrogenase 1, A chain | Mus musculus lactate dehydrogenase 1, | BG064797 | Mm.26504 | Energy/Metabolism |
| 1411 | | (Ldh1), | A chain (Ldh1), mRNA | | | |
| 1412 | H3024A02 | polymerase, gamma (Polg), | Mus musculus polymerase, gamma | BG064799 | Mm.3616 | DNA Replication |
| 1413 | | | (Polg), mRNA | | | |
| 1414 | H3024A06 | alcohol dehydrogenase 5 (Adh5), | Mus musculus alcohol dehydrogenase 5 | BG064803 | Mm.3874 | Energy/Metabolism |
| 1415 | | | (Adh5), mRNA | | | |
| 1416 | H3024A11 | DNA M2 subunit of ribonucleotide | Mouse DNA for M2 subunit of mouse | BG078138 | Mm.99 | Energy/Metabolism |
| 1417 | | reductase (EC 1.17.4.1) | ribonucleotide reductase (EC 1.17.4.1) | | | |
| 1418 | H3024B03 | “Mus musculus chaperonin subunit 3 | | BG064811 | Mm.3576 | Heat Shock/Stress |
| 1419 | | (gamma) (Cct3), mRNA” | | | | |
| 1420 | H3024B04 | “Mus musculus chaperonin subunit 3 | | BG064812 | Mm.3576 | Heat Shock/Stress |
| 1421 | | (gamma) (Cct3), mRNA” | | | | |
| 1422 | H3024B05 | “Mus musculus chaperonin subunit 3 | | BG064813 | Mm.3576 | Heat Shock/Stress |
| 1423 | | (gamma) (Cct3), mRNA” | | | | |
| 1424 | H3024B06 | “Mus musculus chaperonin subunit 3 | | BG064814 | Mm.3576 | Heat Shock/Stress |
| 1425 | | (gamma) (Cct3), mRNA” | | | | |
| 1426 | H3024B12 | | | | | Heat Shock/Stress |
| 1427 | | | | | | |
| 1428 | H3024C02 | “Homo sapiens oxidative-stress | | BG078151 | Mm.52786 | Heat Shock/Stress |
| 1429 | | responsive 1 (OSR1), mRNA” | | | | |
| 1430 | H3024C04 | phosphoglycerate mutase 1 (Pgam1) | Mus musculus phosphoglycerate | BG064823 | Mm.16783 | Energy/Metabolism |
| 1431 | | | mutase 1 (Pgam1) mRNA, complete cds | | | |
| 1432 | H3024C07 | HEXA gene, exons 2-14 | M. musculus HEXA gene, exons 2-14 | BG064825 | Mm.2284 | Energy/Metabolism |
| 1433 | | | | | | |
| 1434 | H3024C11 | “Mus musculus heat shock protein, 86 kDa | | BG064829 | Mm.1843 | Heat Shock/Stress |
| 1435 | | 1 (Hsp86-1), mRNA” | | | | |
| 1436 | H3024E08 | direct IAP binding protein with low PI | Mus musculus direct IAP binding | BG064850 | Mm.46716 | Apoptosis |
| 1437 | | | protein with low PI mRNA, complete | | | |
| | | | cds | | | |
| 1438 | H3024F05 | Mus musculus putative CCAAT binding | | BG064857 | Mm.24169 | Transcription/Chromatin |
| 1439 | | factor 1 (mCBF) mRNA, alternatively | | | | |
| | | spliced transcript mCBF1, complete cds | | | | |
| 1440 | H3024F06 | serine hydroxymethyl transferase 1 | Mus musculus serine hydroxymethyl | BG078187 | Mm.45993 | Energy/Metabolism |
| 1441 | | (soluble) (Shmt1), | transferase 1 (soluble) (Shmt1), mRNA | | | |
| 1442 | H3024F07 | fumarate hydratase (FH), | Homo sapiens fumarate hydratase (FH), | BG064859 | Mm.41502 | Energy/Metabolism |
| 1443 | | | mRNA | | | |
| 1444 | H3024G02 | phosphoribosyl pyrophosphate | Mus musculus phosphoribosyl | BG064866 | Mm.25198 | Energy/Metabolism |
| 1445 | | synthetase 1 (Prps1), | pyrophosphate synthetase 1 (Prps1), | | | |
| | | | mRNA | | | |
| 1446 | H3024G03 | K+ channel, sequence | Rattus norvegicus K+ channel mRNA, | BG078196 | Mm.40482 | Energy/Metabolism |
| 1447 | | | sequence | | | |
| 1448 | H3024G07 | Murine L-myc gene | | BG064871 | Mm.1055 | Signal Transduction |
| 1449 | | | | | | |
| 1450 | H3024H02 | “Mus musculus p53 binding protein 1 | | BG078205 | Mm.25231 | Signal Transduction |
| 1451 | | mRNA, partial cds” | | | | |
| 1452 | H3024H12 | “Mus musculus breast heat shock 73 | | BG064886 | Mm.2944 | Heat Shock/Stress |
| 1453 | | protein (hsc73) mRNA, complete cds” | | | | |
| 1454 | H3025A01 | “Mus musculus interferon alpha | | BG078215 | Mm.21761 | Heat Shock/Stress |
| 1455 | | responsive protein (15 kDa) (Ifrg15), | | | | |
| | | mRNA” | | | | |
| 1456 | H3025C05 | malate dehydrogenase, soluble (Mor2), | Mus musculus malate dehydrogenase, | BG064914 | Mm.3156 | Energy/Metabolism |
| 1457 | | | soluble (Mor2), mRNA | | | |
| 1458 | H3025D10 | Aga = aspartylglucosaminidase [mice, | Aga = aspartylglucosaminidase [mice, | BG064929 | Mm.41591 | Energy/Metabolism |
| 1459 | | liver, brain, Partial, 1191 nt] | liver, brain, mRNA Partial, 1191 nt] | | | |
| 1460 | H3025E07 | ADP-ribosylarginine hydrolase | Mus musculus ADP-ribosylarginine | BG064935 | Mm.20047 | Energy/Metabolism |
| 1461 | | | hydrolase mRNA, complete cds | | | |
| 1462 | H3025F10 | succinate dehydrogenase Ip subunit, | Mus musculus succinate dehydrogenase | BG064949 | Mm.29141 | Energy/Metabolism |
| 1463 | | partial cds | Ip subunit mRNA, partial cds | | | |
| 14641465 | H3026B03 | HSPC145 protein (HSPC145), | Homo sapiens HSPC145 protein | | Mm.29904 | |
| | | | (HSPC145), mRNA | | | |
| 1466 | H3026B04 | phosphoribosyl pyrophosphate | Homo sapiens phosphoribosyl | BG064988 | Mm.27743 | Energy/Metabolism |
| 1467 | | amidotransferase (PPAT), | pyrophosphate amidotransferase | | | |
| | | | (PPAT), mRNA | | | |
| 1468 | H3026B06 | Mm.100588 | | | Mm.100588 | Energy/Metabolism |
| 1469 | | | | | | |
| 1470 | H3026B07 | ADP-ribosylation factor 1 (Arf1), | Mus musculus ADP-ribosylation factor | BG078294 | Mm.6836 | Energy/Metabolism |
| 1471 | | | 1 (Arf1), mRNA | | | |
| 1472 | H3026B11 | ATPase, Cu++ transporting, beta | Mus musculus ATPase, Cu++ | BG078297 | Mm.102506 | Energy/Metabolism |
| 1473 | | polypeptide (Atp7b), | transporting, beta polypeptide (Atp7b), | | | |
| | | | mRNA | | | |
| 1474 | H3026D06 | tyrosine 3-monooxygenase/tryptophan | Mus musculus tyrosine 3- | BG065012 | Mm.3308 | Energy/Metabolism |
| 1475 | | 5-monooxygenase activation protein, eta | monooxygenase/tryptophan 5- | | | |
| | | polypeptide (Ywhah), | monooxygenase activation protein, eta | | | |
| | | | polypeptide (Ywhah), mRNA | | | |
| 1476 | H3026D10 | “Mus musculus heat shock protein, | | BG065015 | Mm.27897 | Heat Shock/Stress |
| 1477 | | DNAJ-like 2 (Hsj2), mRNA” | | | | |
| 1478 | H3026E07 | cDNA FLJ12225 fis, clone | Homo sapiens cDNA FLJ12225 fis, | BG078316 | Mm.12983 | Energy/Metabolism |
| 1479 | | MAMMA1001139, weakly similar to | clone MAMMA101139, weakly | | | |
| | | SRE-2 PROTEIN | similar to SRE-2 PROTEIN | | | |
| 1480 | H3026F02 | M. musculus GSHPx gene | M. musculus GSHPx gene | BG065030 | Mm.1090 | Energy/Metabolism |
| 1481 | | | | | | |
| 1482 | H3026F05 | acetyl-Coenzyme A dehydrogenase, | Mus musculus acetyl-Coenzyme A | BG065033 | Mm.18759 | Energy/Metabolism |
| 1483 | | short chain (Acads), | dehydrogenase, short chain (Acads), | | | |
| | | | mRNA | | | |
| 1484 | H3026F06 | H. sapiens phosphoenolpyruvate | H. sapiens mRNA for | BG078326 | Mm.29856 | Energy/Metabolism |
| 1485 | | carboxykinase | phosphoenolpyruvate carboxykinase | | | |
| 1486 | H3026F07 | “Mus musculus Ccth gene for | | BG065035 | Mm.914 | Heat Shock/Stress |
| 1487 | | chaperonin containing TCP-1 eta | | | | |
| | | subunit, complete cds” | | | | |
| 1488 | H3026F08 | “Mus musculus Ccth gene for | | BG065036 | Mm.914 | Heat Shock/Stress |
| 1489 | | chaperonin containing TCP-1 eta | | | | |
| | | subunit, complete cds” | | | | |
| 1490 | H3026G10 | mannose phosphate isomerase (MPI), | Homo sapiens mannose phosphate | BG078342 | Mm.34830 | Energy/Metabolism |
| 1491 | | | isomerase (MPI), mRNA | | | |
| 1492 | H3026H09 | methionine adenosyltransferase alpha | Homo sapiens methionine | BG065061 | Mm.29815 | Energy/Metabolism |
| 1493 | | subunit gene fragment | adenosyltransferase alpha subunit gene | | | |
| | | | fragment | | | |
| 1494 | H3026H11 | puromycin-sensitive aminopeptidase | Mus musculus puromycin-sensitive | BG065063 | Mm.29824 | Energy/Metabolism |
| 1495 | | (Psa), | aminopeptidase (Psa), mRNA | | | |
| 1496 | H3027A10 | ATPase-like vacuolar proton channel | Mus musculus ATPase-like vacuolar | BG065073 | Mm.30155 | Energy/Metabolism |
| 1497 | | (Atpl) | proton channel (Atpl), mRNA | | | |
| 1498 | H3027B02 | “Homo sapiens SH3-domain binding | | BG078369 | Mm.62046 | Signal Transduction |
| 1499 | | protein 4 (SH3BP4), mRNA” | | | | |
| 1500 | H3027C06 | transient receptor potential-related | Mus musculus transient receptor | BG065092 | Mm.33819 | Energy/Metabolism |
| 1501 | | protein (ChaK), | potential-related protein (ChaK), mRNA | | | |
| 1502 | H3027E05 | uridine phosphorylase (Upp), | Mus musculus uridine phosphorylase | BG065114 | Mm.4610 | Energy/Metabolism |
| 1503 | | | (Upp), mRNA | | | |
| 1504 | H3027E07 | alpha-enolase (2-phospho-D-glycerate | Mouse mRNA for alpha-enolase (2- | BG078408 | Mm.90587 | Energy/Metabolism |
| 1505 | | hydrolase) (EC 4.2.1.11) | phospho-D-glycerate hydrolase) (EC | | | |
| | | | 4.2.1.11) | | | |
| 1506 | H3027E08 | alpha-enolase (2-phospho-D-glycerate | Mouse mRNA for alpha-enolase (2- | BG078409 | Mm.90587 | Energy/Metabolism |
| 1507 | | hydrolase) (EC 4.2.1.11) | phospho-D-glycerate hydrolase) (EC | | | |
| | | | 4.2.1.11) | | | |
| 1508 | H3027E09 | alpha-enolase (2-phospho-D-glycerate | Mouse mRNA for alpha-enolase (2- | BG078410 | Mm.90587 | Energy/Metabolism |
| 1509 | | hydrolase) (EC 4.2.1.11) | phospho-D-glycerate hydrolase) (EC | | | |
| | | | 4.2.1.11) | | | |
| 1510 | H3027F02 | aspartyl-tRNA synthetase (DARS), | Homo sapiens aspartyl-tRNA synthetase | BG078414 | Mm.28693 | Energy/Metabolism |
| 1511 | | | (DARS), mRNA | | | |
| 1512 | H3027F07 | “Murine MAP kinase kinase 6c mRNA, | | BG065128 | Mm.14487 | Signal Transduction |
| 1513 | | complete cds” | | | | |
| 1514 | H3027F12 | “Mus musculus Cctd gene for | | BG078424 | Mm.6821 | Heat Shock/Stress |
| 1515 | | chaperonin containing TCP-1 delta | | | | |
| | | subunit, complete cds” | | | | |
| 1516 | H3027H04 | “Homo sapiens heat shock protein 75 | | BG078439 | Mm.38470 | Heat Shock/Stress |
| 1517 | | (TRAP1), mRNA” | | | | |
| 1518 | H3028A03 | peptidylprolyl isomerase A (Ppia), | Mus musculus peptidylprolyl isomerase | BG078450 | Mm.5246 | Energy/Metabolism |
| 1519 | | | A (Ppia), mRNA | | | |
| 1520 | H3028A04 | peptidylprolyl isomerase A (Ppia), | Mus musculus peptidylprolyl isomerase | BG078451 | Mm.5246 | Energy/Metabolism |
| 1521 | | | A (Ppia), mRNA | | | |
| 1522 | H3028A09 | BTB and CNC homology 2 (Bach2), | Mus musculus BTB and CNC homology | BG065166 | Mm.21908 | Energy/Metabolism |
| 1523 | | | 2 (Bach2), mRNA | | | |
| 1524 | H3028B03 | adenine nucleotide translocase-2 (Ant2) | Mus musculus adenine nucleotide | | Mm.658 | Energy/Metabolism |
| 1525 | | | translocase-2 (Ant2) mRNA, complete | | | |
| | | | cds | | | |
| 1526 | H3028C09 | “Mus musculus adenosine kinase (Adk), | | BG065190 | Mm.19352 | Signal Transduction |
| 1527 | | mRNA” | | | | |
| 1528 | H3028E01 | death associated protein 3 (DAP-3 gene) | Mus musculus mRNA for death | BG078504 | Mm.29028 | Apoptosis |
| 1529 | | | associated protein 3 (DAP-3 gene) | | | |
| 1530 | H3028E04 | “Mus musculus protein phosphatase 2 | | BG078507 | Mm.1034 | Signal Transduction |
| 1531 | | (formerly 2A), regulatory subunit A (PR | | | | |
| | | 65), alpha isoform (Ppp2r1a), mRNA” | | | | |
| 1532 | H3028F04 | “Mus musculus cathepsin L (Ctsl), | | BG078497 | Mm.930 | Signal Transduction |
| 1533 | | mRNA” | | | | |
| 1534 | H3028F05 | kidney ornithine decarboxylase, clone | Mouse kidney ornithine decarboxylase | BG065221 | Mm.15259 | Energy/Metabolism |
| 1535 | | pODC16, 3 end | mRNA, clone pODC16, 3 end | | | |
| 1536 | H3028F06 | “Mus musculus RAB17, member RAS | | BG078499 | Mm.46396 | Signal Transduction |
| 1537 | | oncogene family (Rab17), mRNA” | | | | |
| 1538 | H3028H11 | “Mus musculus cathepsin H (Ctsh), | | BG065250 | Mm.2277 | Signal Transduction |
| 1539 | | mRNA” | | | | |
| 1540 | H3029A03 | peroxisomal trans 2-enoyl CoA | Mus musculus peroxisomal trans 2- | BG065254 | Mm.29988 | Energy/Metabolism |
| 1541 | | reductase | enoyl CoA reductase mRNA, complete | | | |
| | | | cds | | | |
| 1542 | H3029A05 | cellular apoptosis susceptibility protein | Mus musculus cellular apoptosis | BG065256 | Mm.22417 | Apoptosis |
| 1543 | | | susceptibility protein mRNA, complete | | | |
| | | | cds | | | |
| 1544 | H3029B01 | natural resistance associated | Mus musculus natural resistance | BG065264 | Mm.1304 | Energy/Metabolism |
| 1545 | | macrophage protein-2 (Nramp2), C- | associated macrophage protein-2 | | | |
| | | terminal exon alternative splice variant | (Nramp2) mRNA, C-terminal exon | | | |
| | | | alternative splice variant, complete cds | | | |
| 1546 | H3029B02 | “Mus musculus chaperonin subunit 3 | | BG078552 | Mm.27804 | Heat Shock/Stress |
| 1547 | | (gamma) (Cct3), mRNA” | | | | |
| 1548 | H3029C05 | NADH dehydrogenase (ubiquinone) 1, | Homo sapiens NADH dehydrogenase | BG078565 | Mm.1893 | Energy/Metabolism |
| 1549 | | subcomplex unknown, 2 (14.5 kD, | (ubiquinone) 1, subcomplex unknown, 2 | | | |
| | | B14.5b) (NDUFC2), | (14.5 kD, B14.5b) (NDUFC2), mRNA | | | |
| 1550 | H3029C06 | | | | | Heat Shock/Stress |
| 1551 | | | | | | |
| 1552 | H3029E04 | “Mus musculus Son of sevenless | | BG065298 | Mm.6357 | Signal Transduction |
| 1553 | | homolog 1, (Drosophila)(Sos1), | | | | |
| | | mRNA” | | | | |
| 1554 | H3029G12 | nuclear mitotic apparatus protein 1 | Homo sapiens nuclear mitotic apparatus | | Mm.102520 | |
| | | (NUMA1), | protein 1 (NUMA1), mRNA | | | |
| 1555 | H3030A03 | Mouse cDNA for heat shock protein 65 | | BG078626 | Mm.1777 | Heat Shock/Stress |
| 1556 | | | | | | |
| 1557 | H3030B07 | | | | Mm.1776 | Heat Shock/Stress |
| 1558 | | | | | | |
| 1559 | H3030C05 | “Mus musculus activin receptor IIB | | BG065366 | Mm.8940 | Signal Transduction |
| 1560 | | (Acvr2b), mRNA” | | | | |
| 1561 | H3030C06 | “Mus musculus nucleoside diphosphate | | BG078650 | Mm.1260 | Signal Transduction |
| 1562 | | kinase A long form mRNA, complete | | | | |
| | | cds” | | | | |
| 1563 | H3030C11 | H3030C11 | | | | Energy/Metabolism |
| 1564 | | | | | | |
| 1565 | H3030D10 | pyruvate kinase M | Mus musculus mRNA for pyruvate | BG078663 | Mm.2635 | Energy/Metabolism |
| 1566 | | | kinase M, complete cds | | | |
| 1567 | H3030D11 | pyruvate kinase M | Mus musculus mRNA for pyruvate | BG078664 | Mm.2635 | Energy/Metabolism |
| 1568 | | | kinase M, complete cds | | | |
| 1569 | H3030E04 | Mm.26022 | | | Mm.26022 | Energy/Metabolism |
| 1570 | | | | | | |
| 1571 | H3030E05 | glycine decarboxylase (P-protein) | Human glycine decarboxylase (P- | BG078669 | Mm.27953 | Energy/Metabolism |
| 1572 | | | protein) mRNA | | | |
| 1573 | H3030E06 | glycine decarboxylase (P-protein) | Human glycine decarboxylase (P- | BG078670 | Mm.27953 | Energy/Metabolism |
| 1574 | | | protein) mRNA | | | |
| 1575 | H3030E10 | “Mus musculus cathepsin B (Ctsb), | | BG078674 | Mm.22753 | Signal Transduction |
| 1576 | | mRNA” | | | | |
| 1577 | H3030F09 | Homo sapiens protein phosphatase 3 | | AW537455 | Mm.142697 | Matrix/Structural |
| 1578 | | (formerly 2B), catalytic subunit, beta | | | | Proteins |
| | | isoform (calcineurin A beta) (PPP3CB), | | | | |
| | | mRNA | | | | |
| 1579 | H3030G01 | Cochlear (clone 20F5) | M. musculus partial cochlear mRNA | BG078685 | Mm.43671 | Energy/Metabolism |
| 1580 | | | (clone 20F5) | | | |
| 1581 | H3030G08 | ATP synthase alpha subunit | Mouse ATP synthase alpha subunit, | BG078689 | Mm.4069 | Energy/Metabolism |
| 1582 | | | complete cds | | | |
| 1583 | H3030G09 | “Mus musculus Ca2+-independent | | BG065409 | Mm.117103 | Signal Transduction |
| 1584 | | phospholipase A2 long form mRNA, | | | | |
| | | complete cds” | | | | |
| 1585 | H3030H02 | Bckdk | Mus musculus branched chain ketoacid | BG078694 | Mm.8903 | Signal Transduction |
| 1586 | | | dehydrogenase kinase (Bckdk), mRNA | | | |
| 1587 | H3031A11 | “Mus musculus protein kinase C | | BG078710 | Mm.70272 | Signal Transduction |
| 1588 | | substrate 80K-H (Prkcsh), mRNA” | | | | |
| 1589 | H3031B06 | cytosolic aminopeptidase P | Homo sapiens cytosolic | BG078715 | Mm.99776 | Energy/Metabolism |
| 1590 | | | aminopeptidase P mRNA, complete cds | | | |
| 1591 | H3031C05 | PIS1 gene phosphatidylinositol synthase | Rattus norvegicus PIS1 gene for | BG065447 | Mm.28219 | Energy/Metabolism |
| 1592 | | | phosphatidylinositol synthase, complete | | | |
| | | | cds | | | |
| 1593 | H3031C09 | “Mus musculus putative intracellular | | BG078729 | Mm.27063 | Signal Transduction |
| 1594 | | signaling protein (Trip6) mRNA, | | | | |
| | | complete cds” | | | | |
| 1595 | H3031C12 | Mus musculus N-myc downstream | | BG078732 | Mm.4063 | Transcription/Chromatin |
| 1596 | | regulated 1 (Ndr1), mRNA | | | | |
| 1597 | H3031D03 | aldolase 1, A isom (Aldo1), | Mus musculus aldolase 1, A isoform | BG065457 | Mm.16763 | Energy/Metabolism |
| 1598 | | | (Aldo1), mRNA | | | |
| 1599 | H3031E10 | S-adenosylhomocysteine hydrolase | Mus musculus S-adenosylhomocysteine | BG065475 | Mm.2573 | Energy/Metabolism |
| 1600 | | (Ahcy), | hydrolase (Ahcy), mRNA | | | |
| 1601 | H3031E11 | glyceraldehyde-3-phosphate | Mus musculus glyceraldehyde-3- | BG065476 | Mm.5289 | Energy/Metabolism |
| 1602 | | dehydrogenase (Gapd), | phosphate dehydrogenase (Gapd), | | | |
| | | | mRNA | | | |
| 1603 | H3031E12 | fatty acid desaturase 1 (FADS1), | Homo sapiens fatty acid desaturase 1 | BG078755 | Mm.30158 | Energy/Metabolism |
| 1604 | | | (FADS1), mRNA | | | |
| 1605 | H3031F01 | ubiquinol-cytochrome c reductase core | Homo sapiens ubiquinol-cytochrome c | BG065478 | Mm.972 | Energy/Metabolism |
| 1606 | | protein I (UQCRC1), | reductase core porotein I (UQCRC1), | | | |
| | | | mRNA | | | |
| 1607 | H3031F08 | “Mus musculus heat shock protein 40 | | BG065483 | Mm.2982 | Heat Shock/Stress |
| 1608 | | (HSPF1), mRNA” | | | | |
| 1609 | H3031F12 | M. musculus aspartate aminotransferase | M. musculus aspartate aminotransferase | BG078765 | Mm.149089 | Energy/Metabolism |
| 1610 | | gene exon 10 and 3-flank | gene exon 10 and 3-flank | | | |
| 1611 | H3031G06 | “Mus musculus heat shock protein, 110 kDa | | BG065493 | Mm.1032 | Heat Shock/Stress |
| 1612 | | (Hsp110), mRNA” | | | | |
| 1613 | H3031G08 | Mm.93266 | | | Mm.93266 | Energy/Metabolism |
| 1614 | | | | | | |
| 1615 | H3031G11 | ATPase, H+ transporting, lysosomal | Homo sapiens ATPase, H+ transporting, | BG078776 | Mm.22602 | Energy/Metabolism |
| 1616 | | (vacuolar proton pump) 9 kD (ATP6H), | lysosomal (vacuolar proton pump) 9 kD | | | |
| | | | (ATP6H), mRNA | | | |
| 1617 | H3031H09 | 5-3 exoribonuclease 2 (Xrn2), | Mus musculus 5-3 exoribonuclease 2 | BG065507 | Mm.3065 | Energy/Metabolism |
| 1618 | | | (Xrn2), mRNA | | | |
| 1619 | H3032A01 | hypothetical protein PRO1197 | Homo sapiens hypothetical protein | BG065511 | Mm.24565 | Energy/Metabolism |
| 1620 | | (PRO1197), | PRO1197 (PRO1197), mRNA | | | |
| 1621 | H3032A08 | “Mus musculus heat shock 70 kD protein | | BG078795 | Mm.918 | Heat Shock/Stress |
| 1622 | | 5 (glucose-regulated protein, 78 kD) | | | | |
| | | (Hspa5), mRNA” | | | | |
| 1623 | H3032A09 | peroxisomal integral membrane protein | Mus musculus mRNA for peroxisomal | | Mm.306 | Matrix/Structural |
| 1624 | | PMP34 | integral membrane protein PMP34 | | | Proteins |
| 1625 | H3032A12 | glucose-6-phosphate dehydrogenase X- | Mus musculus glucose-6-phosphate | BG078799 | Mm.27210 | Energy/Metabolism |
| 1626 | | linked (G6pdx), | dehydrogenase X-linked (G6pdx), | | | |
| | | | mRNA | | | |
| 1627 | H3032C01 | cytochrome c gene (MC1) | Mouse cytochrome c gene (MC1) | BG078810 | Mm.35389 | Energy/Metabolism |
| 1628 | | | | | | |
| 1629 | H3032C10 | 3-hydroxy-3-methylglutaryl-Coenzyme | Homo sapiens 3-hydroxy-3- | BG078816 | Mm.2226 | Energy/Metabolism |
| 1630 | | A reductase (HMGCR), | methylglutaryl-Coenzyme A reductase | | | |
| | | | (HMGCR), mRNA | | | |
| 1631 | H3032H02 | phosphatidylcholine transfer protein-like | Mus musculus phosphatidylcholine | BG078855 | Mm.28896 | Energy/Metabolism |
| 1632 | | (Pctpl), | transfer protein-like (Pctpl), mRNA | | | |
| 1633 | H3033A10 | R. norvegicus gene 6-phosphofructo-2- | R. norvegicus gene for 6-phosphofructo- | BG078872 | | Energy/Metabolism |
| 1634 | | kinase/fructose-2,6-biphosphatase (EC | 2-kinase/fructose-2,6-biphosphatase (EC | | | |
| | | 2.7.1.105/EC 3.1.3.46) | 2.7.1.105/EC 3.1.3.46) | | | |
| 1635 | H3033B08 | cytochrome c oxidase, subunit IV | Mus musculus cytochrome c oxidase, | BG078879 | Mm.2136 | Energy/Metabolism |
| 1636 | | (Cox4), | subunit IV (Cox4), mRNA | | | |
| 1637 | H3033B11 | serine protease inhibitor 3 (Spi3), | Mus musculus serine protease inhibitor | BG078882 | Mm.147649 | Energy/Metabolism |
| 1638 | | | 3 (Spi3), mRNA | | | |
| 1639 | H3033C02 | “Mus musculus serine/threonine kinase | | BG078885 | Mm.3794 | Signal Transduction |
| 1640 | | (sak-a) mRNA, complete cds” | | | | |
| 1641 | H3033E10 | ectonucleotide | Mus musculus ectonucleotide | BG065640 | Mm.27254 | Energy/Metabolism |
| 1642 | | pyrophosphatase/phosphodiesterase 1 | pyrophosphatase/phosphodiesterase 1 | | | |
| | | (Enpp1), | (Enpp1), mRNA | | | |
| 1643 | H3034B11 | ATP-binding cassette, sub-family B | | | Mm.143731 | Energy/Metabolism |
| 1644 | | (MDR/TAP), member 12 ABC-me | | | | |
| 1645 | H3035A01 | tumor necrosis factor, alpha-induced | Mus musculus tumor necrosis factor, | BG065761 | Mm.4348 | Apoptosis |
| 1646 | | protein 2 (Tnfaip2), | alpha-induced protein 2 (Tnfaip2), | | | |
| | | | mRNA | | | |
| 1647 | H3035B03 | Mm.32746 | | | Mm.32746 | Energy/Metabolism |
| 1648 | | | | | | |
| 1649 | H3035D08 | Mouse gene for beta-2-adrenergic | | BG079067 | Mm.5598 | Signal Transduction |
| 1650 | | receptor | | | | |
| 1651 | H3035F02 | “Mus musculus RNA-binding protein | | BG065817 | Mm.2411 | Signal Transduction |
| 1652 | | isoform G3BP-2a (G3BP2) mRNA, | | | | |
| | | complete cds” | | | | |
| 1653 | H3036B11 | “Mus musculus Rho guanine nucleotide | | BG079136 | Mm.3181 | Signal Transduction |
| 1654 | | exchange factor (GEF) 1 (Arhgef1), | | | | |
| | | mRNA” | | | | |
| 1655 | H3036H01 | “Mus musculus lysophosphatidic acid | | BG079186 | Mm.4772 | Signal Transduction |
| 1656 | | receptor (vzg-1) mRNA, complete cds” | | | | |
| 1657 | H3037B01 | kinesin-like 6 (mitotic centromere- | Homo sapiens kinesin-like 6 (mitotic | | Mm.27828 | Cell Cycle |
| 1658 | | associated kinesin) (KNSL6), | centromere-associated kinesin) | | | |
| | | | (KNSL6), mRNA | | | |
| 1659 | H3037F06 | serine protease inhibitor, Kunitz type 1 | Mus musculus serine protease inhibitor, | BG079254 | Mm.104955 | Energy/Metabolism |
| 1660 | | (Spint1), | Kunitz type 1 (Spint1), mRNA | | | |
| 1661 | H3037G12 | “Mus musculus cAMP-specific | | BG066005 | Mm.154704 | Signal Transduction |
| 1662 | | phosphodiesterase 4A (Pde4a) gene, | | | | |
| | | exons 2 through 8 and PDE4A isoform | | | | |
| | | 1 exon 1” | | | | |
| 1663 | H3038A06 | “Mus musculus interferon alpha | | BG079287 | Mm.21761 | Heat Shock/Stress |
| 1664 | | responsive protein (15 kDa) (Ifrg15), | | | | |
| | | mRNA” | | | | |
| 1665 | H3038C07 | potassium voltage-gated channel, | Homo sapiens potassium voltage-gated | BG079311 | Mm.113278 | Energy/Metabolism |
| 1666 | | delayed-rectifier, subfamily S, member | channel, delayed-rectifier, subfamily S, | | | |
| | | 3 (KCNS3), | member 3 (KCNS3), mRNA | | | |
| 1667 | H3038C09 | phosphoribosyl pyrophosphate | Homo sapiens phosphoribosyl | BG079313 | Mm.27703 | Energy/Metabolism |
| 1668 | | synthetase-associated protein 2 | pyrophosphate synthetase-associated | | | |
| | | (PRPSAP2), | protein 2 (PRPSAP2), mRNA | | | |
| 1669 | H3038E03 | PRKC, apoptosis, WT1, regulator | Homo sapiens PRKC, apoptosis, WT1, | BG079331 | Mm.6617 | Apoptosis |
| 1670 | | (PAWR), | regulator (PAWR), mRNA | | | |
| 1671 | H3038E05 | Mus musculus growth differentiation | | BG079333 | Mm.4213 | Signal Transduction |
| 1672 | | factor 3 (Gdf3), mRNA | | | | |
| 1673 | H3038E10 | methyltransferase COQ3 (COQ3), | Homo sapiens methyltransferase COQ3 | BG066071 | Mm.5662 | Energy/Metabolism |
| 1674 | | | (COQ3), mRNA | | | |
| 1675 | H3039A05 | “Mus musculus regulator of G-protein | | BG079374 | Mm.13264 | Signal Transduction |
| 1676 | | signaling 11 mRNA, partial cds” | | | | |
| 1677 | H3039B09 | “Mus musculus serine/threonine kinase | | BG066121 | Mm.22584 | Signal Transduction |
| 1678 | | receptor associated protein (Strap), | | | | |
| | | mRNA” | | | | |
| 1679 | H3039C11 | “Mus musculus receptor tyrosine kinase | | BG066134 | Mm.2901 | Signal Transduction |
| 1680 | | (Dtk) mRNA, complete cds” | | | | |
| 1681 | H3039D02 | asparaginyl-tRNA synthetase (NARS), | Homo sapiens asparaginyl-tRNA | BG079401 | Mm.29192 | Energy/Metabolism |
| 1682 | | | synthetase (NARS), mRNA | | | |
| 1683 | H3039E07 | isocitrate dehydrogenase 3 (NAD+) beta | Homo sapiens isocitrate dehydrogenase | C78231 | Mm.29590 | Energy/Metabolism |
| 1684 | | (IDH3B), | 3 (NAD+) beta (IDH3B), mRNA | | | |
| 1685 | H3039E08 | “Mus musculus SH3-containing protein | | BG079417 | Mm.4165 | Signal Transduction |
| 1686 | | SH3P2 mRNA, partial cds” | | | | |
| 1687 | H3039F05 | ornithine aminotransferase (Oat), | Mus musculus ornithine | BG079424 | Mm.42187 | Energy/Metabolism |
| 1688 | | | aminotransferase (Oat), mRNA | | | |
| 1689 | H3039G04 | M. musculus seryl-tRNA synthetase | M. musculus seryl-tRNA synthetase | BG079434 | Mm.28688 | Energy/Metabolism |
| 1690 | | (SERS), 5 end | (SERS) mRNA, 5 end | | | |
| 1691 | H3039G12 | 5-3 exoribonuclease 1 (Xrn1), | Mus musculus 5-3 exoribonuclease 1 | BG079441 | Mm.5703 | Energy/Metabolism |
| 1692 | | | (Xrn1), mRNA | | | |
| 1693 | H3039H01 | “Mus musculus calponin 2 (Cnn2), | | BG079442 | Mm.21776 | Signal Transduction |
| 1694 | | mRNA” | | | | |
| 1695 | H3039H05 | Mus musculus putative CCAAT binding | | BG079446 | Mm.24169 | |
| 1696 | | factor 1 (mCBF) mRNA, alternatively | | | | |
| | | spliced transcript mCBF1, complete cds | | | | |
| 1697 | H3040A04 | adenine phosphoribosyltransferase | Mouse adenine | BG079455 | Mm.1786 | Energy/Metabolism |
| 1698 | | (APRT) | phosphoribosyltransferase (APRT), | | | |
| | | | complete cds | | | |
| 1699 | H3040E11 | palmitoyl-protein thioesterase (Ppt), | Mus musculus palmitoyl-protein | | Mm.153740 | |
| 1700 | | | thioesterase (Ppt), mRNA | | | |
| 1701 | H3041A02 | NADH dehydrogenase (ubiquinone) 1 | Homo sapiens NADH dehydrogenase | BG079485 | Mm.28293 | Energy/Metabolism |
| 1702 | | alpha subcomplex, 10 (42 kD) | (ubiquinone) 1 alpha subcomplex, 10 | | | |
| | | (NDUFA10), | (42 kD) (NDUFA10), mRNA | | | |
| 1703 | H3041A04 | lysyl-tRNA synthetase (KARS), | Homo sapiens lysyl-tRNA synthetase | BG066271 | Mm.29949 | Energy/Metabolism |
| 1704 | | | (KARS), mRNA | | | |
| 1705 | H3041B11 | mevalonate pyrophosphate | Rattus norvegicus mevalonate | BG079503 | Mm.28146 | Energy/Metabolism |
| 1706 | | decarboxylase | pyrophosphate decarboxylase mRNA, | | | |
| | | | complete cds | | | |
| 1707 | H3041F09 | glucose transporter (GLUTX1 gene) | Mus musculus mRNA for glucose | BG066327 | Mm.7241 | Energy/Metabolism |
| 1708 | | | transporter (GLUTX1 gene) | | | |
| 1709 | H3041G03 | solute carrier family 15 (H+/peptide | Mus musculus solute carrier family 15 | BG079545 | Mm.63479 | Energy/Metabolism |
| 1710 | | transporter), member 2 (Slc15a2), | (H+/peptide transporter), member 2 | | | |
| | | | (Slc15a2), mRNA | | | |
| 1711 | H3041G08 | protease (prosome, macropain) 26S | Mus musculus protease (prosome, | BG079550 | Mm.665 | Energy/Metabolism |
| 1712 | | subunit, ATPase 5 (Psmc5), | macropain) 26S subunit, ATPase 5 | | | |
| | | | (Psmc5), mRNA | | | |
| 1713 | H3041H03 | major histocompatibility complex | Mus musculus major histocompatibility | BG079557 | Mm.14808 | Energy/Metabolism |
| 1714 | | region NG27, NG28, RPS28, NADH | complex region NG27, NG28, RPS28, | | | |
| | | oxidoreductase, NG29, KIFC1, Fas- | NADH oxidoreductase, NG29, KIFC1, | | | |
| | | binding protein, BING1, tapasin, | Fas-binding protein, BING1, tapasin, | | | |
| | | RalGDS-like, KE2, BING4, beta 1,3- | RalGDS-like, KE2, BING4, beta 1,3- | | | |
| | | galactosyl transfera | galactosyl transfera | | | |
| 1715 | H3042A05 | BCL2-associated athanogene 4 (BAG4), | Homo sapiens BCL2-associated | BG066354 | Mm.27102 | Apoptosis |
| 1716 | | | athanogene 4 (BAG4), mRNA | | | |
| 1717 | H3042C08 | “Mus musculus dual specificity | | BG079592 | Mm.34912 | Signal Transduction |
| 1718 | | phosphatase 10 (Dusp10), mRNA” | | | | |
| 1719 | H3042D02 | brain cDNA, clone MNCb-2243, similar | Mus musculus brain cDNA, clone | BG079597 | Mm.28373 | Apoptosis |
| 1720 | | to Bcl2-associated athanogene 3 (Bag3), | MNCb-2243, similar to Mus musculus | | | |
| | | | Bcl2-associated athanogene 3 (Bag3), | | | |
| | | | mRNA | | | |
| 1721 | H3042F12 | serine protease inhibitor 4 (Spi4), | Mus musculus serine protease inhibitor | BG079624 | Mm.3093 | Energy/Metabolism |
| 1722 | | | 4 (Spi4), mRNA | | | |
| 1723 | H3042G07 | Mouse heat-shock protein hsp84 mRNA | | BG079631 | Mm.2180 | Heat Shock/Stress |
| 1724 | | | | | | |
| 1725 | H3043A03 | “Mus musculus casein kinase II, alpha | | BG066436 | Mm.23692 | Signal Transduction |
| 1726 | | 1, related sequence 4 (Csnk2a1-rs4), | | | | |
| | | mRNA” | | | | |
| 1727 | H3043F09 | Mus musculus GNB3 gene for GTP- | | BG066499 | Mm.22228 | |
| 1728 | | binding protein beta3 subunit, complete | | | | |
| | | cds | | | | |
| 1729 | H3043F12 | ferrochelatase (Fech), | Mus musculus ferrochelatase (Fech), | BG066502 | Mm.1070 | |
| 1730 | | | mRNA | | | |
| 1731 | H3044B01 | “Mus musculus Cctd gene for | | BG079699 | Mm.36431 | Heat Shock/Stress |
| 1732 | | chaperonin containing TCP-1 delta | | | | |
| | | subunit, complete cds” | | | | |
| 1733 | H3044E06 | protease (prosome, macropain) 28 | Mus musculus protease (prosome, | BG066650 | Mm.830 | Energy/Metabolism |
| 1734 | | subunit, alpha (Psme1), | macropain) 28 subunit, alpha (Psme1), | | | |
| | | | mRNA | | | |
| 1735 | H3044G06 | A10, partial cds | Mus musculus A10 mRNA, partial cds | BG066673 | Mm.16898 | Energy/Metabolism |
| 1736 | | | | | | |
| 1737 | H3045B02 | cytosolic aminopeptidase P | Homo sapiens cytosolic | BG079790 | Mm.99776 | Energy/Metabolism |
| 1738 | | | aminopeptidase P mRNA, complete cds | | | |
| 1739 | H3045B03 | Mm.23710 | | | Mm.23710 | Energy/Metabolism |
| 1740 | | | | | | |
| 1741 | H3045B12 | solute carrier family 12, member 7 | Mus musculus solute carrier family 12, | BG079799 | Mm.155195 | Energy/Metabolism |
| 1742 | | (Slc12a7), | member 7 (Slc12a7), mRNA | | | |
| 1743 | H3045D07 | NAALADase II protein | Homo sapiens mRNA for NAALADase | BG079815 | Mm.7060 | Energy/Metabolism |
| 1744 | | | II protein | | | |
| 1745 | H3045D08 | Mm.25054 | | | Mm.25054 | Energy/Metabolism |
| 1746 | | | | | | |
| 1747 | H3045E05 | serine protease OMI (Omi), | Mus musculus serine protease OMI | BG079822 | Mm.21880 | Energy/Metabolism |
| 1748 | | | (Omi), mRNA | | | |
| 1749 | H3046A03 | DKFZP566O084 protein | Homo sapiens DKFZP566O084 protein | BG079861 | Mm.21475 | Energy/Metabolism |
| 1750 | | (DKFZPS66O084), | (DKFZP566O084), mRNA | | | |
| 1751 | H3046A12 | programmed cell death protein 7 | Mus musculus programmed cell death | BG066710 | Mm.29193 | Apoptosis |
| 1752 | | (Pdcd7), | protein 7 (Pdcd7), mRNA | | | |
| 1753 | H3046C10 | isopentenyl-diphosphate delta isomerase | Homo sapiens isopentenyl-diphosphate | BG079889 | Mm.29847 | Energy/Metabolism |
| 1754 | | (IDI1), | delta isomerase (IDI1), mRNA | | | |
| 1755 | H3047B07 | tripeptidyl peptidase II (Tpp2), | Mus musculus tripeptidyl peptidase II | BG066807 | Mm.28867 | Energy/Metabolism |
| 1756 | | | (Tpp2), mRNA | | | |
| 1757 | H3047D01 | Bos taurus creatine kinase | Bos taurus mRNA for creatine kinase, | BG079988 | Mm.970 | Energy/Metabolism |
| 1758 | | | complete cds | | | |
| 1759 | H3047D05 | gene encoding enoyl-CoA hydratase, | H. sapiens gene encoding enoyl-CoA | BG079992 | Mm.24452 | Energy/Metabolism |
| 1760 | | exons 5, 6 & 7 | hydratase, exons 5, 6 & 7 | | | |
| 1761 | H3047F02 | vacuolar-adenosine trisphosphatase (V- | Mus musculus mRNA for vacuolar- | BG079910 | Mm.25079 | Energy/Metabolism |
| 1762 | | ATPase) | adenosine trisphosphatase (V-ATPase), | | | |
| | | | complete cds | | | |
| 1763 | H3047G12 | C7-1 protein (C7-1) | Rattus norvegicus C7-1 protein (C7-1) | C80679 | Mm.21961 | Energy/Metabolism |
| 1764 | | | mRNA, complete cds | | | |
| 1765 | H3048A05 | isoprenylcysteine carboxyl | Homo sapiens isoprenylcysteine | BG080028 | Mm.44565 | Energy/Metabolism |
| 1766 | | methyltransferase (ICMT), | carboxyl methyltransferase (ICMT), | | | |
| | | | mRNA | | | |
| 1767 | H3048B11 | cytochrome c oxidase, subunit VIIc | Mus musculus cytochrome c oxidase, | BG080036 | Mm.24165 | Energy/Metabolism |
| 1768 | | (Cox7c), | subunit VIIc (Cox7c), mRNA | | | |
| 1769 | H3048E06 | small zinc finger-like protein DDP2 | Mus musculus small zinc finger-like | BG080049 | Mm.30718 | Energy/Metabolism |
| 1770 | | (Ddp2) | protein DDP2 (Ddp2) mRNA, complete | | | |
| | | | cds | | | |
| 1771 | H3048G11 | biliverdin reductase B (flavin reductase | Homo sapiens biliverdin reductase B | BG066946 | Mm.24021 | Energy/Metabolism |
| 1772 | | (NADPH)) (BLVRB), | (flavin reductase (NADPH)) (BLVRB), | | | |
| | | | mRNA | | | |
| 1773 | H3049D07 | “Mus musculus Janus kinase 2 (Jak2), | | BG080085 | Mm.25112 | Signal Transduction |
| 1774 | | mRNA” | | | | |
| 1775 | H3049F02 | carbonyl reductase 3 (CBR3), | Homo sapiens carbonyl reductase 3 | BG067014 | Mm.4512 | Energy/Metabolism |
| 1776 | | | (CBR3), mRNA | | | |
| 1777 | H3049G02 | solute carrier family 16 | Mus musculus solute carrier family 16 | BG067025 | Mm.9086 | Energy/Metabolism |
| 1778 | | (monocarboxylic acid transporters), | (monocarboxylic acid transporters), | | | |
| | | member 1 (Slc16a1), | member 1 (Slc16a1), mRNA | | | |
| 1779 | H3049G04 | “Mus musculus B-cell | | BG067027 | Mm.28782 | Signal Transduction |
| 1780 | | leukemia/lymphoma 10 (Bcl10), | | | | |
| | | mRNA” | | | | |
| 1781 | H3049G07 | PGES prostaglandin E synthase | Mus musculus PGES mRNA for | BG067030 | Mm.154682 | Energy/Metabolism |
| 1782 | | | prostaglandin E synthase, complete cds | | | |
| 1783 | H3050A12 | mannosidase 1, beta (Man1b), | Mus musculus mannosidase 1, beta | BG080131 | Mm.103874 | Energy/Metabolism |
| 1784 | | | (Man1b), mRNA | | | |
| 1785 | H3050B11 | small zinc finger-like protein (Tim13) | Mus musculus small zinc finger-like | BG080141 | Mm.142132 | Energy/Metabolism |
| 1786 | | | protein (Tim13) mRNA, complete cds | | | |
| 1787 | H3050C02 | glutathione synthetase (Gss), | Mus musculus glutathione synthetase | BG067069 | Mm.7504 | Energy/Metabolism |
| 1788 | | | (Gss), mRNA | | | |
| 1789 | H3050E05 | putative dimethyladenosine transferase | Homo sapiens putative | BG080168 | Mm.9563 | Energy/Metabolism |
| 1790 | | (HSA9761), | dimethyladenosine transferase | | | |
| | | | (HSA9761), mRNA | | | |
| 1791 | H3050E08 | diaphorase (NADH) (cytochrome b-5 | Homo sapiens diaphorase (NADH) | BG080169 | Mm.22560 | Energy/Metabolism |
| 1792 | | reductase) (DIA1), | (cytochrome b-5 reductase) (DIA1), | | | |
| | | | mRNA | | | |
| 1793 | H3050F12 | hydroxysteroid 17-beta dehydrogenase 4 | Mus musculus hydroxysteroid 17-beta | BG080183 | Mm.9569 | Energy/Metabolism |
| 1794 | | (Hsd17b4), | dehydrogenase 4 (Hsd17b4), mRNA | | | |
| 1795 | H3050G05 | solute carrier family 7 (cationic amino | Mus musculus solute carrier family 7 | BG067114 | Mm.4676 | Energy/Metabolism |
| 1796 | | acid transporter, y+ system), member 2 | (cationic amino acid transporter, y+ | | | |
| | | (Slc7a2), | system), member 2 (Slc7a2), mRNA | | | |
| 1797 | H3050H06 | “Mus musculus interferon regulatory | | BG067127 | Mm.1246 | Heat Shock/Stress |
| 1798 | | factor 1 (Irf1), mRNA” | | | | |
| 1799 | H3050H11 | Mm.25374 | | | Mm.25374 | Energy/Metabolism |
| 1800 | | | | | | |
| 1801 | H3051C06 | | | | Mm.7730 | Heat Shock/Stress |
| 1802 | | | | | | |
| 1803 | H3051C07 | “Mus musculus phospholipase D2 gene, | | BG080231 | Mm.2538 | Signal Transduction |
| 1804 | | exons 13 through 25 and complete cds” | | | | |
| 1805 | H3051D07 | cDNA FLJ13488 fis, clone | Homo sapiens cDNA FLJ13488 fis, | BG080243 | Mm.22363 | |
| 1806 | | PLACE1003915, weakly similar to | clone PLACE1003915, weakly similar | | | |
| | | PROBABLE ARGINYL-TRNA | to PROBABLE ARGINYL-TRNA | | | |
| | | SYNTHETASE, CYTOPLASMIC (EC | SYNTHETASE, CYTOPLASMIC (EC | | | |
| | | 6.1.1.19) | 6.1.1.19) | | | |
| 1807 | H3051E06 | aldehyde reductase 6, renal (Aldr6r- | Mus musculus aldehyde reductase 6, | BG080253 | Mm.21268 | Energy/Metabolism |
| 1808 | | pending), | renal (Aldr6r-pending), mRNA | | | |
| 1809 | H3051F02 | Mus musculus growth differentiation | | BG080261 | Mm.9593 | Signal Transduction |
| 1810 | | factor 9 (Gdf9), mRNA | | | | |
| 1811 | H3051G07 | guanosine monophosphate reductase | Homo sapiens guanosine | BG080277 | Mm.25808 | Energy/Metabolism |
| 1812 | | (GMPR), | monophosphate reductase (GMPR), | | | |
| | | | mRNA | | | |
| 1813 | H3052A03 | potassium channel regulator 1 | Rattus norvegicus potassium channel | BG067226 | | Energy/Metabolism |
| 1814 | | | regulator 1 mRNA, complete cds | | | |
| 1815 | H3052A04 | malate dehydrogenase, soluble (Mor2), | Mus musculus malate dehydrogenase, | BG067227 | Mm.3156 | Energy/Metabolism |
| 1816 | | | soluble (Mor2), mRNA | | | |
| 1817 | H3052B06 | ATP-binding cassette, sub-family B | Mus musculus ATP-binding cassette, | BG080311 | Mm.6404 | Energy/Metabolism |
| 1818 | | (MDR/TAP), member 1 (Abcb1), | sub-family B (MDR/TAP), member 1 | | | |
| | | | (Abcb1), mRNA | | | |
| 1819 | H3052C08 | coproporphyrinogen oxidase (Cpo), | Mus musculus coproporphyrinogen | BG080324 | Mm.35820 | Energy/Metabolism |
| 1820 | | | oxidase (Cpo), mRNA | | | |
| 1821 | H3052D11 | “Mus musculus adenylate kinase 2 | | BG067269 | Mm.29460 | Signal Transduction |
| 1822 | | (Ak2), mRNA” | | | | |
| 1823 | H3052H05 | “Homo sapiens rab3 GTPase-activating | | BG080373 | Mm.28344 | Signal Transduction |
| 1824 | | protein, non-catalytic subunit (150 kD) | | | | |
| | | (RAB3-GAP150), mRNA” | | | | |
| 1825 | H3052H11 | asparaginyl-tRNA synthetase (NARS), | Homo sapiens asparaginyl-tRNA | BG080379 | Mm.29192 | Energy/Metabolism |
| 1826 | | | synthetase (NARS), mRNA | | | |
| 1827 | H3053A12 | “Mus musculus regulator of G-protein | | BG080390 | Mm.28262 | Signal Transduction |
| 1828 | | signaling 2 mRNA, complete cds” | | | | |
| 1829 | H3053B07 | tyrosine hydroxylase (Th), | Mus musculus tyrosine hydroxylase | BG067326 | Mm.140599 | Energy/Metabolism |
| 1830 | | | (Th), mRNA | | | |
| 1831 | H3053C02 | phosphoribosyl pyrophosphate | Homo sapiens phosphoribosyl | BG080402 | Mm.25125 | Energy/Metabolism |
| 1832 | | synthetase-associated protein 1 | pyrophosphate synthetase-associated | | | |
| | | (PRPSAP1), | protein 1 (PRPSAP1), mRNA | | | |
| 1833 | H3053C09 | “Mus musculus nicotinic acetylcholine | | BG080409 | | Signal Transduction |
| 1834 | | receptor alpha 5 subunit (Acra5) | | | | |
| | | mRNA, partial cds” | | | | |
| 1835 | H3053E04 | “Mus musculus calponin 1 (Cnn1), | | BG067357 | Mm.4356 | Signal Transduction |
| 1836 | | mRNA” | | | | |
| 1837 | H3053G12 | “Mus musculus polynucleotide kinase | | BG067384 | Mm.29545 | Signal Transduction |
| 1838 | | 3′-phosphatase (Pnkp), mRNA” | | | | |
| 1839 | H3053H06 | citrin (Slc25a13) | Mus musculus citrin (Slc25a13) | BG083930 | Mm.2124 | Energy/Metabolism |
| 1840 | | | mRNA, complete cds | | | |
| 1841 | H3054B05 | “Mus musculus folate receptor 3 | | BG080476 | Mm.86738 | Signal Transduction |
| 1842 | | (Folbp3) mRNA, complete cds” | | | | |
| 1843 | H3054B12 | Nrf 2 | Mus musculus p45 NF-E2 related factor | BG067417 | Mm.1025 | |
| 1844 | | | 2 (Nrf 2) mRNA, complete cds | | | |
| 1845 | H3054C02 | Gadd45g | Mus musculus growth arrest and DNA- | BG067419 | Mm.9653 | Energy/Metabolism |
| 1846 | | | damage-inducible, gamma (Gadd45g), | | | |
| | | | mRNA | | | |
| 1847 | H3054C06 | “Mus musculus phospholipase c | | BG080489 | Mm.140 | Signal Transduction |
| 1848 | | neighboring(Png), mRNA” | | | | |
| 1849 | H3054C09 | WNT-2 | Mus musculus WNT-2 gene, partial cds; | BG080492 | Mm.43231 | Energy/Metabolism |
| 1850 | | | putative ankyrin-related protein and | | | |
| | | | cystic fibrosis transmembrane | | | |
| | | | conductance regulator (CFTR) genes, | | | |
| | | | section 2 of 2 of the complete cds; and | | | |
| | | | unknown gene | | | |
| 1851 | H3054D05 | “Rattus norvegicus protein associating | | BG080499 | | Heat Shock/Stress |
| 1852 | | with small stress protein PASS1 (Pass1) | | | | |
| | | mRNA, complete cds” | | | | |
| 1853 | H3054F04 | “Mus musculus gene for p70/p85 s6 | | BG067455 | Mm.26901 | Signal Transduction |
| 1854 | | kinase, exon” | | | | |
| 1855 | H3054F05 | lysozyme M | Mouse lysozyme M gene, exon 4 | BG067456 | Mm.654 | Energy/Metabolism |
| 1856 | | | | | | |
| 1857 | H3054F11 | ATP6N1B | Homo sapiens ATPase, H(+)- | BG080527 | Mm.26909 | Energy/Metabolism |
| 1858 | | | transporting, lysosomal, noncatalytic | | | |
| | | | accessory protein 1B (ATP6N1B), | | | |
| | | | mRNA | | | |
| 1859 | H3054F12 | TR2L | Mus musculus TR2L mRNA, partial cds | BG080528 | | Apoptosis |
| 1860 | | | | | | |
| 1861 | H3055A07 | “Homo sapiens mutated in colorectal | | BG080557 | Mm.155210 | Signal Transduction |
| 1862 | | cancers (MCC), mRNA” | | | | |
| 1863 | H3055B08 | major histocompatibility locus class III | Mus musculus major histocompatibility | | Mm.1511 | |
| 1864 | | region: butyrophilin-like protein gene, | locus class III region: butyrophilin-like | | | |
| | | partial cds; Notch4, PBX2, RAGE, | protein gene, partial cds; Notch4, | | | |
| | | lysophatidic acid acyl transferase-alpha, | PBX2, RAGE, lysophatidic acid acyl | | | |
| | | palmitoyl-protein thioestera | transferase-alpha, palmitoyl-protein | | | |
| | | | thioestera | | | |
| 1865 | H3055B10 | inhibitor of kappa light polypeptide | | | Mm.28269 | Energy/Metabolism |
| 1866 | | gene enhancer in B-cells, kinase beta | | | | |
| | | IKK[b]\|IKK2\|IKK-2\|IKK-beta | | | | |
| 1867 | H3055C04 | “Mus musculus Ras-GTPase-activating | | BG080577 | Mm.2038 | Signal Transduction |
| 1868 | | protein SH3-domain binding protein | | | | |
| | | (G3bp-pending), mRNA” | | | | |
| 1869 | H3055C05 | “Homo sapiens inositol 1,3,4- | | BG080578 | | Signal Transduction |
| 1870 | | triphosphate 5/6 kinase (ITPK1), | | | | |
| | | mRNA” | | | | |
| 1871 | H3055C10 | ATP/GTP-binding protein (HEAB), | Homo sapiens ATP/GTP-binding | BG080583 | Mm.21583 | Energy/Metabolism |
| 1872 | | | protein (HEAB), mRNA | | | |
| 1873 | H3055D02 | aldolase B, fructose-bisphosphate | Homo sapiens aldolase B, fructose- | BG080587 | Mm.87581 | Energy/Metabolism |
| 1874 | | (ALDOB), | bisphosphate (ALDOB), mRNA | | | |
| 1875 | H3055E07 | TRAF family member associated NF- | Mus musculus TRAF family member | BG080604 | Mm.1803 | Apoptosis |
| 1876 | | kappa B activator (TANK) | associated NF-kappa B activator | | | |
| | | | (TANK) mRNA, complete cds | | | |
| 1877 | H3055F07 | delta-aminolevulinate dehydratase (Lv), | Mus musculus delta-aminolevulinate | BG080616 | Mm.90076 | Energy/Metabolism |
| 1878 | | | dehydratase (Lv), mRNA | | | |
| 1879 | H3055G02 | dipeptidyl peptidase I precursor | Mus musculus dipeptidyl peptidase I | BG080623 | Mm.684 | Energy/Metabolism |
| 1880 | | | precursor mRNA, complete cds | | | |
| 1881 | H3056A09 | “M. musculus mRNA for inositol 1,4,5- | | BG067589 | Mm.7800 | Signal Transduction |
| 1882 | | trisphosphate receptor (type 2)” | | | | |
| 1883 | H3056C11 | DKFZP564O2082 protein | Homo sapiens DKFZP564O2082 | | Mm.21826 | |
| 1884 | | (DKFZP564O2082), | protein (DKFZP564O2082), mRNA | | | |
| 1885 | H3056D03 | “Homo sapiens calponin 3, acidic | | BG080757 | Mm.22171 | Signal Transduction |
| 1886 | | (CNN3), mRNA” | | | | |
| 1887 | H3056G10 | thioredoxin 2 (TRX2) | Homo sapiens thioredoxin 2 (TRX2) | BG080633 | Mm.3533 | Energy/Metabolism |
| 1888 | | | mRNA, complete cds | | | |
| 1889 | H3056H05 | H3056H05 | | | | Energy/Metabolism |
| 1890 | | | | | | |
| 1891 | H3057C01 | “Mus musculus interferon-related | | BG067699 | Mm.168 | Heat Shock/Stress |
| 1892 | | developmental regulator 1 (Ifrd1), | | | | |
| | | mRNA” | | | | |
| 1893 | H3057C08 | GPX3 | | | Mm.7156 | Energy/Metabolism |
| 1894 | | | | | | |
| 1895 | H3057C10 | bisphosphate 3-nucleotidase 1 (Bpnt1), | Mus musculus bisphosphate 3- | BG067708 | Mm.18096 | Energy/Metabolism |
| 1896 | | | nucleotidase 1 (Bpnt1), mRNA | | | |
| 1897 | H3057D09 | acetyl-Coenzyme A carboxylase beta | Homo sapiens acetyl-Coenzyme A | BG067718 | Mm.88548 | Energy/Metabolism |
| 1898 | | (ACACB), | carboxylase beta (ACACB), mRNA | | | |
| 1899 | H3057E05 | “Mus musculus protein tyrosine | | BG080698 | Mm.4420 | Signal Transduction |
| 1900 | | phosphatase, non-receptor type 21 | | | | |
| | | (Ptpn21), mRNA” | | | | |
| 1901 | H3057F01 | “Mus musculus casein kinase 1, epsilon | | BG080793 | Mm.30199 | Signal Transduction |
| 1902 | | (Csnkle), mRNA” | | | | |
| 1903 | H3057H04 | “Mus musculus HSP40-like protein | | BG080820 | Mm.46746 | Heat Shock/Stress |
| 1904 | | mRNA, partial sequence” | | | | |
| 1905 | H3057H06 | solute carrier family 16 | Mus musculus solute carrier family 16 | C87415 | Mm.5045 | Energy/Metabolism |
| 1906 | | (monocarboxylic acid transporters), | (monocarboxylic acid transporters), | | | |
| | | member 2 (Slc16a2), | member 2 (Slc16a2), mRNA | | | |
| 1907 | H3058A10 | Fas-interacting serine/threonine kinase 3 | Mus musculus Fas-interacting | BG080836 | Mm.29026 | Apoptosis |
| 1908 | | (Fist3) | serine/threonine kinase 3 (Fist3) mRNA, | | | |
| | | | complete cds | | | |
| 1909 | H3058C03 | Bcl2-like 10 (Bcl2110), | Mus musculus Bcl2-like 10 (Bcl2110), | BG080862 | Mm.25988 | Apoptosis |
| 1910 | | | mRNA | | | |
| 1911 | H3058C09 | “Mus musculus Jun oncogene (Jun), | | BG080846 | Mm.482 | Signal Transduction |
| 1912 | | mRNA” | | | | |
| 1913 | H3058D09 | “Mus musculus guanine nucleotide | | BG080868 | Mm.27307 | Signal Transduction |
| 1914 | | binding protein (G protein), gamma 3 | | | | |
| | | subunit (Gng3), mRNA” | | | | |
| 1915 | H3058G06 | transient receptor potential-related | Mus musculus transient receptor | BG080898 | Mm.143646 | Energy/Metabolism |
| 1916 | | protein (ChaK), | potential-related protein (ChaK), mRNA | | | |
| 1917 | H3059A10 | sodium bicarbonate cotransporter isom 3 | Mus musculus sodium bicarbonate | BG067865 | Mm.34957 | Energy/Metabolism |
| 1918 | | (kNBC-3), | cotransporter isoform 3 (kNBC-3), | | | |
| | | | mRNA | | | |
| 1919 | H3059B02 | “Homo sapiens regulator of G-protein | | BG067869 | Mm.31378 | Signal Transduction |
| 1920 | | signalling 12 (RGS12), mRNA” | | | | |
| 1921 | H3059B03 | “Mus musculus protein kinase C delta | | BG080928 | Mm.142839 | Signal Transduction |
| 1922 | | mRNA, complete cds” | | | | |
| 1923 | H3059B07 | “Mus sp. JAK1 protein tyrosine kinase | | BG067874 | Mm.28598 | Signal Transduction |
| 1924 | | mRNA, complete cds” | | | | |
| 1925 | H3059D07 | alanyl-tRNA synthetase (AARS), | Homo sapiens alanyl-tRNA synthetase | BG080951 | Mm.24174 | Energy/Metabolism |
| 1926 | | | (AARS), mRNA | | | |
| 1927 | H3059D11 | KIAA1093 protein, partial cds | Homo sapiens mRNA for KIAA1093 | BG067897 | Mm.22829 | Apoptosis |
| 1928 | | | protein, partial cds | | | |
| 1929 | H3059F12 | “Mus musculus GTP binding protein | | BG067921 | Mm.15793 | Signal Transduction |
| 1930 | | (GTP2) mRNA, complete cds” | | | | |
| 1931 | H3059G11 | intronless glutamine synthetase gene | Mouse intronless glutamine synthetase | BG067932 | Mm.41660 | Energy/Metabolism |
| 1932 | | | gene, complete cds | | | |
| 1933 | H3060A10 | M. musculus arachidonate epidermis- | M. musculus mRNA for arachidonate | BG067951 | Mm.1122 | Energy/Metabolism |
| 1934 | | type 12(S)-lipoxygenase | epidermis-type 12(S)-lipoxygenase | | | |
| 1935 | H3060B09 | “Homo sapiens mitogen-activated | | BG081019 | Mm.24022 | Signal Transduction |
| 1936 | | protein kinase kinase kinase kinase 5 | | | | |
| | | (MAP4K5), mRNA” | | | | |
| 1937 | H3060D04 | Mm.24594 | | | Mm.24594 | Energy/Metabolism |
| 1938 | | | | | | |
| 1939 | H3060H04 | “Mus musculus JNK-binding protein 1 | | BG081077 | Mm.25540 | Signal Transduction |
| 1940 | | (Jnkbp1-pending), mRNA” | | | | |
| 1941 | H3060H07 | Mm.25580 | | | Mm.25580 | Energy/Metabolism |
| 1942 | | | | | | |
| 1943 | H3061B06 | UDP-N-acetyl-alpha-D- | Mus musculus UDP-N-acetyl-alpha-D- | BG068045 | Mm.38441 | Energy/Metabolism |
| 1944 | | galactosamine: polypeptide N- | galactosamine: polypeptide N- | | | |
| | | acetylgalactosaminyltransferase 3 | acetylgalactosaminyltransferase 3 | | | |
| | | (Galnt3), | (Galnt3), mRNA | | | |
| 1945 | H3061E04 | cysteine dioxygenase, type I (CDO1), | Homo sapiens cysteine dioxygenase, | BG081135 | Mm.29996 | Energy/Metabolism |
| 1946 | | | type I (CDO1), mRNA | | | |
| 1947 | H3061H08 | guanine deaminase (Gda), | Mus musculus guanine deaminase | BG081171 | | Energy/Metabolism |
| 1948 | | | (Gda), mRNA | | | |
| 1949 | H3062C04 | spermine synthase (Sms), | Mus musculus spermine synthase (Sms), | BG081202 | | Energy/Metabolism |
| 1950 | | | mRNA | | | |
| 1951 | H3062E02 | isocitrate dehydrogenase | Mus musculus isocitrate dehydrogenase | BG081213 | Mm.18213 | Energy/Metabolism |
| 1952 | | | mRNA, complete cds | | | |
| 1953 | H3062H07 | “Mus musculus inositol 1,4,5- | | BG081243 | Mm.2726 | Signal Transduction |
| 1954 | | triphosphate receptor 1 (Itpr1), mRNA” | | | | |
| 1955 | H3062H08 | Mm.11827 | | | Mm.11827 | Energy/Metabolism |
| 1956 | | | | | | |
| 1957 | H3063A03 | “Mus musculus calpain 7 (Capn7), | | BG081250 | Mm.142370 | Signal Transduction |
| 1958 | | mRNA” | | | | |
| 1959 | H3063A08 | Prsc1 | Mus musculus protease, cysteine, 1 | BG068219 | Mm.17185 | Energy/Metabolism |
| 1960 | | | (Prsc1), mRNA | | | |
| 1961 | H3063A09 | “Mus musculus novel ras effector 1 | | BG068220 | Mm.10133 | Signal Transduction |
| 1962 | | (Norel-pending), mRNA” | | | | |
| 1963 | H3063C09 | beta-1,4-galactosyltransferase VI | Mus musculus beta-1,4- | BG081279 | Mm.26364 | Energy/Metabolism |
| 1964 | | | galactosyltransferase VI mRNA, | | | |
| | | | complete cds | | | |
| 1965 | H3063H10 | LAT2 (Slc7a8) | Mus musculus mRNA for LAT2 protein | BG068299 | Mm.27830 | Energy/Metabolism |
| 1966 | | | (Slc7a8 gene) | | | |
| 1967 | H3064C02 | “Mus musculus large tumor suppressor | | BG081357 | Mm.35642 | Signal Transduction |
| 1968 | | 1 (Lats1) mRNA, partial cds” | | | | |
| 1969 | H3064C04 | kinesin-like 5 (mitotic kinesin-like | Homo sapiens kinesin-like 5 (mitotic | | Mm.28386 | Cell Cycle |
| 1970 | | protein 1) (KNSL5), | kinesin-like protein 1) (KNSL5), mRNA | | | |
| 1971 | H3064E02 | vacuolar adenosine triphosphatase | Mus musculus vacuolar adenosine | BG081377 | Mm.29771 | Energy/Metabolism |
| 1972 | | subunit A gene | triphosphatase subunit A gene, complete | | | |
| | | | cds | | | |
| 1973 | H3064H04 | HMG box protein | Mus musculus HMG box protein | | Mm.41766 | Transcription/Chromatin |
| 1974 | | | mRNA, complete cds | | | |
| 1975 | H3065C08 | “Mus musculus calmodulin dependent | | BG081448 | Mm.24381 | Signal Transduction |
| 1976 | | phosphatase catalytic subunit (Cam-Prp) | | | | |
| | | mRNA, 3′ end” | | | | |
| 1977 | H3065C11 | “Mus musculus Chetk-alpha gene for | | BG081451 | | Signal Transduction |
| 1978 | | choline/ethanolamine kinase-alpha, | | | | |
| | | exon 1 and 5′-flanking region” | | | | |
| 1979 | H3065D10 | “Homo sapiens RAP1A, member of | | BG068432 | Mm.144498 | Signal Transduction |
| 1980 | | RAS oncogene family (RAP1A), | | | | |
| | | mRNA” | | | | |
| 1981 | H3065E07 | UDP-glucose dehydrogenase (Ugdh), | Mus musculus UDP-glucose | BG068439 | Mm.10709 | Energy/Metabolism |
| 1982 | | | dehydrogenase (Ugdh), mRNA | | | |
| 1983 | H3066H07 | heme oxygenase-2 | Mus musculus heme oxygenase-2 | BG081591 | | Energy/Metabolism |
| 1984 | | | mRNA, complete cds | | | |
| 1985 | H3067A07 | potassium channel modulatory factor | Mus musculus potassium channel | BG081603 | Mm.29194 | Energy/Metabolism |
| 1986 | | DEBT-91 (Debt91), | modulatory factor DEBT-91 (Debt91), | | | |
| | | | mRNA | | | |
| 1987 | H3067B08 | “Mus musculus phosphoinositide 3- | | BG081616 | Mm.3058 | Signal Transduction |
| 1988 | | kinase regulatory subunit p85alpha | | | | |
| | | mRNA, complete cds” | | | | |
| 1989 | H3067E02 | “Mus musculus RAS-related C3 | | BG068616 | Mm.29014 | Signal Transduction |
| 1990 | | botulinum substrate 1, guanine | | | | |
| | | nucleotide exchange factor 1 (Racgefl- | | | | |
| | | pending), mRNA” | | | | |
| 1991 | H3068A08 | kinesin-like 5 (mitotic kinesin-like | Homo sapiens kinesin-like 5 (mitotic | | Mm.28386 | Cell Cycle |
| 1992 | | protein 1) (KNSL5), | kinesin-like protein 1) (KNSL5), mRNA | | | |
| 1993 | H3068A11 | ferritin light chain 1 (Ftl1), | Mus musculus ferritin light chain 1 | BG081695 | | Energy/Metabolism |
| 1994 | | | (Ftl1), mRNA | | | |
| 1995 | H3068F03 | ornithine decarboxylase antizyme | Mus musculus ornithine decarboxylase | BG081746 | Mm.104010 | Energy/Metabolism |
| 1996 | | inhibitor (Oazi), | antizyme inhibitor (Oazi), mRNA | | | |
| 1997 | H3069C09 | “Mus musculus Rho-associated coiled- | | BG081800 | Mm.6710 | Signal Transduction |
| 1998 | | coil forming kinase 1 (Rock1), mRNA” | | | | |
| 1999 | H3069D09 | cDNA FLJ12814 fis, clone | Homo sapiens cDNA FLJ12814 fis, | BG081823 | Mm.27228 | Energy/Metabolism |
| 2000 | | NT2RP2002520, weakly similar to | clone NT2RP2002520, weakly similar | | | |
| | | transcription factor RFX-B (RFXB) | to Homo sapiens transcription factor | | | |
| | | | RFX-B (RFXB) mRNA | | | |
| 2001 | H3069G01 | choline/ethanolaminephosphotransferase | Homo sapiens | BG081849 | Mm.14816 | Energy/Metabolism |
| 2002 | | (CEPT1), | choline/ethanolaminephosphotransferase | | | |
| | | | (CEPT1), mRNA | | | |
| 2003 | H3070A09 | RHOA proto-oncogene multi-drug- | Homo sapiens RHOA proto-oncogene | BG081880 | Mm.757 | Energy/Metabolism |
| 2004 | | resistance protein, 3 end | multi-drug-resistance protein mRNA, 3 | | | |
| | | | end | | | |
| 2005 | H3070A12 | quinoid dihydropteridine reductase | Homo sapiens quinoid dihydropteridine | BG081883 | Mm.30204 | Energy/Metabolism |
| 2006 | | (QDPR), | reductase (QDPR), mRNA | | | |
| 2007 | H3070B09 | partial Kcnq1 gene potassium channel | Mus musculus partial Kcnq1 gene for | BG081892 | | Energy/Metabolism |
| 2008 | | protein, exons 10-14 | potassium channel protein, exons 10-14 | | | |
| 2009 | H3070C08 | hydroxysteroid dehydrogenase-1, | Mus musculus hydroxysteroid | BG081903 | Mm.16941 | Energy/Metabolism |
| 2010 | | delta<5>-3-beta (Hsd3b1), | dehydrogenase-1, delta<5>-3-beta | | | |
| | | | (Hsd3b1), mRNA | | | |
| 2011 | H3070E04 | ATP-specific succinyl-CoA synthetase | Mus musculus ATP-specific succinyl- | BG068897 | Mm.19154 | Energy/Metabolism |
| 2012 | | beta subunit (Scs), partial cds | CoA synthetase beta subunit (Scs) | | | |
| | | | mRNA, partial cds | | | |
| 2013 | H3071A03 | ATPase, class VI, type 11A (Atp11a), | Mus musculus ATPase, class VI, type | BG081967 | Mm.148756 | Energy/Metabolism |
| 2014 | | | 11A (Atp11a), mRNA | | | |
| 2015 | H3071G11 | “Homo sapiens RAP2B, member of | | BG082041 | Mm.26939 | Signal Transduction |
| 2016 | | RAS oncogene family (RAP2B), | | | | |
| | | mRNA” | | | | |
| 2017 | H3071H04 | glycerol-3-phosphate acyltransferase | Mouse glycerol-3-phosphate | | Mm.87773 | |
| | | | acyltransferase mRNA, complete cds | | | |
| 2018 | H3072A08 | glyceraldehyde-3-phosphate | Mus musculus glyceraldehyde-3- | BG082061 | | Energy/Metabolism |
| 2019 | | dehydrogenase (Gapd) | phosphate dehydrogenase (Gapd), | | | |
| | | | mRNA | | | |
| 2020 | H3072B11 | “Mus musculus PLC-L2 mRNA for | | BG069051 | Mm.28034 | Signal Transduction |
| 2021 | | phospholipase C-L2, complete cds” | | | | |
| 2022 | H3072F03 | Mm.22651 | | | Mm.22651 | Energy/Metabolism |
| 2023 | | | | | | |
| 2024 | H3072G09 | potassium voltage-gated channel, | Mus musculus potassium voltage-gated | BG069106 | Mm.4489 | Energy/Metabolism |
| 2025 | | subfamily H (eag-related), member 1 | channel, subfamily H (eag-related), | | | |
| | | (Kcnh1), | member 1 (Kcnh1), mRNA | | | |
| 2026 | H3073F09 | Mm.27123 | | | Mm.27123 | Energy/Metabolism |
| 2027 | | | | | | |
| 2028 | H3073F10 | “Mus musculus 80 kDa m-calpain | | BG082209 | Mm.6958 | Signal Transduction |
| 2029 | | subunit (calp80) mRNA, complete cds” | | | | |
| 2030 | H3073G07 | granzyme M | Mus musculus mRNA for granzyme M, | BG082217 | Mm.22302 | Energy/Metabolism |
| 2031 | | | complete cds | | | |
| 2032 | H3074A02 | inhibitor of apoptosis protein 1 | Mus musculus inhibitor of apoptosis | BG069214 | Mm.2026 | Apoptosis |
| 2033 | | | protein 1 mRNA, complete cds | | | |
| 2034 | H3074A03 | NIMA (never in mitosis gene a)-related | Mus musculus NIMA (never in mitosis | | Mm.143817 | Cell Cycle |
| 2035 | | expressed kinase 7 (Nek7), | gene a)-related expressed kinase 7 | | | |
| | | | (Nek7), mRNA | | | |
| 2036 | H3074C12 | dUTPase | Mus musculus dUTPase mRNA, | BG082266 | | Energy/Metabolism |
| 2037 | | | complete cds | | | |
| 2038 | H3074F08 | “Mus musculus protein phosphatase | | BG082298 | Mm.11711 | Signal Transduction |
| 2039 | | type 2A catalytic subunit alpha isoform | | | | |
| | | mRNA, complete cds” | | | | |
| 2040 | H3074G02 | H. sapiens 40 kDa protein kinase related | H. sapiens 40 kDa protein kinase related | | Mm.1700 | |
| 2041 | | to rat ERK2 | to rat ERK2 | | | |
| 2042 | H3074H09 | NAALADase II protein | Homo sapiens mRNA for NAALADase | BG082322 | Mm.26132 | Energy/Metabolism |
| 2043 | | | II protein | | | |
| 2044 | H3075A04 | Mm.26128 | | | Mm.26128 | Energy/Metabolism |
| 2045 | | | | | | |
| 2046 | H3075A09 | esterase-22 = endoplasmic reticulum- | esterase-22 = endoplasmic reticulum- | BG069315 | Mm.29110 | Energy/Metabolism |
| 2047 | | targeting protein of beta-glucuronidase | targeting protein of beta-glucuronidase | | | |
| | | [mice,, 2022 nt] | [mice, mRNA, 2022 nt] | | | |
| 2048 | H3075F01 | ATP-specific succinyl-CoA synthetase | Mus musculus ATP-specific succinyl- | BG069442 | Mm.19154 | Energy/Metabolism |
| 2049 | | beta subunit (Scs), partial cds | CoA synthetase beta subunit (Scs) | | | |
| | | | mRNA, partial cds | | | |
| 2050 | H3075F08 | F1F0-ATP synthase, g subunit | Mus musculus mRNA for F1F0-ATP | BG082389 | Mm.14663 | Energy/Metabolism |
| 2051 | | | synthase, g subunit | | | |
| 2052 | H3075F11 | “Mus musculus heat shock protein 20- | | BG069463 | Mm.21549 | Heat Shock/Stress |
| 2053 | | like protein mRNA, complete cds” | | | | |
| 2054 | H3075G06 | molybdenum cofactor synthesis-step 1 | Mus musculus molybdenum cofactor | BG082399 | Mm.36112 | Energy/Metabolism |
| 2055 | | proteins A and B splice type I (Mocs1) | synthesis-step 1 proteins A and B splice | | | |
| | | s, partial cds, alternatively spliced | type I (Mocs1) mRNAs, partial cds, | | | |
| | | | alternatively spliced | | | |
| 2056 | H3075H02 | peptidyl arginine deiminase, type II | Mus musculus peptidyl arginine | BG082407 | Mm.2296 | Energy/Metabolism |
| 2057 | | (Pdi2), | deiminase, type II (Pdi2), mRNA | | | |
| 2058 | H3076B05 | “Mus musculus Rho interacting protein | | BG069493 | Mm.4620 | Signal Transduction |
| 2059 | | 2 (Rhoip2-pending), mRNA” | | | | |
| 2060 | H3076C12 | fatty-acid-Coenzyme A ligase, long- | Homo sapiens fatty-acid-Coenzyme A | BG069344 | Mm.12166 | Energy/Metabolism |
| 2061 | | chain 4 (FACL4), | ligase, long-chain 4 (FACL4), mRNA | | | |
| 2062 | H3076F04 | “AC005290, complete sequence [Mus | | BG069372 | Mm.1967 | Signal Transduction |
| 2063 | | musculus]” | | | | |
| 2064 | H3076F12 | “Homo sapiens regulator of G protein | | BG082484 | Mm.31378 | Signal Transduction |
| 2065 | | signaling RGS12 (RGS) mRNA, | | | | |
| | | complete cds” | | | | |
| 2066 | H3076G06 | “Mus musculus signal transducer and | | BG069385 | Mm.21935 | Signal Transduction |
| 2067 | | activator of transcription 2 (Stat2), | | | | |
| | | mRNA” | | | | |
| 2068 | H3076G11 | similar to argininosuccinate lyase (H. sapiens) | Homo sapiens similar to | BG082495 | Mm.23869 | Energy/Metabolism |
| 2069 | | (LOC63402), | argininosuccinate lyase (H. sapiens) | | | |
| | | | (LOC63402), mRNA | | | |
| 2070 | H3076H06 | “Mus musculus adenylate kinase 4 | | BG082501 | Mm.142051 | Signal Transduction |
| 2071 | | (Ak4), mRNA” | | | | |
| 2072 | H3076H10 | “Mus musculus casein kinase II, alpha | | BG069401 | Mm.28881 | Signal Transduction |
| 2073 | | 2, polypeptide (Csnk2a2), mRNA” | | | | |
| 2074 | H3077B02 | solute carrier family 12, member 2 | Mus musculus solute carrier family 12, | BG069505 | Mm.4168 | Energy/Metabolism |
| 2075 | | (Slc12a2), | member 2 (Slc12a2), mRNA | | | |
| 2076 | H3077C01 | X28 region near ALD locus containing | Homo sapiens X28 region near ALD | BG069516 | Mm.28056 | Energy/Metabolism |
| 2077 | | dual specificity phosphatase 9 (DUSP9), | locus containing dual specificity | | | |
| | | ribosomal protein L18a (RPL18a), | phosphatase 9 (DUSP9), ribosomal | | | |
| | | Ca2+/Calmodulin-dependent protein | protein L18a (RPL18a), | | | |
| | | kinase I (CAMKI), creatine transporter ( | Ca2+/Calmodulin-dependent protein | | | |
| | | | kinase I (CAMKI), creatine transporter ( | | | |
| 2078 | H3077F08 | ATPase, H(+)-transporting, lysosomal, | Homo sapiens ATPase, H(+)- | BG082574 | Mm.26909 | Energy/Metabolism |
| 2079 | | noncatalytic accessory protein 1B | transporting, lysosomal, noncatalytic | | | |
| | | (ATP6N1B), | accessory protein 1B (ATP6N1B), | | | |
| | | | mRNA | | | |
| 2080 | H3077H01 | hypothetical protein FLJ10726 | Homo sapiens hypothetical protein | | Mm.30718 | |
| 2081 | | (FLJ10726), | FLJ10726 (FLJ10726), mRNA | | | |
| 2082 | H3078G02 | NIMA (never in mitosis gene a)-related | Mus musculus NIMA (never in mitosis | | Mm.143817 | Cell Cycle |
| 2083 | | expressed kinase 7 (Nek7), | gene a)-related expressed kinase 7 | | | |
| | | | (Nek7), mRNA | | | |
| 2084 | H3078H09 | Rat L-gulono-gamma-lactone oxidase | Rat L-gulono-gamma-lactone oxidase | BG082674 | Mm.26207 | Energy/Metabolism |
| 2085 | | | mRNA, complete cds | | | |
| 2086 | H3078H11 | TRPM-2, cytosolic epoxide hydrolase, | Homo sapiens TRPM-2, cytosolic | BG082676 | Mm.26207 | Energy/Metabolism |
| 2087 | | nicotinic acetylcholine receptor alpha2 | epoxide hydrolase, nicotinic | | | |
| | | subunit, and focal adhesion kinase | acetylcholine receptor alpha2 subunit, | | | |
| | | genes, sequence | and focal adhesion kinase genes, | | | |
| | | | complete cds, complete sequence | | | |
| 2088 | H3079D11 | solute carrier family 12, member 2 | Mus musculus solute carrier family 12, | BG069726 | Mm.4168 | Energy/Metabolism |
| 2089 | | (Slc12a2), | member 2 (Slc12a2), mRNA | | | |
| 2090 | H3079D12 | “Mus musculus phospholipase D3 | | BG069727 | Mm.6483 | Signal Transduction |
| 2091 | | (Pld3), mRNA” | | | | |
| 2092 | H3079E06 | “Mus musculus interferon regulatory | | BG069733 | Mm.4179 | Heat Shock/Stress |
| 2093 | | factor 6 (Irf6), mRNA” | | | | |
| 2094 | H3079F01 | cDNA: FLJ22083 fis, clone HEP14459, | Homo sapiens cDNA: FLJ22083 fis, | BG069739 | Mm.22675 | Energy/Metabolism |
| 2095 | | highly similar to HUM3H3M 3- | clone HEP14459, highly similar to | | | |
| | | hydroxy-3-methylglutaryl coenzyme A | HUM3H3M Homo sapiens 3-hydroxy- | | | |
| | | synthase | 3-methylglutaryl coenzyme A synthase | | | |
| | | | mRNA | | | |
| 2096 | H3079F05 | “Mus musculus calcyclin binding | | BG069742 | Mm.10702 | Signal Transduction |
| 2097 | | protein (CACYBP) mRNA, partial cds” | | | | |
| 2098 | H3079G04 | cystathionine beta-synthase (CBS) gene, | Homo sapiens cystathionine beta- | BG082744 | Mm.24225 | Energy/Metabolism |
| 2099 | | major and minor alternative splice | synthase (CBS) gene, major and minor | | | |
| | | products | alternative splice products, complete | | | |
| | | | cds | | | |
| 2100 | H3079G06 | “Mus musculus interferon regulatory | | BG069754 | Mm.3960 | Heat Shock/Stress |
| 2101 | | factor 3 (Irf3), mRNA” | | | | |
| 2102 | H3079G12 | cDNA DKFZp727E011 (from clone | Homo sapiens mRNA; cDNA | BG082752 | Mm.24225 | Energy/Metabolism |
| 2103 | | DKFZp727E011); partial cds | DKFZp727E011 (from clone | | | |
| | | | DKFZp727E011); partial cds | | | |
| 2104 | H3079H11 | M. musculus RPS3a gene | M. musculus RPS3a gene | BG069771 | Mm.6957 | Apoptosis |
| 2105 | | | | | | |
| 2106 | H3080A09 | “Homo sapiens similar to cullin 5 (H. | | BG069781 | Mm.21710 | Signal Transduction |
| 2107 | | sapiens)(LOC63330), mRNA” | | | | |
| 2108 | H3080B11 | TF-1 cell apoptosis related protein-15 | Mus musculus TF-1 cell apoptosis | BG069793 | Mm.28441 | Apoptosis |
| 2109 | | (Tfar15), | related protein-15 (Tfar15), mRNA | | | |
| 2110 | H3080G08 | Mus musculus ribonuclease P2 | | BG069849 | Mm.12440 | Energy/Metabolism |
| 2111 | | (Rnasep2-pending), mRNA | | | | |
| 2112 | H3080G10 | “Mus musculus adenomatosis polyposis | | BG069851 | Mm.7883 | Signal Transduction |
| 2113 | | coli (Apc), mRNA” | | | | |
| 2114 | H3080H10 | Mus musculus RNA guanylyltransferase | | BG069862 | Mm.26153 | Energy/Metabolism |
| 2115 | | and 5-phosphatase (Rngtt), mRNA | | | | |
| 2116 | H3081A12 | fatty acid-Coenzyme A ligase, long | Mus musculus fatty acid-Coenzyme A | BG069876 | Mm.143689 | Energy/Metabolism |
| 2117 | | chain 4 (Facl4), | ligase, long chain 4 (Facl4), mRNA | | | |
| 2118 | H3081D02 | Bcl-2-related ovarian killer protein-like- | Mus musculus Bcl-2-related ovarian | BG082842 | Mm.3295 | Apoptosis |
| 2119 | | pending (Bokl-pending), | killer protein-like-pending (Bokl- | | | |
| | | | pending), mRNA | | | |
| 2120 | H3081E08 | TNF-alpha converting enzyme | Mus musculus TNF-alpha converting | BG082857 | Mm.27681 | Apoptosis |
| 2121 | | precursor,, alternatively spliced | enzyme precursor, mRNA, alternatively | | | |
| | | | spliced, complete cds | | | |
| 2122 | H3081H02 | aldo-keto reductase AKR1C13 | Mus musculus aldo-keto reductase | BG082884 | Mm.27447 | Energy/Metabolism |
| 2123 | | (Akr1c13), | AKR1C13 (Akr1c13), mRNA | | | |
| 2124 | H3082B04 | Mus musculus inducible 6- | | BG070045 | Mm.19669 | Energy/Metabolism |
| 2125 | | phosphofructo-2-kinase mRNA, | | | | |
| | | complete cds | | | | |
| 2126 | H3082C01 | Mus musculus serine protease inhibitor | | BG070053 | Mm.89871 | Energy/Metabolism |
| 2127 | | 1-5 (Spi1-5), mRNA | | | | |
| 2128 | H3082E03 | Mus musculus programmed cell death 8 | | BG082930 | Mm.30050 | Apoptosis |
| 2129 | | (apoptosis inducing factor) (Pdcd8), | | | | |
| | | mRNA | | | | |
| 2130 | H3082E10 | H. sapiens mitogen inducible gene mig- | | | Mm.29842 | Signal Transduction |
| 2131 | | 2, complete CDS | | | | |
| 2132 | H3082F03 | M. musculus (balb-c) gene for | | BG082939 | Mm.4628 | Energy/Metabolism |
| 2133 | | sphingomyelin phosphodiesterase | | | | |
| 2134 | H3083C01 | Homo sapiens kinesin-like 5 (mitotic | | | Mm.28386 | Cell Cycle |
| 2135 | | kinesin-like protein 1) (KNSL5), mRNA | | | | |
| 2136 | H3083D06 | Mus musculus Bcl2-associated | | BG069990 | Mm.688 | Apoptosis |
| 2137 | | athanogene 1 (Bag1), mRNA | | | | |
| 2138 | H3083D09 | Homo sapiens cDNA FLJ14310 fis, | | BG069993 | Mm.7156 | Energy/Metabolism |
| 2139 | | clone PLACE3000271 | | | | |
| 2140 | H3083E02 | “Mus musculus beta-site APP cleaving | | BG069998 | Mm.24044 | Signal Transduction |
| | | enzyme (Bace), mRNA” | | | | |
| 2141 | H3083H02 | “Mus musculus guanine nucleotide | | BG070116 | Mm.5305 | Signal Transduction |
| 2142 | | binding protein, beta-2, related | | | | |
| | | sequence 1 (Gnb2-rs1), mRNA” | | | | |
| 2143 | H3083H05 | Mus musculus mRNA for tumor | | BG070119 | Mm.27681 | Energy/Metabolism |
| 2144 | | necrosis factor alpha converting enzyme | | | | |
| | | (TACE/ADAM17), complete cds | | | | |
| 2145 | H3084E05 | glutathione S-transferase, mu 5 (Gstm5), | Mus musculus glutathione S-transferase, | BG070174 | Mm.667 | Energy/Metabolism |
| 2146 | | | mu 5 (Gstm5), mRNA | | | |
| 2147 | H3084F02 | Mm.24864 | | | Mm.24864 | Energy/Metabolism |
| 2148 | | | | | | |
| 2149 | H3084G03 | Mouse c-fos gene; cellular homolog to | | BG070196 | Mm.5043 | Signal Transduction |
| 2150 | | viral oncogene | | | | |
| 2151 | H3085B01 | M. musculus mRNA for casein kinase I- | | BG083155 | Mm.43737 | Signal Transduction |
| 2152 | | alpha | | | | |
| 2153 | H3085C02 | Mus musculus Traf and Tnf receptor | | BG070243 | Mm.155228 | Apoptosis |
| 2154 | | associated protein (Ttrap-pending), | | | | |
| | | mRNA | | | | |
| 2155 | H3085C04 | Mus musculus solute carrier family 12, | | BG070245 | Mm.4168 | Energy/Metabolism |
| 2156 | | member 2 (Slc12a2), mRNA | | | | |
| 2157 | H3085D10 | Mus musculus guanine deaminase | | BG083182 | Mm.25912 | Energy/Metabolism |
| 2158 | | (Gda), mRNA | | | | |
| 2159 | H3085G03 | Mouse cytochrome beta-558 mRNA, 3 | | BG083209 | Mm.448 | Energy/Metabolism |
| 2160 | | end | | | | |
| 2161 | H3086E09 | Homo sapiens BCL2-associated | | BG070363 | Mm.44239 | Apoptosis |
| 2162 | | athanogene 5 (BAG5), mRNA | | | | |
| 2163 | H3086F10 | B. taurus CI-SGDH mRNA for | | BG083295 | Mm.28058 | Energy/Metabolism |
| 2164 | | ubiquinone oxidoreductase complex | | | | |
| 2165 | H3086H09 | “Mus musculus mRNA for Rho guanine | | BG083317 | Mm.22261 | Signal Transduction |
| 2166 | | nucleotide-exchange factor, splice | | | | |
| | | variant NET1A” | | | | |
| 2167 | H3087A12 | uterine lactotransferrin | Mouse uterine lactotransferrin mRNA | BG070413 | Mm.7612 | Energy/Metabolism |
| 2168 | | | | | | |
| 2169 | H3087C10 | “Mus musculus rho GTPase activating | | BG083351 | | Signal Transduction |
| 2170 | | protein 6 (Arhgap6), mRNA” | | | | |
| 2171 | H3087G09 | “Mus musculus heat shock protein 030 | | BG070480 | Mm.104491 | Heat Shock/Stress |
| 2172 | | (Hsp030), mRNA” | | | | |
| 2173 | H3087H05 | acetoacetyl-coenzyme A thiolase (EC | Human mRNA for acetoacetyl- | BG083405 | Mm.1776 | Energy/Metabolism |
| 2174 | | 2.3.1.9) | coenzyme A thiolase (EC 2.3.1.9) | | | |
| 2175 | H3087H09 | “Mus musculus calcium binding protein | | BG083409 | Mm.1221 | Signal Transduction |
| 2176 | | A6 (calcyclin) (S100a6), mRNA” | | | | |
| 2177 | H3088A07 | glutathione S-transferase, mu 2 (Gstm2), | Mus musculus glutathione S-transferase, | BG070501 | Mm.7446 | Energy/Metabolism |
| 2178 | | | mu 2 (Gstm2), mRNA | | | |
| 2179 | H3088C12 | CPP32 apoptotic protease, partial cds | Mus musculus CPP32 apoptotic | BG083524 | Mm.153450 | Apoptosis |
| 2180 | | | protease mRNA, partial cds | | | |
| 2181 | H3088E07 | “Mus musculus choline kinase (Chk), | | BG070547 | Mm.5262 | Signal Transduction |
| 2182 | | mRNA” | | | | |
| 2183 | H3088F03 | “Mus musculus protein tyrosine | | BG083549 | Mm.4124 | Signal Transduction |
| 2184 | | phosphatase 4a3 (Ptp4a3), mRNA” | | | | |
| 2185 | H3089A06 | Mus musculus ATPase, H+ | | BG083588 | Mm.10727 | Energy/Metabolism |
| 2186 | | transporting, lysosomal (vacuolar | | | | |
| | | proton pump), beta 56/58 kDa, isoform | | | | |
| | | 2 (Atp6b2), mRNA | | | | |
| 2187 | H3089A11 | Mus musculus ATP-binding cassette | | BG083593 | Mm.21629 | Energy/Metabolism |
| 2188 | | protein (Abcf2) mRNA, partial cds | | | | |
| 2189 | H3089F09 | “Mus musculus protein kinase Chk2 | | BG083482 | Mm.126031 | Signal Transduction |
| 2190 | | (Rad53-pending), mRNA” | | | | |
| 2191 | H3089G12 | Mus musculus zinc transporter like 1 | | BG083497 | Mm.28851 | Energy/Metabolism |
| 2192 | | (Zntl1) mRNA, complete cds | | | | |
| 2193 | H3089H11 | Mouse normal c-myc gene and | | BG083596 | Mm.2444 | Signal Transduction |
| 2194 | | translocated homologue from J558 | | | | |
| | | plasmocytoma cells (cDNA sequence) | | | | |
| 2195 | H3090B01 | “Mus musculus mothers-against-dpp- | | BG083610 | Mm.15185 | Signal Transduction |
| 2196 | | related-1 mRNA, complete cds” | | | | |
| 2197 | H3090B05 | uracil-DNA glycosylase gene, | Mus musculus uracil-DNA glycosylase | BG070697 | Mm.112312 | Energy/Metabolism |
| 2198 | | alternatively spliced | gene, complete cds, alternatively spliced | | | |
| 2199 | H3090C08 | ornithine decarboxylase antizyme | Mus musculus ornithine decarboxylase | BG070710 | Mm.6775 | Energy/Metabolism |
| 2200 | | inhibitor (Oazi), | antizyme inhibitor (Oazi), mRNA | | | |
| 2201 | H3090C10 | N-terminal asparagine amidohydrolase | Mus musculus N-terminal asparagine | BG083629 | Mm.22635 | Energy/Metabolism |
| 2202 | | (Ntan1) gene | amidohydrolase (Ntan1) gene, complete | | | |
| | | | cds | | | |
| 2203 | H3090D01 | “Mus musculus mitogen activated | | BG083632 | Mm.4358 | Signal Transduction |
| 2204 | | protein kinase kinase kinase 12 | | | | |
| | | (Map3k12), mRNA” | | | | |
| 2205 | H3091B08 | partial gene encoding | Mus musculus partial gene encoding | BG070787 | Mm.42205 | Energy/Metabolism |
| 2206 | | deoxyribonuclease I and 5 UTR | deoxyribonuclease I and 5 UTR | | | |
| 2207 | H3091D08 | “Mus musculus guanine nucleotide | | BG083726 | Mm.133949 | Signal Transduction |
| 2208 | | binding protein, alpha 14 (Gna14), | | | | |
| | | mRNA” | | | | |
| 2209 | H3091D11 | tumor necrosis factor (ligand) | Mus musculus tumor necrosis factor | BG070891 | Mm.22585 | Apoptosis |
| 2210 | | superfamily, member 19 (Tnfsf19- | (ligand) superfamily, member 19 | | | |
| | | pending), | (Tnfsf19-pending), mRNA | | | |
| 2211 | H3091E02 | 5(3)-deoxyribonucleotidase (Dnt- | Mus musculus 5(3)- | BG070894 | Mm.41722 | Energy/Metabolism |
| 2212 | | pending), | deoxyribonucleotidase (Dnt-pending), | | | |
| | | | mRNA | | | |
| 2213 | H3091F08 | N-terminal Asn amidase (Ntan1), | Mus musculus N-terminal ASn amidase | BG070912 | Mm.22635 | Energy/Metabolism |
| 2214 | | | (Ntan1), mRNA | | | |
| 2215 | H3091G12 | solute carrier family 1, member 7 | Mus musculus solute carrier family 1, | BG070928 | Mm.1056 | Energy/Metabolism |
| 2216 | | (Slc1a7), | member 7 (Slc1a7), mRNA | | | |
| 2217 | H3091H05 | “Mus musculus H-ras gene, partial cds” | | BG070933 | Mm.6793 | Signal Transduction |
| 2218 | | | | | | |
| 2219 | H3091H07 | deiodinase, iodothyronine type III | Rattus norvegicus deiodinase, | BG070934 | Mm.154427 | Energy/Metabolism |
| 2220 | | (Dio3), | iodothyronine type III (Dio3), mRNA | | | |
| 2221 | H3092E03 | Balb/c cytochrome c oxidase subunit | Mus musculus Balb/c cytochrome c | BG083822 | Mm.43415 | Energy/Metabolism |
| 2222 | | VIaL | oxidase subunit VIaL mRNA, complete | | | |
| | | | cds | | | |
| 2223 | H3092E04 | caspase 8 associated protein 2 | Mus musculus caspase 8 associated | BG070820 | Mm.22279 | Apoptosis |
| 2224 | | (Casp8ap2), | protein 2 (Casp8ap2), mRNA | | | |
| 2225 | H3092G01 | “Mus musculus p38delta MAP kinase | | BG083840 | Mm.27970 | Signal Transduction |
| 2226 | | mRNA, complete cds” | | | | |
| 2227 | H3092H01 | vacuolar adenosine triphosphatase | Mus musculus vacuolar adenosine | BG070846 | Mm.29771 | Energy/Metabolism |
| 2228 | | subunit A gene | triphosphatase subunit A gene, complete | | | |
| | | | cds | | | |
| 2229 | H3092H09 | “Mus musculus mitogen-activated | | BG083857 | | Signal Transduction |
| 2230 | | protein kinase 8 interacting protein 2 | | | | |
| | | (Mapk8ip2), mRNA” | | | | |
| 2231 | H3092H10 | glycogenin 1 (Gyg1), | Mus musculus glycogenin 1 (Gyg1), | BG070855 | Mm.6375 | Energy/Metabolism |
| 2232 | | | mRNA | | | |
| 2233 | H3093B07 | chloride channel 5 (Clcn5), | Mus musculus chloride channel 5 | BG083875 | Mm.104172 | Energy/Metabolism |
| 2234 | | | (Clcn5), mRNA | | | |
| 2235 | H3093D09 | Tom22 | Homo sapiens mRNA for Tom22, | BG083900 | Mm.9326 | Energy/Metabolism |
| 2236 | | | complete cds | | | |
| 2237 | H3093F02 | uridine monophosphate kinase (UMPK), | Homo sapiens uridine monophosphate | BG083915 | | Energy/Metabolism |
| 2238 | | | kinase (UMPK), mRNA | | | |
| 2239 | H3093G07 | “Homo sapiens Putative prostate cancer | | BG071015 | Mm.24156 | Signal Transduction |
| 2240 | | tumor suppressor (N33), mRNA” | | | | |
| 2241 | H3093G08 | GST-5 = glutathione S-transferase-sperm | GST-5 = glutathione S-transferase-sperm | BG071016 | Mm.11346 | Energy/Metabolism |
| 2242 | | antigen MSAg-5 fusion protein {3 | antigen MSAg-5 fusion protein {3 | | | |
| | | region} [mice, testis, Recombinant | region} [mice, testis, mRNA | | | |
| | | Partial, 860 nt] | Recombinant Partial, 860 nt] | | | |
| 2243 | H3093H01 | mannosyl (alpha-1,6-)-glycoprotein | Homo sapiens mannosyl (alpha-1,6-)- | BG084016 | Mm.24293 | Energy/Metabolism |
| 2244 | | beta-1,2-N- | glycoprotein beta-1,2-N- | | | |
| | | acetylglucosaminyltransferase | acetylglucosaminyltransferase | | | |
| | | (MGAT2), | (MGAT2), mRNA | | | |
| 2245 | H3094C01 | Mus musculus gene for prostacyclin | | BG084049 | | Energy/Metabolism |
| 2246 | | synthase, 5-flanking region and partial | | | | |
| | | cds | | | | |
| 2247 | H3094D01 | Mus musculus cytochrome P450 side | | BG071067 | Mm.108678 | Energy/Metabolism |
| 2248 | | chain cleavage enzyme 11a1 | | | | |
| | | (Cyp11a1), mRNA | | | | |
| 2249 | H3094D02 | “Mus musculus GNB1 gene for G | | BG071068 | Mm.2344 | Signal Transduction |
| 2250 | | protein beta1 subunit, exon 12 and | | | | |
| | | complete cds” | | | | |
| 2251 | H3094D04 | Mus musculus GLUT4 vesicle protein | | BG071070 | Mm.29010 | Energy/Metabolism |
| 2252 | | mRNA, partial cds | | | | |
| 2253 | H3094E12 | | | | Mm.26092 | Energy/Metabolism |
| 2254 | | | | | | |
| 2255 | H3094G04 | Homo sapiens cDNA FLJ12225 fis, | | BG083967 | Mm.12983 | Energy/Metabolism |
| 2256 | | clone MAMMA1001139, weakly | | | | |
| | | similar to SRE-2 PROTEIN | | | | |
| 2257 | H3094G10 | Homo sapiens mitogen-activated protein | | | Mm.29588 | |
| 2258 | | kinase kinase kinase kinase 3 | | | | |
| | | (MAP4K3), mRNA | | | | |
| 2259 | H3094H06 | Mus musculus Bcl2-interacting killer- | | BG071119 | Mm.17912 | Apoptosis |
| 2260 | | like (Biklk), mRNA | | | | |
| 2261 | H3095D11 | Mus musculus mitogen activated protein | | | | |
| 2262 | | kinase kinase kinase 12 (Map3k12), | | | | |
| | | mRNA | | | | |
| 2263 | H3095E06 | Mus musculus glutathione S-transferase | | BG084006 | Mm.282 | Energy/Metabolism |
| | | like (Gsttl-pending), mRNA | | | | |
| 2264 | H3095F10 | Homo sapiens calcineurin binding | | BG071186 | Mm.154496 | |
| 2265 | | protein 1 (KIAA0330), mRNA | | | | |
| 2266 | H3095G05 | Rattus norvegicus Fucosidase, alpha-L- | | BG084074 | Mm.21580 | Energy/Metabolism |
| 2267 | | 1, tissue (Fuca), mRNA | | | | |
| 2268 | H3095H12 | Mus musculus carbonic anhydrase 4 | | BG071207 | Mm.1641 | Energy/Metabolism |
| | | (Car4), mRNA | | | | |
| 2269 | H3096A03 | cytochrome c oxidase, subunit IV | Mus musculus cytochrome c oxidase, | BG084085 | Mm.2136 | Energy/Metabolism |
| 2270 | | (Cox4), | subunit IV (Cox4), mRNA | | | |
| 2271 | H3096D01 | “Mus musculus protein phosphatase 4, | | BG071232 | Mm.28082 | Signal Transduction |
| 2272 | | catalytic subunit (Ppp4c), mRNA” | | | | |
| 2273 | H3096E12 | clone TSIP1 p53-induced apoptosis | Mus musculus clone TSIP1 p53- | BG084126 | Mm.142872 | Apoptosis |
| 2274 | | differentially expressed sequence | induced apoptosis differentially | | | |
| | | | expressed mRNA sequence | | | |
| 2275 | H3096F11 | HNK-1 sulfotransferase (HNK-1ST), | Homo sapiens HNK-1 sulfotransferase | BG084134 | Mm.26817 | Energy/Metabolism |
| 2276 | | | (HNK-1ST), mRNA | | | |
| 2277 | H3097A02 | Homo sapiens leucine aminopeptidase | | BG084150 | Mm.34832 | Energy/Metabolism |
| 2278 | | (LOC51056), mRNA | | | | |
| 2279 | H3097A03 | Mus musculus cytochrome c oxidase, | | BG084151 | Mm.2136 | Energy/Metabolism |
| 2280 | | subunit IV (Cox4), mRNA | | | | |
| 2281 | H3097A05 | Homo sapiens sulfite oxidase (SUOX), | | BG084153 | Mm.23352 | Energy/Metabolism |
| 2282 | | mRNA | | | | |
| 2283 | H3097B01 | Mus musculus branched chain | | BG084158 | Mm.4606 | Energy/Metabolism |
| 2284 | | aminotransferase 1, cytosolic (Bcat1), | | | | |
| | | mRNA | | | | |
| 2285 | H3097B03 | Mus musculus solute carrier family 11 | | BG084160 | Mm.36460 | Energy/Metabolism |
| 2286 | | (proton-coupled divalent metal ion | | | | |
| | | transporters), member 2 (Slc11a2), | | | | |
| | | mRNA | | | | |
| 2287 | H3097C12 | | | | Mm.22409 | Energy/Metabolism |
| 2288 | | | | | | |
| 2289 | H3097F02 | Mus musculus sialyltransferase 8 | | BG084201 | Mm.4954 | Energy/Metabolism |
| 2290 | | (alpha-2, 8-sialytransferase) B (Siat8b), | | | | |
| | | mRNA | | | | |
| 2291 | H3097F06 | Homo sapiens FACL5 for fatty acid | | BG084205 | Mm.30263 | Energy/Metabolism |
| 2292 | | coenzyme A ligase 5 (LOC51703), | | | | |
| | | mRNA | | | | |
| 2293 | H3097G07 | “Mus musculus retinoblastoma-binding | | BG084218 | Mm.24216 | Signal Transduction |
| 2294 | | protein 9 (Rbbp9), mRNA” | | | | |
| 2295 | H3097H08 | “Mus musculus retinoblastoma binding | | BG084231 | Mm.12145 | Signal Transduction |
| 2296 | | protein 4 (Rbbp4), mRNA” | | | | |
| 2297 | H3098A08 | B. taurus PSST subunit of the NADH: | B. taurus mRNA for PSST subunit of | BG084240 | Mm.28712 | Energy/Metabolism |
| 2298 | | ubiquinone oxidoreductase complex | the NADH: ubiquinone oxidoreductase | | | |
| | | | complex | | | |
| 2299 | H3098D02 | “Mus musculus protein tyrosine | | BG084265 | Mm.2817 | Signal Transduction |
| 2300 | | phosphatase, receptor-type, F interacting | | | | |
| | | protein, binding protein 2 (Ppfibp2), | | | | |
| | | mRNA” | | | | |
| 2301 | H3098D12 | “Mus musculus mitogen activated | | AU041108 | Mm.1059 | Signal Transduction |
| 2302 | | protein kinase kinase 1 (Map2k1), | | | | |
| | | mRNA” | | | | |
| 2303 | H3098E11 | “Mus musculus chaperonin subunit 2 | | BG084277 | Mm.40849 | Heat Shock/Stress |
| 2304 | | (beta) (Cct2), mRNA” | | | | |
| 2305 | H3098H02 | carbonyl reductase (LOC51181), | Homo sapiens carbonyl reductase | BG084296 | | Energy/Metabolism |
| 2306 | | | (LOC51181), mRNA | | | |
| 2307 | H3099A05 | 5-nucleotidase (purine), cytosolic type B | Homo sapiens 5-nucleotidase (purine), | BG084308 | Mm.29601 | Energy/Metabolism |
| 2308 | | (NT5B), | cytosolic type B (NT5B), mRNA | | | |
| 2309 | H3099C01 | solute carrier family 34 (sodium | Mus musculus solute carrier family 34 | BG084323 | | Energy/Metabolism |
| 2310 | | phosphate), member 2 (Slc34a2), | (sodium phosphate), member 2 | | | |
| | | | (Slc34a2), mRNA | | | |
| 2311 | H3099D04 | cytochrome c oxidase Vb subunit gene | Mouse cytochrome c oxidase Vb | BG071472 | Mm.16769 | Energy/Metabolism |
| 2312 | | | subunit gene, complete cds | | | |
| 2313 | H3099E08 | cDNA FLJ13488 fis, clone | Homo sapiens cDNA FLJ13488 fis, | BG084341 | Mm.22363 | |
| 2314 | | PLACE1003915, weakly similar to | clone PLACE1003915, weakly similar | | | |
| | | PROBABLE ARGINYL-TRNA | to PROBABLE ARGINYL-TRNA | | | |
| | | SYNTHETASE, CYTOPLASMIC (EC | SYNTHETASE, CYTOPLASMIC (EC | | | |
| | | 6.1.1.19) | 6.1.1.19) | | | |
| 2315 | H3100C01 | rod cGMP phosphodiesterase delta | Mus musculus rod cGMP | BG084383 | Mm.12925 | Energy/Metabolism |
| 2316 | | subunit (Pde6d) gene | phosphodiesterase delta subunit (Pde6d) | | | |
| | | | gene, complete cds | | | |
| 2317 | H3100C10 | potassium intermediate/small | Homo sapiens potassium | BG084391 | Mm.17734 | Energy/Metabolism |
| 2318 | | conductance calcium-activated channel, | intermediate/small conductance | | | |
| | | subfamily N, member 2 (KCNN2), | calcium-activated channel, subfamily | | | |
| | | | N, member 2 (KCNN2), mRNA | | | |
| 2319 | H3100E04 | inhibitor of apoptosis protein 1 | Mus musculus inhibitor of apoptosis | BG084405 | Mm.2026 | Apoptosis |
| 2320 | | | protein 1 mRNA, complete cds | | | |
| 2321 | H3101A08 | ribosomal protein L23 (Rpl23), | Mus musculus ribosomal protein L23 | BG084450 | Mm.12144 | Energy/Metabolism |
| 2322 | | | (Rpl23), mRNA | | | |
| 2323 | H3101C10 | glyceraldehyde-3-phosphate | Mus musculus glyceraldehyde-3- | BG071626 | Mm.5289 | Energy/Metabolism |
| 2324 | | dehydrogenase (Gapd), | phosphate dehydrogenase (Gapd), | | | |
| | | | mRNA | | | |
| 2325 | H3101D05 | phosphatidylethanolamine N- | Mus musculus | BG071633 | Mm.2731 | Energy/Metabolism |
| 2326 | | methyltransferase (Pemt), | phosphatidylethanolamine N- | | | |
| | | | methyltransferase (Pemt), mRNA | | | |
| 2327 | H3101E05 | solute carrier family 34 (sodium | Mus musculus solute carrier family 34 | BG071644 | Mm.3786 | Energy/Metabolism |
| 2328 | | phosphate), member 2 (Slc34a2), | (sodium phosphate), member 2 | | | |
| | | | (Slc34a2), mRNA | | | |
| 2329 | H3101G05 | “Mus musculus DNA J protein (Dnaj3), | | AU044106 | Mm.29186 | Heat Shock/Stress |
| | | mRNA” | | | | |
| 2330 | H3101H02 | “Rattus norvegicus mytonic dystrophy | | BG071671 | Mm.38330 | Signal Transduction |
| 2331 | | kinase-related Cdc42-binding kinase | | | | |
| | | (MRCK) mRNA, complete cds” | | | | |
| 2332 | H3102A07 | phosphatidylserine synthase 1 (Ptdss1), | Mus musculus phosphatidylserine | BG071685 | Mm.9440 | Energy/Metabolism |
| 2333 | | | synthase 1 (Ptdss1), mRNA | | | |
| 2334 | H3102C06 | choline transporter (CHOT1), | Rattus norvegicus choline transporter | BG071707 | Mm.28056 | Energy/Metabolism |
| 2335 | | | (CHOT1), mRNA | | | |
| 2336 | H3102D02 | apoptotic chromatin condensation | Mus musculus apoptotic chromatin | BG071714 | Mm.30236 | Apoptosis |
| 2337 | | inducer in the nucleus (Acinus- | condensation inducer in the nucleus | | | |
| | | pending), | (Acinus-pending), mRNA | | | |
| 2338 | H3102D09 | “Mus musculus retinoblastoma binding | | BG084566 | Mm.1603 | Signal Transduction |
| 2339 | | protein 7 (Rbbp7), mRNA” | | | | |
| 2340 | H3102E01 | potassium channel modulatory factor | Mus musculus potassium channel | BG071725 | Mm.29194 | Energy/Metabolism |
| 2341 | | DEBT-91 (Debt91), | modulatory factor DEBT-91 (Debt91), | | | |
| | | | mRNA | | | |
| 2342 | H3102F08 | N-acylsphingosine amidohydrolase 1 | Mus musculus N-acylsphingosine | BG071742 | Mm.22547 | Energy/Metabolism |
| 2343 | | (Asah1), | amidohydrolase 1 (Asah1), mRNA | | | |
| 2344 | H3102G07 | “Mus musculus protein tyrosine | | BG071749 | Mm.147428 | Signal Transduction |
| 2345 | | phosphatase, non-receptor type 11 | | | | |
| | | (Ptpn11), mRNA” | | | | |
| 2346 | H3102H03 | isoleucine-tRNA synthetase (IARS), | Homo sapiens isoleucine-tRNA | BG084601 | Mm.21118 | Energy/Metabolism |
| 2347 | | | synthetase (IARS), mRNA | | | |
| 2348 | H3102H07 | 2,3-bisphosphoglycerate mutase | Mus musculus 2,3-bisphosphoglycerate | BG084605 | Mm.22706 | Energy/Metabolism |
| 2349 | | (Bpgm), | mutase (Bpgm), mRNA | | | |
| 2350 | H3102H09 | ribonuclease H1 (Rnaseh1), | Mus musculus ribonuclease H1 | BG071762 | Mm.10152 | Energy/Metabolism |
| 2351 | | | (Rnaseh1), mRNA | | | |
| 2352 | H3103A05 | “Mus musculus phospholipase A2, | | BG084614 | Mm.4186 | Signal Transduction |
| 2353 | | group 4 (Pla2g4), mRNA” | | | | |
| 2354 | H3103B07 | Mus musculus BCL2/adenovirus E1B | | BG084628 | Mm.142818 | Apoptosis |
| 2355 | | 19 kDa-interacting protein 3-like | | | | |
| | | (Bnip31), mRNA | | | | |
| 2356 | H3103D01 | “Mus musculus protein phosphatase 1, | | BG071790 | Mm.7793 | Signal Transduction |
| 2357 | | catalytic subunit, gamma isoform | | | | |
| | | (Ppp1cc), mRNA” | | | | |
| 2358 | H3103D11 | “Mus musculus regulator of G-protein | | BG071811 | Mm.44606 | Signal Transduction |
| 2359 | | signaling 17 (Rgs17), mRNA” | | | | |
| 2360 | H3103E12 | Mus musculus Bcl2-like 10 (Bcl2l10), | | BG071824 | Mm.25988 | Apoptosis |
| 2361 | | mRNA | | | | |
| 2362 | H3103F06 | “Mus musculus small GTPase (Rab11a) | | BG071829 | Mm.1387 | Signal Transduction |
| 2363 | | gene, complete cds” | | | | |
| 2364 | H3104A11 | Mus musculus thioredoxin-like (32 kD) | | BG071869 | Mm.19169 | Energy/Metabolism |
| 2365 | | (Txnl), mRNA | | | | |
| 2366 | H3104D01 | Mouse mRNA for beta-1,4- | | BG071892 | Mm.15622 | Energy/Metabolism |
| 2367 | | galactosyltransferase | | | | |
| 2368 | H3104E06 | Mus musculus ATP-binding cassette | | BG071908 | Mm.41002 | Energy/Metabolism |
| 2369 | | protein (Abcc1b) mRNA, partial cds | | | | |
| 2370 | H3105A09 | Pseudomonas aeruginosa PA01, section | Pseudomonas aeruginosa PA01, section | BG071952 | | Energy/Metabolism |
| 2371 | | 341 of 529 of the genome | 341 of 529 of the complete genome | | | |
| 2372 | H3105A10 | cDNA FLJ12989 fis, clone | Homo sapiens cDNA FLJ12989 fis, | BG084872 | Mm.154589 | Energy/Metabolism |
| 2373 | | NT2RP3000085, moderately similar to | clone NT2RP3000085, moderately | | | |
| | | ACETYL-/PROPIONYL-COENZYME | similar to ACETYL-/PROPIONYL- | | | |
| | | A CARBOXYLASE ALPHA CHAIN | COENZYME A CARBOXYLASE | | | |
| | | [CONTAINS: BIOTIN | ALPHA CHAIN [CONTAINS: | | | |
| | | CARBOXYLASE (EC 6.3.4.14); | BIOTIN CARBOXYLASE (EC | | | |
| | | BIOTIN CARBOXYL CARRIER | 6.3.4.14); BIOTIN CARBOXYL | | | |
| | | | CARRIER | | | |
| 2374 | H3105B07 | “Mus musculus Cctz-2 gene for | | BG084881 | Mm.29622 | Heat Shock/Stress |
| 2375 | | chaperonin containing TCP-1 zeta-2 | | | | |
| | | subunit, exon 1, 2, 3” | | | | |
| 2376 | H3105C03 | CGI-30 protein (LOC51611), | Homo sapiens CGI-30 protein | BG084888 | Mm.5915 | Energy/Metabolism |
| 2377 | | | (LOC51611), mRNA | | | |
| 2378 | H3105C04 | natural killer cell BY55 precursor | Mus musculus natural killer cell BY55 | BG084889 | Mm.11526 | Apoptosis |
| 2379 | | (By55-pending), | precursor (By55-pending), mRNA | | | |
| 2380 | H3105C10 | “Mus musculus mRNA for LIMK2b, | | BG071976 | Mm.42927 | Signal Transduction |
| 2381 | | complete cds” | | | | |
| 2382 | H3105D09 | masc apoptosis-associated speck-like | Mus musculus masc mRNA for | BG071986 | Mm.24163 | Apoptosis |
| 2383 | | protein containing CARD | apoptosis-associated speck-like protein | | | |
| | | | containing CARD, complete cds | | | |
| 2384 | H3105E08 | tyrosine 3-monooxygenase/tryptophan | Mus musculus tyrosine 3- | BG084914 | Mm.14722 | Energy/Metabolism |
| 2385 | | 5-monooxygenase activation protein, | monooxygenase/tryptophan 5- | | | |
| | | theta polypeptide (Ywhaq), | monooxygenase activation protein, | | | |
| | | | theta polypeptide (Ywhaq), mRNA | | | |
| 2386 | H3105G04 | “Mus musculus mitogen-activated | | BG084932 | Mm.987 | Signal Transduction |
| 2387 | | protein kinase kinase kinase kinase 4 | | | | |
| | | (Map4k4), mRNA” | | | | |
| 2388 | H3105G10 | pyrroline 5-carboxylate reductase isom | Homo sapiens pyrroline 5-carboxylate | BG072021 | Mm.29379 | Energy/Metabolism |
| 2389 | | (P5CR2), | reductase isoform (P5CR2), mRNA | | | |
| 2390 | H3107D11 | H3107D11 | | | | Energy/Metabolism |
| 2391 | | | | | | |
| 2392 | H3107E03 | H3107E03 | | | | Energy/Metabolism |
| 2393 | | | | | | |
| 2394 | H3107G10 | alpha-N-acetylglucosaminidase | Mus musculus alpha-N- | BG072192 | Mm.6142 | Energy/Metabolism |
| 2395 | | (Sanfilippo disease IIIB) (Naglu), | acetylglucosaminidase (Sanfilippo | | | |
| | | | disease IIIB) (Naglu), mRNA | | | |
| 2396 | H3107G12 | carnitine octanoyltransferase (COT), | Homo sapiens carnitine | BG085029 | Mm.28197 | Energy/Metabolism |
| 2397 | | | octanoyltransferase (COT), mRNA | | | |
| 2398 | H3108C03 | 3-5 exonuclease TREX1 | Mus musculus 3-5 exonuclease TREX1 | BG072232 | Mm.17632 | Energy/Metabolism |
| 2399 | | | mRNA, complete cds | | | |
| 2400 | H3108E02 | “Homo sapiens mitogen-activated | | BG072253 | Mm.18856 | Signal Transduction |
| 2401 | | protein kinase 6 (MAPK6), mRNA” | | | | |
| 2402 | H3108E05 | “Mus musculus RAS, dexamethasone- | | BG072256 | Mm.3903 | Signal Transduction |
| 2403 | | induced 1 (Rasd1), mRNA” | | | | |
| 2404 | H3108F08 | TF-1 cell apoptosis related protein-15 | Mus musculus TF-1 cell apoptosis | BG085103 | Mm.28441 | Apoptosis |
| 2405 | | (Tfar15), | related protein-15 (Tfar15), mRNA | | | |
| 2406 | H3108H04 | “Mus musculus adenylyl cyclase type 9 | | BG072288 | Mm.4294 | Signal Transduction |
| 2407 | | mRNA, complete cds” | | | | |
| 2408 | H3108H12 | laminin B1 | Mouse laminin B1 mRNA, complete | BG085129 | Mm.148395 | Apoptosis |
| 2409 | | | cds | | | |
| 2410 | H3109A04 | “Mus musculus receptor (calcitonin) | | BG072299 | Mm.1574 | Signal Transduction |
| 2411 | | activity modifying protein 2 (Ramp2), | | | | |
| | | mRNA” | | | | |
| 2412 | H3109F07 | ATPase, Ca++ transporting, cardiac | Mus musculus ATPase, Ca++ | BG085195 | Mm.42255 | Energy/Metabolism |
| 2413 | | muscle, slow twitch 2 (Atp2a2), | transporting, cardiac muscle, slow | | | |
| | | | twitch 2 (Atp2a2), mRNA | | | |
| 2414 | H3110B08 | tyrosine 3-monooxygenase/tryptophan | Mus musculus tyrosine 3- | BG085234 | Mm.14722 | Energy/Metabolism |
| 2415 | | 5-monooxygenase activation protein, | monooxygenase/tryptophan 5- | | | |
| | | theta polypeptide (Ywhaq), | monooxygenase activation protein, | | | |
| | | | theta polypeptide (Ywhaq), mRNA | | | |
| 2416 | H3110B12 | “Homo sapiens casein kinase I alpha | | BG072410 | Mm.43737 | Signal Transduction |
| 2417 | | isoform (CSNK1A1) mRNA, complete | | | | |
| | | cds” | | | | |
| 2418 | H3110F10 | epoxide hydrolase 1, microsomal | Mus musculus epoxide hydrolase 1, | BG072453 | Mm.9075 | Energy/Metabolism |
| 2419 | | (Ephx1), | microsomal (Ephx1), mRNA | | | |
| 2420 | H3110H11 | “Mouse brain calmodulin-dependent | | BG072475 | Mm.293 | Signal Transduction |
| 2421 | | phosphatase (calcineurin) catalytic | | | | |
| | | subunit mRNA, 3′ end” | | | | |
| 2422 | H3111A02 | brain cDNA, clone MNCb-1429, similar | Mus musculus brain cDNA, clone | BG085276 | Mm.30215 | Energy/Metabolism |
| 2423 | | to peroxiredoxin V (PrxV) gene | MNCb-1429, similar to Mus musculus | | | |
| | | | peroxiredoxin V (PrxV) gene | | | |
| 2424 | H3111A08 | “Mus musculus signal transducer and | | BG085280 | | Signal Transduction |
| 2425 | | transcription activator 5a (Stat5a) gene, | | | | |
| | | partial cds” | | | | |
| 2426 | H3111F09 | Rat Y-b3 glutathione-S-transferase | Rat Y-b3 glutathione-S-transferase | BG072517 | Mm.29640 | Energy/Metabolism |
| 2427 | | | mRNA, complete cds | | | |
| 2428 | H3111G06 | arginase type II (Arg2), | Mus musculus arginase type II (Arg2), | BG085317 | Mm.3506 | Energy/Metabolism |
| 2429 | | | mRNA | | | |
| 2430 | H3111G12 | zinc transporter like 2 (Zntl2) | Mus musculus zinc transporter like 2 | BG085322 | Mm.28490 | Energy/Metabolism |
| 2431 | | | (Zntl2) mRNA, complete cds | | | |
| 2432 | H3112A04 | Mus musculus carnitine | | BG085333 | Mm.29499 | Matrix/Structural |
| 2433 | | palmitoyltransferase 2 (Cpt2), mRNA | | | | Proteins |
| 2434 | H3112B05 | “Homo sapiens calcium-regulated heat- | | BG072550 | Mm.33981 | Heat Shock/Stress |
| 2345 | | stable protein (24 kD) (CRHSP-24), | | | | |
| | | mRNA” | | | | |
| 2436 | H3112B07 | Rat mRNA for 3-oxoacyl-CoA thiolase | | BG085346 | Mm.142498 | Energy/Metabolism |
| 2437 | | | | | | |
| 2438 | H3112F06 | “Mus musculus Cctb gene for | | BG085384 | Mm.3670 | Heat Shock/Stress |
| 2439 | | chaperonin containing TCP-1 beta | | | | |
| | | subunit, complete cds” | | | | |
| 2440 | H3112G09 | Mus musculus ATPase, H+ | | BG085399 | Mm.1081 | Energy/Metabolism |
| 2441 | | transporting, lysosomal (vacuolar | | | | |
| | | proton pump), 42 kDa (Atp6d), mRNA | | | | |
| 2442 | H3113B07 | 13 kDa differentiation-associated protein | Homo sapiens 13 kDa differentiation- | BG085428 | Mm.27886 | Energy/Metabolism |
| 2443 | | (LOC55967), | associated protein (LOC55967), mRNA | | | |
| 2444 | H3113B08 | F1F0ATP synthase complex E subunit | Mus musculus F1F0ATP synthase | | Mm.19370 | Energy/Metabolism |
| 2445 | | (Atp5k) gene | complex E subunit (Atp5k) gene, | | | |
| | | | complete cds | | | |
| 2446 | H3113E03 | “Mus musculus MAP kinase-interacting | | BG085457 | Mm.6797 | Signal Transduction |
| 2447 | | serine/threonine kinase 2 (Mknk2), | | | | |
| | | mRNA” | | | | |
| 2448 | H3113F04 | “Mus musculus kappaB-Ras1 mRNA, | | BG072675 | Mm.25648 | Signal Transduction |
| 2449 | | complete cds” | | | | |
| 2450 | H3113F06 | ribonuclease/angiogenin inhibitor | Homo sapiens ribonuclease/angiogenin | BG085471 | Mm.6586 | Energy/Metabolism |
| 2451 | | (RNH), | inhibitor (RNH), mRNA | | | |
| 2452 | H3113G07 | “Homo sapiens phospholipase C, | | BG085484 | Mm.22370 | Signal Transduction |
| 2453 | | gamma 2 (phosphatidylinositol- | | | | |
| | | specific) (PLCG2), mRNA” | | | | |
| 2454 | H3113G08 | ornithine decarboxylase antizyme 3 | Mus musculus ornithine decarboxylase | BG085485 | Mm.154432 | Energy/Metabolism |
| 2455 | | (Oaz3), | antizyme 3 (Oaz3), mRNA | | | |
| 2456 | H3113H12 | “Mus musculus manic fringe homolog | | BG072704 | Mm.517 | Signal Transduction |
| 2457 | | (Drosophila)(Mfng), mRNA” | | | | |
| 2458 | H3114A05 | glutathione peroxidase 4 (Gpx4), | Mus musculus glutathione peroxidase 4 | BG072706 | Mm.2400 | Energy/Metabolism |
| 2459 | | | (Gpx4), mRNA | | | |
| 2460 | H3114A06 | spermidine/spermine N1-acetyl | Mus musculus spermidine/spermine | BG085499 | Mm.2734 | Energy/Metabolism |
| 2461 | | transferase (Sat), | N1-acetyl transferase (Sat), mRNA | | | |
| 2462 | H3114C12 | cytosolic class 3 aldehyde | Mus musculus cytosolic class 3 | BG085522 | Mm.14798 | Energy/Metabolism |
| 2463 | | dehydrogenase (Adh4) gene, 5 flanking | aldehyde dehydrogenase (Adh4) gene, 5 | | | |
| | | region sequence | flanking region sequence | | | |
| 2464 | H3114D10 | M. musculus RPS3a gene | M. musculus RPS3a gene | BG072742 | Mm.6957 | Apoptosis |
| 2465 | | | | | | |
| 2466 | H3114F11 | Mus musculus interferon gamma | | BG085554 | Mm.153463 | Signal Transduction |
| 2467 | | receptor 2 (Ifngr2), mRNA | | | | |
| 2468 | H3114G12 | “Mus musculus copper chaperone for | | BG072776 | Mm.21414 | Heat Shock/Stress |
| 2469 | | superoxide dismutase (Ccsd) mRNA, | | | | |
| | | complete cds” | | | | |
| 2470 | H3114H06 | PIG-M mannosyltransferase | Rattus norvegicus PIG-M mRNA for | BG085572 | Mm.26612 | Energy/Metabolism |
| 2471 | | | mannosyltransferase, complete cds | | | |
| 2472 | H3115A05 | “Mus musculus phospholipase C, delta | | BG085579 | Mm.23963 | Signal Transduction |
| | | (Plcd), mRNA” | | | | |
| 2473 | H3115B07 | Mus musculus S100A9 gene for | | BG072801 | Mm.2128 | Signal Transduction |
| 2474 | | S100A9 protein exons 1-3 | | | | |
| 2475 | H3115C02 | clone UWGC: mbac82 from 14D1-D2 | Mus musculus clone UWGC: mbac82 | BG072808 | Mm.353 | Energy/Metabolism |
| 2476 | | (T-Cell Receptor Alpha Locus), | from 14D1-D2 (T-Cell Receptor Alpha | | | |
| | | sequence | Locus), complete sequence | | | |
| 2477 | H3115C11 | “Mus musculus protein phosphatase 2A | | BG072817 | Mm.3785 | Signal Transduction |
| 2478 | | B′alpha3 regulatory subunit mRNA, | | | | |
| | | partial cds” | | | | |
| 2479 | H3115D08 | F1F0-ATP synthase, g subunit | Mus musculus mRNA for F1F0-ATP | BG072826 | Mm.14663 | Energy/Metabolism |
| 2480 | | | synthase, g subunit | | | |
| 2481 | H3115H06 | ribosomal protein L29 | | | Mm.2235 | Energy/Metabolism |
| 2482 | | | | | | |
| 2483 | H3115H10 | ATP synthase gamma-subunit precursor | Mus musculus mRNA for ATP synthase | BG072870 | Mm.12677 | Energy/Metabolism |
| 2484 | | | gamma-subunit precursor, complete cds | | | |
| 2485 | H3116D11 | cytochrome c oxidase, subunit VIIa 3 | Mus musculus cytochrome c oxidase, | BG072912 | Mm.2151 | Energy/Metabolism |
| 2486 | | (Cox7a3), | subunit VIIa 3 (Cox7a3), mRNA | | | |
| 2487 | H3116E02 | “Mus musculus adenylate cyclase | | BG085699 | Mm.44245 | Signal Transduction |
| 2488 | | activating polypeptide 1 receptor 1 | | | | |
| | | (Adcyap1r1), mRNA” | | | | |
| 2489 | H3116G12 | H3116G12 | | | | Energy/Metabolism |
| 2490 | | | | | | |
| 2491 | H3117A12 | kinesin-related mitotic motor protein | Mus musculus mRNA for kinesin- | | Mm.42203 | |
| 2492 | | | related mitotic motor protein | | | |
| 2493 | H3117C04 | “Homo sapiens MEBP-1 mRNA for | | BG072981 | Mm.6803 | Heat Shock/Stress |
| 2494 | | MAPK-ERK binding protein-1, | | | | |
| | | complete cds” | | | | |
| 2495 | H3117G06 | Rat glycogen phosphorylase brain | Rat glycogen phosphorylase brain | BG073025 | Mm.30072 | Energy/Metabolism |
| 2496 | | isozyme, 5 end of cds | isozyme mRNA, 5 end of cds | | | |
| 2497 | H3118E12 | apoptosis inhibitor bcl-x (bcl-x) gene, | Mus musculus apoptosis inhibitor bcl-x | AW548292 | Mm.148197 | Apoptosis |
| | | exon 3 and cds | (bcl-x) gene, exon 3 and complete cds | | | |
| 2498 | H3118H09 | mastocytoma N-deacetylase/N- | Mus musculus mastocytoma N- | BG085858 | Mm.4084 | Energy/Metabolism |
| 2499 | | sulfotransferase (Mndns), | deacetylase/N-sulfotransferase (Mndns), | | | |
| | | | mRNA | | | |
| 2500 | H3119A04 | H+ ATP synthase subunit c | Mus musculus H+ ATP synthase | BG085863 | Mm.258 | Energy/Metabolism |
| 2501 | | | subunit c mRNA, complete cds | | | |
| 2502 | H3119B04 | | | | | Heat Shock/Stress |
| 2503 | | | | | | |
| 2504 | H3119B06 | Na, K-ATPase beta-3 subunit (Atp1b3) | Mus musculus Na, K-ATPase beta-3 | BG073136 | Mm.424 | Energy/Metabolism |
| 2505 | | gene, exon 7 and cds | subunit (Atp1b3) gene, exon 7 and | | | |
| | | | complete cds | | | |
| 2506 | H3119B09 | cytochrome b-561 (Cyb561), | Mus musculus cytochrome b-561 | BG085878 | Mm.18942 | Energy/Metabolism |
| 2507 | | | (Cyb561), mRNA | | | |
| 2508 | H3119G03 | “Mus musculus protein tyrosine | | BG073186 | Mm.28909 | Signal Transduction |
| 2509 | | phosphatase 4a1 (Ptp4a1), mRNA” | | | | |
| 2510 | H3119G08 | glutathione S-transferase, alpha 4 | Mus musculus glutathione S-transferase, | BG073190 | Mm.2662 | Energy/Metabolism |
| 2511 | | (Gsta4), | alpha 4 (Gsta4), mRNA | | | |
| 2512 | H3119G12 | cDNA DKFZp564A126 (from clone | Homo sapiens mRNA; cDNA | BG085933 | Mm.22710 | Energy/Metabolism |
| 2513 | | DKFZp564A126); partial cds | DKFZp564A126 (from clone | | | |
| | | | DKFZp564A126); partial cds | | | |
| 2514 | H3119H03 | Mus musculus FVB/N collagen pro- | | BG073196 | Mm.22621 | Matrix/Structural |
| 2515 | | alpha-1 type I chain mRNA, complete | | | | Proteins |
| | | cds | | | | |
| 2516 | H3119H04 | fatty-acid-Coenzyme A ligase, long- | Homo sapiens fatty-acid-Coenzyme A | BG073197 | Mm.27944 | Energy/Metabolism |
| 2517 | | chain 3 (FACL3), | ligase, long-chain 3 (FACL3), mRNA | | | |
| 2518 | H3119H06 | “Homo sapiens casein kinase 1, gamma | | BG085938 | Mm.29873 | Signal Transduction |
| 2519 | | 2 (CSNK1G2), mRNA” | | | | |
| 2520 | H3120A10 | isocitrate dehydrogenase | Mus musculus isocitrate dehydrogenase | BG085953 | Mm.2966 | Energy/Metabolism |
| 2521 | | | mRNA, complete cds | | | |
| 2522 | H3120D01 | “Mus musculus MEK kinase 3, mRNA, | | BG085978 | Mm.27041 | Signal Transduction |
| 2523 | | partial cds” | | | | |
| 2524 | H3120F08 | geranylgeranyl diphosphate synthase 1 | Mus musculus geranylgeranyl | BG086008 | Mm.4076 | Energy/Metabolism |
| 2525 | | (Ggps1), | diphosphate synthase 1 (Ggps1), | | | |
| | | | mRNA | | | |
| 2526 | H3120H05 | full length insert cDNA clone ZC24D06 | Homo sapiens full length insert cDNA | BG086026 | | Energy/Metabolism |
| 2527 | | | clone ZC24D06 | | | |
| 2528 | H3121A04 | protein phosphatase type-1 catalytic | Homo sapiens protein phosphatase | BG086037 | Mm.4572 | Energy/Metabolism |
| 2529 | | subunit delta isom (PPCS1D) | type-1 catalytic subunit delta isoform | | | |
| | | | (PPCS1D) mRNA, complete cds | | | |
| 2530 | H3121C02 | “Mus musculus follistatin-like (Fstl), | | BG086054 | Mm.22763 | Signal Transduction |
| 2531 | | mRNA” | | | | |
| 2532 | H3121E04 | retinal short-chain | Mus musculus retinal short-chain | BG086076 | Mm.14063 | Energy/Metabolism |
| 2533 | | dehydrogenase/reductase 1 (Rsdr1- | dehydrogenase/reductase 1 (Rsdr1- | | | |
| | | pending), | pending), mRNA | | | |
| 2534 | H3122B07 | “Mus musculus heat-responsive protein | | BG073398 | Mm.2607 | Heat Shock/Stress |
| 2535 | | 12 (Hrsp12), mRNA” | | | | |
| 2536 | H3122D07 | “Mus musculus heat shock protein, 74 kDa, | | BG073420 | Mm.2849 | Heat Shock/Stress |
| 2537 | | A (Hspa9a), mRNA” | | | | |
| 2538 | H3122E12 | ATP synthase beta subunit (ATPSB) | Human ATP synthase beta subunit | BG073436 | Mm.17869 | Energy/Metabolism |
| 2539 | | gene | (ATPSB) gene, complete cds | | | |
| 2540 | H3122F01 | ATP synthase beta subunit (ATPSB) | Human ATP synthase beta subunit | BG073437 | Mm.17869 | Energy/Metabolism |
| 2541 | | gene | (ATPSB) gene, complete cds | | | |
| 2542 | H3122F04 | ATP-binding cassette, sub-family D | Mus musculus ATP-binding cassette, | BG086172 | Mm.87806 | Energy/Metabolism |
| 2543 | | (ALD), member 4 (Abcd4), | sub-family D (ALD), member 4 | | | |
| | | | (Abcd4), mRNA | | | |
| 2544 | H3122F12 | sterol-carrier protein X | Mus musculus sterol-carrier protein X | BG086180 | Mm.1779 | Matrix/Structural |
| 2545 | | | mRNA, complete cds | | | Proteins |
| 2546 | H3122G01 | sterol-carrier protein X | Mus musculus sterol-carrier protein X | BG086181 | Mm.1779 | Matrix/Structural |
| 2547 | | | mRNA, complete cds | | | Proteins |
| 2548 | H3122H03 | brain cDNA, clone MNCb-0663, similar | Mus musculus brain cDNA, clone | BG086194 | Mm.28124 | |
| 2549 | | to augmenter of liver regeneration (Alr) | MNCb-0663, similar to Mus musculus | | | |
| | | | augmenter of liver regeneration (Alr) | | | |
| | | | mRNA | | | |
| 2550 | H3123C04 | “Mus musculus GNB3 gene for GTP- | | BG073494 | Mm.35708 | Signal Transduction |
| 2551 | | binding protein beta3 subunit, complete | | | | |
| | | cds” | | | | |
| 2552 | H3123D10 | BCL2/adenovirus E1B 19 kD-interacting | Homo sapiens BCL2/adenovirus E1B | BG073508 | Mm.21795 | Apoptosis |
| 2553 | | protein 1 (BNIP1), | 19 kD-interacting protein 1 (BNIP1), | | | |
| | | | mRNA | | | |
| 2554 | H3123G10 | mannoside | Mus musculus mannoside | BG073537 | Mm.2672 | Energy/Metabolism |
| 2555 | | acetylglucosaminyltransferase 1 | acetylglucosaminyltransferase 1 | | | |
| | | (Mgat1), | (Mgat1), mRNA | | | |
| 2556 | H3123H05 | “Homo sapiens tumor protein p53- | | BG086271 | Mm.23595 | Signal Transduction |
| 2557 | | binding protein, 2 (TP53BP2), mRNA” | | | | |
| 2558 | H3123H07 | novel cell death-regulatory protein | Mus musculus novel cell death- | BG086273 | Mm.21162 | Apoptosis |
| 2559 | | GRIM19 (Grim19) | regulatory protein GRIM19 (Grim19) | | | |
| | | | mRNA, complete cds | | | |
| 2560 | H3124B02 | “Mus musculus cold inducible RNA- | | BG086286 | Mm.17898 | Heat Shock/Stress |
| 2561 | | binding protein (Cirbp), mRNA” | | | | |
| 2562 | H3124D02 | ferrochelatase (Fech), | Mus musculus ferrochelatase (Fech), | BG086309 | Mm.1070 | Energy/Metabolism |
| 2563 | | | mRNA | | | |
| 2564 | H3124D04 | caspase 9 (Casp9), | Mus musculus caspase 9 (Casp9), | BG086310 | Mm.102926 | Apoptosis |
| 2565 | | | mRNA | | | |
| 2566 | H3124D06 | DNA fragmentation factor, 40 kD, beta | Mus musculus DNA fragmentation | BG086312 | Mm.38853 | Apoptosis |
| 2567 | | subunit (Dffb), | factor, 40 kD, beta subunit (Dffb), | | | |
| | | | mRNA | | | |
| 2568 | H3124D11 | mitotic arrest deficient 1-like 1 | Mus musculus mitotic arrest deficient | | Mm.27250 | Cell Cycle |
| 2569 | | (Mad111), | 1-like 1 (Mad111), mRNA | | | |
| 2570 | H3124E02 | hepatocyte growth factor-like protein | Mouse hepatocyte growth factor-like | BG073592 | Mm.21989 | Energy/Metabolism |
| 2571 | | gene | protein gene, complete cds | | | |
| 2572 | H3124F04 | microsomal glutathione S-transferase | Mus musculus microsomal glutathione | BG086330 | Mm.14796 | Energy/Metabolism |
| 2573 | | (Gst), | S-transferase (Gst), mRNA | | | |
| 2574 | H3124H01 | NADH dehydrogenase (ubiquinone) 1 | Homo sapiens NADH dehydrogenase | BG086348 | Mm.8688 | Energy/Metabolism |
| 2575 | | beta subcomplex, 10 (22 kD, PDSW) | (ubiquinone) 1 beta subcomplex, 10 | | | |
| | | (NDUFB10), | (22 kD, PDSW) (NDUFB10), mRNA | | | |
| 2576 | H3125A06 | “Mus musculus glycogen synthase | | BG086363 | | Signal Transduction |
| 2577 | | kinase 3 beta (Gsk3b), mRNA” | | | | |
| 2578 | H3125A09 | glutathione peroxidase 3 (Gpx3), | Mus musculus glutathione peroxidase 3 | BG073718 | Mm.7156 | Energy/Metabolism |
| 2579 | | | (Gpx3), mRNA | | | |
| 2580 | H3125B05 | “Homo sapiens heat shock transcription | | BG086370 | Mm.75856 | Heat Shock/Stress |
| 2581 | | factor 2 binding protein (HSF2BP), | | | | |
| | | mRNA” | | | | |
| 2582 | H3125C08 | ribonuclease III (RN3) | Homo sapiens ribonuclease III (RN3) | BG086378 | | Energy/Metabolism |
| 2583 | | | mRNA, complete cds | | | |
| 2584 | H3125C10 | major histocompatibility locus class III | Mus musculus major histocompatibility | BG086380 | Mm.1457 | Energy/Metabolism |
| 2585 | | regions Hsc70t gene, partial cds; | locus class III regions Hsc70t gene, | | | |
| | | smRNP, G7A, NG23, MutS homolog, | partial cds; smRNP, G7A, NG23, MutS | | | |
| | | CLCP, NG24, NG25, and NG26 genes; | homolog, CLCP, NG24, NG25, and | | | |
| | | and unknown genes | NG26 genes, complete cds; and | | | |
| | | | unknown genes | | | |
| 2586 | H3125D09 | “Homo sapiens suppression of | | BG086389 | Mm.4908 | Heat Shock/Stress |
| 2587 | | tumorigenicity 13 (colon carcinoma) | | | | |
| | | (Hsp70-interacting protein) (ST13), | | | | |
| | | mRNA” | | | | |
| 2588 | H3125E01 | cytosolic acyl coenzyme A thioester | Homo sapiens cytosolic acyl coenzyme | BG086391 | Mm.142740 | Energy/Metabolism |
| 2589 | | hydrolase (HBACH), | A thioester hydrolase (HBACH), | | | |
| | | | mRNA | | | |
| 2590 | H3125F03 | thioredoxin (Txn), | Mus musculus thioredoxin (Txn), | BG086400 | Mm.1275 | Energy/Metabolism |
| 2591 | | | mRNA | | | |
| 2592 | H3125F07 | protein translocase, JM26 protein, UDP- | Homo sapiens protein translocase, | | Mm.27393 | Matrix/Structural |
| 2593 | | galactose translocator, pim-2 | JM26 protein, UDP-galactose | | | Proteins |
| | | protooncogene homolog pim-2h, and | translocator, pim-2 protooncogene | | | |
| | | shal-type potassium channel genes; | homolog pim-2h, and shal-type | | | |
| | | JM12 protein and transcription | potassium channel genes, complete cds; | | | |
| | | | JM12 protein and transcription | | | |
| 2594 | H3125H05 | M. musculus RPS3a gene | M. musculus RPS3a gene | | Mm.16228 | Apoptosis |
| 2595 | | | | | | |
| 2596 | H3126B12 | “Mus musculus protein kinase inhibitor, | | BG086437 | Mm.10091 | Signal Transduction |
| 2597 | | gamma (Pkig), mRNA” | | | | |
| 2598 | H3126C12 | protease (prosome, macropain) 28 | Mus musculus protease (prosome, | BG073636 | Mm.830 | Energy/Metabolism |
| 2599 | | subunit, alpha (Psme1), | macropain) 28 subunit, alpha (Psme1), | | | |
| | | | mRNA | | | |
| 2600 | H3126F10 | spermine synthase (Sms), | Mus musculus spermine synthase (Sms), | BG086479 | Mm.3553 | Energy/Metabolism |
| 2601 | | | mRNA | | | |
| 2602 | H3127G05 | palmitoyl-protein thioesterase (Ppt), | Mus musculus palmitoyl-protein | BG086559 | Mm.43660 | Energy/Metabolism |
| 2603 | | | thioesterase (Ppt), mRNA | | | |
| 2604 | H3127G08 | cDNA FLJ13974 fis, clone | Homo sapiens cDNA FLJ13974 fis, | BG086562 | Mm.41565 | Energy/Metabolism |
| 2605 | | Y79AA1001581, weakly similar to | clone Y79AA1001581, weakly similar | | | |
| | | ACETYL-COENZYME A | to ACETYL-COENZYME A | | | |
| | | SYNTHETASE (EC 6.2.1.1) | SYNTHETASE (EC 6.2.1.1) | | | |
| 2606 | H3128D01 | s-adenosylmethionine synthetase | Mouse s-adenosylmethionine | BG086604 | Mm.14064 | Energy/Metabolism |
| 2607 | | | synthetase mRNA | | | |
| 2608 | H3128D02 | thioredoxin interacting factor (Vdup1) | Mus musculus thioredoxin interacting | BG086605 | Mm.77432 | Energy/Metabolism |
| 2609 | | gene | factor (Vdup1) gene, complete cds | | | |
| 2610 | H3129C03 | solute carrier family 4 (anion | Mus musculus solute carrier family 4 | BG086676 | Mm.7248 | Energy/Metabolism |
| 2611 | | exchanger), member 1 (Slc4a1), | (anion exchanger), member 1 (Slc4a1), | | | |
| | | | mRNA | | | |
| 2612 | H3129C06 | protein-L-isoaspartate (D-aspartate) O- | Mus musculus protein-L-isoaspartate | BG086679 | Mm.25169 | Energy/Metabolism |
| 2613 | | methyltransferase 1 (Pcmt1), | (D-aspartate) O-methyltransferase 1 | | | |
| | | | (Pcmt1), mRNA | | | |
| 2614 | H3129D08 | “Homo sapiens candidate tumor | | BG086692 | Mm.43482 | Signal Transduction |
| 2615 | | suppressor p33 ING1 homolog | | | | |
| | | (LOC51147), mRNA” | | | | |
| 2616 | H3129E06 | OXA1L gene sequence | Homo sapiens OXA1L gene complete | BG086699 | Mm.142455 | Apoptosis |
| 2617 | | | sequence | | | |
| 2618 | H3129E10 | “Mus musculus Pro-rich, PH, SH2 | | BG086702 | Mm.8538 | Signal Transduction |
| 2619 | | domain-containing signaling mediator | | | | |
| | | (PSM) mRNA, complete cds” | | | | |
| 2620 | H3129E12 | Mus musculus IFN alpha-treated | | BG086704 | Mm.23836 | Heat Shock/Stress |
| 2621 | | embryonic fibroblast mRNA | | | | |
| 2622 | H3129F04 | clone TA-9 ATP synthase b chain | Mus musculus clone TA-9 ATP | | Mm.30112 | Energy/Metabolism |
| 2623 | | homolog, partial cds | synthase b chain homolog mRNA, | | | |
| | | | partial cds | | | |
| 2624 | H3129G02 | serine palmitoyltransferase, long chain | Mus musculus serine | BG074002 | Mm.6505 | Energy/Metabolism |
| 2625 | | base subunit 1 (Sptlc1), | palmitoyltransferase, long chain base | | | |
| | | | subunit 1 (Sptlc1), mRNA | | | |
| 2626 | H3129G06 | peroxisomal assembly protein PEX3P | Mus musculus peroxisomal assembly | BG086720 | | Energy/Metabolism |
| 2627 | | (Pex3) gene, promoter and exon 1 | protein PEX3P (Pex3) gene, promoter | | | |
| | | | and exon 1 | | | |
| 2628 | H3129H03 | hydroxylacyl-Coenzyme A | Mus musculus hydroxylacyl-Coenzyme | BG086728 | Mm.2491 | Energy/Metabolism |
| 2629 | | dehydrogenase (Hadh), | A dehydrogenase (Hadh), mRNA | | | |
| 2630 | H3129H05 | K—Cl cotransporter KCC3 | Homo sapiens K—Cl cotransporter | BG086729 | Mm.21915 | Energy/Metabolism |
| 2631 | | | KCC3 mRNA, complete cds | | | |
| 2632 | H3130B10 | SERCA2 gene, alternative transcripts | Mus musculus SERCA2 gene, | BG074044 | Mm.42255 | Energy/Metabolism |
| 2633 | | | alternative transcripts | | | |
| 2634 | H3130B11 | “Mouse Cu—Zn superoxide dismutase | | BG074045 | Mm.5274 | Heat Shock/Stress |
| 2635 | | mRNA, complete cds” | | | | |
| 2636 | H3130C02 | “Mus musculus calcineurin inhibitor | | BG086761 | Mm.56 | Signal Transduction |
| 2637 | | mRNA, complete cds, alternatively | | | | |
| | | spliced” | | | | |
| 2638 | H3130D06 | Na, K-ATPase beta subunit (ATP1B) | Human Na, K-ATPase beta subunit | BG086774 | | Energy/Metabolism |
| 2639 | | gene, exons 1 and 2 | (ATP1B) gene, exons 1 and 2 | | | |
| 2640 | H3130D08 | NADH/NADPH mitogenic oxidase | Rattus norvegicus NADH/NADPH | | Mm.2866 | |
| 2641 | | subunit p65-mox | mitogenic oxidase subunit p65-mox | | | |
| | | | mRNA, complete cds | | | |
| 2642 | H3130H07 | 3-hydroxy-3-methylglutaryl-Coenzyme | | | Mm.10633 | |
| 2643 | | A synthase 2 mHS | | | | |
| 2644 | H3131A03 | “Mus musculus heat shock protein, 86 kDa | | BG074109 | Mm.1843 | Heat Shock/Stress |
| 2645 | | 1 (Hsp86-1), mRNA” | | | | |
| 2646 | H3131A05 | ATP synthase gamma-subunit precursor | Mus musculus mRNA for ATP synthase | BG074111 | Mm.12677 | Energy/Metabolism |
| 2647 | | | gamma-subunit precursor complete cds | | | |
| 2648 | H3131A07 | enoyl coenzyme A hydratase 1, | Mus musculus enoyl coenzyme A | BG074113 | Mm.2112 | Energy/Metabolism |
| 2649 | | peroxisomal (Ech1), | hydratase 1, peroxisomal (Ech1), | | | |
| | | | mRNA | | | |
| 2650 | H3131A12 | MA-3 (apoptosis-related gene) | Mouse MA-3 (apoptosis-related gene) | BG086831 | Mm.26035 | Apoptosis |
| 2651 | | | mRNA, complete cds | | | |
| 2652 | H3131B04 | sterol-carrier protein X | Mus musculus sterol-carrier protein X | BG086835 | Mm.1779 | Matrix/Structural |
| 2653 | | | mRNA, complete cds | | | Proteins |
| 2654 | H3131C10 | dolichyl-di-phosphooligosaccharide- | Mus musculus dolichyl-di- | BG074138 | Mm.7236 | Energy/Metabolism |
| 2655 | | protein glycotransferase (Ddost), | phosphooligosaccharide-protein | | | |
| | | | glycotransferase (Ddost), mRNA | | | |
| 2656 | H3131D05 | solute carrier family 39 (iron-regulated | Mus musculus solute carrier family 39 | BG074144 | Mm.28756 | Energy/Metabolism |
| 2657 | | transporter), member 1 (Slc39a1), | (iron-regulated transporter), member 1 | | | |
| | | | (Slc39a1), mRNA | | | |
| 2658 | H3131G02 | caspase 12 (Casp12), | Mus musculus caspase 12 (Casp12), | BG086881 | Mm.42163 | Apoptosis |
| 2659 | | | mRNA | | | |
| 2660 | H3131H01 | H. sapiens gene for rho GDP | | BG086892 | Mm.30016 | Signal Transduction |
| 2661 | | dissociation inhibitor (GDI) | | | | |
| 2662 | H3132C10 | “Mus musculus neuroblastoma ras | | BG086925 | Mm.734 | Signal Transduction |
| 2663 | | oncogene (Nras), mRNA” | | | | |
| 2664 | H3132F06 | “Homo sapiens pleiomorphic adenoma | | BG074245 | Mm.23923 | Signal Transduction |
| 2665 | | gene-like 2 (PLAGL2), mRNA” | | | | |
| 2666 | H3132G04 | “Homo sapiens ER-associated DNAJ; | | BG086956 | Mm.37516 | Heat Shock/Stress |
| 2667 | | ER-associated Hsp40 co-chaperone; | | | | |
| | | hDj9; ERj3 (LOC51726), mRNA” | | | | |
| 2668 | H3132G09 | vacuolar ATPase subunit H | Homo sapiens vacuolar ATPase subunit | BG086960 | Mm.27082 | Energy/Metabolism |
| 2669 | | | H mRNA, complete cds | | | |
| 2670 | H3132H07 | “Mus musculus tumor susceptibility | | BG086965 | Mm.22688 | Signal Transduction |
| 2671 | | protein 101 (tsg101) gene, complete | | | | |
| | | cds” | | | | |
| 2672 | H3133A06 | glutathione S-transferase, mu 1 (Gstm1), | Mus musculus glutathione S-transferase, | BG086970 | Mm.2011 | Energy/Metabolism |
| 2673 | | | mu 1 (Gstm1), mRNA | | | |
| 2674 | H3133B05 | cytochrome c oxidase subunit VIb | Homo sapiens cytochrome c oxidase | BG074277 | Mm.400 | Energy/Metabolism |
| 2675 | | (COX6B), | subunit VIb (COX6B), mRNA | | | |
| 2676 | H3133C08 | “Mus musculus RAB7, member RAS | | BG086994 | Mm.4268 | Signal Transduction |
| 2677 | | oncogene family (Rab7), mRNA” | | | | |
| 2678 | H3133D04 | NADH dehydrogenase (ubiquinone) | Homo sapiens NADH dehydrogenase | BG087002 | Mm.2206 | Energy/Metabolism |
| 2679 | | flavoprotein 2 (24 kD) (NDUFV2), | (ubiquinone) flavoprotein 2 (24 kD) | | | |
| | | | (NDUFV2), mRNA | | | |
| 2680 | H3133D06 | | | | Mm.21535 | Heat Shock/Stress |
| 2681 | | | | | | |
| 2682 | H3133E07 | glycine amidinotransferase (L- | Homo sapiens glycine | BG087011 | Mm.29975 | Energy/Metabolism |
| 2683 | | arginine: glycine amidinotransferase) | amidinotransferase (L-arginine: glycine | | | |
| | | (GATM), | amidinotransferase) (GATM), mRNA | | | |
| 2684 | H3133H01 | “Mus musculus heat shock 70 protein | | BG087043 | Mm.2144 | Heat Shock/Stress |
| 2685 | | (Hsc70) gene, complete cds” | | | | |
| 2686 | H3133H05 | M. musculus arylsulfatase A | M. musculus mRNA for arylsulfatase A | BG087046 | Mm.620 | Energy/Metabolism |
| 2687 | | | | | | |
| 2688 | H3133H07 | voltage-dependent anion channel 3 | Mus musculus voltage-dependent anion | BG087048 | Mm.133962 | Energy/Metabolism |
| 2689 | | (Vdac3), | channel 3 (Vdac3), mRNA | | | |
| 2690 | H3134C07 | DNA, DLEC1 to ORCTL4 gene region, | Homo sapiens DNA, DLEC1 to | BG087037 | Mm.42275 | Energy/Metabolism |
| 2691 | | section 1/2 (DLEC1, ORCTL3, | ORCTL4 gene region, section 1/2 | | | |
| | | ORCTL4 genes) | (DLEC1, ORCTL3, ORCTL4 genes, | | | |
| | | | complete cds) | | | |
| 2692 | H3134D06 | GrpE-like 2, mitochondrial mt- | | | Mm.12959 | Heat Shock/Stress |
| 2693 | | GrpE#2 | | | | |
| 2694 | H3135A03 | NADH dehydrogenase (ubiquinone) 1 | Homo sapiens NADH dehydrogenase | BG087056 | Mm.19834 | Energy/Metabolism |
| 2695 | | alpha subcomplex, 8 (19 kD, PGIV) | (ubiquinone) 1 alpha subcomplex, 8 | | | |
| | | (NDUFA8), | (19 kD, PGIV) (NDUFA8), mRNA | | | |
| 2696 | H3135B06 | cDNA DKFZp434D175 (from clone | Homo sapiens mRNA; cDNA | BG087066 | Mm.30110 | Energy/Metabolism |
| 2697 | | DKFZp434D175); partial cds | DKFZp434D175 (from clone | | | |
| | | | DKFZp434D175); partial cds | | | |
| 2698 | H3135B10 | “Mus musculus PL-6 (P16), mRNA” | | BG087070 | Mm.27312 | Signal Transduction |
| 2699 | | | | | | |
| 2700 | H3135D06 | galactose-1-phosphate uridyltransferase | Mus musculus galactose-1-phosphate | BG087088 | Mm.2420 | Energy/Metabolism |
| 2701 | | (GALT) gene | uridyltransferase (GALT) gene, | | | |
| | | | complete cds | | | |
| 2702 | H3135F04 | Mm.87124 | | | Mm.87124 | Energy/Metabolism |
| 2703 | | | | | | |
| 2704 | H3135F06 | | | | | Heat Shock/Stress |
| 2705 | H3135G03 | serine protease inhibitor 3 (Spi3), | Mus musculus serine protease inhibitor | BG087117 | Mm.2623 | Energy/Metabolism |
| 2706 | | | 3 (Spi3), mRNA | | | |
| 2707 | H3135G07 | protoporphyrinogen oxidase (Ppox), | Mus musculus protoporphyrinogen | BG087120 | Mm.4730 | Energy/Metabolism |
| 2708 | | | oxidase (Ppox), mRNA | | | |
| 2709 | H3135H04 | rhodanese (Tst) | Mus musculus rhodanese (Tst) mRNA, | | Mm.15312 | |
| 2710 | | | complete cds | | | |
| 2711 | H3135H10 | D-dopachrome tautomerase gene | Mus musculus D-dopachrome | BG087134 | Mm.5731 | Energy/Metabolism |
| 2712 | | | tautomerase gene, complete cds | | | |
| 2713 | H3136A06 | calcium-sensitive chloride conductance | Mus musculus calcium-sensitive | BG087141 | Mm.20897 | Energy/Metabolism |
| 2714 | | protein-1 (mCLCA1) | chloride conductance protein-1 | | | |
| | | | (mCLCA1) mRNA, complete cds | | | |
| 2715 | H3136B09 | branched chain ketoacid dehydrogenase | Mus musculus branched chain ketoacid | BG087153 | Mm.25848 | Energy/Metabolism |
| 2716 | | E1, alpha polypeptide (Bckdha), | dehydrogenase E1, alpha polypeptide | | | |
| | | | (Bckdha), mRNA | | | |
| 2717 | H3136C07 | BCL2/adenovirus E1B 19 kDa- | Mus musculus BCL2/adenovirus E1B | BG087162 | Mm.2159 | Apoptosis |
| 2718 | | interacting protein 1, NIP3 (Bnip3), | 19 kDa-interacting protein 1, NIP3 | | | |
| | | | (Bnip3), mRNA | | | |
| 2719 | H3136D05 | serine protease | Mus musculus serine protease mRNA, | BG087171 | Mm.24276 | Energy/Metabolism |
| 2720 | | | complete cds | | | |
| 2721 | H3136E12 | gene uncoupling protein-2 | Mus musculus gene for uncoupling | | Mm.144413 | |
| 2722 | | | protein-2, complete cds | | | |
| 2723 | H3136F07 | guanine monphosphate synthetase | Homo sapiens guanine monphosphate | BG087194 | Mm.28299 | Energy/Metabolism |
| 2724 | | (GMPS), | synthetase (GMPS), mRNA | | | |
| 2725 | H3136F10 | serine protease inhibitor 3 (Spi3), | Mus musculus serine protease inhibitor | BG087196 | | Energy/Metabolism |
| 2726 | | | 3 (Spi3), mRNA | | | |
| 2727 | H3136G10 | solute carrier family 12, member 2 | Mus musculus solute carrier family 12, | BG074576 | Mm.4168 | Energy/Metabolism |
| 2728 | | (Slc12a2), | member 2 (Slc12a2), mRNA | | | |
| 2729 | H3136H12 | “Mus musculus IkB kinase-beta (Ikkb) | | BG087220 | Mm.28269 | Signal Transduction |
| 2730 | | mRNA, complete cds” | | | | |
| 2731 | H3137A09 | pyruvate decarboxylase (Pcx), | Mus musculus pyruvate decarboxylase | BG087228 | Mm.1845 | Energy/Metabolism |
| 2732 | | | (Pcx), mRNA | | | |
| 2733 | H3137B06 | ornithine aminotransferase (Oat), | Mus musculus ornithine | BG087236 | Mm.13694 | Energy/Metabolism |
| 2734 | | | aminotransferase (Oat), mRNA | | | |
| 2735 | H3137B09 | “Homo sapiens Rho guanine nucleotide | | BG087239 | Mm.41604 | Signal Transduction |
| 2736 | | exchange factor (GEF) 4 (ARHGEF4), | | | | |
| | | mRNA” | | | | |
| 2737 | H3137C10 | ATP-binding cassette, sub-family A | Homo sapiens ATP-binding cassette, | BG087249 | Mm.38313 | Energy/Metabolism |
| 2738 | | (ABC1), member 3 (ABCA3), | sub-family A (ABC1), member 3 | | | |
| | | | (ABCA3), mRNA | | | |
| 2739 | H3137D07 | ATP-binding cassette, sub-family E | Mus musculus ATP-binding cassette, | BG087256 | Mm.5831 | Energy/Metabolism |
| 2740 | | (OABP), member 1 (Abce1), | sub-family E (OABP), member 1 | | | |
| | | | (Abce1), mRNA | | | |
| 2741 | H3137D08 | VLCAD gene | Mus musculus VLCAD gene | BG074630 | Mm.18630 | Energy/Metabolism |
| 2742 | | | | | | |
| 2743 | H3137D09 | solute carrier family 4 (anion | Mus musculus solute carrier family 4 | BG074631 | Mm.4580 | Energy/Metabolism |
| 2744 | | exchanger), member 2 (Slc4a2), | (anion exchanger), member 2 (Slc4a2), | | | |
| | | | mRNA | | | |
| 2745 | H3137E02 | NADH dehydrogenase (ubiquinone) Fe—S | Homo sapiens NADH dehydrogenase | BG087262 | Mm.21669 | Energy/Metabolism |
| 2746 | | protein 2 (49 kD) (NADH-coenzyme | (ubiquinone) Fe—S protein 2 (49 kD) | | | |
| | | Q reductase) (NDUFS2), | (NADH-coenzyme Q reductase) | | | |
| | | | (NDUFS2), mRNA | | | |
| 2747 | H3137E03 | anion exchanger 3 brain and anion | Mus musculus anion exchanger 3 brain | BG074636 | Mm.5053 | Energy/Metabolism |
| 2748 | | exchanger 3 cardiac isoms (Slc4a3) | and anion exchanger 3 cardiac isoforms | | | |
| | | gene, alternatively spliced | (Slc4a3) gene, complete cds, | | | |
| | | | alternatively spliced | | | |
| 2749 | H3137G05 | ribosomal protein L17 isolog | Homo sapiens ribosomal protein L17 | BG087283 | Mm.44225 | Energy/Metabolism |
| 2750 | | (LOC63875), | isolog (LOC63875), mRNA | | | |
| 2751 | H3137G09 | Mouse mRNA for protein tyrosine | | BG087286 | Mm.4220 | Signal Transduction |
| 2752 | | phosphatase PTPT9 | | | | |
| 2753 | H3138B02 | neuronal apoptosis inhibitory protein 6 | Mus musculus neuronal apoptosis | BG087312 | Mm.13437 | Apoptosis |
| 2754 | | (Naip6) gene; and Naip3 gene, exons 2-9 | inhibitory protein 6 (Naip6) gene, | | | |
| | | and 11-16 | complete cds; and Naip3 gene, exons 2-9 | | | |
| | | | and 11-16 | | | |
| 2755 | H3138C04 | phosphoglucomutase 1 (PGM1), | Homo sapiens phosphoglucomutase 1 | BG087325 | Mm.22403 | Energy/Metabolism |
| 2756 | | | (PGM1), mRNA | | | |
| 2757 | H3138D04 | hypothetical protein FLJ10545 | Homo sapiens hypothetical protein | BG074710 | Mm.9870 | Energy/Metabolism |
| 2758 | | (FLJ10545), | FLJ10545 (FLJ10545), mRNA | | | |
| 2759 | H3138D07 | voltage-dependent anion channel 2 | Mus musculus voltage-dependent anion | BG087336 | Mm.569 | Energy/Metabolism |
| 2760 | | (Vdac2), | channel 2 (Vdac2), mRNA | | | |
| 2761 | H3138E10 | aldehyde dehydrogenase 5 (ALDH5), | Homo sapiens aldehyde dehydrogenase | BG087349 | Mm.24457 | Energy/Metabolism |
| 2762 | | | 5 (ALDH5), mRNA | | | |
| 2763 | H3138F05 | ADP-ribosylation factor 4 (Arf4), | Mus musculus ADP-ribosylation factor | BG087356 | Mm.1486 | Energy/Metabolism |
| 2764 | | | 4 (Arf4), mRNA | | | |
| 2765 | H3138G02 | solute carrier family 22 (organic cation | Mus musculus solute carrier family 22 | BG087364 | Mm.42253 | Energy/Metabolism |
| 2766 | | transporter), member 5 (Slc22a5), | (organic cation transporter), member 5 | | | |
| | | | (Slc22a5), mRNA | | | |
| 2767 | H3138G09 | alpha glucosidase 2, alpha neutral | Mus musculus alpha glucosidase 2, | BG087370 | Mm.3196 | Energy/Metabolism |
| 2768 | | subunit (G2an), | alpha neutral subunit (G2an), mRNA | | | |
| 2769 | H3138H04 | long-chain acyl-CoA synthetase | Homo sapiens mRNA for long-chain | BG074754 | Mm.28962 | Energy/Metabolism |
| 2770 | | | acyl-CoA synthetase, complete cds | | | |
| 2771 | H3138H07 | 78 kDa gastrin-binding protein | Human 78 kDa gastrin-binding protein | BG087380 | Mm.16773 | Energy/Metabolism |
| 2772 | | | mRNA, complete cds | | | |
| 2773 | H3138H10 | M. musculus mRNA for cathepsin D | | BG087383 | Mm.2147 | Signal Transduction |
| 2774 | | | | | | |
| 2775 | H3139B06 | “Mus musculus protein tyrosine | | BG087401 | Mm.5400 | Signal Transduction |
| 2776 | | phosphatase, non-receptor type 13 | | | | |
| | | interacting protein (Ptpn13ip), mRNA” | | | | |
| 2777 | H3139C01 | ubi-d4/requiem gene, exons 2 through | Mus musculus ubi-d4/requiem gene, | BG087408 | Mm.2651 | Apoptosis |
| 2778 | | 11 and partial cds | exons 2 through 11 and partial cds | | | |
| 2779 | H3139E01 | “Mus musculus heat shock 70 protein | | BG087426 | | Heat Shock/Stress |
| 2780 | | (Hsc70) gene, complete cds” | | | | |
| 2781 | H3139F01 | sterol O-acyltransferase 1 (Soat1), | Mus musculus sterol O-acyltransferase 1 | BG087432 | Mm.28099 | Energy/Metabolism |
| 2782 | | | (Soat1), mRNA | | | |
| 2783 | H3140A02 | solute carrier family 9 | Mus musculus solute carrier family 9 | BG087460 | Mm.4312 | Energy/Metabolism |
| 2784 | | (sodium/hydrogen exchanger), member | (sodium/hydrogen exchanger), member | | | |
| | | 1 (Slc9a1), | 1 (Slc9a1), mRNA | | | |
| 2785 | H3140A08 | “Mus musculus mitogen activated | | BG087465 | Mm.4437 | Signal Transduction |
| 2786 | | protein kinase 14 (Mapk14), mRNA” | | | | |
| 2787 | H3140A09 | CPT I = carnitine palmitoyltransferase I | CPT I = carnitine palmitoyltransferase I | | Mm.18522 | Energy/Metabolism |
| 2788 | | {3 region} [mice, JVS, hearts, Partial, | {3 region} [mice, JVS, hearts, mRNA | | | |
| | | 678 nt, segment 1 of 2] | Partial, 678 nt, segment 1 of 2] | | | |
| 2789 | H3140B07 | major histocompatibility locus class III | Mus musculus major histocompatibility | | Mm.1511 | |
| 2790 | | region: butyrophilin-like protein gene, | locus class III region: butyrophilin-like | | | |
| | | partial cds; Notch4, PBX2, RAGE, | protein gene, partial cds; Notch4, | | | |
| | | lysophatidic acid acyl transferase-alpha, | PBX2, RAGE, lysophatidic acid acyl | | | |
| | | palmitoyl-protein thioestera | transferase-alpha, palmitoyl-protein | | | |
| | | | thioestera | | | |
| 2791 | H3140C11 | clone RP11-486I22, sequence | Homo sapiens clone RP11-486I22, | BG074864 | Mm.3479 | Energy/Metabolism |
| 2792 | | | complete sequence | | | |
| 2793 | H3140D05 | ectonucleotide | Homo sapiens ectonucleotide | BG087491 | Mm.30145 | Energy/Metabolism |
| 2794 | | pyrophosphatase/phosphodiesterase 5 | pyrophosphatase/phosphodiesterase 5 | | | |
| | | (putative function) (ENPP5), | (putative function) (ENPP5), mRNA | | | |
| 2795 | H3140D07 | peroxiredoxin 3 (Prdx3), | Mus musculus peroxiredoxin 3 (Prdx3), | BG087493 | Mm.29821 | Energy/Metabolism |
| 2796 | | | mRNA | | | |
| 2797 | H3140D12 | electron-transfer-flavoprotein, alpha | Homo sapiens electron-transfer- | BG087498 | Mm.26949 | Energy/Metabolism |
| 2798 | | polypeptide (glutaric aciduria II) | flavoprotein, alpha polypeptide | | | |
| | | (ETFA), | (glutaric aciduria II) (ETFA), mRNA | | | |
| 2799 | H3140E04 | protein tyrosine phosphatase (70zshp) | Mouse protein tyrosine phosphatase | BG074880 | Mm.1484 | Energy/Metabolism |
| 2800 | | | (70zshp) mRNA, complete cds | | | |
| 2801 | H3140E08 | glutamate dehydrogenase (Glud), | Mus musculus glutamate dehydrogenase | BG087506 | Mm.10600 | Energy/Metabolism |
| 2802 | | | (Glud), mRNA | | | |
| 2803 | H3141F03 | solute carrier family 12, member 7 | Mus musculus solute carrier family 12, | BG087669 | Mm.24510 | Energy/Metabolism |
| 2804 | | (Slc12a7), | member 7 (Slc12a7), mRNA | | | |
| 2805 | H3141G03 | chs HMG CoA synthase, partial cds | Mus musculus chs HMG CoA synthase | BG087678 | Mm.10633 | Matrix/Structural |
| 2806 | | | mRNA, partial cds | | | Proteins |
| 2807 | H3142A02 | “Mus musculus protein phosphatase 2a, | | BG087694 | Mm.7418 | Signal Transduction |
| 2808 | | catalytic subunit, beta isoform (Ppp2cb), | | | | |
| | | mRNA” | | | | |
| 2809 | H3142A08 | ubiquinol-cytochrome c reductase core | Homo sapiens ubiquinol-cytochrome c | BG075002 | Mm.988 | Energy/Metabolism |
| 2810 | | protein II (UQCRC2), | reductase core protein II (UQCRC2), | | | |
| | | | mRNA | | | |
| 2811 | H3142E09 | “Mus musculus tumor-suppressing | | BG087715 | Mm.139675 | Signal Transduction |
| 2812 | | subchromosomal transferable fragment | | | | |
| | | 4 (Tssc4), mRNA” | | | | |
| 2813 | H3142G01 | | | | Mm.31203 | Heat Shock/Stress |
| 2814 | H3142G02 | methylenetetrahydrofolate | Homo sapiens | BG087719 | Mm.29584 | Energy/Metabolism |
| 2815 | | dehydrogenase (NADP+ dependent), | methylenetetrahydrofolate | | | |
| | | methenyltetrahydrofolate | dehydrogenase (NADP+ dependent), | | | |
| | | cyclohydrolase, myltetrahydrofolate | methenyltetrahydrofolate | | | |
| | | synthetase (MTHFD1), | cyclohydrolase, formyltetrahydrofolate | | | |
| | | | synthetase (MTHFD1), mRNA | | | |
| 2816 | H3141D09 | peroxisome membrane protein PEX2 | Mus musculus peroxisome membrane | BG075110 | Mm.16453 | Energy/Metabolism |
| 2817 | | | protein PEX2 mRNA, complete cds | | | |
| 2818 | H3143D10 | “Mouse cAMP-dependent protein kinase | | BG075111 | Mm.19111 | Signal Transduction |
| 2819 | | alpha subunit gene, exon 10” | | | | |
| 2820 | H3143D12 | presenilin-associated protein | Homo sapiens presenilin-associated | BG087589 | Mm.29650 | Energy/Metabolism |
| 2821 | | | protein mRNA, complete cds | | | |
| 2822 | H3143H07 | “Mus musculus osmotic stress protein | | BG075154 | Mm.4150 | Heat Shock/Stress |
| 2823 | | 94 (Osp94) mRNA, complete cds” | | | | |
| 2824 | H3144B03 | NADH-ubiquinone dehydrogenase 1 | Homo sapiens NADH-ubiquinone | BG087636 | Mm.4537 | Energy/Metabolism |
| 2825 | | beta subcomplex | dehydrogenase 1 beta subcomplex | | | |
| | | | mRNA, complete cds | | | |
| 2826 | H3144C01 | glyoxalase I (GLO1), | Homo sapiens glyoxalase I (GLO1), | BG087642 | Mm.17362 | Energy/Metabolism |
| 2827 | | | mRNA | | | |
| 2828 | H3144C11 | “Mus musculus heat shock 70 kD protein | | BG087650 | Mm.24162 | Heat Shock/Stress |
| 2829 | | 5 (glucose-regulated protein, 78 kD) | | | | |
| | | (Hspa5), mRNA” | | | | |
| 2830 | H3144E06 | neuronal cell death related gene in | Rattus norvegicus neuronal cell death | AW555571 | Mm.19440 | Apoptosis |
| | | neuron-7 (DN-7) | related gene in neuron-7 (DN-7) | | | |
| | | | mRNA, complete cds | | | |
| 2831 | H3144F09 | “Homo sapiens RAB7, member RAS | | BG075214 | Mm.34027 | Signal Transduction |
| 2832 | | oncogene family-like 1 (RAB7L1), | | | | |
| | | mRNA” | | | | |
| 2833 | H3144G02 | “Homo sapiens phosphodiesterase 5A, | | BG087768 | Mm.103149 | Signal Transduction |
| 2834 | | cGMP-specific (PDE5A), mRNA” | | | | |
| 2835 | H3144H01 | mitofilin, partial cds | Homo sapiens mitofilin mRNA, partial | | Mm.10706 | |
| 2836 | | | cds | | | |
| 2837 | H3145A03 | “Mus musculus partial Prkar1a gene for | | BG075240 | Mm.30039 | Signal Transduction |
| 2838 | | cAMP-dependent protein kinase | | | | |
| | | regulatory subunit RIalpha, exons 8-10 | | | | |
| | | and 3′UTR” | | | | |
| 2839 | H3145A06 | zinc transporter ZnT-3 (ZnT-3) | Mus musculus zinc transporter ZnT-3 | BG075243 | Mm.1396 | Energy/Metabolism |
| 2840 | | | (ZnT-3) mRNA, complete cds | | | |
| 2841 | H3145E01 | H. sapiens gene phosphate carrier | H. sapiens gene for phosphate carrier | BG075286 | Mm.5246 | Energy/Metabolism |
| 2842 | | | | | | |
| 2843 | H3145E12 | hyaluronidase 2 (Hyal2), | Mus musculus hyaluronidase 2 (Hyal2), | BG075294 | Mm.4834 | Energy/Metabolism |
| 2844 | | | mRNA | | | |
| 2845 | H3145F11 | | | | | Heat Shock/Stress |
| 2846 | | | | | | |
| 2847 | H3145H03 | M. musculus mRNA for mitogen | | BG075319 | Mm.8385 | Signal Transduction |
| 2848 | | activated protein kinase (erk-1) | | | | |
| 2849 | H3145H09 | peroxiredoxin 4 (Prdx4), | Mus musculus peroxiredoxin 4 (Prdx4), | BG075324 | Mm.19127 | Energy/Metabolism |
| 2850 | | | mRNA | | | |
| 2851 | H3145H10 | peroxiredoxin 4 (Prdx4), | Mus musculus peroxiredoxin 4 (Prdx4), | BG087872 | Mm.19127 | Energy/Metabolism |
| 2852 | | | mRNA | | | |
| 2853 | H3146A04 | “Mus musculus myo-inositol 1- | | BG075331 | Mm.29357 | Signal Transduction |
| 2854 | | phosphate synthase A1 (IsynA1) | | | | |
| | | mRNA, complete cds” | | | | |
| 2855 | H3146B10 | cDNA FLJ13847 fis, clone | Homo sapiens cDNA FLJ13847 fis, | BG087895 | Mm.24210 | Energy/Metabolism |
| 2856 | | THYRO1000852, highly similar to | clone THYRO1000852, highly similar | | | |
| | | branched-chain amino acid | to Human branched-chain amino acid | | | |
| | | aminotransferase (ECA40) | aminotransferase (ECA40) mRNA | | | |
| 2857 | H3146F01 | RHOA proto-oncogene multi-drug- | Homo sapiens RHOA proto-oncogene | BG087931 | Mm.757 | Energy/Metabolism |
| 2858 | | resistance protein, 3 end | multi-drug-resistance protein mRNA, 3 | | | |
| | | | end | | | |
| 2859 | H3146G06 | cDNA: FLJ22871 fis, clone KAT02533 | Homo sapiens cDNA: FLJ22871 fis, | | Mm.30065 | Energy/Metabolism |
| 2860 | | | clone KAT02533 | | | |
| 2861 | H3146H06 | CTL2 gene | Homo sapiens CTL2 gene | BG087957 | Mm.28209 | Energy/Metabolism |
| 2862 | | | | | | |
| 2863 | H3147B08 | “Mus musculus RAB11B, member RAS | | BG087979 | Mm.35727 | Signal Transduction |
| 2864 | | oncogene family (Rab11b), mRNA” | | | | |
| 2865 | H3147D12 | apoptosis inhibitory protein 5 (Api5), | Mus musculus apoptosis inhibitory | BG088003 | Mm.692 | Apoptosis |
| 2866 | | | protein 5 (Api5), mRNA | | | |
| 2867 | H3147E03 | “Mus musculus signal transducer and | | BG088006 | Mm.25298 | Signal Transduction |
| 2868 | | activator of transcription 3 interacting | | | | |
| | | protein 1 (Stat3ip1-pending), mRNA” | | | | |
| 2869 | H3147E04 | “Mus musculus heat shock protein, 84 kDa | | BG088007 | Mm.2180 | Heat Shock/Stress |
| 2870 | | 1 (Hsp84-1), mRNA” | | | | |
| 2871 | H3147E05 | “Mus musculus heat shock protein, 84 kDa | | BG088008 | Mm.2180 | Heat Shock/Stress |
| 2872 | | 1 (Hsp84-1), mRNA” | | | | |
| 2873 | H3147E06 | “Mus musculus heat shock protein, 84 kDa | | BG088009 | Mm.2180 | Heat Shock/Stress |
| 2874 | | 1 (Hsp84-1), mRNA” | | | | |
| 2875 | H3148B04 | ferredoxin reductase (Fdxr), | Mus musculus ferredoxin reductase | BG088056 | Mm.4719 | Energy/Metabolism |
| 2876 | | | (Fdxr), mRNA | | | |
| 2877 | H3148E08 | ornithine decarboxylase antizyme gene | Mus musculus ornithine decarboxylase | BG088091 | Mm.683 | Energy/Metabolism |
| 2878 | | | antizyme gene, complete cds | | | |
| 2879 | H3148F05 | NADH dehydrogenase (ubiquinone) Fe—S | Homo sapiens NADH dehydrogenase | BG088100 | Mm.2041 | Energy/Metabolism |
| 2880 | | protein 1 (75 kD) (NADH-coenzyme | (ubiquinone) Fe—S protein 1 (75 kD) | | | |
| | | Q reductase) (NDUFS1), | (NADH-coenzyme Q reductase) | | | |
| | | | (NDUFS1), mRNA | | | |
| 2881 | H3148G06 | “Mus musculus MgcRacGAP mRNA | | BG088110 | Mm.27141 | Signal Transduction |
| 2882 | | for GTPase activating protein, complete | | | | |
| | | cds” | | | | |
| 2883 | H3149B10 | “Mus musculus protein kinase C, zeta | | BG088144 | Mm.28561 | Signal Transduction |
| 2884 | | (Pkcz), mRNA” | | | | |
| 2885 | H3149B11 | membrane protein, palmitoylated (55 kDa) | Mus musculus membrane protein, | | Mm.2814 | Matrix/Structural |
| 2886 | | (Mpp1), | palmitoylated (55 kDa) (Mpp1), mRNA | | | Proteins |
| 2887 | H3149C10 | triosephosphate isomerase (Tpi), | Mus musculus triosephosphate | BG075608 | Mm.4222 | Energy/Metabolism |
| 2888 | | | isomerase (Tpi), mRNA | | | |
| 2889 | H3149E11 | sodium channel, voltage-gated, type II, | Homo sapiens sodium channel, voltage- | BG088175 | | Energy/Metabolism |
| 2890 | | beta polypeptide (SCN2B), | gated, type II, beta polypeptide | | | |
| | | | (SCN2B), mRNA | | | |
| 2891 | H3149H04 | 5-3 exoribonuclease 2 (Xrn2), | Mus musculus 5-3 exoribonuclease 2 | BG088199 | Mm.3065 | Energy/Metabolism |
| 2892 | | | (Xrn2), mRNA | | | |
| 2893 | H3150C08 | “Mus musculus Ste-20 related kinase | | BG088221 | Mm.29404 | Signal Transduction |
| 2894 | | (Spak-pending), mRNA” | | | | |
| 2895 | H3150D02 | “Mus musculus presenilin 1 (Psen1), | | BG088227 | Mm.998 | Signal Transduction |
| 2896 | | mRNA” | | | | |
| 2897 | H3150F06 | farnesyltransferase, CAAX box, alpha | Mus musculus farnesyltransferase, | BG075707 | Mm.3496 | Energy/Metabolism |
| 2898 | | (Fnta), | CAAX box, alpha (Fnta), mRNA | | | |
| 2899 | H3150G03 | cDNA: FLJ21905 fis, clone HEP03764 | Homo sapiens cDNA: FLJ21905 fis, | BG088257 | Mm.4290 | Energy/Metabolism |
| 2900 | | | clone HEP03764 | | | |
| 2901 | H3151C06 | “Homo sapiens PTPL1-associated | | BG088297 | | Signal Transduction |
| 2902 | | RhoGAP 1 (PARG1), mRNA” | | | | |
| 2903 | H3151D08 | defender against cell death 1 (Dad1), | Mus musculus defender against cell | BG075765 | Mm.2547 | Apoptosis |
| 2904 | | | death 1 (Dad1), mRNA | | | |
| 2905 | H3151D11 | prosaposin (psap\SGP-1) gene | Mus musculus prosaposin (psap\SGP-1) | BG088310 | Mm.3363 | Energy/Metabolism |
| 2906 | | | gene, complete cds | | | |
| 2907 | H3151E12 | “Mus musculus downstream of tyrosine | | BG088318 | Mm.156 | Signal Transduction |
| 2908 | | kinase 1 (Dok1), mRNA” | | | | |
| 2909 | H3151F06 | M. musculus xanthine dehydrogenase | M. musculus mRNA for xanthine | BG075778 | Mm.11223 | Energy/Metabolism |
| 2910 | | | dehydrogenase | | | |
| 2911 | H3152A02 | Rat beta-galactoside-alpha 2,6- | Rat beta-galactoside-alpha 2,6- | BG075800 | Mm.1944 | Energy/Metabolism |
| 2912 | | sialyltransferase | sialyltransferase mRNA | | | |
| 2913 | H3152D06 | 5-aminolevulinic acid synthase, 3 end | Mouse 5-aminolevulinic acid synthase | BG088378 | Mm.1217 | Energy/Metabolism |
| 2914 | | | mRNA, 3 end | | | |
| 2915 | H3152G07 | MAD2 (mitotic arrest deficient, yeast, | Homo sapiens MAD2 (mitotic arrest | | Mm.9648 | Cell Cycle |
| 2916 | | homolog)-like 2 (MAD2L2), | deficient, yeast, homolog)-like 2 | | | |
| | | | (MAD2L2), mRNA | | | |
| 2917 | H3152H05 | ATPase, class II, type 9A (Atp9a), | Mus musculus ATPase, class II, type 9A | BG075876 | Mm.10288 | Energy/Metabolism |
| 2918 | | | (Atp9a), mRNA | | | |
| 2918 | H3152H07 | NADH dehydrogenase (ubiquinone) 1 | Homo sapiens NADH dehydrogenase | BG088418 | Mm.28058 | Energy/Metabolism |
| 2919 | | beta subcomplex, 5 (16 kD, SGDH) | (ubiquinone) 1 beta subcomplex, 5 | | | |
| | | (NDUFB5), | (16 kD, SGDH) (NDUFB5), mRNA | | | |
| 2920 | H3153A02 | “Mus musculus calpain small subunit | | BG088424 | Mm.6534 | Signal Transduction |
| 2921 | | gene, exons 3 through 9 and partial cds” | | | | |
| 2922 | H3153A07 | “Homo sapiens zinedin (ZIN), mRNA” | | BG088429 | Mm.21612 | Signal Transduction |
| 2923 | | | | | | |
| 2924 | H3153B11 | Mm.666 | | | Mm.666 | Energy/Metabolism |
| 2925 | | | | | | |
| 2926 | H3153C09 | Mus musculus tissue inhibitor of | | BG088451 | Mm.19191 | Matrix/Structural |
| 2927 | | metalloproteinase 2 (Timp2), mRNA | | | | Proteins |
| 2928 | H3153C10 | “Homo sapiens protein phosphatase 2 | | BG088452 | Mm.21428 | Signal Transduction |
| 2930 | | (formerly 2A), regulatory subunit A (PR | | | | |
| | | 65), beta isoform (PPP2R1B), mRNA” | | | | |
| 2931 | H3153D05 | “Rattus norvegicus Diacylglycerol | | BG075920 | Mm.102207 | Signal Transduction |
| 2932 | | kinase 90 kDa (Dagk), mRNA” | | | | |
| 2933 | H3153D07 | p75NTR-associated cell death executor | Mus musculus p75NTR-associated cell | BG088461 | Mm.90787 | Apoptosis |
| 2934 | | (Nade) | death executor (Nade) mRNA, complete | | | |
| | | | cds | | | |
| 2935 | H3153E04 | NADH dehydrogenase (ubiquinone) 1 | Homo sapiens NADH dehydrogenase | BG088469 | Mm.29683 | Energy/Metabolism |
| 2936 | | beta subcomplex, 7 (18 kD, B18) | (ubiquinone) 1 beta subcomplex, 7 | | | |
| | | (NDUFB7), | (18 kD, B18) (NDUFB7), mRNA | | | |
| 2937 | H3153E08 | M. musculus mRNA for MAP kinase- | | BG088472 | Mm.29725 | Signal Transduction |
| 2938 | | activated protein kinase 2 | | | | |
| 2939 | H3153F08 | apoptotic cell clearance receptor | Mus Musculus apoptotic cell clearance | BG088494 | Mm.38825 | Apoptosis |
| 2940 | | PtdSerR | receptor PtdSerR mRNA, complete cds | | | |
| 2941 | H3153G02 | endonuclease G-like 1 (ENDOGL1), | Homo sapiens endonuclease G-like 1 | BG088499 | Mm.11669 | Energy/Metabolism |
| 2942 | | | (ENDOGL1), mRNA | | | |
| 2943 | H3153G08 | “Mus musculus RAS-related C3 | | BG088482 | Mm.1972 | Signal Transduction |
| 2944 | | botulinum substrate 2 (Rac2), mRNA” | | | | |
| 2945 | H3153H05 | peroxisomal delta3, delta2-enoyl- | Mus musculus peroxisomal delta3, | BG075962 | Mm.28883 | Energy/Metabolism |
| 2946 | | Coenzyme A isomerase (Peci-pending), | delta2-enoyl-Coenzyme A isomerase | | | |
| | | | (Peci-pending), mRNA | | | |
| 2947 | H3154A03 | “Mus musculus RAN, member RAS | | BG088511 | Mm.7521 | Signal Transduction |
| 2948 | | oncogene family (Ran), mRNA” | | | | |
| 2949 | H3154C11 | M. musculus glutamyl-tRNA synthetase | M. musculus mRNA for glutamyl-tRNA | BG088535 | Mm.27190 | Energy/Metabolism |
| 2950 | | | synthetase | | | |
| 2951 | H3154E07 | “Mus musculus growth arrest specific 6 | | BG088548 | Mm.3982 | Signal Transduction |
| 2952 | | (Gas6), mRNA” | | | | |
| 2953 | H3154F02 | asparagine synthetase | Mus musculus asparagine synthetase | BG088553 | Mm.2942 | Energy/Metabolism |
| 2954 | | | mRNA, complete cds | | | |
| 2955 | H3155B01 | beclin 1 (coiled-coil, myosin-like | Mus musculus beclin 1 (coiled-coil, | BG088587 | Mm.30040 | Apoptosis |
| 2956 | | BCL2-interacting protein) (Becn1), | myosin-like BCL2-interacting protein) | | | |
| | | | (Becn1), mRNA | | | |
| 2957 | H3155B07 | NADH dehydrogenase (ubiquinone) Fe—S | Homo sapiens NADH dehydrogenase | BG076060 | Mm.30113 | Energy/Metabolism |
| 2958 | | protein 3 (30 kD) (NADH-coenzyme | ubiquinone) Fe—S protein 3 (30 kD) | | | |
| | | Q reductase) (NDUFS3), | (NADH-coenzyme Q reductase) | | | |
| | | | (NDUFS3), mRNA | | | |
| 2959 | H3155C04 | “Rattus norvegicus mRNA for inositol | | BG076067 | Mm.22360 | Signal Transduction |
| 2960 | | hexakisphosphate kinase, complete cds” | | | | |
| 2961 | H3155C10 | UDP glucuronosyltransferase (UGT1- | Mus musculus UDP | BG076072 | Mm.42472 | Energy/Metabolism |
| 2962 | | 06) | glucuronosyltransferase (UGT1-06) | | | |
| | | | mRNA, complete cds | | | |
| 2963 | H3155E04 | NDUFV1 gene, exons 6-10 | Homo sapiens NDUFV1 gene, exons 6-10 | BG076088 | Mm.29842 | Energy/Metabolism |
| 2964 | | | | | | |
| 2965 | H3155F02 | | | | | Heat Shock/Stress |
| 2966 | | | | | | |
| 2967 | H3155G03 | Rat endoplasmic reticulum alpha- | Rat endoplasmic reticulum alpha- | BG088635 | Mm.33138 | Energy/Metabolism |
| 2968 | | mannosidase | mannosidase mRNA, complete cds | | | |
| 2969 | H3156A10 | H3156A10 | | | | Apoptosis |
| 2970 | | | | | | |
| 2971 | H3156C03 | “Mus musculus protein phosphatase 1D | | BG088678 | Mm.61848 | Signal Transduction |
| 2972 | | magnesium-dependent, delta isoform | | | | |
| | | (Ppm1d), mRNA” | | | | |
| 2973 | H3156C05 | cDNA DKFZp586F2224 (from clone | Homo sapiens mRNA; cDNA | BG076153 | Mm.28030 | Energy/Metabolism |
| 2974 | | DKFZp586F2224) | DKFZp586F2224 (from clone | | | |
| | | | DKFZp586F2224) | | | |
| 2975 | H3156C08 | “Mus musculus metaxin (Mtx), mRNA” | | BG076156 | Mm.22508 | Heat Shock/Stress |
| 2976 | | | | | | |
| 2977 | H3156H04 | H3156H04 | | | | Energy/Metabolism |
| 2978 | | | | | | |
| 2979 | H3157B03 | catalase 1 (Cas1), | Mus musculus putative phosphatase | BG076224 | Mm.1458 | Energy/Metabolism |
| 2980 | | | (Pps), mRNA | | | |
| 2981 | H3157B10 | “Mus musculus heat shock protein, | | BG088755 | Mm.27897 | Heat Shock/Stress |
| 2982 | | DNAJ-like 2 (Hsj2), mRNA” | | | | |
| 2983 | H3157C04 | caspase 6 (Casp6), | Mus musculus catalase 1 (Cas1), mRNA | BG076235 | Mm.4215 | Energy/Metabolism |
| 2984 | | | | | | |
| 2985 | H3157C09 | M. musculus aspartate aminotransferase | Mus musculus caspase 6 (Casp6), | BG076240 | Mm.28814 | Apoptosis |
| 2986 | | gene exon 10 and 3-flank | mRNA | | | |
| 2987 | H3157D01 | M. musculus aspartate aminotransferase | M. musculus aspartate aminotransferase | BG076244 | Mm.18916 | Matrix/Structural |
| 2988 | | gene exon 10 and 3-flank | gene exon 10 and 3-flank | | | Proteins |
| 2989 | H3157D12 | microsomal glutathione S-transferase 3 | Homo sapiens microsomal glutathione | BG088778 | Mm.29823 | Energy/Metabolism |
| 2990 | | (MGST3), | S-transferase 3 (MGST3), mRNA | | | |
| 2991 | H3157E04 | “Mus musculus phosphatidylinositol 3- | | BG088781 | Mm.41943 | Signal Transduction |
| 2992 | | kinase, catalytic, alpha polypeptide | | | | |
| | | (Pik3ca), mRNA” | | | | |
| 2993 | H3157E05 | solute carrier family 30 (zinc | Mus musculus solute carrier family 30 | BG076257 | Mm.27801 | Energy/Metabolism |
| 2994 | | transporter), member 4 (Slc30a4), | (zinc transporter), member 4 (Slc30a4), | | | |
| | | | mRNA | | | |
| 2995 | H3157H01 | sterol O-acyltransferase 1 (Soat1), | Mus musculus sterol O-acyltransferase 1 | BG088808 | Mm.28099 | Matrix/Structural |
| 2996 | | | (Soat1), mRNA | | | Proteins |
| 2997 | H3158B04 | ornithine decarboxylase antizyme | Mus musculus ornithine decarboxylase | BG088828 | Mm.6775 | Energy/Metabolism |
| 2998 | | inhibitor (Oazi), | antizyme inhibitor (Oazi), mRNA | | | |
| 2999 | H3158C04 | Mus musculus maternal embryonic | | BG076315 | Mm.1178 | Matrix/Structural |
| 3000 | | message 3 (Mem3), mRNA | | | | Proteins |
| 3001 | H3158C07 | | | | | Heat Shock/Stress |
| 3002 | | | | | | |
| 3003 | H3158E03 | methylenetetrahydrofolate | Mus musculus | BG076333 | Mm.443 | Energy/Metabolism |
| 3004 | | dehydrogenase (NAD+ dependent), | methylenetetrahydrofolate | | | |
| | | methenyltetrahydrofolate | dehydrogenase (NAD+ dependent), | | | |
| | | cyclohydrolase (Mthfd2), | methenyltetrahydrofolate | | | |
| | | | cyclohydrolase (Mthfd2), mRNA | | | |
| 3005 | H3158F09 | cDNA: FLJ22903 fis, clone KAT05624 | Homo sapiens cDNA: FLJ22903 fis, | BG088873 | Mm.20841 | Energy/Metabolism |
| 3006 | | | clone KAT05624 | | | |
| 3007 | H3158F10 | “Mus musculus Ccth gene for | | BG076350 | Mm.28037 | Heat Shock/Stress |
| 3008 | | chaperonin containing TCP-1 eta | | | | |
| | | subunit, complete cds” | | | | |
| 3009 | H3158G12 | “Mus musculus metallothionein-like 5, | | BG076362 | Mm.56287 | Heat Shock/Stress |
| 3010 | | testis-specific (tesmin) (Mtl5), mRNA” | | | | |
| 3011 | H3158H01 | “Mus musculus protein phosphatase 5 | | BG076363 | Mm.3294 | Signal Transduction |
| 3012 | | (PP5) mRNA, complete cds” | | | | |
| 3013 | H3159A03 | Mm.10331 | | | Mm.10331 | Apoptosis |
| 3014 | | | | | | |
| 3015 | H3159C05 | “Mus musculus Rab3D (rab3d) gene, | | BG076387 | Mm.29968 | Signal Transduction |
| 3016 | | complete cds” | | | | |
| 3017 | H3159D05 | “Homo sapiens phosphorylase kinase, | | BG088924 | Mm.28827 | Signal Transduction |
| 3018 | | beta (PHKB), mRNA” | | | | |
| 3019 | H3159F12 | “Mus musculus phosphofructokinase-1 | | BG088948 | Mm.26550 | Signal Transduction |
| 3020 | | A isozyme (Pfka) mRNA, complete | | | | |
| | | cds” | | | | |
| 3021 | H3159G05 | glutathione S-transferase, mu 2 (Gstm2), | Mus musculus glutathione S-transferase, | BG088952 | Mm.14601 | Energy/Metabolism |
| 3022 | | | mu 2 (Gstm2), mRNA | | | |
| 3023 | P1G4 | Ant 1 | | | | |
| 3024 | mFRDA | Frataxin | | NM_008044 | | Energy/Metabolism |
| 3025 | mSOD2 | Mn SOD (SOD2) | | NM_013671 | | Energy/Metabolism |
| 3026 | P1G9 | mtDNA-12s rRNA | | | | |
| 3027 | P1G10 | mtDNA-16s rRNA | | | | |
| 3028 | P1H10 | mtDNA-ATPase 6 | | | | |
| 3029 | P1H11 | mtDNA-ATPase 8 | | | | |
| 3030 | P1G11 | mtDNA-COX 1 | | | | |
| 3031 | P1G12 | mtDNA-COX 2 | | | | |
| 3032 | P1H1 | mtDNA-COX 3 | | | | |
| 3033 | P1H9 | mtDNA-Cyt b | | | | |
| 3034 | P1H2 | mtDNA-NADH 1 | | | | |
| 3035 | P1H3 | mtDNA-NADH 2 | | | | |
| 3036 | P1H4 | mtDNA-NADH 3 | | | | |
| 3037 | P1H5 | mtDNA-NADH 4 | | | | |
| 3038 | P1H6 | mtDNA-NADH 4L | | | | |
| 3039 | P1H7 | mtDNA-NADH 5 | | | | |
| 3040 | P1H8 | mtDNA-NADH 6 | | | | |

TABLE 5

| | | | IMAGE Clone | | Res. |
|---|---|---|---|---|---|
| Num | Gene | Genbank Acc. # | ID | Unigene Cluster | Gen.Clone |
| 1 | mtDNA-12s rRNA | V00711 | In House-Clone | | |
| 2 | mtDNA-16s rRNA | V00711 | In House-Clone | | |
| 3 | mtDNA-ATPase 6 | V00711 | In House-Clone | | |
| 4 | mtDNA-ATPase 8 | V00711 | In House-Clone | | |
| 5 | mtDNA-COX 1 | V00711 | In House-Clone | | |
| 6 | mtDNA-COX 2 | V00711 | In House-Clone | | |
| 7 | mtDNA-COX 3 | V00711 | In House-Clone | | |
| 8 | mtDNA-Cyt b | V00711 | In House-Clone | | |
| 9 | mtDNA-NADH 1 | V00711 | In House-Clone | | |
| 10 | mtDNA-NADH 2 | V00711 | In House-Clone | | |
| 11 | mtDNA-NADH 3 | V00711 | In House-Clone | | |
| 12 | mtDNA-NADH 4 | V00711 | In House-Clone | | |
| 13 | mtDNA-NADH 4L | V00711 | In House-Clone | | |
| 14 | mtDNA-NADH 5 | V00711 | In House-Clone | | |
| 15 | mtDNA-NADH 6 | V00711 | In House-Clone | | |
| 16 | 2′-5′)oligoadenylate synthetase 1 | X04958, | AI449562 | Mm.14301 | 577664 |
| | | M33863 | | | |
| 17 | 14-3-3 protein beta | AF058797.1 | AA624796 | Mm.34454 | 1039350 |
| 18 | 14-3-3 protein epsilon | D87663.1 | AA240827 | Mm.678 | 670982 |
| 19 | 14-3-3 protein eta | U57311 | AI325059 | Mm.3308 | 558536 |
| 20 | 14-3-3 protein gamma | AF058799 | | Mm.10802 | 464892 |
| 21 | 14-3-3 protein theta (tau) | U57312 | AA422980 | Mm.14722 | 803843 |
| 22 | 14-3-3 protein zeta | U79231 | AA671451 | Mm.3360 | 1039129 |
| 23 | 25-hydroxyvitamin D3 24-hydroxylase | D49438 | AA240836 | Mm.6575 | 657172 |
| | precursor | | | | |
| 24 | 25-hydroxyvitamin D3 24-hydroxylase | D49438 | AI226268 | Mm.6575 | 658678 |
| | precursor | | | | |
| 25 | 2-amino-3ketobutyrate-coenzyme A ligase | AF093403 | AI037094 | Mm.18618 | 315143 |
| 26 | 2-oxoglutarate dehydrogenase E1 | U02971 | W98443 | | 424460 |
| | component | | | | |
| 27 | 3,2-trans-enoyl-CoA isom, mito precursor | Z14049 | AI194961 | | 1886651 |
| 28 | 38g cent. | | In House-Clone | | |
| 29 | 3-beta hydroxy-5-ene steroid | M27137 | AA274685 | | 766591 |
| | dehydrogenase type I | | | | |
| 30 | 3-beta hydroxy-5-ene steroid | M75886 | AI266804 | Mm.30433 | 1891212 |
| | dehydrogenase type II | | | | |
| 31 | 3-beta hydroxy-5-ene steroid | M77015 | AA209060 | Mm.335 | 676577 |
| | dehydrogenase type III | | | | |
| 32 | 3-methyl-2-oxobutanoate dehydrogenase | L16992 | | Mm.12819 | 422840 |
| | (lipoamide) | | | | |
| 33 | 41.2a cent. | | In House-Clone | | |
| 34 | 44b cent. | | In House-Clone | | |
| 35 | 45.2b cent. | | In House-Clone | | |
| 36 | 55e cent. | | In House-Clone | | |
| 37 | 5-aminolevulinate synthase precursor | M15268 | AA189529 | Mm.1217 | 635215 |
| | (EST) | | | | |
| 38 | 65c cent. | | In House-Clone | | |
| 39 | 66a cent. | | In House-Clone | | |
| 40 | 67 cent. | | In House-Clone | | |
| 41 | a-amylase | V00719 | AI325237 | Mm.7074 | 608852 |
| 42 | acatyl-CoA acetyltransferase 1 | L42293 | | Mm.28099 | 472233 |
| 43 | acatyl-CoA acetyltransferase 2 | AF078751 | AA239043 | | 694062 |
| 44 | Aconitase (iron responsive element) | X61147 | AA212704 | | 677092 |
| 45 | Aconitase (iron responsive element) | X61147 | AA238899 | | 697949 |
| 46 | Acyl-CoA dehyd, med-chain specific | U07159 | AA104184 | Mm.10530 | 568149 |
| | precursor (MCAD) | | | | |
| 47 | Acyl-CoA dehyd, short-chain specific | L11163 | AI050239 | Mm.18759 | 1379035 |
| | precursor | | | | |
| 48 | Acyl-CoA dehyd, very-long-chain specific | U41497 | AA250410 | Mm.18630 | 670916 |
| | precursor | | | | |
| 49 | Acyl-CoA dehydrog, long-chain spec. | U21489 | AA254905 | Mm.2445 | 719580 |
| | precursor (LCAD) | | | | |
| 50 | adapt | | In House-Clone | | |
| 51 | Adenine nucleotide translocator 2, | | AA033138.1 | | 465520 |
| | fibroblast (Ant2) | | | | |
| 52 | adenylate kinase isoenzyme 2 (EST) | AI155541 | AA061587 | Mm.29460 | 483322 |
| 53 | Adenylosuccinate synthase | | AA388461 | | 749837 |
| 54 | adenylyl cyclase type VII | U12919 | W65619 | | 387280 |
| 55 | ADP, ATP carrier protein, fibroblast | U27316 | | Mm.658 | 585992 |
| | isoform 2 (ant2) | | | | |
| 56 | ADP, ATP carrier protein, heart isoform T1 | U27315 | AA717872 | Mm.16228 | 1152250 |
| 57 | adrenodoxin precursor | L29123 | AA461849 | Mm.1061 | 851558 |
| 58 | Alcohol dehydrogenase 5 | | AA183192 | | 636207 |
| 59 | Alcohol dehydrogenase I | | AA221141 | | 695105 |
| 60 | Aldehyde dehydrogenase | U07235 | AA122975 | | 579570 |
| 61 | aldehyde dehydrogenase (NAD+) 2 | U07235 | AI503977 | Mm.2621 | 1001020 |
| | precursor | | | | |
| 62 | Alpha-1 protease inhibitor | U38477 | AA212578 | Mm.16672 | 676745 |
| 63 | Ant 1 | | In House-Clone | | |
| 64 | Antioxidant protein 1 | M28723 | W91307 | | 423832 |
| 65 | Antioxidant protein 1 | M28723 | | Mm.29821 | 599017 |
| 66 | Antioxidant protein-2 (AO2) | | AA243957 | Mm6587 | 694088 |
| 67 | Apoptosis inhibitor 2 | | AA144490 | | 597715 |
| 68 | Apoptosis inhibitor 3 | | AA097958 | | 550702 |
| 69 | apoptosis regulator BAX, membrane | L22472 | AI323521 | Mm19904 | 557643 |
| | isoform alpha | | | | |
| 70 | Apoptosis-inducing factor | AF100927 | AA866777 | | 1434491 |
| 71 | ASC | | In House-Clone | | |
| 72 | ATP synth lipid-binding protein P1 | L19737 | AI481739 | Mm.258 | 888863 |
| | precursor (subunit 9) | | | | |
| 73 | ATP synthase A Channel | AA106406 | AA106406 | Mm.5293 | 519329 |
| 74 | ATP synthase F0 component | L19737 | AA139793 | | 580898 |
| 75 | ATP synthase F0 component | | AA269701 | | 735887 |
| 76 | ATP/GTP binding protein | | AA184876 | | 642977 |
| 77 | ATPase, Ca++ transporting, cardiac | | W34420.1 | | 318735 |
| | muscle, fast twitch 1 | | | | |
| 78 | ATPase, Ca2+ transporting, heart | | W34420 | | 318735 |
| 79 | ATPase-like vacule proton channel | | AA276030 | | 776055 |
| 80 | ATP-binding cassette transporter 7 | U43892 | AU019072 | Mm.4739 | 1920872 |
| 81 | Bcl-2 protein | U10102 | AA051441 | | 478723 |
| 82 | Branched chain alpha ketoacid dehydrog. | | | Mm.8903 | 578018 |
| | Kinase | | | | |
| 83 | branched chain alpha-ketoacid dehyd chain | L47335 | | Mm.25848 | 314098 |
| | E1-alpha | | | | |
| 84 | Branched-chain a-ketoacid dehydrog. E1b | | AA050586 | | 476163 |
| 85 | Branched-chain amino transferase I | | AA003372 | | 426976 |
| 86 | BZP | | In House-Clone | | |
| 87 | C II 3 | | In House-Clone | | |
| 88 | C IV is | | In House-Clone | | |
| 89 | Calcium channel, voltage-dependent, L | | AA511037.1 | | 890932 |
| | type, alpha 2 delta subunit | | | | |
| 90 | calm 1 | | In House-Clone | | |
| 91 | Calmodulin 3 | | AA109041.1 | | 571789 |
| 92 | carbonate dehydratase, hepatic | X51971 | AI256540 | Mm.35538 | 1889415 |
| 93 | Carbonic Anhydrase IV | U37091 | AA71129 | Mm.1614 | 1167078 |
| 94 | carbonyl reductase (NADPH) - mouse | X07411, | W11423 | Mm.21454 | 317572 |
| | | D26123 | | | |
| 95 | carnitine O-acetyltransferase (carnitine | X85983 | AI528757 | Mm.20396 | 602213 |
| | acetylase) | | | | |
| 96 | carnitine O-palmitoyltransferase II | U01163 | | Mm.29499 | 580316 |
| | precursor | | | | |
| 97 | Carnitine palmitoyltransferase 2 | | | Mm.29499 | 580316 |
| 98 | Caspase 2 | | AA200808 | | 639403 |
| 99 | Caspase I | | AA098139 | | 550766 |
| 100 | Catalase | | AA239490 | Mm 4215 | 678773 |
| 101 | CCAAT/enhancer binding protein | | AA271223.1 | | 738252 |
| | C/EBP), alpha | | | | |
| 102 | CEBP | | In House-Clone | | |
| 103 | Cellular apoptosis succep. Protein | | AA471761 | | 874148 |
| 104 | chaperonin-10 | U09659 | | Mm.12970 | 422572 |
| 105 | CI 18K | | In House-Clone | | |
| 106 | CI B8 | | In House-Clone | | |
| 107 | col 1 | | In House-Clone | | |
| 108 | col 3 | | In House-Clone | | |
| 109 | coproporphyrinogen III oxidase precursor | D16333 | W71884 | Mm.35820 | 390487 |
| 110 | coproporphyrinogen oxidase | D16333 | AA108600 | | 570602 |
| 111 | coproporphyrinogen oxidase | D16333 | AA259342 | | 734795 |
| 112 | coproporphyrinogen oxidase | D16333 | W53951 | | 367358 |
| 113 | Core binding factor beta | | AA146442.1 | | 596552 |
| 114 | creatine kinase | Z13968, | AA690010 | Mm.970 | 1167886 |
| | | Z13969 | | | |
| 115 | Creatine Kinase, muscle | X03233 | AA166212 | Mm.2375 | 608246 |
| 116 | Creatine kinase-complete | M74149 | AA270310 | Mm.16831 | 736251 |
| 117 | Creatine kinase-mitochondrial | Z13968 | AI528837 | Mm.970 | 607301 |
| 118 | cyt C oxydase polypeptide VIIa- | X58486 | AA960158 | Mm.2151 | 1248366 |
| | liver/heart precursor | | | | |
| 119 | cyto C oxydase polypeptide VIa-heart | U08439 | AA415934 | Mm.21050 | 846138 |
| | precursor | | | | |
| 120 | cyto C oxydase polypeptide VIa-liver | L06465 | | Mm.19094 | 533628 |
| | precursor | | | | |
| 121 | cytochrome c oxidase chain IV precursor | X54691, | AA260009 | Mm.2136 | 746546 |
| | | M37829 | | | |
| 122 | cytochrome c oxidase chain Va precursor | X15963 | AI131665 | Mm.360 | 1884978 |
| 123 | cytochrome c oxidase chain Vb precursor | X53157 | AI035302 | Mm.16769 | 1432845 |
| 124 | cytochrome c oxidase chain VIb | NM_001863 | AI503861 | | 991287 |
| | (HUMAN) | | | | |
| 125 | cytochrome c oxidase chain VIc | M20153 | AA062417 | | 516889 |
| 126 | cytochrome c oxidase chain VIIc | X52940 | AA031250 | Mm.14831 | 464966 |
| 127 | cytochrome c oxidase chain VIII | U37721 | | Mm.14022 | 481408 |
| 128 | Cytochrome C oxidase sub VII | | AA050684 | | 476180 |
| 129 | cytochrome C oxydase polypeptide VIII-H | U15541 | AI323348 | Mm.3841 | 463967 |
| | precursor | | | | |
| 130 | cytochrome C, somatic | X01756 | AA221965 | Mm.35389 | 658678 |
| 131 | cytochrome C-type heme lyase (CCHL) | U36778 | | Mm.3988 | 400735 |
| 132 | cytochrome-b5 reductase (HUMAN) | NM_000398 | AA816039 | | 1120651 |
| 133 | CYTOSOLIC BRANCH CHAIN | | AA286063 | | 776036 |
| | AMINOTRANSFERASE | | | | |
| 134 | DD 43 | | In House-Clone | | |
| 135 | DD 47 | | In House-Clone | | |
| 136 | DD 48 | | In House-Clone | | |
| 137 | DD 53 | | In House-Clone | | |
| 138 | DD 64 | | In House-Clone | | |
| 139 | DD 68 | | In House-Clone | | |
| 140 | DD 69 | | In House-Clone | | |
| 141 | DD 73 | | In House-Clone | | |
| 142 | DD 83 | | In House-Clone | | |
| 143 | DD 84 | | In House-Clone | | |
| 144 | DD10a | | In House-Clone | | |
| 145 | DD11b | | In House-Clone | | |
| 146 | DD14 | | In House-Clone | | |
| 147 | DD15c | | In House-Clone | | |
| 148 | DD16c | | In House-Clone | | |
| 149 | DD17c | | In House-Clone | | |
| 150 | DD19a | | In House-Clone | | |
| 151 | DD23ba | | In House-Clone | | |
| 152 | DD24d | | In House-Clone | | |
| 153 | DD25c | | In House-Clone | | |
| 154 | DD26f | | In House-Clone | | |
| 155 | DD2a | | In House-Clone | | |
| 156 | DD33a/pgf | | In House-Clone | | |
| 157 | DD39 | | In House-Clone | | |
| 158 | DD4c | | In House-Clone | | |
| 159 | DD7a | | In House-Clone | | |
| 160 | defender against cell death 1 | | AA033006 | | 464622 |
| 161 | DiGeorge syndrome chromosome region 6 | | W54234.1 | | 356181 |
| 162 | dihydrolipoamide dehydrogenase (E3) | J03490 | AA548170 | Hs.74635 | 994825 |
| | (HUMAN) | | | | |
| 163 | dihydrolipoamide transacylase precursor | L42996 | AA254971 | Mm.3636 | 719973 |
| 164 | Dimethyl glycine dehydrogenase | | AA288418 | | 748958 |
| 165 | DNA polymerase gamma | U53584 | | Mm.3616 | 575332 |
| 166 | Dynamin | | AA266438 | | 317587 |
| 167 | Dynamin 2 | | W13111 | | 457445 |
| 168 | E1B 19k/Bcls-binding prot. Homolog | | AA105295 | | 571367 |
| | (NIP3) | | | | |
| 169 | ER V | | In House-Clone | | |
| 170 | ERV-1 | U40494 | AI413376 | Mm.28124 | 367232 |
| 171 | EST highly similar to S-100 prot a-chain | | AA466432 | | 872869 |
| 172 | ESTs | | AA253853.1 | | 660997 |
| 173 | ESTs | | AA268402.1 | | 721970 |
| 174 | ESTs | | W14142.1 | | 329863 |
| 175 | ESTs, Highly similar to CREATINE | | AA038095.1 | | 472860 |
| | KINASE, SARCOMERIC MITO. | | | | |
| | PRECUR. | | | | |
| 176 | ESTs, Highly similar to CREATINE | | AI322288.1 | | 336085 |
| | KINASE, SARCOMERIC MITO. | | | | |
| | PRECUR. | | | | |
| 177 | ESTs, Highly similar to | | W13931.1 | | 330218 |
| | DERMATOPONTIN [Bos taurus] | | | | |
| 178 | ESTs, Highly similar to MATERNAL | | AA104976.1 | | 533314 |
| | EFFECT PROTEIN STAUFEN | | | | |
| 179 | ESTs, Highly similar to NADH- | | AA288040.1 | | 748891 |
| | UBIQUINONE OXIDOREDUCTASE 13 | | | | |
| | KD-B SUB | | | | |
| 180 | ESTs, Highly similar to NADH- | | AA222463.1 | | 671212 |
| | UBIQUINONE OXIDOREDUCTASE | | | | |
| | SGDH SUB PREC. | | | | |
| 181 | ESTs, Highly similar to NUCLEAR | | AA060802.1 | | 482952 |
| | FACTOR NF-KAPPA-B P100 SUBUNIT | | | | |
| 182 | ESTs, Highly similar to PINCH | | AA289280.1 | | 790449 |
| | PROTEIN | | | | |
| 183 | ESTs, Highly similar to PTERIN-4- | | W14332.1 | | 331681 |
| | ALPHA-CARBINOLAMINE | | | | |
| | DEHYDRATASE | | | | |
| 184 | ESTs, Highly similar to PUTATIVE | | W66757.1 | | 387449 |
| | REGULATORY PROTEIN TSC-22 | | | | |
| 185 | ESTs, Highly similar to SUCCINATE | | AA108475.1 | | 572339 |
| | DEHYDROGENASE | | | | |
| 186 | ESTs, Highly similar to (define not | | AA190123.1 | | 642467 |
| | available 4588044) | | | | |
| 187 | ESTs, Highly similar to putative | | AA184876.1 | | 642977 |
| | ATP/GTP-binding protein | | | | |
| 188 | ESTs, Weakly similar to (define not | | AA467585.1 | | 833160 |
| | available 3668141) [H. sapiens] | | | | |
| 189 | ESTs, Weakly similar to survival motor | | AA538419.1 | | 932748 |
| | neuron [M. musculus] | | | | |
| 190 | ESTs, Weakly similar to vesicle membrane | | AA259674.1 | | 735186 |
| | protein | | | | |
| 191 | Excitatory amino acid transporter 3 | D43797 | AA065502 | Mm.24741 | 524046 |
| 192 | ferredoxin--NADP+ reductase precursor | D49920 | AA879949 | Mm.4719 | 1230740 |
| 193 | ferrochelatase precursor | M59288 | | Mm.1070 | 635652 |
| 194 | Friedreich ataxia | S75712 | AA930748 | | 1150363 |
| 195 | Fructose 1-6 bis-phosphate | D42083 | AI385602 | Mm.2974 | 336727 |
| 196 | Fructose bis-phosphate aldolase | W53351 | AI553136 | Mm.7729 | 902910 |
| 197 | Fructose Bisphosphate aldolase A | Y00516 | AA518639 | | 903419 |
| 198 | Fuzzy onion homolog (mouse) | | AA674474 | | 1093002 |
| 199 | GAMMA-ADAPTIN | | AA238435.1 | | 693837 |
| 200 | GAPD | | In House-Clone | | |
| 201 | GILZ | | In House-Clone | | |
| 202 | Gluamate receptor, ionotropic, kainate 5 | | AA261334 | Mm.2879 | 733368 |
| | (gamma 2) | | | | |
| 203 | Gluamate receptor, ionotropic, NMDA1 | | W44130 | Mm.3292 | 354244 |
| | (Zeta 1) | | | | |
| 204 | Gluamate receptor, ionotropic, NMDA2C | | AI256808 | Mm.39090 | 1852361 |
| | epsilon 3) | | | | |
| 205 | Glucose dependent insulinotropic | U34295 | AA871367 | Mm.5115 | 1096156 |
| | polypeptide | | | | |
| 206 | Glucose Phospahate Isomerase I complex | M14220 | AA276216 | | 776210 |
| 207 | Glucose Phospahate Isomerase I complex | M14220 | W29397 | | 337413 |
| 208 | Glucose-6-phosphate isomerase | L09104 | AI327180 | Mm.589 | 437357 |
| 209 | glutamate dehydrogenase (NAD(P)+) | X57024 | AA543797 | Mm.10600 | 949005 |
| | precursor | | | | |
| 210 | glutamate oxaloacetate transaminase-2 | X06917, | | Mm.18916 | 617490 |
| | | J02622 | | | |
| 211 | Glutaryl-CoA dehydrogenase precursor | U18992 | | Mm.2475 | 573351 |
| | (GCD) | | | | |
| 212 | Glutathione peroxidase-3 (plasma Gpx) | U13705 | AA097557 | Mm7156 | 552393 |
| 213 | Glutathione peroxidase-4 (phospholipid | | AA034666 | Mm2400 | 466963 |
| | Gpx) | | | | |
| 214 | Glutathione peroxidase-heart isoform | AF045769.1 | AI327053 | Mm.2400 | 420345 |
| | (Gpx4) | | | | |
| 215 | Glutathione peroxidase-plasma isoform | U13705.1 | AI042912 | Mm.7156 | 1432410 |
| 216 | Glyceraldehyde 3-phosphate | | AA122891 | | 579715 |
| | dehydrogenase | | | | |
| 217 | Glycerol 3-phosphate acyltransferase | M77003 | AA209041 | | 676437 |
| 218 | Glycerophosphate dehydrogenase | M13366 | AI414023 | Mm.10669 | 303389 |
| 219 | Glycogen Phosphorylase (RAT) | J03080 | AA240684 | | 656882 |
| 220 | GP4 | | In House-Clone | | |
| 221 | Gpx | | In House-Clone | | |
| 222 | GTP2 | | In House-Clone | | |
| 223 | GTP-binding protein | | AA509565 | | 890444 |
| 224 | H+-transporting ATP synthase chain alpha | L01062 | AI573940 | Mm.4069 | 1920315 |
| 225 | H+-transporting ATP synthase chain beta | AF030559 | AI452208 | Mm.17869 | 576006 |
| 226 | H+-transporting ATP synthase chain delta | AA940343 | AA896697 | Mm.22514 | 1279053 |
| | (EST) | | | | |
| 227 | H+-transporting ATP synthase chain e | U59283 | AI115240 | | 1885128 |
| 228 | H+-transporting ATP synthase chain e | U59283 | M481625 | | 876416 |
| 229 | H+-transporting ATP synthase chain | U43893 | AA210528 | | 640931 |
| | gamma | | | | |
| 230 | heat shock protein 60 precursor | X55023, | AA154729 | Mm.1777 | 540881 |
| | | X53584 | | | |
| 231 | heat shock protein 70 precursor | D11089 | AI132204 | Mm.2849 | 1481709 |
| 232 | heat shock protein, 70 K (hsp68) | M12573 | AA647374 | Mm.6388 | 1108306 |
| | (fragment) | | | | |
| 233 | Heat shock protein, 84 kDa 1 | M18186 | | Mm.2180 | 538585 |
| 234 | Hexokinase | | W11571 | | 318642 |
| 235 | Hexokinase | | W74835 | | 389177 |
| 236 | hexokinase I (3′-seq) | J05277 | AI661880 | Mm.5290 | 717383 |
| 237 | hexokinase I (5′-seq) | J05277 | AA197916 | Mm.5290 | 654873 |
| 238 | high mobility group protein homolog | | AA048831.1 | | 478865 |
| | HMG4 (Hmg4) mRNA, complete cds | | | | |
| 239 | HK-40s Ribosomal protein S15 | | AA033398 | | 466295 |
| 240 | HK-40s Ribosomal protein S4 | | AA000082 | | 425352 |
| 241 | HK-A111Acient ubiquinating protein | | W82989 | | 405768 |
| 242 | HK-A16060s Ribosomal protein L15 | | AA068842 | | 532770 |
| 243 | HK-A17360s Ribosomal protein L3 | | AA108363 | | 570533 |
| 244 | HK-A21060s Ribosomal protein L1A | | AA170607 | | 619006 |
| 245 | HK-A216RNA splicing protein | | AA183061 | | 636766 |
| 246 | HK-A262E2F transcription factor | | AA396123 | | 751755 |
| 247 | HK-A272Capping protein | | AA414612 | | 779754 |
| 248 | HK-A316Actin-gamma (smooth muscle) | M26689 | AA710883 | Mm.16562 | 1166855 |
| 249 | HK-A97Hypozanthine phosphoribosyl | J00423 | W48168 | Mm.18675 | 355084 |
| | transferase | | | | |
| 250 | HK-Actin-alpha (skeletal muscle) | M12347 | AI035279 | Mm.4581 | 1480709 |
| 251 | HK-Actin-gamma | M21495 | AI314957 | Mm.29913 | 192859 |
| 252 | HK-Alkaline Phosphatase | | AA032457 | | 465052 |
| 253 | HK-b-actin | X03672 | AA079937 | Mm.297 | 536615 |
| 254 | HK-calcium binding protein Cab45 | U45977 | AI266799 | Mm.30149 | 1891202 |
| 255 | HK-DNA ligase I | | W66626 | | 388245 |
| 256 | HK-Glyceraldehyde 3-phosphate | W14827 | AA119563 | Mm.5289 | 538210 |
| | dehydrogenase (G3PDH) | | | | |
| 257 | HK-Glyceraldehyde phosphate | M32599 | AA466618 | Mm.5289 | 817984 |
| | dehydrogenase | | | | |
| 258 | HK-HPRT | | In House-Clone | | |
| 259 | HK-Hypozanthine phosphoribosyl | J00423 | AI256193 | Mm.18675 | 1890233 |
| | transferase | | | | |
| 260 | HK-Murine ornithine decarboxylase | M10624 | AI325192 | Mm.15259 | 608003 |
| 261 | HK-Myosin 1 | L00923 | AI098184 | Mm.3390 | 1481939 |
| 262 | HK-Phospholipase A2 (14-3-3 zeta/delta) | D78647 | AA1714341 | Mm.3360 | 617315 |
| 263 | HK-Ribosomal Protein S29 | L31609 | AA032465 | Mm.35816 | 465138 |
| 264 | HK-Ubiquitin | X51703 | AI181949 | Mm.235 | 1451597 |
| 265 | House mouse; Musculus domesticus | | W82212.1 | | 403728 |
| | mRNA for LN1, complete cds | | | | |
| 266 | Huntingtin | AI876894 | AI876894 | — | 1922060 |
| 267 | hydroxymethylglutaryl-CoA lyase | S65036 | AA838929 | | 1261134 |
| 268 | hydroxymethylglutaryl-CoA synthase, | U12790, | | Mm.10633 | 518481 |
| | mitochondrial | U12791 | | | |
| 269 | IG ALPHA CHAIN C REGION | | AA098196.1 | | 551003 |
| 270 | IMAGE EST | | AA009059.1 | | 441176 |
| 271 | IMAGE EST | | AA028306 | | 464099 |
| 272 | IMAGE EST | | AA035899.1 | | 468817 |
| 273 | IMAGE EST | | AA051664.1 | | 479709 |
| 274 | IMAGE EST | | AA118290 | | 574435 |
| 275 | IMAGE EST | | AA200984.1 | | 639212 |
| 276 | IMAGE EST | | AA203878 | | 640734 |
| 277 | IMAGE EST | | AA215024 | | 652207 |
| 278 | IMAGE EST | | AA221250 | | 670393 |
| 279 | IMAGE EST | | AA245545 | | 699280 |
| 280 | IMAGE EST | | AA250652 | | 697537 |
| 281 | IMAGE EST | | AA266097.1 | | 716941 |
| 282 | IMAGE EST | | AA275684 | | 775722 |
| 283 | IMAGE EST | | AA388512 | | 790857 |
| 284 | IMAGE EST | | AA466026.1 | | 809016 |
| 285 | IMAGE EST | | AA519027.1 | | 904900 |
| 286 | IMAGE EST | | W08090.1 | | 331768 |
| 287 | IMAGE EST | | W09924 | | 315773 |
| 288 | IMAGE EST | | W09924.1 | | 315773 |
| 289 | IMAGE EST | | W15031 | | 330502 |
| 290 | IMAGE EST | | W41309.1 | | 351420 |
| 291 | IMAGE EST | | W89418.1 | | 420553 |
| 292 | IMAGE EST-CCAAT enhancer binding | | AA271223 | | 738252 |
| | protein | | | | |
| 293 | IMAGE EST-Glucocorticoid-induced | | W66757 | | 387449 |
| | leucine zipper GILZ protein | | | | |
| 294 | IMAGE EST-homolog of Unc33 | | W08090 | | 331768 |
| | (C. Elegans)/Collaspin reponse mediated | | | | |
| | prot. 2 | | | | |
| 295 | IMAGE EST-sarcoplasmic creatine kinase | | W18057 | | 336085 |
| 296 | IMAGE EST-Sim to gamma sarcoglycan | | W41309 | | 351420 |
| 297 | IMAGE EST-yeast bile transporter | | AA473289 | | 803488 |
| 298 | Interferon gamma receptor | | AA541842.1 | | 920516 |
| 299 | Interleukin 1 receptor-associated kinase | | AA276835.1 | | 777580 |
| 300 | isocitrate dehydrogenase (NADP) | U51167 | | Mm.2966 | 571468 |
| 301 | Kin 17 | | In House-Clone | | |
| 302 | Lactate Dehydrogenase- | M27554 | AA880398 | | 1277670 |
| 303 | Lactate Dehydrogenase-A4 | M17516 | AI506641 | Mm.26504 | 1024774 |
| 304 | Lactate Dehydrogenase-sperm specific | M17587, | AA110449 | Mm.16563 | 516582 |
| | | L10389 | | | |
| 305 | M. musculus mRNA for fibromodulin | | W63981.1 | | 374228 |
| 306 | M. musculus mRNA for GTP-binding | | AA020462.1 | | 455401 |
| | protein | | | | |
| 307 | MAD homolog 4 (Drosophila) | | AA030901.1 | | 466551 |
| 308 | Malate dehydrogenase (cyto) | | W13686 | | 318346 |
| 309 | Malate dehydrogenase (mito) | M16229 | AA266087 | | 717095 |
| 310 | malate dehydrogenase precursor, | X07295, | | Mm.21743 | 407143 |
| | mitochondrial | M16229 | | | |
| 311 | MAP KINASE PHOSPHATASE-1 | | AA125367.1 | | 575665 |
| 312 | Maternal embryonic message 3 | | AA388122.1 | | 775464 |
| 313 | MCK | | In House-Clone | | |
| 314 | mDP 6 | | In House-Clone | | |
| 315 | metal response element DNA-binding | | AA545607.1 | | 945218 |
| | protein M96 mRNA, complete cds | | | | |
| 316 | methylenetetrahydrofolate dehydrogenase | J04627 | | Mm.443 | 440345 |
| | (NAD+) | | | | |
| 317 | methylmalonyl-CoA mutase alpha chain | X51941 | | Mm.4299 | 571282 |
| | precursor | | | | |
| 318 | Microtubule-associated protein 4 | | AA003769.1 | | 437523 |
| 319 | Mito matrix prot P1 precursor (hsp60) | | AA184322 | | 633625 |
| 320 | Mitochondrial LON protease | AA061310 | AA061310 | — | 514859 |
| 321 | mitochondrial transcription factor A - | U57939 | | Mm.276 | 539693 |
| | mouse | | | | |
| 322 | mitochondrial uncoupling protein | M21247 | AI131780 | Mm.4177 | 1498957 |
| 323 | monoamine oxidase A - mouse | S78615 | AI643185 | | 864614 |
| 324 | Mouse breast heat shock 73 prot (hsc73) | M19141 | | Mm.2944 | 538418 |
| 325 | Mouse calcineurin catalytic subunit | | AA245461.1 | | 699236 |
| | mRNA, complete cds | | | | |
| 326 | Mouse Circadian output locomotor cycles | AF000998 | AI156715 | Mm.3552 | 1494023 |
| | kaput | | | | |
| 327 | Mouse heatshock protein 27 | U03560.1 | AA596241 | Mm.13849 | 1052188 |
| 328 | Mouse heatshock protein 86 | J04633 | AI649095 | | 1970053 |
| 329 | Mouse med. Chain acyl-CoA | U07159 | AA061679 | | 483333 |
| | dehydrogenase | | | | |
| 330 | Mouse mHox protein | L06502 | W17990 | Mm.3869 | 335936 |
| 331 | Mouse Skd3 mRNA | U09874 | | Mm.3990 | 602340 |
| 332 | mRNA for sarco/endoplasmic reticulum | | AA222567.1 | | 695695 |
| | Ca2+-ATPase (SERCA2) | | | | |
| 333 | mTF 1 | | In House-Clone | | |
| 334 | Mus musculus Balb/c zinc finger protein | | W11161.1 | | 316427 |
| | PZF (Pzf) mRNA, complete cds | | | | |
| 335 | Mus musculus calcium-binding protein | | AA466432.1 | | 872869 |
| | S100A1 mRNA, complete cds | | | | |
| 336 | Mus musculus cytoplasmic protein Ndr1 | | AA473269.1 | | 803416 |
| | (Ndr1) mRNA, complete cds | | | | |
| 337 | Mus musculus FGF-binding protein (FGF- | | AA403432.1 | | 717457 |
| | BP) mRNA, complete cds | | | | |
| 338 | Mus musculus GTP binding protein | | AA509565.1 | | 890444 |
| | (GTP2) mRNA, complete cds | | | | |
| 339 | Mus musculus hemin-sensitive initiation | | AA036546.1 | | 466971 |
| | factor 2 alpha kinase mRNA, complete cds | | | | |
| 340 | Mus musculus mRNA for glutamate | | AI043222 | Mm.42021 | 1431493 |
| | receptor channel alpha 4 subunit | | | | |
| 341 | Mus musculus mRNA for GM3 synthase, | | AA274576.1 | | 748275 |
| | complete cds | | | | |
| 342 | Mus musculus PAF acetylhydrolase | | AI324436.1 | | 536464 |
| | mRNA, complete cds | | | | |
| 343 | Mus musculus pantophysin gene, complete | | AA271505.1 | | 737944 |
| | cds | | | | |
| 344 | Mus musculus rab6/rab5-associated | | W77711.1 | | 401958 |
| | protein (rab6) mRNA, partial cds | | | | |
| 345 | Mus musculus skeletal muscle LIM | | AA047966.1 | | 477066 |
| | protein (FLH1) mRNA, complete cds | | | | |
| 346 | Mus musculus Stra13 mRNA, complete | | AA064241.1 | | 480896 |
| | cds | | | | |
| 347 | mWS3 | | In House-Clone | | |
| 348 | Myeloid cell leukemia sequence 1 | U35623, | AA387843 | Mm.1639 | 761106 |
| | | AF063886 | | | |
| 349 | Myostatin | | AA052179.1 | | 418993 |
| 350 | NAD(P)+ transhydrogenase (B-specific) | Z49204 | AI323702 | Mm.3842 | 580717 |
| | precursor | | | | |
| 351 | NADH dehydrogenase mwfe | | W83104 | | 404499 |
| 352 | NADH-ubiquinone oxidoreductase 13 kDa | L38438 | AA397301 | | 599804 |
| | subunit | | | | |
| 353 | NADH-ubiquinone oxidoreductase 13 kDa | U59509 | AA035972 | | 468848 |
| | subunit | | | | |
| 354 | NADP transhydrogenase | Z49204 | AI323702 | Mm.3842 | 580717 |
| 355 | Nitric Oxide Synthase-2 | | AA512708 | Mm2893 | 922250 |
| 356 | Nitric Oxide Synthase-3 | | AA177240 | Mm12837 | 620940 |
| 357 | Nuclear respiratory factor-1 | AF098077 | AI594316 | | 1006311 |
| 358 | Nuclear respiratory factor-2 | U20532 | | Mm.1025 | 635541 |
| 359 | ornithine carbamoyltransferase precursor | X07092 | AI266937 | Mm.2611 | 1891345 |
| 360 | ornithine-oxo-acid transaminase precursor | X64837 | AI196410 | Mm.13694 | 1887672 |
| 361 | p63a | | In House-Clone | | |
| 362 | PACD | | In House-Clone | | |
| 363 | PEBP2 | | In House-Clone | | |
| 364 | Peripheral myelin protein, 22 kDa | | AA416246.1 | | 846064 |
| 365 | peripheral-type benzodiazepine receptor 1 | U12419 | AA068577 | | 524463 |
| 366 | Perox. Proliferator receptor (PPAR) | U01841 | W34083 | Mm.3020 | 317536 |
| | Gamma | | | | |
| 367 | Peroxisomal/Mitochondrial dienoyl-CoA | | W29607 | | 338088 |
| | isomerase | | | | |
| 368 | Phosphofructokinase 1 | J03928, | AI480449 | Mm.1166 | 862787 |
| | | AF123533 | | | |
| 369 | phospholipase A2, platelet, synovial fluid | X74266 | AA871547 | Mm.4675 | 1096251 |
| 370 | phosphoprotein phosphatase | M81475 | AI449151 | Mm.1567 | 619279 |
| 371 | PMP 35 | L27842 | AI573377 | Mm.16453 | 534171 |
| 372 | probable aconitate hydratase, | AI385870 | AA275929 | Mm.30065 | 775753 |
| | mitochondrial (EST) | | | | |
| 373 | Procollagen Type 1 | | AA073604 | | 536306 |
| 374 | Procollagen, type I, alpha 1 | | AA073604.1 | | 536306 |
| 375 | Procollagen, type III, alpha 1 | | W89883.1 | | 420322 |
| 376 | Procollagen, type VI, alpha 1 | | W33786.1 | | 352450 |
| 377 | Protein Phosphatase inhibitor 2 (IPP2) | AA041826 | AA041826 | Mm.29617 | 475407 |
| 378 | protoporphyrinogen oxidase | U25114, | | Mm.4730 | 482868 |
| | | D45185 | | | |
| 379 | pyruvate carboxylase | L09192 | AI303529 | Mm.1845 | 1888741 |
| 380 | Pyruvate decarboxylase | | AA308254 | | 473778 |
| 381 | Pyruvate dehydrogenase | M76727 | AA423301 | | 820409 |
| 382 | pyruvate dehydrogenase (lipoamide) | M76728 | AI323722 | Mm.4223 | 513684 |
| 383 | pyruvate dehydrogenase (lipoamide) | M76727 | AA466268 | Mm.34775 | 888842 |
| 384 | Pyruvate dehydrogenase E1 a subunit | L13318 | AA238899 | | 888842 |
| | (human) | | | | |
| 385 | pyruvate kinase | D38379 | AI035313 | Mm.2635 | 1432851 |
| 386 | Pyruvate kinase | D63764 | AI195164 | Mm.8359 | 1886895 |
| 387 | Pyruvate kinase | | AA475121 | | 873690 |
| 388 | Pyruvate kinase-like protein | | W17814 | | 334876 |
| 389 | RAB1 | | In House-Clone | | |
| 390 | RAB1, member RAS oncogene family | | AA175510.1 | | 619501 |
| 391 | Rat NRBF1 | | AA259674 | | 735186 |
| 392 | Rnase P-complex (RS Williams work) | U31228 | AI614577 | | 523232 |
| 393 | S100 | | In House-Clone | | |
| 394 | sarc. | | In House-Clone | | |
| 395 | Sim to 6-Phosphofructo-2-kinase (human) | M19938 | AA397024 | | 693346 |
| 396 | Sim to acetyl CoA acetyl transferase (hum) | D90228 | AA272067 | | 761668 |
| 397 | Sim to acetyl Coenzyme A synthetase | | AA109675 | | 570550 |
| 398 | Sim to acetyl Coenzyme A synthetase | | AA537637 | | 949423 |
| 399 | Sim to a-ketoglutarate (hum) | D10523 | W13320 | | 329728 |
| 400 | Sim to Aldehyde dehydrogense (HUM) | | | Mm.24457 | 423605 |
| 401 | Sim to arginyl-tRNA synthetase (Sac Cer) | | | Mm.22363 | 576572 |
| 402 | Sim to ATP synthase epsilon (Bov) | | AA108733 | | 571214 |
| 403 | Sim to ATP synthase F chain | PIR: A54211 | W82194 | | 403660 |
| 404 | Sim to ATP synthase lipid binding (Hum) | X69908 | AA239148 | | 698109 |
| 405 | Sim to b-enolase (human) | X56832 | W11965 | | 316967 |
| 406 | Sim to branched-chain a-ketoacid | TR: G924921 | AA059497 | | 480575 |
| | dehydrogenase kinase | | | | |
| 407 | Sim to carbon catabolite repressor prot. | | AA051133 | | 438774 |
| | (Sac) | | | | |
| 408 | Sim to carbon catabolite repressor prot. | | AA404014 | | 717197 |
| | (Sac) | | | | |
| 409 | Sim to carnitine/acylcarnitine carrier | | AA245413 | | 699181 |
| 410 | Sim to citrate synthase (Por) | | W14146 | | 329884 |
| 411 | Sim to cytochrome b5 (outer mito mem) | | AA203975 | | 640762 |
| 412 | Sim to cytochrome c oxidase VI B (Hum) | X13923 | AA139624 | | 581175 |
| 413 | Sim to cytochrome c oxidase VII | X80899 | AA050684 | | 476180 |
| 414 | Sim to electron trans flavoprotein a sub | J04058 | AA060723 | | 481934 |
| | (Hum) | | | | |
| 415 | Sim to electron trans flavoprotein b sub | X71129 | W18161 | | 333641 |
| | (Hum) | | | | |
| 416 | Sim to enolase a subunit (Hum) | M14328 | AA204262 | | 643854 |
| 417 | Sim to Fructose 1-6-bisphosphate (Hum) | L10320 | AA276043 | | 776124 |
| 418 | Sim to glucose dehydrogenase (Bac. Sub) | | AA241896 | | 680935 |
| 419 | Sim to Glutamate/Malate trans (BOV) | | | Mm.28466 | 582075 |
| 420 | Sim to Glutathione-S-transferase (RAT) | | | Mm.27395 | 317849 |
| 421 | Sim to glycerol 3-phosphate | | W41175 | | 351221 |
| | dehydrogenase 1 (rat) | | | | |
| 422 | Sim to glycogen phosphorylase (Hum) | J03544 | W16286 | | 334236 |
| 423 | Sim to hepatocyte gluc transporter | | AA002666 | | 426758 |
| 424 | Sim to mito RNA pol (HUM) | | AI892781 | Mm.34645 | 608625 |
| 425 | Sim to Mito. 2-oxoglutarate/malate carrier | X66114 | W54000 | | 367801 |
| | (Human) | | | | |
| 426 | Sim to mito. Elongation factor TS (Bov) | | AA245481 | | 699237 |
| 427 | Sim to NADH-ubiquin. oxidoreduct. 13 kd | | AA288040 | | 748891 |
| | sub. | | | | |
| 428 | Sim to NADH-ubiquin. oxidoreduct. 49 kd | | AA109715 | | 572585 |
| | sub. | | | | |
| 429 | Sim to NADH-ubiquin. oxidoreduct. 9 kd | | AA521758 | | 903911 |
| | sub. | | | | |
| 430 | Sim to NADH-ubiquin. oxidoreduct. ashl. | | W83085 | | 404593 |
| 431 | Sim to NADH-ubiquin. oxidoreduct. b14 | | AA462323 | | 871020 |
| | sub. | | | | |
| 432 | Sim to NADH-ubiquin. oxidoreduct. | | W54068 | | 367925 |
| | B14.5 sub. | | | | |
| 433 | Sim to NADH-ubiquin. oxidoreduct. b15 | | AA434897 | | 818906 |
| | sub. | | | | |
| [symbol] | [symbol] idoreduct. b17 | | W54448 | | 367651 |
| [symbol] | [symbol] idoreduct. b22 | | AA415725 | | 846155 |
| [symbol] | [symbol] idoreduct. b9 | | W83574 | | 406509 |
| [symbol] | [symbol] idoreduct. Kfy1 | | W97248 | | 423071 |
| [symbol] | [symbol] idoreduct. mn11 | | AA267638 | | 723360 |
| [symbol] | [symbol] idoreduct. sgdh | | AA222463 | | 671212 |
| [symbol] | [symbol] idoreduct. 15 kd | | AA014507 | | 439668 |
| [symbol] | [symbol] somal prot. S14 | | W89487 | | 419614 |
| [symbol] | [symbol] ctase | | AA241313 | | 653324 |
| [symbol] | [symbol] ctase | | AA259674 | | 735186 |
| [symbol] | [symbol] hyltransferase, | | AA208877 | | 676311 |
| [symbol] | [symbol] se | | | Mm.26793 | 578465 |
| [symbol] | [symbol] enase | M32246 | AA108475 | | 572339 |
| [symbol] | [symbol] | | W11644 | | 318134 |
| [symbol] | [symbol] r mito. mem. | Q01852 | AA498767 | | 888708 |
| [symbol] | [symbol] uctase | | AA087137 | | 493604 |
| [symbol] | [symbol] uctase, core prot. | | AA108590 | | 572127 |
| [symbol] | [symbol] drogenase | | AA067191 | | 523796 |
| [symbol] | [symbol] hate uridyltrans | TR: G881394 | AA473123 | | 805218 |
| [symbol] | [symbol] at synaptic | | AA220458 | | 695279 |
| [symbol] | [symbol] | | In House-Clone | | |
| [symbol] | [symbol] L1)/RPB- | | AA047966 | | 477066 |
| [symbol] | [symbol] | | In House-Clone | | |
| [symbol] | [symbol] | | In House-Clone | | |
| [symbol] | [symbol] | X15684 | AA275871 | | 776543 |
| [symbol] | [symbol] ein | M88463 | AA497767 | | 917403 |
| [symbol] | [symbol] le selenoprotein | S49657, | AI482284 | Mm.554 | 917537 |
| | | M29603 | | | |
| [symbol] | [symbol] tein precursor, | L36062 | AA389406 | Mm.3436 | 569013 |
| [symbol] | [symbol] cursor | M62361 | | Mm.1779 | 580813 |
| [symbol] | [symbol] Cyt B560 | TR: G1019861 | AA137762 | | 580040 |
| [symbol] | [symbol] ) precursor | Z18857, | AA415267 | Mm.2597 | 791140 |
| | | L35525 | | | |
| [symbol] | [symbol] u/Zn Sod) | | AA039044 | Mm5274 | 474545 |
| [symbol] | [symbol] xtracellular | | AI314465 | Mm2407 | 1907770 |
| [symbol] | [symbol] evisiae) | | AI323032.1 | | 477535 |
| [symbol] | [symbol] | | AA220458.1 | | 695279 |
| 469 | Surfeit locus protein 1 | M14689 | AA274488 | Mm.6874 | 748268 |
| 470 | tetrahydrofolylpolyglutamate synthase | U32197 | AA030778 | Mm.3830 | 463571 |
| | precursor | | | | |
| 471 | Thioredoxin | | AA242573 | | 681159 |
| 472 | Thioredoxin | | | Mm.3533 | 579774 |
| 473 | Thioredoxin Reductase | | AI529082 | Mm 25543 | 1887250 |
| 474 | thiosulfate sulfurtransferase | U35741 | AI196763 | Mm.15312 | 1887427 |
| 475 | Thiosulfate sulfurtransferase (mito) Tst1 | U35741 | AI195057 | Mm.15312 | 188763 |
| 476 | TIMP2 | | In House-Clone | | |
| 477 | Tissue inhibitor of metalloproteinase 2 | | AA444490.1 | | 831964 |
| 478 | Tissue inhibitor of metalloproteinase 2 | | AA518165.1 | | 902923 |
| 479 | TOAD | | In House-Clone | | |
| 480 | transforming protein bcl-2-alpha | L31532 | AA867214 | Mm.5155 | 1265430 |
| 481 | transforming protein bcl-2-beta | M16506 | AA867214 | | 1265430 |
| 482 | transforming protein bcl-w (3′ seq) | AF030769.1 | AA563148 | | 975210 |
| 483 | transforming protein bcl-w (5′-seq) | AF030769.1 | AA667328 | | 1139352 |
| 484 | Transketolase (Tkt) | U05809 | AI132421 | Mm.9307 | 1481358 |
| 485 | Type II Peroxiredoxin 1 | | W83228 | Mm42948 | 405943 |
| 486 | Uncoupling Prot homology (UCPH) | | AA260521 | | 748122 |
| 487 | Uncoupling Protein 2 | | | Mm.12556 | 423616 |
| 488 | Uncoupling protein 3 | | AA062091 | | 482847 |
| 489 | Unknown EST | | | | 608265 |
| 490 | uracil-DNA glycosylase | U55040 | W48179 | | 355462 |
| 491 | voltage-dependent anion channel 1 | U230840 | AA244874 | | 680076 |
| 492 | voltage-dependent anion channel 2 | U30838 | AI507203 | Mm.569 | 931442 |
| 493 | voltage-dependent anion channel 3 | U30839 | AA616007 | Mm.38513 | 1066900 |

### Example 3

Identification of Mutations Causing Disease

The mitochondrial respiratory complex I is assembled from seven mtDNA genes and thirty-six nDNA genes. Patients with complex I defects have phenotypes ranging from midlife-onset optic atrophy to lethal childhood Leigh's disease. Mitochondrial biology expression profiles were determined for patients with a variety of complex I defects. Samples are collected from a variety of patients with complex I defects. Each sample is reverse transcribed, labeled, and hybridized, together with standard target, to a human array comprising probes selected from Example 1. The hybridization measurements are analyzed, leading to the identification of several novel mtDNA mutations and dominant and recessive nDNA mutations.

### Example 4

Profile for Complex IV Leigh's Syndrome

The mitochondrial biology expression profile was determined for a complex IV Leigh's syndrome (LS) patient. LS is a subacute neurodegenerative condition characterized by necrotic lesions in the brain stem, basal ganglia, thalamus and spinal cord. Death is usually within 2 years of onset of symptoms that may include motor and/or intellectual retardation, abnormal breathing rhythm, nystagmus, opthalmoparesis, optic atrophy, ataxia, and dystonia. The Leigh's syndrome patient had a typical complex IV cytochrome c oxidase deficiency associated with surfeit 1 (SURF-1) gene mutations. This patient was from a consanguineous marriage and was homozygous for a nonsense mutation in the SURF-I gene. Expression profiling of muscle and cultured cell samples from this patient using a human array of Example 1 was performed, in comparison to a control reference standard. NDUFS8 expression was not significantly altered. However, many nuclear and mitochondrially encoded complex I genes were down-regulated, including mtDNA transcripts ND4, NDLA, and ND6. Nuclear genes SURF-1, SOD2, 70 kD heat shock protein, voltage dependent anion channel (VDAC4), adenine nucleotide translocase 2 (ANT2), and glutathione peroxidase 3 were down-regulated.

### Example 5

Profile for Complex I Leigh's Syndrome

Mitochondrial biology expression profiles were determined for twelve complex I Leigh's syndrome patients (Procaccio, VF (2001) EuroMit5 Abstract). Sequencing of all 43 genes known to be part of complex I, of each patient, identified one patient as a compound heterozygote for two missense mutations in the 23 kD NADH dehydrogenase (NDUFS8) gene of complex I. This patient had a respiratory complex I defect apparent in skeletal muscle and cultured lymphoblastoid cells. Samples were collected from cultured lymphoblastoid cells from this patient and control reference lymphoblastoid cells. Samples were reverse transcribed and differentially labeled and hybridized to a human array comprising probes selected from Example 1. The expression profile was determined using a hierarchical clustering method. Mitochondrial biology expression profiles from the other patients were similarly determined using appropriate samples and controls. Expression profiles of all patients were characteristic of complex I deficiencies, including down-regulation of all mtDNA and some nDNA complex I genes and up-regulation of the adenine nucleotide translocator genes (ANTI and ANT2).

### Example 6

Diagiosis of Complex IV Leigh's Syndrome

The mitochondrial biology expression profile for Leigh's syndrome SURF-1 nonsense mutations, as determined in Example 4, is used to diagnose patients. Samples are collected from patients and mitochondrial biology expression microarray-tested using a human array containing probes for at least SURF-1, ND4, NDL4, ND6, SOD2, 70 kD heat shock protein, VDAC4, ANT2, and glutathione peroxidase 3.

### Example 7

A Mouse MitoChip

A mouse Mitochip was printed with probes for 452 genes. Some of these genes were represented by two or more probes, providing internal controls for the reproducibility of gene expression quantitation. An additional 37 control spots were included on the array. Of these, 25 were probes for housekeeping genes to allow normalization between samples. The remaining 12 spots were various controls for hybridization and positioning. Table 2 lists the functional categories and number for all of the housekeeping genes on this array. The cDNA clones that represent each gene were either from the I.M.A.G.E. consortium or cloned by The Center for Molecular Medicine and published in (Murdock et al., 1999). A complete annotation of each gene was compiled and GenBank accession numbers and Unigene cluster numbers were determined. Table 5 provides a list of the probes on this array.

### Example 8

Profile of Sod2 Heterozygote Mutant Mice at Various Ages

Oxidative stress has been implicated in aging and degenerative disease. Mitochondria are thought to be the main source of reactive oxygen species such as superoxide anion. Mitochondrial superoxide anion is normally detoxified by manganese superoxide dismustase (MnSOD, the Sod2 gene). However, when, free radical metabolism is perturbed, oxidative damage to protein, DNA, and lipids may occur. To demonstrate the effects of increased superoxide anion toxicity on mitochondrial physiology with age, the mitochondrial biology expression profiles of mice with a 50% reduction in MnSOD (Sod2 +/−) were determined at various ages. Samples were collected from young (5 months), middle-aged (10-14 months), and old (20-25 months) wild-type and Sod2 +/− mice. Samples were reverse transcribed and differentially labeled from the corresponding controls. The labeled mutant sample and the corresponding labeled control were hybridized with the mouse array of Example 2. Relative to the control mice, the old Sod2 +/− mice showed induction of antioxidant and apoptosis genes including glutathione peroxidase 3, apoptosis inhibitory factor 3, caspase 1, and the peripheral benzodiazepine receptor.

### Example 9

Profile of Sod2 Homozygote Mutant Mice

Manganese superoxide dismutase (MnSOD, the Sod2 gene) is a gene expression product involved in mitochondrial biology. Sod2 −/− animals die soon after birth due to the superoxide inactivation of mitochondrial iron-sulfur center enzymes resulting in dilated cardiomyopathy. The mitochondrial biology expression profile of Sod2 −/− mice is determined using the mouse MitoChip of Example 2. RNA samples are collected from Sod2 −/− mice and Sod2 +/+ mice. The Sod2 −/− sample is reverse transcribed and labeled with Cy3 phosphoramidite. The Sod2 +/+ sample is reverse transcribed and labeled with Cy5 phosphoramidite. The labeled samples are incubated with a mouse array under conditions of high stringency hybridization. The hybridization of both samples is measured with a microarray reader. The hybridization measurements are recorded.

### Example 10

Profile of GP×I Mutant Mice

Glutathione peroxidase 1 (GP×1) is an expressed sequence involved in mitochondrial biology. GP×1 −/− animals show mild growth inhibition and reduced OXPHOS efficiency. The mitochondrial biology expression profile of GP×1 −/− mice is determined using a mouse array of Example 2. RNA samples are collected from GP×1 −/− mice and GP×1 +/+ mice. The GP×1 −/− sample is reverse transcribed and labeled with Cy3 phosphoramidite. The GP×1 +/+ sample is reverse transcribed and labeled with Cy5 phosphoramidite. The labeled samples are incubated with a mouse array under conditions of high stringency hybridization. The hybridization of both samples is measured with a microarray reader. The hybridization measurements are recorded.

### Example 11

Profile of Sod2 Heterozygote GP×1 Homozygote Doubly Mutant Mice

The mitochondrial biology expression profile of Sod2 −/+ plus GP×1 −/− mice is determined using a mouse array of Example 2. RNA samples are collected from Sod2 +plus GP×1 −/− mice and Sod2 +/+ plus GP×1 +/+ mice. The Sod2 −/+ plus GP×1 −/− sample is reverse transcribed and labeled with Cy3 phosphoramidite. The Sod2 +/+ plus GP×1 +/+ sample is reverse transcribed and labeled with CyS phosphoramidite. The labeled samples are incubated with a mouse array under conditions of high stringency hybridization. The hybridization of both samples is measured with a microarray reader. The hybridization measurements are recorded.

### Example 12

Profile of Mutant Mice Overexpressing Sod2 and/or GP×1

The mitochondrial biology expression profiles are determined using a mouse array, for mice overexpressing MnSOD and for mice overexpressing MnSOD plus GP×1.

### Example 13

Profile of ρ⁰ Mutant Cell Line

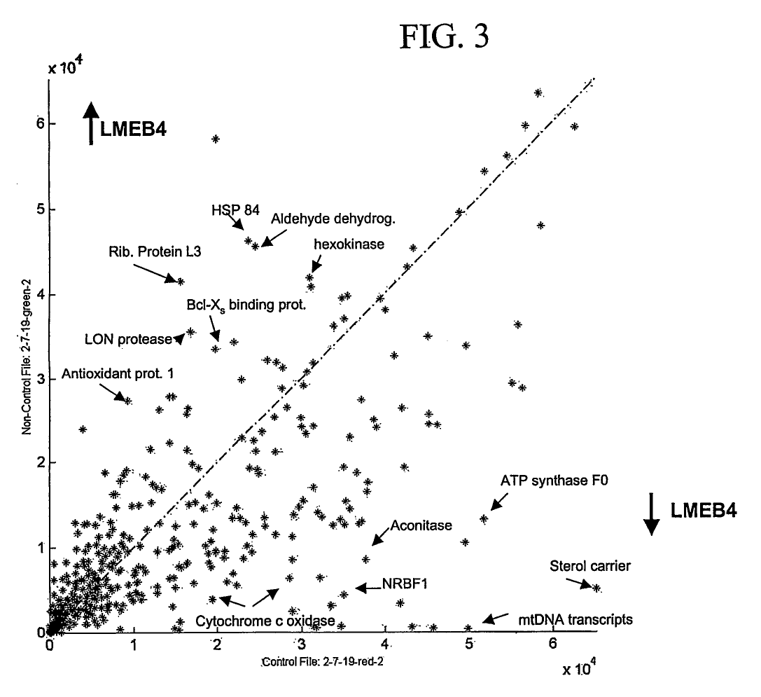

A mouse array of Example 2 was used to determine the mitochondrial biology expression profile of the mouse mutant cell line ρ⁰, the most extreme case of mitochondrial dysfunction. The LMEB4 (ρ⁰) cell line was profiled against its parental LM(TK)-cell line. The mouse mutant cell line ρ⁰ lacks mitochondrial DNA. To maintain the LMEB4 cell line in culture, it must be grown in media supplemented with glucose, pyruvate, and uridine (GUP media). A scatter plot of the gene expression ratios is shown in FIG. 3. Samples from the ρ⁰ cell line and from the LM(TK) cell line were reverse transcribed and differentially labeled using a standard two-color fluorescent system, and hybridized to a mouse array of Example 2. Mouse array analysis confirmed that all mtDNA-encoded transcripts were absent from the LMEB4 cells, and that there was a reduction in NDNA OXPHOS gene expression, aconitase, and nuclear receptor binding factor 1 (NRBF1). There was an increase in expression of key glycolytic genes, mitochondrial ribosomal proteins, the LON protease, heat shock protein 84 (HSP 84), Bcl-X binding protein, and antioxidant protein 1. Invariably, the nuclear-encoded OXPHOS complex subunits were also down-regulated between 3 and 38-fold with a mean of 4.5 (the mean was calculated excluding the single outlying complex I subunit NADH-dehydrogenase mwfe which was down-regulated 38-fold). Mitochondrial transport proteins such as the Glutamate-malate transporter were down-regulated as was the mitochondrial protein import subunit gene Tim17 and several amino acid metabolism genes. By contrast, glycolytic genes such as pyruvate kinase, glucose phosphate isomerase and glucose-6-phosphate dehydrogenase were up-regulated 2 to 3-fold. Phosphofructokinase was up 1.6-fold. Anti-apoptotic genes such as apoptosis inhibitor 2 and 3 were up-regulated as was the pro-apoptotic Bcl-Xs binding protein BNIP3 and Caspase 2. The other Bcl protein family members that are on the array were not changed significantly. The multi-function mitochondrial LON protease was up-regulated 2. 1-fold.

### Example 14

Profile of CAP^{R} Mutant Cell Line

A mouse array of Example 2 was used to determine the mitochondrial biology expression profile of the mouse mutant cell line harboring a mutation for chloramphenicol resistance (CAP^{R}), and the CAP^{R} 501-1 cell line having a mtDNA mutation in the 16S rRNA gene. The CAP^{R} mutation in chimeric mice causes cataracts, reduced photoreceptor response, vacuolization of the retinal pigment epithelium, and hamartomatous outgrowths of the optic nerve head. Mice inheriting the CAP^{R} mutation showed a marked increase in embryonic lethality, and those that were born died within two weeks with growth retardation, dilated cardiomyopathy, and mitochondrial abnormalities. CAP^{R} 501-1 was compared to the CAPS LM(TK)-cell line. These two cell lines are both derived from mouse L929 cells. Samples from the CAP^{R} cell line and from wild-type cells were reverse transcribed and differentially labeled with a standard two-color fluorescent system, and hybridized to a mouse array of Example 2. The CAP^{R} cell line had up-regulation of all thirteen mtDNA transcripts, but down-regulation of multiple nDNA OXPHOS genes. The CAP^{R} 501-1 cell line versus the LM(TK)-gene expression scatter plot showed that all mtDNA transcripts were up-regulated 3.1 to 3.5-fold while the nuclear encoded OXPHOS subunits were down-regulated 2.1 to 5.3-fold. Procollagen type III and VI were also up-regulated 3.5 to 4-fold.

### Example 15

Profile of Treatment to Cell Line

Mouse arrays of this invention were used to demonstrate how treatment changes, such as changing cell culture conditions, affect gene expression. The control cell line LM(TK)-grown in standard medium was profiled against a culture of LM(TK)-cells grown in media supplemented with glucose, pyruvate, and uridine (LM(TK)-(GUP)). Samples from the treated fibroblast cell line and from untreated fibroblast cells were reverse transcribed and differentially labeled with a standard two-color fluorescent system, and hybridized to a mouse array of Example 2. Treatment resulted in a down-regulation of the LON protease and HSP 84. The scatter plot of this experiment showed that other than the same core group of genes that were up-regulated in the NZB cell line mentioned in Example 17, few genes were significantly different in their expression. The hybridization spots of three genes that showed the highest differences were HSP70, the LON protease, and E.T.F. The 70 kDa heat shock protein (HSP70) was down-regulated 3.4-fold. HSP70 has been shown to be a chaperone protein involved in mitochondrial protein import that forms an ATP-dependent motor with the inner mitochondrial membrane translocase and the polypeptide in transit (Voos, W. et al., “Mechanisms of protein translocation into mitochondria,” [ 1999] Biochimica et Biophysica Acta 1422:235-54). The entire HSP70 control spot was of medium intensity, while the experimental spot was only medium intensity in the center. The LON protease was down-regulated 9.7 fold in LM(TK)-cells grown in GUP. The control LON protease spot was of medium high intensity over the entire spot and of low intensity in the experimental spot. The electron transfer flavoprotein (ETF), which shuttles electrons gathered during fatty acid metabolism to the electron transport chain, was down-regulated 3.8 fold. The E.T.F control spot was high intensity and the experimental spot very low intensity. Some of the nuclear encoded OXPHOS subunits as well as several proteins involved in amino acid metabolism were down-regulated 1.5 to 2-fold with mean ratio of 1.65. Since most of these genes fell below the +/−1.7 ratio cutoff, further analysis was needed to determine if the expression pattern was significant. There were no differences in mtDNA transcript levels and no consistent pattern of up-regulation of glycolytic genes.

### Example 16

Profile of Sod2 Mutant Mice After Treatment and Before Symptoms

Treatment of Sod2 mutant mice with MnTBAP prevents cardiac and liver pathology, however after 12 days the MnTBAP-treated mutant animals develop a prominent movement disorder which leads to debilitation by three weeks, in association with spongiform changes and gliosis in the cortex and specific brain stem nuclei associated with motor function. It is thought that the severe neuropathology results from poor exchange of MnTBAP across the blood brain barrier. The mitochondrial biology expression profile of MnTBAP-untreated, Sod2 mutant mice and MnTBAP-treated, Sod2 mutant mice was determined using the mouse array of Example 2. Samples were collected from 8 day old Sod2 mice without MnTBAP treatment, 8 day old Sod2 mice with MnTBAP treatment, and 12 day old Sod2 mice with MnTBAP treatment. Samples were also collected from age-matched controls. About 20 genes were found to be differentially expressed in all three groups of Sod2 knockout mice compared to the corresponding age-matched controls. The about 20 genes included bioenergetic genes such as the mitochondrial creating phosphokinase, antioxidant enzymes like the glutathione peroxidase 3, and apoptotic factors including caspase I and apoptosis inhibitor factor 3. The excitatory amino acid transporter 3, fiataxin, and one EST of unknown function were also induced. Mitochondrial biology expression profiling demonstrated changes in expression before neuropathic changes were manifested.

### Example 17

Organ-Specific Profiles of Mutant Mice

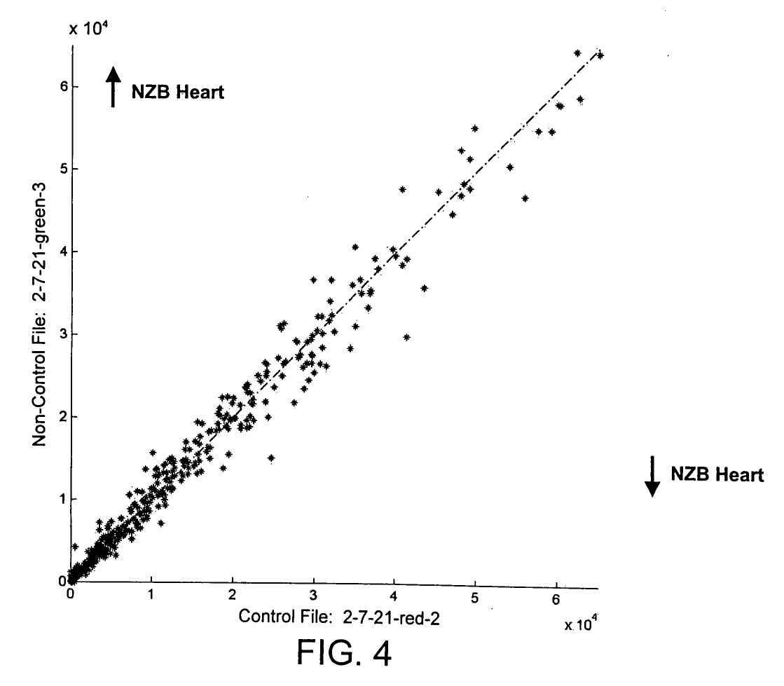

The NZB mouse line mtDNA and the “common haplotype” mtDNAs (129/Sv, C57B1/6J, C3H, BALB/c, and others which are thought to have arisen as the progeny of a single female (Ferris et al.,1982) differ by 108 nucleotides, and these polymorphic differences have been used to monitor the segregation of heteroplasmic populations of mtDNAs in mice created by embryo fusion techniques (Jenuth, J. P. et al., “Random genetic drift in the female germline explains the rapid segregation of mammalian mitochondrial DNA,” [1996] Nat Genet 14:146-51; Jenuth, J. P. et al., “Tissue-specific selection for different mtDNA genotypes in heteroplasmic mice,” (1997) Nat Genet 16:93-5; Meirelles, F. V., and Smith, L. C., “Mitochondrial genotype segregation in a mouse heteroplasmic lineage produced by embryonic karyoplast transplantation,” (1997) Genetics 145:445-51; Meirelles, F. V. and Smith, L. C., “Mitochondrial genotype segregation during preimplantation development in mouse heteroplasmic embryos,” [1998] Genetics 148:877-83). Tissues from the NZB and CAP^{R} mice were profiled on a mouse array. Messenger RNA was isolated from the brain, liver, spleen, kidney, heart, and skeletal muscle of a male mouse heteroplasmic for the NZB mtDNA and a male mouse that was 80% chimeric for ES cell-derived CAP^{R} cells as defined by coat color. Due to the severity of the CAP^{R} mutation it was not possible to analyze the mitochondrial gene expression changes in mice that were homoplasmic for the CAP^{R} mtDNA. Control mRNA for each of the tissue samples was isolated from sex, age, and nuclear background-matched control mice. All of the tissue samples were genotyped to determine the levels of heteroplasmy for the NZB and CAP^{R} mtDNA in each of the tissues. Equal levels of the NZB and “common” mtDNA were found in the six tissues analyzed from the NZB mtDNA-positive mice. The six tissues from the CAP^{R} chimera had varying levels of CAP^{R} mtDNA with the kidney and spleen having the highest amounts, 65% and 50% CAP^{R} mtDNA, respectively. The heart contained approximately 20% CAP^{R} mtDNA, while brain, liver, and muscle all contained between 5% and 10% CAP^{R} mtDNA. Analysis of the NZB-mtDNA tissue samples did not reveal any differentially expressed genes in the heart, liver, brain, and kidney. A scatter plot from the NZB heart is shown in FIG. 4. The scatter plots from the liver, brain, and kidney are virtually identical in that nearly every gene has an expression ratio of 1. Analysis of the NZB-mtDNA spleen and muscle showed several genes that were differentially expressed in the two tissues. The NZB-mtDNA muscle showed a 1.5 to 2.1-fold reduction in all mtDNA transcripts, pyruvate dehydrogenase was down 2.2-fold, and there was a general trend for nuclear-encoded OXPHOS subunits to be down-regulated 1.4 to 1.8-fold. The vesicular transport protein, pantophysin, was down-regulated 4-fold and the glycogenolysis rate-limiting enzyme, glycogen phosphorylase, was down 3-fold. There were not any genes that were significantly up-regulated in the muscle. A similar pattern of mtDNA-encoded gene expression was observed in the NZB-mtDNA spleen with all transcripts down 1.8 to 2-fold. However, there were no differences in nuclear OXPHOS subunit expression levels like that observed in the NZB-mtDNA skeletal muscle. In contrast to the NZB-mtDNA muscle, several genes were up-regulated in the spleen. In direct opposition to the results in the NZB cell line, both probes of the heme biosynthesis gene coproporphyrinogen oxidase III derected up-regulation 3-fold in the spleen. The integral membrane protein SURF 4 was up 2-fold and the amino acid metabolism gene 2-amino-3-ketobutyrate CoA ligase was up 4.8-fold. Glycogen phosphorylase, down 3-fold in the muscle, was up 4.8 fold in the spleen. The muscle and spleen results suggest that the polymorphisms between the NZB and “common” mtDNA may have a functional consequence in some tissues but not others. Analysis of the CAP^{R} tissue samples did not show any genes to be differentially expressed in the kidney, heart, muscle, liver, or spleen. The kidney, having the highest percentage of mutant mtDNAs, had expression ratios around 1 for nearly every gene. The two outliers on the kidney scatter plot that appear to be down-regulated can be explained by hybridization artifacts causing a high background in the control sample. The CAP^{R} brain sample was the only tissue that had any differentially expressed genes. Skd 3 was up-regulated 2.2-fold, glutathione peroxidase was up 2.4-fold and apoptosis-inhibitor 3 was up 2.4-fold. Although no genes were down-regulated in the brain more than 1.8-fold, closer analysis of the brain samples did reveal a trend that was not observed in any of the other tissues. Several nuclear-encoded OXPHOS subunits were down-regulated between 1.3 and 1.6-fold. These included five Complex I subunits, three Complex IV subunits and five Complex V subunits as well as VDAC 1 and 3. None of the Complex II and III subunits or mtDNA transcripts followed this trend. Principal component analysis of NZB and CAP^{R} mouse tissues, separately and together with the cell lines, was performed.

### Example 18

Identification of Genes for Mitochondrial Arrays

Mice mutant in mitochondrial biology were used to identify genes involved in mitochondrial biology. Mice deficient in the heart/muscle isoform of the adenine nucleotide translocator (ANTI) exhibit many hallmarks of human oxidative phosphorylation (OXPHOS) disease, including dramatic proliferation of skeletal mitochondria. Samples were collected from the gastrocnemius muscle of ANTI and wild-type mice, reverse transcribed and differentially labeled, and hybridized with a mouse microarray chip (Mouse Unigene 1, Incyte Genomics Inc., Palo Alto, California) containing over 8000 sequence-verified cDNAs. Analysis of the hybridization results identified more than 150 differentially expressed genes. Gene sequences that were not previously recognized as being involved in mitochondrial biology were used to generate probes that were placed on the mouse array of Example 2. Homologous human gene sequences were used to generate probes that were placed on the human array of Example 1.

### Example 19

Profile of Age-Related Changes in Chimpanzee Using Human Mitochondrial Array

Age-related changes in the mitochondrial biology expression profile in chimpanzees are determined using a human array of Example 1. Samples from young adult chimpanzee muscle and samples from most-mortem tissues of older chimps are reverse-transcribed, differentially labeled, and hybridized with a human array of Example 1.

### Example 20

Profile of Putative Neutral Variant Mutant Mouse

The NZB cell line was profiled to examine the changes in mitochondrial gene expression resulting from a more neutral set of mtDNA polymorphisms. As mentioned previously, the NZB mtDNA contains 108 sequence differences compared to the “common” mouse mtDNA genotype found in LM(TK). While these differences were reported to be neutrally polymorphic (Jenuth et al., [1996] Nature Genetics 14:146-151; Meirelles and Smith [1997] Genetics 145:445451), the only evidence to support that hypothesis is that transgenic mice containing a high percentage of NZB mitochondria have no overt phenotypes (Levy, S. E., “Genetic Alteration of the Mouse Mitochondrial Genome and Effects on Gene Expression,” (2000) Ph.D. Thesis, Emory University; Jenuth et al. [1997] Nature Genetics 16:93-95; Meirelles and Smith [1998] Genetics 148:877-883). An NZB cybrid cell line was profiled on a mouse mitochondrial array. The scatter plot of gene expression ratios between the NZB cell line and the parental LM(TK)-(without GUP supplementation) shows that both probes of the fatty acid metabolism gene Acyl-CoA dehydrogenase (medium-chain) detected up-regulation 3.6-fold. Procollagen III and VI were up-regulated 6.2 and 6.8-fold, respectively. Two independent probes of the coproporphyrinogen oxidase III gene that is involved in heme biosynthesis detected down regulation 2.6 and 2.3-fold. Also down-regulated was the peripheral-type benzodiazepine receptor. This receptor has been implicated in a variety of mitochondrial functions including the regulation of mitochondrial protein import under conditions of oxidative stress, calcium homeostasis, and steroidogenesis (Culty, M. et al., “In vitro studies on the role of the peripheral-type benzodiazepine receptor in steroidogenesis,” [1999] J. Steroid Biochemistry & Molecular Biology 69:123-30; Wright, G., and Reichenbecher, V. “The effects of superoxide and the peripheral benzodiazepine receptor ligands on the mitochondrial processing of manganese-dependent superoxide dismutase,” [1999] Experimental Cell Research 246:443-50). The glycolytic genes glyceraldehyde-3-phosphate dehydrogenase and glucose-6-phosphate isomerase were up-regulated 1.7 and 2.1-fold, respectively. Glycolytic genes were also up-regulated in the NZB cell line. This indicates that the sequence polymorphisms between the NZB and “common” mtDNAs are not entirely neutral and cause changes in mitochondrial function when combined with the LM(TK)-nucleus. Thus, the NZB mtDNA does not appear to be completely interchangeable with the “common” mtDNA genome. An interesting group of genes that were up-regulated in the NZB cell line were the pro-inflammatory genes Caspase 1 and platelet activating factor (PAF) acetylhydrolase, the mitochondrial RNA polymerase, and glutathione peroxidase 3.

### Example 21

Hierarchical Clustering of Cell Lines

Principal component analysis (PCA) and hierarchical clustering were performed on the cell line data (Examples 13-15 and 20) to group genes based on similarities in their expression patterns over all the samples. PCA analysis was used to reduce the dimensionality of the data by calculating three principal axes that encompass as much of the variability in all of the samples as possible. Each of the samples was then plotted on those axes in three-dimensional space. The PCA results revealed that the NZB cell line clustered away from the other cell lines, consistent with it having fewer differentially expressed genes in common with the other samples. The LMEB4 ρ0, 501-1 and LM(TK)-(GUP) cell lines all arrayed along one common principle axis, probably due to the commonality of a down-regulation of nuclear OXPHOS genes. However, they were divergent in the other two axes. The LM(TK)-(GUP) and NZB did share one axis, possibly due to a partial reduction in OXPHOS genes and a concomitant induction of glycolytic gene expression. However, both showed few differences when compared to the CAP R 501-1 and LMEB4 ρ0 samples. A hierarchical clustering algorithm was used to group genes with similar expression profiles across all of the samples. Both genes as well as samples were clustered together using a Euclidean distance measurement and average linkage. The clustering results revealed seven groups of genes with similar expression patterns in the cell line samples. Certain classes of genes were found to change together. Similar to the PCA analysis, the expression changes seen in the LM(TK)-(GUP) and NZB samples clustered closest together with the CAP R 501-1 and LMEB4 ρ0 samples branching successively further away. The Group 1 genes are involved in fatty acid metabolism. Group 2 genes, mainly down-regulated in the LM(TK)-(GUP) sample, include malate dehydrogenase, lactate dehydrogenase, glucose phosphate isomerase, and several amino acid metabolism genes. Group 3 genes are diverse clusters of genes that change in expression coordinately across the 5 samples. It includes some nuclear-encoded OXPHOS subunits, a few antioxidant and transport proteins as well as pyruvate kinase and a GTP-binding protein. Group 4 is a small, diverse cluster of genes mainly up-regulated in the CAP R 501-1 cell line. This group includes several of the same genes found to be up-regulated in the Ant1(−/−) mouse by differential display analysis (Murdock et al., 1999). Also in this group are two NADP-transhydrogenases, carbonate dehydratase and cytochrome b5 reductase. Group 5 is almost exclusively nuclear-encoded OXPHOS subunits. The voltage-dependent anion channel (VDAC) genes and several antioxidant proteins also cluster in this group. Group 6 is composed almost entirely of mtDNA-encoded transcripts. Group 7 is the heterogeneous group of genes that were up-regulated in each of the samples analyzed and included Caspase 1, PAF acetylhydrolase, the mitochondrial RNA polymerase, and glutathione peroxidase 3. Hierarchical clustering packages are available in the art, i.e. Expression Profiler (http://ep.ebi.ac.uk/EP/ from the European Bioinformatics Institute, Cambridge, UK). PCA is described in Bioinformatics 2001, volume 17, number 9, pages 763-774.

The following examples describe the preparation of a mitochondrial biology expression array, sample preparation, hybridization, scanning, and data normalization.

### Example 22

PCR Amplification

PCR amplifications were performed with standard PCR techniques. Probes were made my amplifying clones using a universal primer set (Forward primer 5′-CTGCAAGGCG ATTAAGTTGGGTAAC-3′ Reverse primer 5′-GTGAGCGGATAACAATAATCAC ACAGGAAACAGC-3′) in a 100 μl PCR reaction containing PCR buffer (10 mM Tris, 1.5 mM MgC12, 50 mM KCl, pH8.3), 0.2 mM dNTPs, 0.2 mM each primer, 1.25 U Taq (Sigma, St Louis, Mo.). 0.5-1 μl of bacterial culture was added to each PCR reaction and thermal cycling was done as follows: 4 minutes at 94 C followed by 35 cycles of 15 seconds at 94 C, 30 seconds at 66 C and 1 minute 30 seconds at 72 C. Following cycling, reactions were held at 72 C for 4 minutes to complete all extension reactions. All PCR products were confirmed by agarose gel electrophoresis through a 1.5% gel. After satisfactory amplification, products were quantitated by UV 260/280 ratio and desiccated in a Savant Speed-Vac (Holbrook, N.Y.). Dried products were then resuspended in 3×SSC (450 mM NaCl, 40 mM sodium citrate) at a concentration of 400-600 ng/μl for arraying.

### Example 23

Slide Preparation

Before arraying probes, the glass microscope slides for the arrays were coated with poly-Lysine to provide a substrate for DNA binding. Standard glass microscope slides (Gold Seal, Beckton-Dickson, Franklin Lakes, N.J.) were cleaned in a solution of 2.5 M NaOH, 60% ethanol for two hours. After cleaning, slides were rinsed five times in fresh water. The slides were then soaked in a solution of 0.01 % poly-L-lysine, .01× PBS for 1 hour followed by rinsing in fresh water. After rinsing, the slides were dried in a vacuum oven at 45° C. for 15 minutes.

### Example 24

Printing

Arrays were printed onto poly-L-lysine coated glass slides using the GMS 417 Arrayer (Affymetrix/Genetic Microsystems, Woburn, Mass.). The arrays were printed using a 4-pin print head with a spot size of 150 μm (approximately 33 μpL of volume per spot) and a center-to-center spot spacing of 375 μm. A humidity level of 65-70% was maintained during the printing of the arrays by a custom humidifier system. After printing, the arrays were allowed to dry for 1 hour at room temperature. The arrays were then processed by rehydrating over a warm solution of 1×SSC for 5 minutes followed by rapid drying on a 95° C. heat block. Following drying, the DNA was crosslinked to the slide by exposing the arrays to 65 mJ of ultraviolet energy (Stratalinker, Stratagene, La Jolla, Calif.). To block non-specific interactions on the arrays during hybridization, the slides were then treated with a solution of 60 mM succinic anhydride and 40 mM sodium borate in 1-methyl-2-pyrrolidinone for 15 minutes at room temperature. The arrays were then denatured in 95° C. water for 2 minutes and dehydrated by rapid immersion in 95% ethanol. The arrays were then dried by centrifugation at 20 ×g for 5 minutes.

### Example 25

Sample Preparation

Total RNA preparations were performed using the TRizol reagent (Life Technologies, Gaithersburg, Md.) as per the manufacture's directions. For cell culture samples, a 90% confluent 225ml flask was lysed directly in the flask with 18 ml of TRIzol. At least three flasks were pooled for each cell line to reduce any variability caused by culture conditions. For each mouse tissue, RNA was isolated from approximately 500 mg of tissue that was mechanically homogenized. in 6ml of TRizol. Following the isolation of total RNA, poly-A+ MRNA was isolated using Qiagen Oligotex (Valencia, Calif.) as per the manufacture's directions.

### Example 26

Reverse Translation Labeling, and Hybridization

To produce targets for hybridization to the MitoChip arrays, 2 μg of poly-A+ RNA was labeled with fluorescent nucleotides by reverse transcription. The poly-A+ RNA was mixed with 3 mg of anchored oligo-dT and incubated at 70° C. for 10 minutes followed by 10 minutes on ice. The denatured and annealed RNA was then reverse transcribed in a 30 μl reaction mix containing reaction buffer (50 mM Tris-HCl, 75 mM KCl, 3 mM MgC12 pH 8.3), 10 mM dithio-threatol, 500 μM dATP,dGTP,dTTP, 300 μM dCTP, 20 U SuperScript reverse transcriptase (Life Technologies, Gaithersburg, Md.) and 100 μM of either Cy5-dCTP (control samples) or Cy3-dCTP (experimental samples). The reactions were incubated at 42° C. for 2 hours. Following incubation, 15 μl of 0.1 M NaOH was added to degrade the remaining template RNA and the sample incubated at 70° C. for 10 minutes. The reaction was neutralized by the addition of 15 μl of 0.1 M HCl followed by 440 μl of TE buffer (10 mM Tris, 1 mM EDTA, pH 7.4). The synthesized cDNA was purified by size-exclusion filtration using Microcon YM-3 centrifugal filter devices (Millipore, Bedford, Mass.). After purification, 10 μg of poly-A RNA (Sigma, St Louis, Mo.) and 10 μg of yeast transfer RNA (tRNA) (Life Technologies, Gaithersburg, Md.) was added. The final sample volume was adjusted to 12 μl and 525 mM NaCl, 52.5 mM sodium citrate, 0.25% SDS. The sample was denatured at 100° C. for 2 minutes and added to the array. The sample and the array were hybridized under high stringency hybridization conditions. The sample and array were covered by a 22 mm×22 mm coverslip and placed in a humidified hybridization chamber (Corning, Acton, Mass.) and incubated at 65° C. for 12-16 hours. Following hybridization, the arrays were washed with successive 5-minute washes in 2×SSC, 0.1%SDS; 1×SSC; and 0.1×SSC. After the final wash, the arrays were dried by centrifugation at 20×g and scanned using the GMS 418 Array Scanner (Affymetrix/Genetic Microsystems, Woburn, Mass.).

### Example 27

Array Scanning and Data Analyses

Scanned arrays were saved as 16-bit TIFF files and analyzed using Biodiscovery's Imagene software (Los Angeles, Calif.). Data mining and clustering analysis was performed using Biodiscovery's GeneSight software. Prior to data analysis, all cell culture samples were normalized using all spots on the array. All mouse tissue samples were normalized to the housekeeping genes on the mouse array. Local background was calculated for each individual spot and any spot with a signal intensity less than 3 times over background or that had poor morphology was excluded from the data analyses. Only differential expression values of greater than 1.7 were considered significant. All data mining and clustering analysis performed using GeneSight was on expression ratio data that was transformed by taking the natural log (ln) of all values and normalized by Z-score. The data is transformed because of the non-Gaussian distribution of the expression ratio values. Because the ratios are bounded on the lower limit by zero, a non-Gaussian distribution is normally observed. To allow for additional statistical manipulations, the data was transformed for a more uniform distribution. The Z-score normalization method involved subtracting the mean from every observation and dividing by its standard deviation, effectively normalizing each spot to all other spots on the array.

### Example 28

Sample Hybridization to Mitochondrial Array

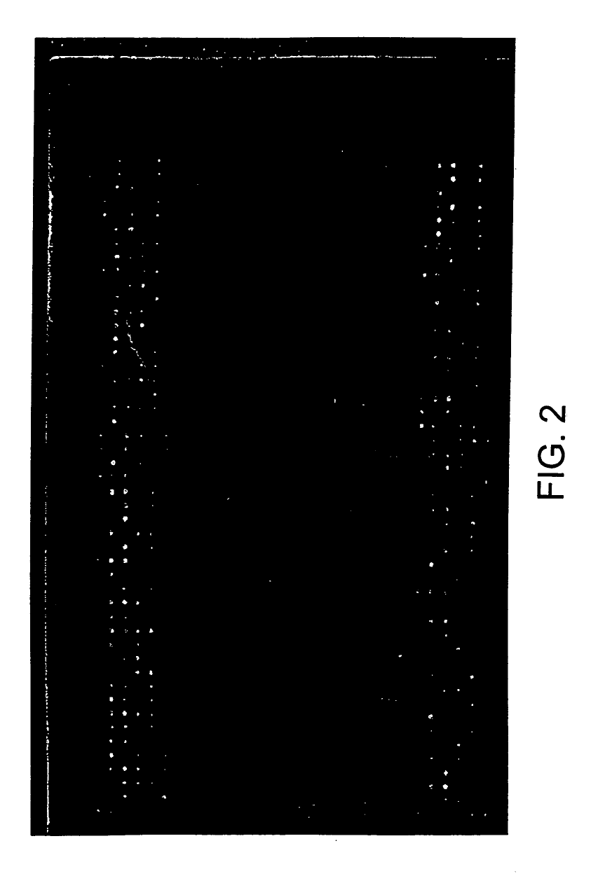

Control cDNA samples were prepared from mRNA isolated from the LM(TK)-cell line and labeled with the CyS dye. Each experimental MRNA sample was labeled with the Cy3 dye, combined with the Cy5 control sample and the mixture used to hybridize the array. A representative image of a hybridized array is shown in FIG. 2. Any spot on an array that appeared red was due to hybridization of a large proportion of the CyS-labeled control LM(TK)-sample and any sample that was green was due to the hybridization of a large proportion of the Cy3-labeled experimental sample. Any spot that is yellow is an about equal co-hybridization of the two targets. The fluorescence ratio was quantitated for each spot, permitting calculation of the relative abundance of each gene's MRNA in the two samples.

### Example 29

Normalization

The two fluorescent dyes that were used to label the cDNA produced during the reverse transcription of the MRNA have different structures and different emission maxima. Therefore, the two images that represent the hybridization of each of the fluorescently labeled samples were normalized to each other to account for the differences in dye behavior prior to calculating the expression ratios between the two images. One image was normalized to the other by averaging all of the spots in each image to derive a constant that was then applied to each spot. Alternatively, a predetermined set of genes that were expressed equally in the two samples under all conditions could have been used. The expression ratios of these genes were used to calculate a constant that was then applied to all spots on the array. A set of 25 housekeeping genes in Table 2 was included on a mouse array for normalization and both of these methods were used in the analysis of the mouse cell line and tissue samples. Housekeeping gene expression in the cultured cells was much more variable than in the tissue samples. Because of the variability in the housekeeping gene expression patterns in the cell line samples, normalization was done using all of the spots on the array. The expression of the housekeeping genes was much more consistent in the tissue samples and normalization using either the housekeeping genes or the average of all of the genes gave similar results.

### Example 30

Clones useful for making control probes for the arrays of this invention are listed in

Table 6. Sequences of the genes useful for making the control probes are provided in the sequence listings hereof.

TABLE 6

| SEQ | | | GenBank |
|---|---|---|---|
| ID NO. | Gene Name | Complete Gene Name | Accession No. |
| 3041 | Beta Actin | Human beta actin | X63432 |
| 3042 | Plant control CAB | Arabidopsis photosystem | X56062 |
| | | I chlorophyll a/b | |
| | | binding protein | |
| 3043 | Plant control rbcL | Arabidopsis ribulose-1,5- | U91966 |
| | | bisphosphate | |
| | | carboxylase/oxygenase | |
| | | large subunit | |
| 3044 | Plant control | Arabidopsis RUBISCO | X14212 |
| | RUBISCO | activase | |

It will be appreciated by those of ordinary skill in the art that samples, sample collection techniques, sample preparation techniques, probes, probe generation techniques, genes involved in mitochondrial biology, hybridization techniques, array printing techniques, physiological conditions, cell lines, mutant strains, organisms, tissues, solid substrates, and methods of data analyses other than those specifically disclosed herein are available in the art and can be employed in the practice of this invention. All art-known functional equivalents are intended to be encompassed within the scope of this invention.

### REFERENCE TO SEQUENCE LISTINGS

Tables 3-5 list sequence information on the clones that are useful for making probes for practicing the methods of this invention. Clone identification numbers are usually from NIA (National Institutes of Aging, National Institutes of Health, Bethesda, Md., USA), ResGen Invitrogen (Carlsband, Calif., USA) or IMAGE Consortium, LLNL (Livermore, Calif., USA). Gene names and descriptions are provided for the gene interrogated by a probe made from the corresponding clone. GenBank Accession Number and Unigene Cluster ID are provided where available. The functions of certain genes are included in Table 4. Sequences of the 5′ and 3′ ends of the clones listed in Tables 34 are provided when available. If no 5′ or 3′ sequence was available, gene sequence from the GenBank Accession No. provided for that clone is listed in some cases. The GenBank sequence may be larger than the sequence of the clone. The instant invention may be practiced without the sequence information provided herein using the clones or GenBank listings. Other sequences derived from the genes interrogated by probes generated from clones listed in Tables 3-5 are useful for making equivalent probes using information known in the art, i.e., unique segments of such genes may be used.

The sequence listings that correspond to the clones listed in Table 3, covering human probes SEQ ID NOS: 1-994, contain information including: the sequence identification number; a GenBank Accession No. corresponding to the listed sequence or the gene interrogated by the probe containing the listed sequence; another GenBank Accession No. in parentheses which is associated with the listed sequence in Table 3; a Research Genetics (ResGen Invitrogen, Carlsbad, Calif., USA) Clone ID No. identifying the clone from which the sequence was derived; the name of the gene from which the clone was derived; a description of the gene; the Unigene Cluster ID No. of the gene; the IMAGE Clone ID No., which is often the same as the ResGen Clone ID No., and information in parentheses identifying the sequence as 5′ or 3′ of the clone; the length of the insert of the clone; the source of the clone; the type of clone, such as cDNA; and the nucleic acid sequence.

Sequence listings for control probes are provided as SEQ ID NOS:3041-3044.

The sequence listings that correspond to the clones listed in Table 4, covering mouse probes SEQ ID NOS:995-3040, contain information including: the sequence identification number; a GenBank Accession No. corresponding to the listed sequence; the 5′ and/or 3′ sequence of the corresponding clone, or the gene from which the corresponding clone was derived; the name and description of the gene from which the corresponding clone was derived; the Unigene Cluster ID No. of the gene from which the corresponding clone was derived; the name of the clone from which the instant sequence was derived; additional description of the gene; a set of titles usually including Clone Name, Rearray Sequence, Parent Sequence, Other EST, and Blast Link; a list of names including, in order of the above-mentioned titles, the name of the clone from which the sequence was derived, the name of the sequence with a suffix identifying it as the 5′ (−5) or 3′ (−3) sequence of the clone, the name of the parent sequence, and the name of another EST (expressed sequence tag), if it exists, which would be the other of the 3′ or 5′ sequence; the length of the sequence provided; and the nucleic acid sequence.

We claim:

1. An array comprising at least two isolated nucleotide molecules, each molecule having a sequence capable of uniquely hybridizing to a nucleic acid molecule which is an expression product of a gene involved in mitochondrial biology.

2. An array comprising two or more isolated nucleic acid molecules or spots, each spot comprising a plurality of isolated nucleic acid molecules, each molecule having a sequence consisting essentially of a sequence selected from the group consisting of the sequences of human probe set #1, SEQ ID NOS: 1 to 994, or mouse probe set #2, SEQ ID NOS: 995 to 3040, and sequences having at least 70% homology to the foregoing sequences.

3. The array of claim 2 printed on a glass slide.

4. The array of claim 2 comprising more than about ten spots.

5. The array of claim 2 comprising more than about twenty-five spots.

6. The array of claim 2 comprising all of the isolated nucleic acid molecules having the sequences of human probe set #1, SEQ ID NOS: 1 to 994.

7. The array of claim 2 comprising all of the isolated nucleic acid molecules having the sequences of mouse probe set #2, SEQ ID NOS: 995 to 3040.

8. The array of claim 2 also comprising one or more spots comprising control nucleic acid molecules, SEQ ID NOS:3041-3044.

9. A method for determining an expression profile of a sample containing nucleic acid comprising:
a) providing the sample;
b) providing an array of claim 2;
c) contacting said array with said sample under conditions allowing selective hybridization; and
d) measuring hybridization of nucleic acid in said sample to said array to produce an expression profile.

10. The method of claim 9 wherein said sample is from a mouse or a human.

11. A method for determining an expression profile of a first labeled sample containing nucleic acid relative to a second, differently labled sample containing nucleic acid comprising:
a) providing the first labeled sample;
b) providing the second, differently labeled sample;
c) providing an array of claim 2;
d) contacting the array with the first sample and the second sample under conditions allowing selective hybridization;
e) measuring hybridization of said first and said second samples to said array; and
f) comparing the hybridization of said first sample to the hybridization of said second sample to produce an expression profile.

12. The method of claim 11 wherein said second sample is a reference or a standard.

13. A method for determining an expression profile diagnostic of an energy-metabolism-related physiological condition comprising:
a) providing a labeled first sample from a first group of one or more individuals with said physiological condition;
b) providing a differently labeled second sample from a second group of one or more individuals without said physiological condition;
c) providing an array of claim 2;
d) contacting the array with the first sample and the said second sample under conditions allowing selective hybridization;
e) measuring hybridization of said first and said second samples to said array; and
f) comparing the hybridization of said first sample to the hybridization of said second sample to produce an expression profile diagnostic of said physiological condition.

14. A method of making an array comprising:
a) providing a prepared substrate; and
b) printing two or more spots in known positions on said substrate, each spot comprising a plurality of isolated nucleic acid molecules, each molecule having a sequence consisting essentially of a sequence selected from the group consisting of human probe set #1, SEQ ID NOS: 1 to 994, mouse probe set #2, SEQ ID NOS: 995 to 3040, and sequences having at least 70% homology to the foregoing sequences.

15. The method of claim 14 wherein said array comprises all of said isolated nucleic acid molecules in human probe set #1, SEQ ID NOS: 1 to 994.

16. The method of claim 14 wherein said array comprises all of said isolated nucleic acid molecules in mouse probe set #2, SEQ ID NOS: 995 to 3040.

17. A method of diagnosing a first individual with Complex IV Leigh's Syndrome comprising detecting in a first sample from said first individual at least about a 1.7-fold decrease in the amount of expression of genes comprising ND4, NDL4, ND6, SURF-1, SOD2, 70 kD heat shock protein, VDAC4, ANT2, and glutathione peroxidase 3 compared to the amount of expression of said genes in a second sample from a second individual without Complex IV Leigh's Syndrome.

18. A library of at least two isolated nucleic acid molecules, each molecule having a sequence consisting essentially of a sequence selected from the group consisting of human probe set #1, SEQ ID NOS: 1 to 994, mouse probe set #2, SEQ ID NOS: 995 to 3040, and sequences having at least 70% homology to the foregoing sequences.

19. An array comprising at least two spots, each spot comprising a plurality of isolated nucleic acid molecules, each molecule comprising a sequence with at least 70% homology to a sequence selected from the group consisting of human probe set #1, SEQ ID NOS: 1 to 994.

20. An array comprising at least two spots, each spot comprising a plurality of isolated nucleic acid molecules, each molecule comprising a sequence with at least 70% homology to a sequence selected from the group consisting of mouse probe set #2, SEQ ID NOS: 995 to 3040.
